(19) United States
(12) Patent Application Publication
(10) Pub. No.: US 2022/0291182 A1
(43) Pub. Date: Sep. 15, 2022

(54) UNIVERSAL ODOR CODE SYSTEMS AND ODOR ENCODING DEVICES

(71) Applicant: KONIKU INC., San Rafael, CA (US)

(72) Inventors: F. Kennedy MCDANIEL, San Rafael, CA (US); Marius GUERARD, San Rafael, CA (US); Oshiorenoya E. AGABI, San Rafael, CA (US)

(21) Appl. No.: 17/271,557

(22) PCT Filed: Apr. 10, 2019
(86) PCT No.: PCT/US19/26859

§ 371 Date: Feb. 25, 2021

Related U.S. Application Data
(60) Provisional application No. 62/655,682, filed on Apr. 10, 2018.

(30) Foreign Application Priority Data
Feb. 23, 2019 (US) PCT/US2019/023787

(51) Int. Cl. G01N 33/00; G06N 3/06; G01N 21/64; G01N 27/02; G01N 33/50; C12N 5/0793

(52) U.S. Cl. CPC G01N 33/0001; G06N 3/061; G01N 21/6428; C12N 2510/00; G01N 33/5058; C12N 5/0619; G01N 2021/6439; G01N 27/02

(57) ABSTRACT

A universal odor code system encodes an olfactory stimulus into olfactory receptor space as quantitative measures of responses by olfactory receptors, producing an odor code profile. A mapping function maps an odor code profile into a formula of elements that approximates or recreates an odor code profile of a target olfactory stimulus.

### CROSS-REFERENCE TO RELATED APPLICATIONS

This application claims the benefit of the priority date of U.S. provisional application 62/655,682, filed Apr. 10, 2018, and International Application PCT/US2019/023787, filed Mar. 23, 2019, both of which are incorporated herein by reference in their entirety.

### BACKGROUND

Currently, the only way to characterize a smell or taste, is by the language, which is a hard exercise. People without training can usually able to identify common odors only about half the time, without even describing the emotions they feel while smelling/tasting. There can be large vocabulary variations between the different domains involving smell or taste (e.g., food, perfumery, cosmetic, flowers, and wine) and large variations from the translations of these words between the different languages.

### SUMMARY

Recognized herein is a need for a universal odor code system for encoding, evaluating, and comparing odor information. There is also a need for assessing or predicting the emotions people are feeling when tasting or smelling.

A universal odor code system and an odor encoding device for encoding, evaluating, and comparing odor information, and a method for assessing or predicting emotions that people are feeling when tasting or smelling are described.

Disclosed here is a method for encoding an olfactory stimulus, comprising: a) contacting the olfactory stimulus with an artificial array comprising one or more chambers, wherein the one or more chambers comprise one or more cells expressing one or more cell-surface receptors, wherein the one or more cell-surface receptors generate one or more signals in response to a binding event between the olfactory stimulus and the one or more cell-surface receptors; b) recording an intensity of one or more signals of one of the cells; and c) encoding the olfactory stimulus by creating an reference signal, wherein the reference signal comprises the intensity of the one or more signals.

In some cases, the one or more cells are neurons. In some cases, the neurons are human neurons. In some cases, the one or more cells are modified to express the one or more cell-surface receptors. For example, the one or more cells are modified by introducing mRNAs that encode the one or more cell-surface receptors into the one or more cells. In some cases, the one or more cells are genetically modified to express the one or more cell-surface receptors. For example, the one or more cells are genetically modified by using CRISPR gene editing methods. In some cases, at least one of the one or more cell-surface receptors is an odorant receptor. In some cases, at least one of the one or more cells expresses one odorant receptor. In some cases, at least one of the one or more cells expresses a plurality of odorant receptors. For example, at least one of the one or more cells expresses at least 2, 3, 4, 5, 6, 7, 8, 9, 10, 20, 40, 60, 80, or 100 odorant receptors. In another example, the one or more cells expresses on average at least 2, 3, 4, 5, 6, 7, 8, 9, 10, 20, 40, 60, 80, or 100 odorant receptors. In some cases, the odorant receptors comprise OR1A1, MOR106-1, OR51E1, OR10J5, OR51E2, MOR9-1, MOR18-1, MOR272-1, MOR31-1, MOR136-1; any fragment thereof, or any combination thereof. In some cases, the one or more signals are electrical signals, optical signals, or a combination thereof. For example, the one or more signals are electrical signals. In another example, the one or more signals are optical signals. In another example, the one or more signals are a combination of electrical and optical signals. In some cases, the one or more signals are electrical signals comprising an action potential. In some cases, the one or more signals are electrical signals comprising an excited signal that is below a threshold for an action potential. In some cases, the one or more signals are electrical signals comprising a cell membrane depolarization. In some cases, the intensity of one or more signals is detected by a detector. In some cases, the intensity of one or more signals is proportional of the amount of the olfactory stimulus.

In some cases, the method further comprises applying one or more attributes of the olfactory stimulus and the reference signal to a machine learning algorithm. In some cases, the machine learning algorithm comprises Support Vector Machine (SVM), Naïve Bayes (NB), Quadratic Discriminant Analysis (QDA), K-Nearest Neighbors (KNN), Linear Discriminant Analysis (LDA), Multilayer Perceptron (MLP), or any combination thereof. In some cases, the machine learning algorithm can predict a reference signal of an olfactory stimulus by using one or more attributes of the olfactory stimulus.

In another aspect, disclosed here is a method for replicating an olfactory stimulus, comprising: a) contacting the olfactory stimulus with an artificial array comprising one or more chambers, wherein the one or more chambers comprise one or more cells expressing one or more cell-surface receptors, wherein the one or more cell-surface receptors generate one or more signals in response to a binding event between the olfactory stimulus and the one or more cell-surface receptors; b) recording an intensity of one or more signals of one of the cells; c) encoding the olfactory stimulus by creating a target signal, wherein the target signal comprises the intensity of the one or more signals; and d) replicating the target signal of the olfactory stimulus by mixing two or more reference olfactory stimuli, each of which has a reference signal, wherein the reference signals of the two or more reference olfactory stimuli have a combined signal that is similar to the target signal.

In some cases, the one or more cells are neurons. In some cases, the neurons are human neurons. In some cases, the one or more cells are modified to express the one or more cell-surface receptors. For example, the one or more cells are modified by introducing mRNAs that encode the one or more cell-surface receptors into the one or more cells. In some cases, the one or more cells are genetically modified to express the one or more cell-surface receptors. For example, the one or more cells are genetically modified by using CRISPR gene editing methods. In some cases, at least one of the one or more cell-surface receptors is an odorant receptor. In some cases, at least one of the one or more cells expresses one odorant receptor. In some cases, at least one of the one or more cells expresses a plurality of odorant receptors. For example, at least one of the one or more cells expresses at least 2, 3, 4, 5, 6, 7, 8, 9, 10, 20, 40, 60, 80, or 100 odorant receptors. In another example, the one or more cells expresses on average at least 2, 3, 4, 5, 6, 7, 8, 9, 10, 20, 40, 60, 80, or 100 odorant receptors. In some cases, the odorant receptors comprise OR1A1, MOR106-1, OR51E1, OR10J5, OR51E2, MOR9-1, MOR18-1, MOR272-1, MOR31-1, MOR136-1; any fragment thereof, or any combination thereof. In some cases, the one or more signals are electrical signals, optical signals, or a combination thereof. For example, the one or more signals are electrical signals. In another example, the one or more signals are optical signals. In another example, the one or more signals are a combination of electrical and optical signals. In some cases, the one or more signals are electrical signals comprising an action potential. In some cases, the one or more signals are electrical signals comprising an excited signal that is below a threshold for an action potential. In some cases, the one or more signals are electrical signals comprising a cell membrane depolarization. In some cases, the intensity of one or more signals is detected by a detector. In some cases, the intensity of one or more signals is proportional of the amount of the olfactory stimulus.

In some cases, the method further comprises applying one or more attributes of the olfactory stimulus and the reference signal to a machine learning algorithm. In some cases, the machine learning algorithm comprises Support Vector Machine (SVM), Naïve Bayes (NB), Quadratic Discriminant Analysis (QDA), K-Nearest Neighbors (KNN), Linear Discriminant Analysis (LDA), Multilayer Perceptron (MLP), or any combination thereof. In some cases, the machine learning algorithm can predict a reference signal of an olfactory stimulus by using one or more attributes of the olfactory stimulus.

In another aspect, disclosed here is a method for decoding an olfactory stimulus, comprising: a) contacting the olfactory stimulus with an artificial array comprising one or more chambers, wherein the one or more chambers comprise one or more cells expressing one or more cell-surface receptors, wherein the one or more cell-surface receptors generate one or more signals in response to a binding event between the olfactory stimulus and the one or more cell-surface receptors; b) recording an intensity of one or more signals of one of the cells; c) encoding the olfactory stimulus by creating a target signal, wherein the target signal comprises the intensity of the one or more signals; and d) decoding the olfactory stimulus to comprise one or more reference olfactory stimuli, wherein the one or more reference olfactory stimuli have a combined signal that is similar to the target signal.

In some cases, each of the one or more reference olfactory stimuli has a reference signal. In some cases, the decoding the olfactory stimulus comprises combining the reference signal of the one or more reference olfactory stimuli to match a signal that is similar to the target signal. In some cases, the one or more cells are neurons. In some cases, the neurons are human neurons. In some cases, the one or more cells are modified to express the one or more cell-surface receptors. For example, the one or more cells are modified by introducing mRNAs that encode the one or more cell-surface receptors into the one or more cells. In some cases, the one or more cells are genetically modified to express the one or more cell-surface receptors. For example, the one or more cells are genetically modified by using CRISPR gene editing methods. In some cases, at least one of the one or more cell-surface receptors is an odorant receptor. In some cases, at least one of the one or more cells expresses one odorant receptor. In some cases, at least one of the one or more cells expresses a plurality of odorant receptors. For example, at least one of the one or more cells expresses at least 2, 3, 4, 5, 6, 7, 8, 9, 10, 20, 40, 60, 80, or 100 odorant receptors. In another example, the one or more cells expresses on average at least 2, 3, 4, 5, 6, 7, 8, 9, 10, 20, 40, 60, 80, or 100 odorant receptors. In some cases, the odorant receptors comprise OR1A1, MOR106-1, OR51E1, OR10J5, OR51E2, MOR9-1, MOR18-1, MOR272-1, MOR31-1, MOR136-1; any fragment thereof, or any combination thereof. In some cases, the one or more signals are electrical signals, optical signals, or a combination thereof. For example, the one or more signals are electrical signals. In another example, the one or more signals are optical signals. In another example, the one or more signals are a combination of electrical and optical signals. In some cases, the one or more signals are electrical signals comprising an action potential. In some cases, the one or more signals are electrical signals comprising an excited signal that is below a threshold for an action potential. In some cases, the one or more signals are electrical signals comprising a cell membrane depolarization. In some cases, the intensity of one or more signals is detected by a detector. In some cases, the intensity of one or more signals is proportional of the amount of the olfactory stimulus.

In some cases, the method further comprises applying one or more attributes of the olfactory stimulus and the reference signal to a machine learning algorithm. In some cases, the machine learning algorithm comprises Support Vector Machine (SVM), Naïve Bayes (NB), Quadratic Discriminant Analysis (QDA), K-Nearest Neighbors (KNN), Linear Discriminant Analysis (LDA), Multilayer Perceptron (MLP), or any combination thereof. In some cases, the machine learning algorithm can predict a reference signal of an olfactory stimulus by using one or more attributes of the olfactory stimulus.

In another aspect, disclosed here is a method for stratifying an olfactory stimulus into a reference emotional state, comprising: a) contacting the olfactory stimulus with an artificial array comprising one or more chambers, wherein the one or more chambers comprise one or more cells expressing one or more cell-surface receptors, wherein the one or more cell-surface receptors generate one or more signals in response to a binding event between the olfactory stimulus and the one or more cell-surface receptors; b) recording an intensity of one or more signals of one of the cells; c) encoding the olfactory stimulus by creating a reference signal, wherein the reference signal comprises the intensity of the one or more signals; and d) stratifying the olfactory stimulus into the reference emotional state, wherein the reference emotional state is determined by a smelling assay on a subject.

In some cases, the one or more cells are neurons. In some cases, the neurons are human neurons. In some cases, the one or more cells are modified to express the one or more cell-surface receptors. For example, the one or more cells are modified by introducing mRNAs that encode the one or more cell-surface receptors into the one or more cells. In some cases, the one or more cells are genetically modified to express the one or more cell-surface receptors. For example, the one or more cells are genetically modified by using CRISPR gene editing methods. In some cases, at least one of the one or more cell-surface receptors is an odorant receptor. In some cases, at least one of the one or more cells expresses one odorant receptor. In some cases, at least one of the one or more cells expresses a plurality of odorant receptors. For example, at least one of the one or more cells expresses at least 2, 3, 4, 5, 6, 7, 8, 9, 10, 20, 40, 60, 80, or 100 odorant receptors. In another example, the one or more cells expresses on average at least 2, 3, 4, 5, 6, 7, 8, 9, 10, 20, 40, 60, 80, or 100 odorant receptors. In some cases, the odorant receptors comprise OR1A1, MOR106-1, OR51E1, OR10J5, OR51E2, MOR9-1, MOR18-1, MOR272-1, MOR31-1, MOR136-1; any fragment thereof, or any combination thereof. In some cases, the one or more signals are electrical signals, optical signals, or a combination thereof. For example, the one or more signals are electrical signals. In another example, the one or more signals are optical signals. In another example, the one or more signals are a combination of electrical and optical signals. In some cases, the one or more signals are electrical signals comprising an action potential. In some cases, the one or more signals are electrical signals comprising an excited signal that is below a threshold for an action potential. In some cases, the one or more signals are electrical signals comprising a cell membrane depolarization. In some cases, the intensity of one or more signals is detected by a detector. In some cases, the intensity of one or more signals is proportional of the amount of the olfactory stimulus.

In some cases, the method further comprises applying one or more attributes of the olfactory stimulus and the reference signal to a machine learning algorithm. In some cases, the machine learning algorithm comprises Support Vector Machine (SVM), Naïve Bayes (NB), Quadratic Discriminant Analysis (QDA), K-Nearest Neighbors (KNN), Linear Discriminant Analysis (LDA), Multilayer Perceptron (MLP), or any combination thereof. In some cases, the machine learning algorithm can predict a reference signal of an olfactory stimulus by using one or more attributes of the olfactory stimulus.

In some cases, the smelling assay is performed by analyzing a linguistic expression of the subject in response to the olfactory stimulus. In some cases, the linguistic expression is spoken, written, or signed. In some cases, the linguistic expression is translated into text. In some cases, the subject is asked to state the subject's emotional state. In some cases, the subject is asked to assign the subject's emotional state to a numerical level. In some cases, the subject is asked to assign the subject's emotional state to one or more images corresponding to the reference emotional state.

In some cases, the method further comprises detecting a physiological signal from the subject in response to the olfactory stimulus using a sensor. In some cases, the physiological signal is selected from the group comprising facial expressions, micro expressions, brain signals, electroencephalography (EEG) signals, functional magnetic resonance imaging (fMRI) signals, body odors, pupil dilation, skin conductance, skin potential, skin resistance, skin temperature, respiratory frequency, blood pressure, blood flow, saliva, and any combination thereof. In some cases, the sensor is connected to the subject. In some cases, the sensor is an EEG electrode. In some cases, the physiological signal is similar to a reference physiological signal corresponding to the reference emotional state.

In another aspect, disclosed here is a method for assessing an emotional state of a subject in response to an olfactory stimulus, comprising: a) contacting the olfactory stimulus with an artificial array comprising one or more chambers, wherein the one or more chambers comprise one or more cells expressing one or more cell-surface receptors, wherein the one or more cell-surface receptors generate one or more signals in response to a binding event between the olfactory stimulus and the one or more cell-surface receptors; b) recording an intensity of one or more signals of one of the cells; c) encoding the olfactory stimulus by creating a target signal, wherein the target signal comprises the intensity of the one or more signals; and d) stratifying the olfactory stimulus into a reference emotional state, wherein the target signal is similar to a reference signal corresponding to the reference emotional state.

In some cases, the subject is a human. In some cases, the one or more cells are neurons. In some cases, the neurons are human neurons. In some cases, the one or more cells are modified to express the one or more cell-surface receptors. For example, the one or more cells are modified by introducing mRNAs that encode the one or more cell-surface receptors into the one or more cells. In some cases, the one or more cells are genetically modified to express the one or more cell-surface receptors. For example, the one or more cells are genetically modified by using CRISPR gene editing methods. In some cases, at least one of the one or more cell-surface receptors is an odorant receptor. In some cases, at least one of the one or more cells expresses one odorant receptor. In some cases, at least one of the one or more cells expresses a plurality of odorant receptors. For example, at least one of the one or more cells expresses at least 2, 3, 4, 5, 6, 7, 8, 9, 10, 20, 40, 60, 80, or 100 odorant receptors. In another example, the one or more cells expresses on average at least 2, 3, 4, 5, 6, 7, 8, 9, 10, 20, 40, 60, 80, or 100 odorant receptors. In some cases, the odorant receptors comprise OR1A1, MOR106-1, OR51E1, OR10J5, OR51E2, MOR9-1, MOR18-1, MOR272-1, MOR31-1, MOR136-1; any fragment thereof, or any combination thereof. In some cases, the one or more signals are electrical signals, optical signals, or a combination thereof. For example, the one or more signals are electrical signals. In another example, the one or more signals are optical signals. In another example, the one or more signals are a combination of electrical and optical signals. In some cases, the one or more signals are electrical signals comprising an action potential. In some cases, the one or more signals are electrical signals comprising an excited signal that is below a threshold for an action potential. In some cases, the one or more signals are electrical signals comprising a cell membrane depolarization. In some cases, the intensity of one or more signals is detected by a detector. In some cases, the intensity of one or more signals is proportional of the amount of the olfactory stimulus.

In some cases, the method further comprises applying one or more attributes of the olfactory stimulus and the reference signal to a machine learning algorithm. In some cases, the machine learning algorithm comprises Support Vector Machine (SVM), Naïve Bayes (NB), Quadratic Discriminant Analysis (QDA), K-Nearest Neighbors (KNN), Linear Discriminant Analysis (LDA), Multilayer Perceptron (MLP), or any combination thereof. In some cases, the machine learning algorithm can predict a reference signal of an olfactory stimulus by using one or more attributes of the olfactory stimulus.

In some cases, the smelling assay is performed by analyzing a linguistic expression of the subject in response to the olfactory stimulus. In some cases, the linguistic expression is spoken, written, or signed. In some cases, the linguistic expression is translated into text. In some cases, the subject is asked to state the subject's emotional state. In some cases, the subject is asked to assign the subject's emotional state to a numerical level. In some cases, the subject is asked to assign the subject's emotional state to one or more images corresponding to the reference emotional state.

In some cases, the method further comprises detecting a physiological signal from the subject in response to the olfactory stimulus using a sensor. In some cases, the physiological signal is selected from the group comprising facial expressions, micro expressions, brain signals, electroencephalography (EEG) signals, functional magnetic resonance imaging (fMRI) signals, body odors, pupil dilation, skin conductance, skin potential, skin resistance, skin temperature, respiratory frequency, blood pressure, blood flow, saliva, and any combination thereof. In some cases, the sensor is connected to the subject. In some cases, the sensor is an EEG electrode. In some cases, the physiological signal is similar to a reference physiological signal corresponding to the reference emotional state.

In some cases, at least one of the one or more cells expressing one or more cell-surface receptors is connected to one or more transmitting cells. In some cases, at least one of the one or more cells expressing one or more cell-surface receptors is connected to the one or more transmitting cells via physical contact. In some cases, at least one of the one or more cells expressing one or more cell-surface receptors is connected to the one or more transmitting cells via a synapse. In some cases, the one or more signals are transmitted to the one or more transmitting cells by neurotransmitters. In some cases, the intensity of the one or more signals of one of the cells is measured from an intensity of a signal from the one or more transmitting cells.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 provides a schematic illustration of a cell-based sensor device comprising an array of cells in contact with a micro electrode array (MEA).

FIGS. 2A-B show schematic cross-sectional views of electrode structures for use with an embodiment of the invention. FIG. 2A: cross-sectional view of electrode structure comprising a plurality of protrusions. FIG. 2B: cross-sectional view of electrode comprising a plurality of depressions.

FIGS. 3A-B show schematic views of electrode structures for use with an embodiment of the invention. FIG. 3A: front view of electrode structure comprising a plurality of depressions. FIG. 3B: front view of electrode structure comprising a plurality of protrusions.

FIG. 4A-B show a non-limiting example of a cell-based sensor device of the present disclosure. FIG. 4A: top view. FIG. 4B: side view.

FIGS. 5A-B show a non-limiting example of an air-sampling device comprising microfluidic channels and a semi-permeable gas exchange membrane. FIG. 5A: top view. FIG. 5B: side view.

FIGS. 6A-B show a non-limiting example of a cell-based sensor device comprising an integrated gas exchange membrane. FIG. 6A: top view. FIG. 6B: side view.

FIG. 7 shows a non-limiting example of an air sampling device comprising a gas perfusion chamber with a micro bubbler.

FIG. 8 shows a non-limiting example of an air sampling device comprising an atomizer.

FIG. 9 shows a schematic illustration of an artificial neural network (ANN).

FIG. 10 shows a schematic illustration of a deep learning neural network (DNN).

FIG. 11 provides a schematic illustration of the functionality of a node within a layer of an artificial neural network or deep learning neural network.

FIG. 12 shows a computer control system that is programmed or otherwise configured to implement the methods provided herein.

FIGS. 13A-B show a non-limiting example of a cell-based sensor device comprising an integrated, texturized semi-permeable gas exchange membrane. FIG. 13A: top view. FIG. 13B: side view.

FIG. 14 shows an overview of a “smart tunnel” system configuration, including a four-stage detection system and built-in neural sensor panels.

FIG. 15 shows a non-limiting schematic illustration of one of the four detection stages of the “smart tunnel” system configuration illustrated in FIG. 14.

FIG. 16 shows a non-limiting example of detecting compound that triggers a single OR.

FIG. 17 shows a non-limiting example of detecting a mixture of compounds.

FIG. 18 shows a non-limiting example of determining a mixture of compounds that triggers a single OR.

FIG. 19 shows a non-limiting example of mapping emotions to every hOR or some combinations of hORs.

FIG. 20 shows a non-limiting example of predicting emotions based on one or more compounds.

FIG. 21 shows an exemplary method for assessing a physiological state of a subject in response to a stimulus.

FIG. 22 shows an exemplary emotional state flower of a human subject.

FIG. 23 shows an exemplary mapping between a list of compounds and their corresponding emotions based on biological optimum and cultural influence.

FIG. 24 shows a non-limiting example of a neural system of a human subject for sensing an odor.

FIG. 25 shows a non-limiting example of the relationship between the percentage of mixture overlap allowing discrimination and the number of discriminable mixtures.

FIG. 26 shows a non-limiting example of the numeric scale of the smells.

FIG. 27 shows an example of an overview of decoding an odor.

FIG. 28 shows a non-limiting example of a portion of an odor encoding device.

FIG. 29 shows a non-limiting example of the expression of olfactory receptors on a neuron.

FIG. 30 shows a non-limiting example of mapping an odor with olfactory receptors.

FIG. 31 shows an overview of correlating physiological responses of humans to smell with the activation profile of each olfactory receptor.

FIG. 32 shows a non-limiting example of correlating physiological responses of humans to smell with the activation profile of each olfactory receptor through the odor encoding device.

FIG. 33 shows a non-limiting example of detecting human physiological states through brain imaging.

FIG. 34 shows another non-limiting example of correlating physiological responses of humans to smell with the activation profile of each olfactory receptor through the odor encoding device.

FIG. 35 shows a non-limiting example of correlating physiological responses of humans to smell with the activation profile of each olfactory receptor through the odor encoding device and relevant algorithms.

FIG. 36 shows a non-limiting example of predicting, copying or reproducing any smell.

FIG. 37 shows non-limiting examples of user interfaces of an application related to the disclosure herein.

FIG. 38 shows a non-limiting example of a continuous learning process of the relevant algorithm.

FIG. 39 shows another example of mapping between a list of compounds and their corresponding emotions based on biological optimum and cultural influence.

FIG. 40 shows a non-limiting example of a human's neural system responding to an odor.

FIG. 41 shows a non-limiting example of an olfactory receptor.

FIG. 42 shows that an olfactory receptor has a DNA code.

FIG. 43 shows that the DNA can make the neuron to produce receptors.

FIG. 44 shows another non-limiting example of a human's neural response to an odor.

FIG. 45 shows another non-limiting example of a human's neural responses to an odor.

FIG. 46 shows a non-limiting example of mapping an odor with olfactory receptors.

FIG. 47 shows a non-limiting example of mapping an odor with olfactory receptors in vertical bar format.

FIG. 48 shows a non-limiting example of a human's emotion states.

FIG. 49 shows a non-limiting example of a dog's neural response to an odor.

FIG. 50 shows a non-limiting example of detecting neural responses to amine.

FIG. 51 shows a non-limiting example that receptors can be designed to bind biogenic amines specifically.

FIG. 52 shows a non-limiting example that human specimen can be measured to generate a diagnostic output via live cell assay.

FIG. 53 shows a non-limiting example of mapping an odor with olfactory receptors through dimensions of odor quality.

FIG. 54 shows a non-limiting example of the odor encoding device.

FIG. 55 shows a non-limiting example of improvement of the odor encoding device.

FIG. 56 shows that the application of the universal odor code system can be accessible through phone, computer, and any chosen store on tablets.

FIG. 57 shows a non-limiting example of detecting amines through trace amine-associated receptors.

FIG. 58 shows that synthetic biology can increase the sensitivity and specificity of the trace amine-associated receptors.

FIG. 59 shows a diagram of methods of encoding and decoding olfactory stimuli.

FIG. 60 shows partition of black and white dots by a support vector machine.

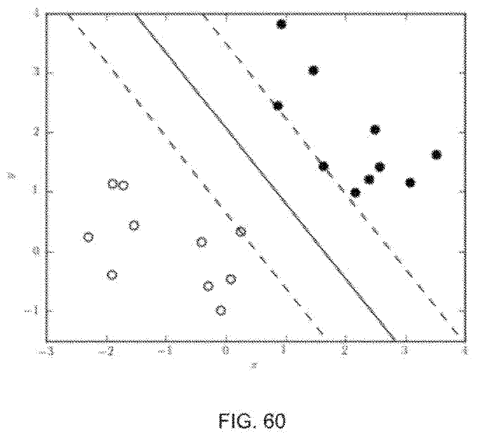

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### I. Definitions

As used herein, the singular forms “a”, “an”, and “the” include plural references unless the context clearly dictates otherwise. Any reference to “or” herein is intended to encompass “and/or” unless otherwise stated.

As used herein, unless otherwise indicated, the phrase “signal A is similar to signal B” means that the intensity of the signal A is within about ±50%, about ±40%, about ±30%, about ±20%, about ±10%, about ±5%, about ±4%, about ±3%, about ±2%, or about ±1% of the intensity of the signal B from at least 10% of the corresponding cells on the array, such as at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the corresponding cells on the array.

The term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2%, or 1% from that value. For example, the amount “about 10” includes amounts from 9 to 11.

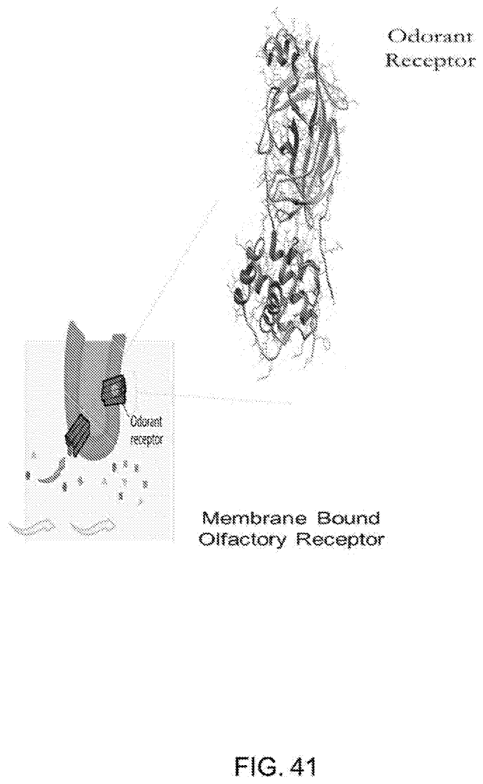

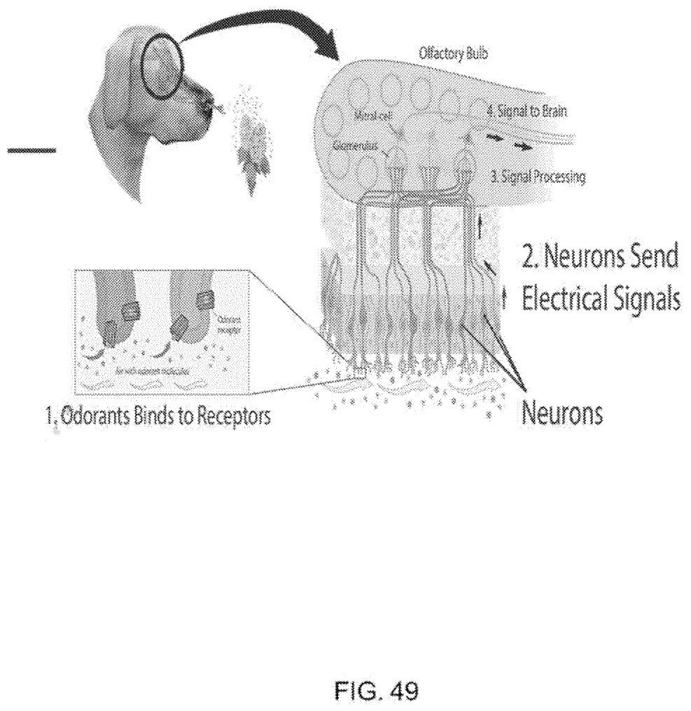

The term “cell” as used herein, generally refers to one or more cells. A cell may be obtained or isolated from a subject. A cell may be obtained or isolated from a tissue. A subject may be an animal such as a human, a mouse, a rat, a pig, a dog, a rabbit, a sheep, a horse, a chicken or other animal. A cell may be a mammalian cell. A cell may be a neuron, an astrocyte or a cell from a cultured cell line, such as a CHO (Chinese hamster ovary) cell, a human or mouse embryonic kidney cell (e.g., HEK-239), or a HeLa cell. A cell may be a pluripotent stem cell. A cell may be genetically engineered to express an olfactory receptor on its surface. It may further be engineered to express a protein that produces a detectable signal, such as a fluorescent or luminescent protein. A cell may be a neuron. FIG. 49 shows a non-limiting example of a dog's neural response to an odor. A dog's nose may serve a plurality of purposes, for example, breathing, and sample collection. Particles of explosives may bind to the dog's nose neurons (olfactory sensory neurons) which fire electrical signals. The dog may perceive the electrical signal and may tell its handlers. FIG. 41 shows a non-limiting example of an olfactory receptor. The dog's nose neuron may a little sensor sticking on its surface called an odorant receptor. This receptor may only respond to a whole chemical molecule. The whole chemical molecule may be a receptor ligand pair.

A neuron may be a central neuron, a peripheral neuron, a sensory neuron, an interneuron, a motor neuron, a multipolar neuron, a bipolar neuron, or a pseudo-unipolar neuron. A cell may be a neuron supporting cell, such as a Schwann cell. A cell may be one of the cells of a blood-brain barrier system. A cell may be a cell line, such as a neuronal cell line. A cell may be a primary cell, such as cells obtained from a brain of a subject. A cell may be a population of cells that may be isolated from a subject, such as a tissue biopsy, a cytology specimen, a blood sample, a fine needle aspirate (FNA) sample, or any combination thereof. A cell may be obtained from a bodily fluid such as urine, milk, sweat, lymph, blood, sputum, amniotic fluid, aqueous humour, vitreous humour, bile, cerebrospinal fluid, chyle, chyme, exudates, endolymph, perilymph, gastric acid, mucus, pericardial fluid, peritoneal fluid, pleural fluid, pus, rheum, saliva, sebum, serous fluid, smegma, sputum, tears, vomit, or other bodily fluid. A cell may comprise cancerous cells, non-cancerous cells, tumor cells, non-tumor cells, healthy cells, or any combination thereof. A cell may be a modified cell, such as a genetically modified cell. A modified cell may comprise an addition of one of more cell-surface receptors, such as modified cell-surface receptors. The modified cell-surface receptors may be modified to increase or decrease their ability to bind to a large set of compounds, a small set of compounds, or a specific compound. A modified cell may comprise a deletion of one or more cell-surface receptors.

The term “tissue” as used herein, generally refers to any tissue sample. A tissue may be a sample suspected or confirmed of having a disease or condition. A tissue may be a sample that is genetically modified. A tissue may be a sample that is healthy, benign, or otherwise free of a disease. A tissue may be a sample removed from a subject, such as a tissue biopsy, a tissue resection, an aspirate (such as a fine needle aspirate), a tissue washing, a cytology specimen, a bodily fluid, or any combination thereof. A tissue may comprise cancerous cells, tumor cells, non-cancerous cells, or a combination thereof. A tissue may comprise neurons. A tissue may comprise brain tissue, spinal tissue, or a combination thereof. A tissue may comprise cells representative of a blood-brain barrier. A tissue may comprise a breast tissue, bladder tissue, kidney tissue, liver tissue, colon tissue, thyroid tissue, cervical tissue, prostate tissue, lung tissue, heart tissue, muscle tissue, pancreas tissue, anal tissue, bile duct tissue, a bone tissue, uterine tissue, ovarian tissue, endometrial tissue, vaginal tissue, vulvar tissue, stomach tissue, ocular tissue, nasal tissue, sinus tissue, penile tissue, salivary gland tissue, gut tissue, gallbladder tissue, gastrointestinal tissue, bladder tissue, brain tissue, spinal tissue, a blood sample, or any combination thereof.

The term “receptor” as used herein, generally refers to a receptor of a cell. The receptor may be a cell-surface receptor. A receptor can be an olfactory receptor, e.g., a human olfactory receptor. A cell-surface receptor may be a G coupled protein receptor. A receptor may bind to one or more compounds. A receptor may have a different binding affinity to for each compound to which it binds. A receptor may be modified, such as genetically modified. A receptor may be modified to change the number of compounds to which it may bind. A receptor may be modified to increase the number of different compounds to which it may bind. A receptor may be modified to decrease the number of different compounds to which it may bind. A receptor may bind 1 compound. A receptor may bind 2, 3, 4, 5, 6, 7, 8, 9, 10, 15, 20, 30, 40, 50, 60, 70, 80, 90, 100 compounds or more. A receptor may bind less than 10 compounds. A receptor may bind less than 5 compounds. A receptor may bind at least 5 compounds. A receptor may bind at least 10 compounds. A receptor may bind at least 20 compounds. A receptor may be any receptor or any combination of the receptors listed in Table 1b, Table 2, Table 3, or Table 4. A receptor may be any receptor listed in Table 1b, Table 2, Table 3, Table 4, or any combination thereof, that further comprises a modification.

The term “modification” as used herein, generally refers to a modification to a cell, a modification to a protein, or a modification to a cell receptor. A modification to a cell may include adding one or more receptors, such as modified receptors, to the cell. A modification to a cell may include removing one or more receptors from a cell. A modification to a cell may include modifying one or more receptors that are expressed on the cell. A modification to a protein or cell receptor may include a genetic modification, an enzymatic modification, or a chemical modification. A modification to a protein or cell receptor may include a post-translational modification such as an acylation modification, an acetylation modification, a formylation modification, an alkylation modification, a methylation modification, an arginylation modification, a polyglutamylation modification, a polyglycylation modification, a butyrylation modification, a gamma-carboxylation modification, a glycosylation modification, a malonylation modification, a hydroxylation modification, an iodination modification, a nucleotide addition modification, an oxidation modification, a phosphate ester modification, a propionylation modification, a pyroglutamate formation modification, an S-glutathionylation modification, an S-nitrosylation modification, an S-sulfenylation modification, a succinylation modification, a sulfation modification, a glycation modification, a carbamylation modification, a carbonylation modification, a biotinylation modification, a pegylation modification, or any combination thereof.

The term “compound” as used herein, generally refers to a composition that may produce a signal in a cell, such as an electrical signal. A compound may be a mixture (sometimes referred to a composition). A compound may comprise an odorant. A compound may comprise a compound that binds an odorant receptor or a modified odorant receptor. A compound may comprise a volatile compound. A compound may comprise an organic volatile compound. A compound may comprise a neurotoxin or a toxin. A compound may comprise any compound or mixture thereof the odorant of Table 2a. A compound may comprise a carcinogen. A compound may comprise a chemical weapon, such as a mustard gas, a sarin gas, or a combination thereof. A compound may comprise an illegal substance as defined in 42 United States Code § 12210. A compound may comprise a drug or a pharmaceutical composition or salt thereof. A compound may comprise a protein, a peptide, a nucleic acid, an antibody, an aptamer, a small molecule. A compound may comprise a cell or a cellular fragment. A compound may comprise a tissue or tissue fragment. A compound may comprise a naturally-derived composition or a synthetic composition. A compound may be an explosive compound, such as trinitrotoluene (TNT). A compound may be volatile marker or taggant for an explosive material. A compound may be a precursor to the compound (such as a chemical precursor), a degradation product of the compound, or a metabolite of the compound, or any combination thereof. The terms “compound,” “stimulus,” and “element” may be used interchangeably.

The term “sample” as used herein, generally refers to a sample that may or may not comprise one or more compounds. A sample may be tissue or fluid sample obtained from a subject, such as a human subject. A sample may be a fluid or gas sample obtained from an air space, such as an outdoor air space, an air space adjacent to a deployment of a chemical weapon, or an air space in a residential or commercial setting (i.e., an indoor or enclosed environment). A sample may be a blood sample obtained from a subject. A sample may be a soil sample, such as a sample obtained near a fracking system or oil rig system. A sample may be a sample that may comprise a compound that is an environmental hazard or a health hazard. A sample may be a liquid sample obtained from a water system, such as a river, a stream, a lake, an ocean, or others. A sample may be a food sample or a container system that houses a food sample. A pattern or fingerprint of the systems described herein, may confirm a ripeness of a single piece of food, such as a fruit, or a set of fruit.

The term “signal” as used herein, generally refers to a signal in response to a binding event, for example, a compound binding to a cell-surface receptor of a cell. The signal may be an electrical signal. The signal may be a voltage or a current measurement. The signal may be a change in a cell membrane potential. The signal may be a membrane depolarization. The signal may be an action potential. The signal may be an electrical signal that is subthreshold of an action potential. The signal may be a magnitude of a change in a cell membrane potential, or a magnitude of an action potential. The signal may be the number of action potentials or a train of action potentials. The signal may be a signal measured over a period of time. Information from a signal may be imported into a matrix to form a fingerprint or a pattern of signals. The fingerprint or pattern of signals may be a unique fingerprint. The signal may be a measurement of an amplitude, a period, or a frequency, of a combination thereof of an electrical signal. The signal may be a time length of a refractory period following an action potential. The signal may be a peak voltage of an action potential. The signal may be a time to a peak voltage of an action potential. The signal may be a peak voltage of a membrane depolarization. The signal may be an optical signal, such as fluorescence or luminescence produced by a protein.

An optical signal may be produced in a number of ways. In one embodiment, a fluorescent or luminescent protein can be placed under the control of a cAMP-responsive element (CRE) and placed in a cell or extracellular environment. When an OR is stimulated and transuces a signal that generated cAMP, this will result in production of the protein, whose production can be detected. In another embodiment, production of cAMP can be detected by an ex vivo enzymatic assay that uses light as a reporter.

### II. The Odor Encoding Device

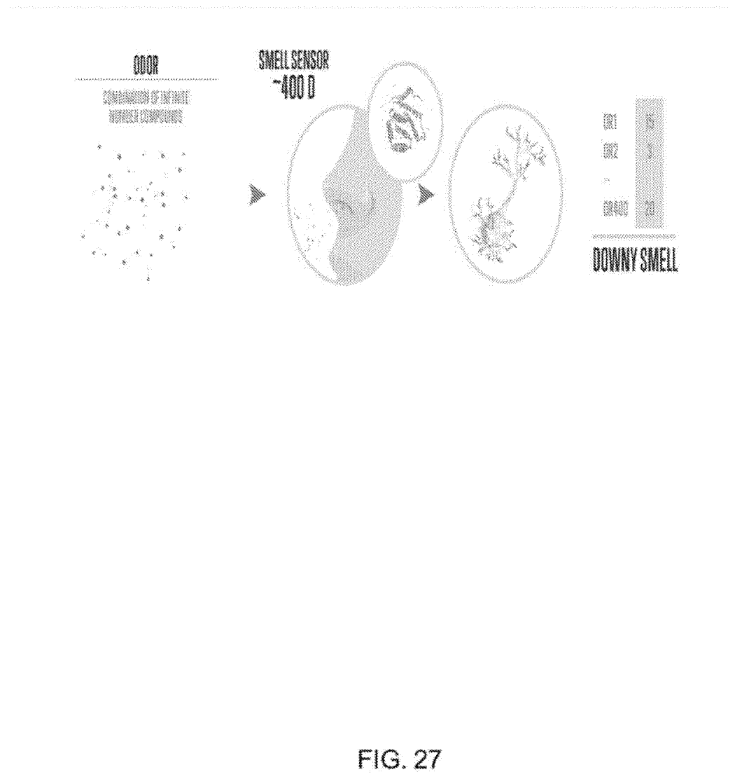

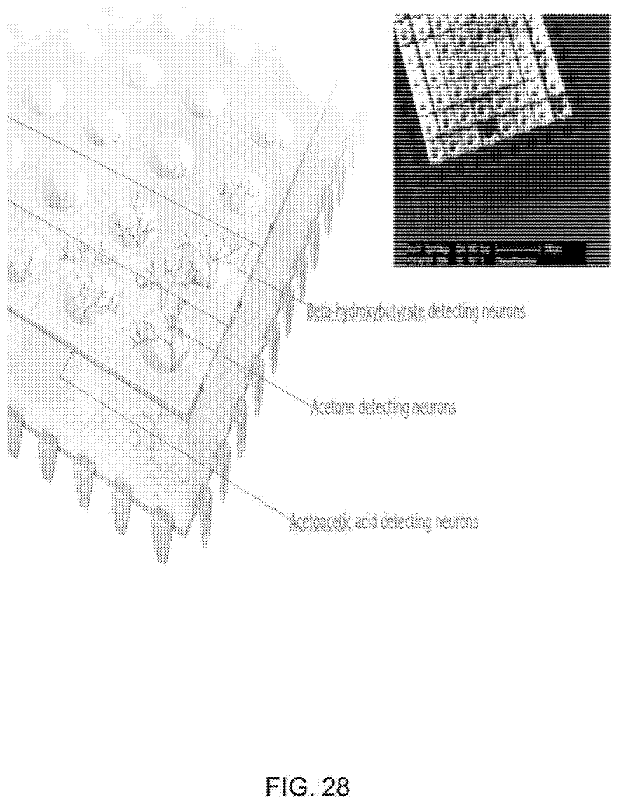

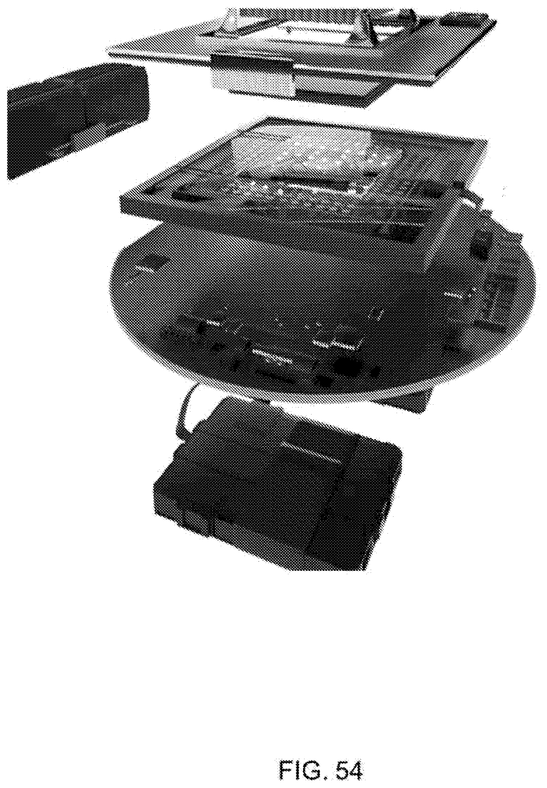

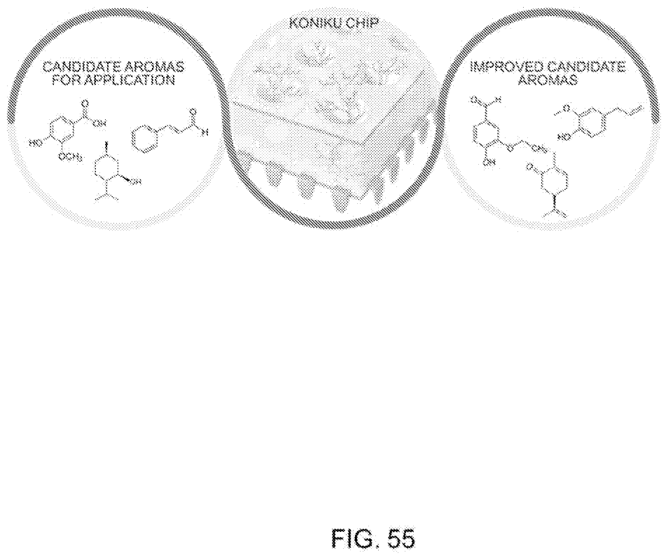

The odor encoding device may be a cell-based sensor device. The odor encoding device may encode or decode an odor. FIG. 27 shows an example of an overview of decoding an odor. FIG. 28 shows a non-limiting example of a portion of an odor encoding device. FIG. 54 shows a non-limiting example of the odor encoding device. FIG. 55 shows a non-limiting example of improvement of the odor encoding device. Recently, microfluidic devices that provide for the culturing of cells in carefully-controlled microenvironments that more closely mimic the in vivo environment have been described in the literature. For example, see the cell culture modules and systems described in co-pending patent applications published as US 2017/0015964 A1 and WO 2017/015148 A1. The ability to culture cells and maintain their viability for prolonged periods of time in carefully-controlled microenvironments has application in a variety of fields including basic biomedical research, tissue engineering, biosensor-based detection systems, etc.

Disclosed herein are cell-based sensor devices, sensor panels comprising arrays of one or more cell-based sensor devices, detection systems comprising one or more sensor panels, and methods of use thereof. The disclosed detection systems take advantage of the binding specificity inherent in cell surface receptor-ligand binding interactions and the signal amplification inherent in the signaling pathways of excitable cells to achieve sensitive and specific detection of compounds, e.g., volatile compounds present in air samples drawn from outdoor or indoor (enclosed) environments.

In a first aspect of the present disclosure, cell-based sensor devices are described that comprise a plurality of chambers, wherein each chamber comprises at least one cell expressing one or more cell surface receptors, and at least one electrode configured to measure electrical signals positioned within the chamber. In some embodiments, the cell(s) within each chamber of the device are bathed in a cell culture medium that is continuously, periodically, or randomly perfused through each chamber of the plurality of chambers in order to maintain the viability of the cells therein. Binding events between a compound (or mixture of compounds) introduced into the medium bathing the cells and one or more of the cell surface receptors may give rise to signals, e.g., electrical signals (e.g., changes in cell surface electrostatic potentials or cell membrane depolarizations) or optical signals, that are detected by the electrode in the corresponding chamber. In some embodiments, cells in different chambers may comprise different cell surface receptors, or may comprise the same cell surface receptor expressed at different levels, such that the plurality of electrodes associated with the plurality of chambers in the device detect a pattern of electrical signals in response to a binding event that may be recorded and/or processed. In some embodiments, the cell-based sensor device may comprise a processor for processing the patterns of electrical signals detected by the plurality of electrodes. In some embodiments, the processor may be external to the cell-based sensor device. In some embodiments, machine learning-based processing of the patterns of electrical signals may be used to improve the sensitivity and/or specificity of the cell-based sensor device for detection of specific compounds or mixtures of compounds. Some aspects of the disclosed cell-based sensor devices have been described in co-pending PCT Application No. PCT/US17/58895.

In a second aspect of the present disclosure, sensor panels are described which comprise one or more cell-based sensor devices. In some embodiments, the sensor panels may comprise two or more individual cell-based sensor devices, wherein each cell-based sensor device has been designed and/or optimized (e.g., by virtue of choosing the types of cells and/or cell surface receptors expressed in each of the plurality of chambers within each cell-based sensor device) to detect a different compound or mixture of compounds, such that the sensor panel is designed and/or optimized to detect two or more different compounds or mixtures of compounds. In some embodiments, each cell-based sensor device may comprise a processor for processing the patterns of electrical signals detected by plurality of electrodes in each device. In some embodiments, the sensor panel may comprise a processor for processing the patterns of electrical signals detected by the plurality of electrodes in all cell-based sensor devices of the panel. In some embodiments, machine learning-based processing of the patterns of electrical signals recorded by the plurality of electrodes in each of the cell-based sensor devices of the panel is used to improve the sensitivity and/or specificity of the sensor panel for detection of specific compounds or mixtures of compounds, while minimizing signal cross-talk between the individual cell-based sensor devices.

In a third aspect of the present disclosure, detection systems are described which comprise two or more sensor panels. In some embodiments, the two or more sensor panels may comprise the same complement of cell-based sensor devices, i.e., a set of cell-based sensor devices designed and/or optimized for detection of the same set of compounds or mixtures of compounds. In some embodiments, the two or more sensor panels may comprise different complements of cell-based sensor devices, i.e., sets of cell-based sensor devices designed and/or optimized for detection of a different set of compounds or mixtures of compounds. In some embodiments, the two or more sensor panels of the detection system may be positioned at known locations within a defined outdoor or indoor (enclosed) environment. In some embodiments, the detection system may further comprise two or more air sampling devices, wherein each air sampling device is in fluid communication with one of the two or more sensor panels, and wherein each air sampling device is configured to facilitate the transfer compounds present in the air to a liquid medium that bathes the cells in each of the chambers in each cell-based sensor device of the corresponding sensor panel. In some embodiments, the detection system may comprise a controller configured to receive the electrical signals measured by the plurality of electrodes in each cell-based sensor device of the two or more sensor panels. In some embodiments, the controller stores and processes a pattern of signals, e.g., electrical signals or optical signals, associated with a compound or mixture of compounds that is generated by at least one of the cell-based sensor devices in each of the two or more sensor panels (which are positioned at known locations) to identify the compound or mixture of compounds and provide a spatial location of a source of the compound or mixture of compounds within an outdoor or indoor (enclosed) environment.

A. Recombinant Cells Expressing Olfactory Receptors

In nature, olfactory receptors (ORs, also known as “odorant receptors”) are located on the cilia of olfactory receptor cells; with each receptor cell expressing a single odorant receptor gene. The olfactory receptors are linked to the stimulatory guanine nucleotide binding protein (G-protein) Golf When stimulated, the Golf protein can activate adenylate cyclase to produce the second messenger cAMP, and subsequent events lead to depolarization of the cell membrane and signal propagation. Although each receptor cell only expresses one type of receptor, each cell is electrophysiologically responsive to a wide but circumscribed range of stimuli. This implies that a single receptor accepts a range of molecular entities.

OR proteins are retained in the endoplasmic reticulum (ER) and subsequently degraded in the proteosome (see, e.g., Lu, M. et al. (2003) Traffic 4: 416-433; McClintock, T. S. (1997) Brain Res. Mol. Brain Res. 48: 270-278). Accordingly, it has been difficult to express ORs on the surface of heterologous cells to assay their ligand-binding specificity (i.e., the selectivity of the different ORs for chemical stimuli; see, e.g., Mombaerts, P. (2004) Nature Rev. Neurosci. 5: 263-278).

Addition of exogenous peptide sequences (e.g., the 20 N-terminal amino acids of rhodopsin) to the N-terminus of certain ORs has facilitated their expression on the surface of heterologous cells (see, e.g., Hatt, H. et al. (1999) Cell. Mol. Biol. 45: 285-291; Krautwurst, D. et al. (1998) Cell 95: 917-926); but, for most ORs, sequence modifications do not reliably promote cell-surface expression.

Thus, continued progress in understanding olfactory coding has been hampered by the inability to functionally express ORs in heterologous cells in order to identify cognate ligands. However, three transmembrane proteins, that promote functional cell surface expression of ORs in heterologous cells, have been identified. U.S. Patent Application Publication No. 2006/0057640; herein incorporated by reference in its entirety. These “chaperone” proteins (REEP1, RTP1 and RTP2) are expressed specifically by olfactory neurons in the olfactory epithelium. REEP1 and RTP1 interact with OR proteins.

Any number of cells can be engineered to express olfactory receptors. These include, for example, neurons, astrocytes and various cell lines, such as mouse kidney cells.

To facilitate analysis of odorant-OR interactions, a cell line named Hana3A was established. This line, derived from the 293T cell line, a mouse kidney cell line, stably expresses exogenous REEP1, RTP1 and RTP2 and also stably expresses an exogenous alpha subunit of the OR-binding G protein Golf (Gaolf). See, e.g., Belluscio, L. et al. (1998) Neuron 20: 69-81; Jones, D. T. and Reed, R. R. (1989) Science 244: 790-795. When Hana3A cells are transfected with sequences encoding ORs, enhanced cell-surface expression of the exogenous OR is observed. See, e.g., US Patent Application Publication No. 2006/0057640.

Accordingly, in some embodiments, OR activation is measured in Hana3A cells transfected with sequences encoding the OR under study, or a functional fragment thereof. In these systems, activation of the OR under study results in activation of the Golf G-protein, which in turn results in activation of adenylate cyclase and resultant production of a second messenger such as cyclic AMP in the OR-transfected cell. Second messenger (e.g., cyclic AMP) levels are then determined as a measure of OR activation.

There are a number of methods for measuring cAMP levels in cells. For example, cAMP levels can be measured directly using, for example, the cAMP-Glo Assay (Promega, Madison, Wis.), a cAMP competitive ELISA (Abcam, Cambridge, Mass.), the colorimetric cAMP direct immunoassay kit (Biovision, Milpitas, Calif.) and the cAMP-Screen Direct System (Applied Biosystems). Additional cAMP assay systems are available and are known to those of skill in the art.

In other embodiments, levels of a reporter, whose expression is dependent on, and proportional to, cAMP concentration are determined. cAMP-dependent expression of a reporter can be achieved, for example, using sequences encoding the reporter that are operably linked to, and under the transcriptional control of, a cAMP-sensitive promoter. cAMP-sensitive (or cAMP-dependent) promoters can include the CRE (cAMP response element) sequence and/or the AP-2 sequence. See, for example, Roesler et al. (1988) J. Biol. Chem. 263:9063-9066.

Reporter molecules are known in the art and include, without limitation, enzymatic reporters, fluorescent reporters, luminescent reporters, immunological reporters and ion channel reporters. Enzymatic reporters include, for example, β-galactosidase, β-glucuronidase (GUS), glutathione-S-transferase (GST), horseradish peroxidase (HRP), alkaline phosphatase (AP), acetylcholinesterase, catalase and chloramphenicol acetyl transferase (CAT).

Examples of fluorescent reporters include, for example, green fluorescent protein (GFP) from Aequorea victoria or Renilla reniformis, and active variants thereof (e.g., blue fluorescent protein, yellow fluorescent protein, cyan fluorescent protein, etc.); red fluorescent protein (RFP) fluorescent proteins from Hydroid jellyfishes, Copepod, Ctenophora, Anthrozoas, and Entacmaea quadricolor, and active variants thereof; phycobiliproteins and active variants thereof, and modified fluorescent proteins as are known in the art.

Other fluorescent reporters include, for example, small molecules such as CPSD (Disodium 3-(4-methoxyspiro {1,2-dioxetane-3,2′-(5′-chloro)tricyclo [3.3.1.1^{3,7}]decan}-4-yl)phenyl phosphate, ThermoFisher Catalog #T2141).

Bioluminescent reporters include, for example, aequorin (and other Ca⁺² regulated photoproteins), luciferase based on luciferin substrate, luciferase based on Coelenterazine substrate (e.g., Renilla, Gaussia, and Metridina), luciferase from Cypridina, and active variants thereof. The bioluminescent reporter can be, for example, North American firefly luciferase, Japanese firefly luciferase, Italian firefly luciferase, East European firefly luciferase, Pennsylvania firefly luciferase, Click beetle luciferase, railroad worm luciferase, Renilla luciferase, Gaussia luciferase, Cypridina luciferase, Metrida luciferase, OLuc, and red firefly luciferase, all of which are commercially available from, e.g., ThermoFisher Scientific and/or Promega.

Immunological reporters include any peptide sequence for which a specifically-binding antibody is available, for example, His₆, hemagglutinin and myc.

Ion channel reporters, include, for example, cAMP activated cation channels. The reporter or reporters may also include a Positron Emission Tomography (PET) reporter, a Single Photon Emission Computed Tomography (SPECT) reporter, a photoacoustic reporter, an X-ray reporter, and an ultrasound reporter.

For certain luminescence assays, a CRE-Luciferase cassette (e.g., from Stratagene, La Jolla, Calif.) is used, and luciferase is detected using, e.g., the Dual-Glo system (Promega, Madison, Wis.). See, e.g., Whissell-Buechy et al. (1973) Nature 242:271-273. Optionally, an internal control containing sequences encoding Renilla luciferase under the transcriptional control of a SV40 promoter (e.g., from Promega, Madison, Wis.) is used for standardization. Additional cAMP-dependent reporter systems using a luciferase reporter that quantify OR activation based on cAMP production are described by Saito et al., (2004) Cell 119:679; Zhuang et al. (2007) J Biol. Chem. 282:15284; Katada et al. (2003) Biochem. Biophys. Res. Commun. 305:964; and Zhuang & Matsunami (2008) Nat. Protoc. 3:1402.

In additional embodiments, cAMP-dependent expression of a fluorescent protein (e.g., GFP) is used to measure OR activation, for example, by including in the cell used for OR assay a cassette containing sequences encoding the fluorescent protein operably linked to a cAMP-dependent promoter (e.g., a promoter containing a CRE element).

See also U.S. Patent Application Nos. 2006/0057640, 2008/0081345, 2010/0143337 and 2013/0004983 for additional examples of methods for measuring OR activation.

Increases in intracellular cAMP concentration can also result in influx of calcium ions (Ca²⁺) into the cell. Thus, another method for measuring OR activation is to measure intracellular Ca²⁺ levels. Accordingly, in certain embodiments, cells are loaded with a Ca²⁺-sensitive dye (e.g., fluo-4 and/or fura-red). When calcium concentration inside the cell is upregulated upon stimulation of the OR with ligands, the fluo-4 signal increases whereas the fura-red signal decreases, thereby allowing ratiometric measurements of intracellular calcium concentration. Wong et al. (2002) Nat. Neurosci. 5:1302-1308.

In additional embodiments for measuring Ca²⁺, cells used for assaying OR activation contain a calmodulin/GFP fusion protein. In the absence of Ca²⁺, the GFP portion of the fusion protein is folded in a way that prevents fluorescence. Release of Ca²⁺ ions into the cytoplasm (either from the extracellular environment or from the endoplasmic reticulum) results in binding of Ca²⁺ ions to the calmodulin portion of the fusion protein, causing a conformational change in the fusion protein that allows the GFP portion to fluoresce. It will be clear to those skilled in the art that fluorescent proteins other than GFP can be used in such fusion proteins.

In some embodiments, second messenger assays measure fluorescent signals from reporter molecules that respond to intracellular changes (e.g., Ca²⁺ concentration, membrane potential, pH, IP₃, cAMP levels, arachidonic acid release) due to stimulation of membrane receptors and ion channels (e.g., ligand gated ion channels; see Denyer et al. (1998) Drug Discov. Today 3:323 and Gonzales et al. (1999) Drug. Discov. Today 4:431-439). Examples of reporter molecules include, but are not limited to, FRET (florescence resonance energy transfer) systems (e.g., Cuo-lipids and oxonols, EDAN/DABCYL), calcium sensitive indicators (e.g., Fluo-3, FURA 2, INDO 1, and FLUO3/AM, BAPTA AM), chloride-sensitive indicators (e.g., SPQ, SPA), potassium-sensitive indicators (e.g., PBFI), sodium-sensitive indicators (e.g., SBFI), and pH sensitive indicators (e.g., BCECF).

In general, the host cells are loaded with the indicator prior to exposure to the test compound or odorant. Responses of the host cells to treatment with the compounds can be detected by methods known in the art, including, but not limited to, fluorescence microscopy, confocal microscopy (e.g., FCS systems), flow cytometry, microfluidic devices, FLIPR systems (see, e.g., Schroeder & Neagle (1996) J. Biomol. Screening 1:75), and plate-reading systems. In some embodiments, the response (e.g., increase in fluorescence intensity) caused by a compound or odorant of unknown activity is compared to the response generated by a known agonist and expressed as a percentage of the maximal response of the known agonist. The maximum response caused by a known agonist is defined as a 100% response.

Additional methods for measuring ion channel activation by cAMP are known in the art.

In certain embodiments, cells used for assaying OR activation comprise a muscarinic acetylcholine receptor (e.g., M1, M2, M3, M4 and/or M5). In certain embodiments, the cells used for assaying OR activation comprise a Type 3 muscarinic acetylcholine receptor M3 (e.g., encoded by the human gene CHRM3), which enhances the response of an OR to its cognate ligand(s). U.S. Patent Application Publication No. 2013/0004983.

In certain embodiments, cells used for assaying OR activation comprise a RTP1S polypeptide or functional fragment thereof.

In certain embodiments, cells used for assaying OR activation comprise an olfactory GTP-GDP exchange factor Ric-8b. Von Dannecker et al., (2006) Proc. Natl. Acad. Sci. USA 103:9310; Saito et al. (2004) Cell 119:679; Zhuang et al. (2007) J. Biol. Chem. 282:15284.

In certain embodiments, cells used for assaying OR activation comprise heat shock protein 70 (HSP70) or the HSP70 homologue HSC70T.

In certain embodiments, cells used for assaying OR activation comprise one or more of an alpha, beta or gamma subunit of a G-protein.

In certain embodiments, cells used for assaying OR activation comprise an adenylate cyclase polypeptide or functional fragment thereof.

In certain embodiments, cells used for assaying OR activation comprise any one of REEP1, RTP1, RTP1S, RTP2, Gaolf, Ric-8b, HSP70, HSC70T, adenylate cyclase or the Type 3 muscarinic acetylcholine receptor M3, or any combination of one or more of these molecules. Functional fragments of the foregoing molecules are also contemplated.

In additional embodiments, OR sequences can be fused, at their amino- and/or carboxy-terminal ends, to sequences which target the OR to the host cell secretory apparatus for insertion into the cell membrane and/or to sequences that stabilize the receptor in the membrane.

In certain embodiments, assays for OR activation are conducted using cell extracts or membrane fractions from any of the cells described herein. Methods for making cell extracts and cell membrane fractions are known in the art. For example, cells are lysed in a blender with glass beads; cell debris is removed by centrifugation at, for example, 600×g, and a membrane fraction is obtained by ultracentrifugation at, for example, 104,300×g. See, for example, U.S. Patent Application Publication No. 2017/0242004 and WO 2019/036432.

In additional embodiments, eukaryotic cells other than 293T or Hana3 can be used. For example, a fungal cell can be used. The fungal cell can be from the Aspergillus, Trichoderma, Saccharomyces, Chrysosporium, Klyuveromyces, Candida, Pichia, Debaromyces, Hansenula, Yarrowia, Zygosaccharomyces, Schizosaccharomyces, Penicillium, or Rhizopus genera. The fungal cell can be a Saccharomyces cerevisiae. A eukaryotic cell derived from a mammal, for example, a human cell, or a cell derived from a non-human mammal such as a monkey, a mouse, a rat, a pig, a horse, or a dog can be used. Plant cells, algal cells and Archael cells can also be used.

B. Cell-Based Sensor Devices:

As noted above, disclosed herein are cell-based sensor devices and methods for use thereof. In some embodiments, the cell-based sensor devices of the present disclosure may comprise a single chamber within which at least one cell expressing one or more cell surface receptors and at least one electrode configured to measure electrical signals are positioned. In some embodiments, the cell-based sensor devices may comprise a plurality of chambers (e.g., an array of chambers), wherein each chamber comprises at least one cell expressing one or more cell surface receptors, and at least one electrode configured to measure electrical signals positioned within the chamber. In some embodiments, the number of chambers within the cell-based sensor device may range from 1 to about 100, or more. In some embodiments, the number of chambers in the cell-based sensor device may be at least 1, at least 5, at least 10, at least 20, at least 30, at least 40, at least 50, at least 60, at least 70, at least 80, at least 90, or at least 100 chambers. In some embodiments, the number of chambers in the cell-based sensor device may be at most 100, at most 90, at most 80, at most 70, at most 60, at most 50, at most 40, at most 30, at most 20, at most 10, at most 5, or at most 1 chamber. Any of the lower and upper values described in this paragraph may be combined to form a range included within the present disclosure, for example, the number of chambers within the cell-based sensor device may range from about 5 to about 20. Those of skill in the art will recognize that number of chambers within the cell-based sensor device may have any value within this range, e.g., 16 chambers. In some embodiments, the plurality of chambers within the cell-based sensor device may be organized as an array of chambers, e.g., and m×n array, where m is the number of rows of chambers and n is the number of columns of chambers in the array.

In some embodiments, the cell-based sensor device may further comprise inlet ports, outlet ports, fluid channels (e.g., inlet channels, outlet channels, perfusion channels, etc.), valves, membranes (e.g., gas exchange membranes, filter membranes, dialysis membranes, or ion exchange membranes), etc., that are fluidically coupled to one or more of the chambers within the cell-based sensor device. In some embodiments, the cell-based sensor device may further comprise a gas exchange membrane comprising a polytetrafluoroethylene (PTFE) membrane having a pore size in the range of 0.2 to 0.5 micrometers.

Any of a variety of cell types known to those of skill in the art may be used in the cell-based sensor devices of the present disclosure. In some embodiments, each chamber of a cell-based sensor device may comprise a single cell. In some embodiments, each chamber of a cell-based sensor device may comprise two cells, three cells, four cells, five cells, ten cells, twenty cells, thirty cells, forty cells, fifty cells, or more. In some embodiments, each chamber of a plurality of chambers within a cell-based sensor device may comprise the same cell or set of cells. In some embodiments, a subset of chambers or all of the chambers of a plurality of chambers with a cell-based sensor device may comprise a different cell or set of cells.

Typically, the cell(s) within each chamber of the device are bathed in a cell culture medium that is continuously, periodically, or randomly perfused through each chamber of the plurality of chambers in order to maintain the viability of the cells therein. The medium may include one or more components, including but not limited to, sodium chloride, glycine, l-alanine, 1-serine, a neuroactive inorganic salt, 1-aspartic acid, 1-glutamic acid, or any combination thereof. A medium may further include one or more of a pH modulating agent, an amino acid, a vitamin, a supplemental agent, a protein, an energetic substrate, a light-sensitive agent, or any combination thereof. A medium may further include one or more buffering agents. A medium may further include one or more antioxidants.

Typically, the composition and perfusion rate of the cell culture medium, as well as and other operational parameters, e.g., temperature, pH of the medium, CO₂ concentration in the medium, etc., are optimized to maintain cell viability of the cell(s) within the chamber(s) of the cell-based sensor device. In some embodiments, the life span of the cells within the device may range from about 1 week to about 1 year. In some embodiments, the life span of cells with the device may be at least 1 week, at least 2 weeks, at least 1 month, at least 2 months, at least 3 months, at least 4 months, at least 5 months, at least 6 months, at least 7 months, at least 8 months, at least 9 months, at least 10 months, at least 11 months, at least 1 year, at least 1.2 years, at least 1.4 years, at least 1.6 years, at least 1.8 years, or at least 2 years.

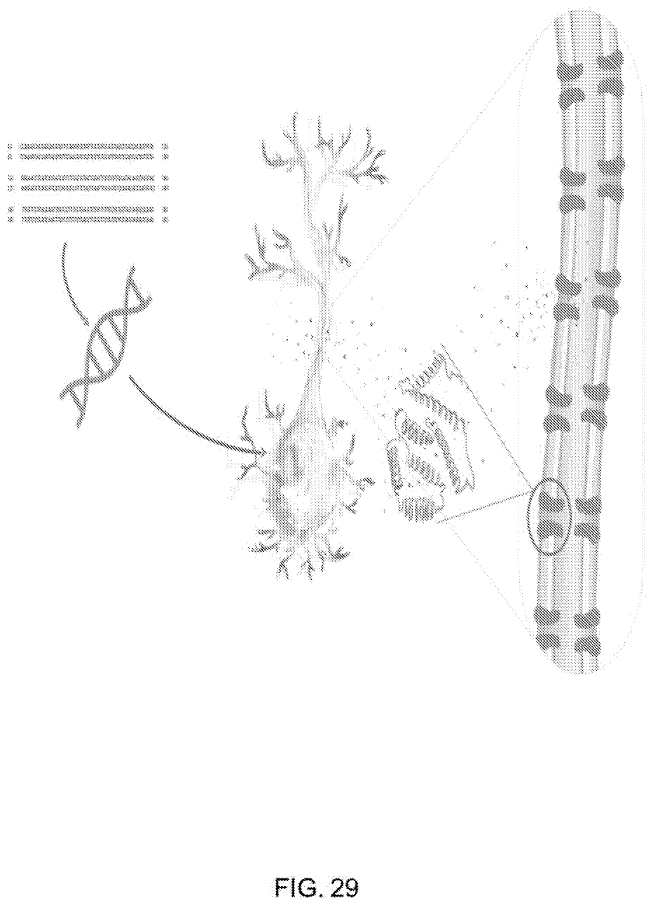

In a preferred embodiment, the cells within the chamber(s) of a cell-based sensor device may comprise other excitable cells, e.g., neurons, astrocytes, embryonic kidney cells or other cells that have been genetically-engineered to express one or more types of cell surface receptor. FIG. 29 shows a non-limiting example of the expression of olfactory receptors on a neuron. Any of a variety of cell surface receptors known to those of skill in the art may be used in the disclosed cell-based sensor device. Examples include, but are not limited to, odorant receptors, taste receptors, light-sensitive ion channels or other photoreceptor proteins, etc. Specific examples of suitable cell surface receptors will be described in more detail below. In some embodiments, the type of neuron used may be the same for each chamber in the plurality of chambers within the cell-based sensor device. In some embodiments, the type of neuron used may be different for different chambers of the plurality of chambers within the cell-based sensor device. In some embodiments, the type of neuron used in the sensor device may be selected base on a low level of naturally occurring cell surface receptors in order to minimize random and or background electrical signal generation. In some embodiments, the neuron used in the sensor device may be a neuron that has been modified, e.g., genetically modified, to suppress or eliminate the expression of naturally occurring cell surface receptors.

In another preferred embodiment, the cell-based sensor devices of the present disclosure may comprise an array of neurons that may be engineered to express cell surface receptors (i.e., odorant receptors) to detect volatile or water-soluble odorant compounds. Each neuron within the array may express a single type of chemical sensing protein receptor or multiple types of chemical sensing protein receptors that detect a set of ligands (e.g., odorant compounds). Upon binding of a ligand such as an odorant compound to a cell surface receptor, activation of a series of intracellular signaling proteins or pathways may trigger an action potential by the neuron.

Compounds in fluid or gaseous samples may be introduced to the cell-based sensor device either by mixing with the medium that bathes the cells in the device, or by passive diffusion (e.g., in the case of volatile compounds present in an air sample) through a semi-permeable membrane that is integrated with the sensor device. In some embodiments, the use of an air sampling device may be used to facilitate the introduction of compounds into the cell-based sensor device, as will be discussed in more detail below.

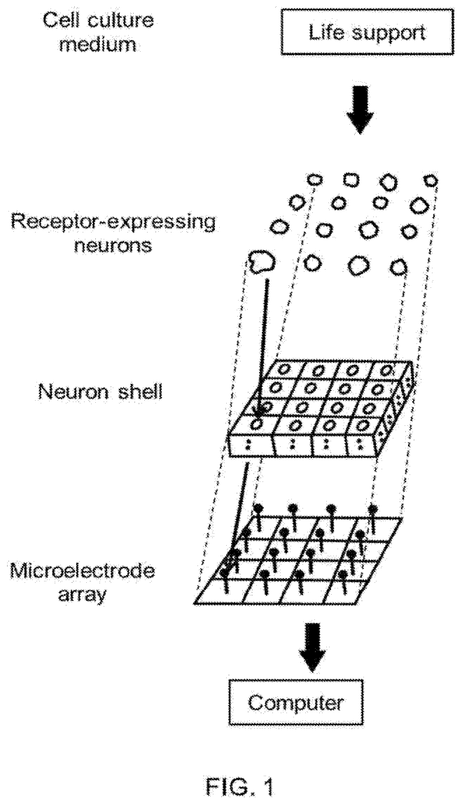

Binding events between a compound (or mixture of compounds) introduced into the medium bathing the cells and one or more of the cell surface receptors present in the cells within the device may give rise to electrical signals, e.g., changes in cell surface electrostatic potentials or cell membrane depolarizations, that are detected by the electrode in each corresponding chamber. In some embodiments, the plurality electrodes associated with the plurality of chambers within the cell-based sensor device (i.e., one or more electrodes per chamber) may comprise a microelectrode array (MEA). FIG. 1 provides a schematic illustration of a cell-based sensor device of the present disclosure that comprises neurons that have been genetically-engineered to express selected cell surface receptors, where the neurons are located within an array of chambers (i.e., “neuron shell”) that is in contact with the MEA. In cell-based sensor devices comprising, e.g., neurons, each neuron cell may be associated with (e.g., in close proximity to, connected to, or penetrated by) an electrode in the microelectrode array (MEA), which may permit the detection of depolarization of the neuron membrane following the binding of, e.g., an odorant to the cell surface receptor. This electrical signal generated by the cell may be detected by the electrode and transferred to a processor or computer input device, e.g., a data acquisition board comprising an analog to digital converter. In aggregate, the cells of the cell-based sensor device may differentially detect an array of compounds, which collectively may yield a “fingerprint” of electrical signals used to detect and identify compounds or mixtures of compounds. In some embodiments, the cell-based sensors of the present disclosure may provide qualitative data for the detection and identification of specific compounds or mixtures of compounds. In some embodiments, the cell-based sensors of the present disclosure may provide quantitative data for the detection and identification of specific compounds or mixtures of compounds, for example, the sensor data may provide an measure of the concentration of a specific compound present in an air sample, or the relative concentrations of a mixture of compounds present in an air sample.

In some embodiments, the cell-based sensor device may comprise an array of m×n cells (i.e., within an array of m×n chambers). A single odorant may bind to a cell expressing a single type of odorant receptor. The binding event may then activate a signaling pathway within the cell. If the cell is a neuron, then it may trigger an action potential which can be detected by the electrode inserted in or in close proximity to the cell. If the binding event does not trigger a full action potential, the electrode inserted in or in close proximity to the cell may permit detection of a sub-threshold level electrical signal.

In some embodiments, an array of cells within the sensor device may comprise cells each expressing, e.g., a unique odorant receptor. An odorant may bind differentially across the cells such that each cell generates a different electrical signal, e.g., a different electrical signal level having an amplitude that ranges between zero and that for a full action potential, or a different electrical signal frequency, e.g., a different burst frequency.

Through repeated delivery of a single odorant or set of odorants with known characteristics to the cell-based sensor device, a series of relative signals generated across the array of cells may be observed, detected, or collected. The signal values may be contained within a matrix comprising the different levels of electrical signal detected for each cell, based on sub-threshold and full-threshold electrical signals generated by the neurons.

As noted, the signal levels may be represented in a matrix where each element may represent a real valued amplitude, aij, which may represent the sub-threshold signal level or that for a full on/off action potential, and i and j represent the position coordinates of the cell/electrode combination in the array of the sensor device:

$${a}_{00} {a}_{01} {a}_{02} {a}_{03} … {a}_{0 n}$$

$${a}_{10} {a}_{11} {a}_{12} {a}_{13} … {a}_{1 n}$$

$${a}_{20} {a}_{21} {a}_{22} {a}_{23} … {a}_{2 n}$$

$${a}_{30} {a}_{31} {a}_{32} {a}_{33} … {a}_{3 n}$$

$$…$$

$${a}_{m 0} {a}_{m 1} {a}_{m 2} {a}_{m 3} … {a}_{mn}$$

In some embodiments, a compound may bind to different receptors at different rates (i.e., with different kinetics), since the binding of a ligand to G protein coupled receptors (GPCRs) requires three-dimensional coordination between the molecular features of the ligand and those within the binding site of the receptor. Some receptor binding sites may or may not recognize particular moieties or chemical substituents (e.g., OH, CH3, NH2, or COOH groups, etc.) which may decorate the compound of interest; rather it may be the combination of molecular features of the compound that provide a given ligand the “shape” or conformation that enables binding within a given GPCR binding pocket. Thus, in some cases, different parts, e.g., specific moieties or functional groups, of the ligand may bind to different receptors at different rates or with different affinities and trigger different signals in different cells on the array. In some embodiments, calibration of the sensor device using calibration curves generated by exposing the sensor device to a series of compounds at varying concentration may be used to correct for systematic biases due, for example, to differences in the solubility of the compounds in the liquid medium bathing the cells.

In some embodiments, a single compound may give rise to a fixed set of signal values in the signal level matrix, with a range of amplitude variation across all non-zero values. This may be used as a signal fingerprint for that particular compound.

In some embodiments, a set of compounds (related or unrelated) may have a particular signal fingerprint when mapped against a particular set of receptors in a cell-based sensor device. This signal fingerprint for a set of compounds may represent an overlapping set of the signal fingerprints for binding of individual compounds. That is, one may expect the individual compounds in the set of compounds to bind to more than one receptor in different ways. The entire set may be additive across the array. However, the signals generated by binding of some compounds may mask the signals generated by others. Each combination of compounds may yield a unique signal fingerprint or signature generated by the array of cells within the sensor device.

In some embodiments, there may be a single electrode in each chamber (or microwell) of the cell-based sensor device. In some embodiments there may be two or more electrodes in each chamber of the cell-based sensor device. In some embodiments, there may be at least one electrode, at least two electrodes, at least three electrodes, at least four electrodes, at least five electrodes, at least six electrodes, at least seven electrodes, at least eight electrodes, at least nine electrodes, or at least ten electrodes in each chamber of the plurality of chambers within the sensor device. In some embodiments, a single ground electrode may be placed in contact with the culture medium bathing the cells within the device. In some embodiments, at least one of the electrodes in each chamber of the plurality of chambers within the device may be a ground electrode.

In some embodiments, the electrodes used in the cell-based sensor devices of the present disclosure may comprise two-dimension (i.e., planar) electrodes or three dimensional (e.g., hemispherical) electrodes fabricated from any of a variety of materials known to those of skill in the art. Examples include, but are not limited to, metals, metal alloys, and metal oxides, e.g., aluminum, gold, lithium, copper, graphite, carbon, titanium, brass, silver, platinum, palladium, cesium carbonate, molybdenum (VI) oxide, indium tin oxide (ITO), or any combination thereof.

In some embodiments, the surface of the electrode may comprise a chemically modified gold surface, wherein proteins like laminins, non-specific DNA, peptides, conductive polymers, other chemicals or compounds, or any combination thereof are grafted to the surface to improve neural adhesion and signal quality.

In some embodiments, modifying an electrode surface with a plurality of protrusions, a plurality of recesses, or by adding surface roughness may increase the surface area of the electrode and enhance contact between a cell and the electrode, thereby improving the electrical connection between the cell and the electrode.

In some embodiments, a three-dimensional electrode may comprise a spherical shape, a hemispherical shape, a mushroom shape (i.e., comprising ahead portion and a support portion), a rod-like shape, a cylindrical shape, a conical shape, a patch shape, or any combination thereof.

In some embodiments, the width of an electrode (e.g., the width of the narrowest portion of a two-dimensional electrode, or the base or support portion of a three-dimensional electrode) may range from about 1 micrometer (μm) to about 50 micrometers (μm). In some embodiments, the width of an electrode may be at least 1 μm, at least 5 μm, at least 10 μm, at least 20 μm, at least 30 μm, at least 40 μm, or at least 50 μm. In some embodiments, the width of an electrode may be at most 50 μm, at most 40 μm, at most 30 μm, at most 20 μm, at most 10 m, at most 5 μm, or at most 1 μm. Any of the lower and upper values described in this paragraph may be combined to form a range included within the present disclosure, for example, the width of an electrode may range from about 10 to about 30 μm. Those of skill in the art will recognize that the width of an electrode may have any value within this range, e.g., about 22.5 μm.

In some embodiments, the thickness or height of an electrode (i.e., the thickness of a two-dimensional electrode, or the height of a three-dimensional electrode relative to the substrate on which it is fabricated) may range from about 0.1 micrometer (m) to about 50 micrometers (m). In some embodiments, the thickness or height of an electrode may be at least 0.1 μm, at least 1 μm, at least 5 μm, at least 10 μm, at least 20 μm, at least 30 μm, at least 40 μm, or at least 50 μm. In some embodiments, the thickness or height of an electrode may be at most 50 μm, at most 40 μm, at most 30 μm, at most 20 μm, at most 10 μm, at most 5 μm, at most 1 μm, or at most 0.1 μm. Any of the lower and upper values described in this paragraph may be combined to form a range included within the present disclosure, for example, the thickness or height of an electrode may range from about 0.1 to about 10 μm. Those of skill in the art will recognize that the thickness or height of an electrode may have any value within this range, e.g., about 28.6 μm.

In some embodiments, an electrode may have a surface density of protrusions ranging from about 0.0001 protrusions per square micrometer (pro/μm²) to about 10 protrusions per square micrometer (pro/μm²). In some embodiments, the surface density of protrusions on an electrode may be at least 0.0001, at least 0.0005, at least 0.001, at least 0.002, at least 0.003, at least 0.004, at least 0.005, at least 0.006, at least 0.007, at least 0.008, at least 0.009, at least 0.01, at least 0.02, at least 0.03, at least 0.04, at least 0.05, at least 0.06, at least 0.07, at least 0.08, at least 0.09, at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 2, at least 3, at least 4, at least 5, at least 6, at least 7, at least 8, at least 9, or at least 10 protrusions per square micrometer. In some embodiments, the surface density of protrusions on an electrode may be at most 10, at most 9, at most 8, at most 7, at most 6, at most 5, at most 4, at most 3, at most 2, at most 1.5, at most 1.4, at most 1.3, at most 1.2, at most 1.1, at most 1, at most 0.9, at most 0.8, at most 0.7, at most 0.6, at most 0.5, at most 0.4, at most 0.3, at most 0.2, at most 0.1, at most 0.09, at most 0.08, at most 0.07, at most 0.06, at most 0.05, at most 0.04, at most 0.03, at most 0.02, at most 0.01, at most 0.009, at most 0.008, at most 0.007, at most 0.006, at most 0.005, at most 0.004, at most 0.003, at most 0.002, at most 0.001, at most 0.0005, or at most 0.0001 protrusions per square micrometer. Any of the lower and upper values described in this paragraph may be combined to form a range included within the present disclosure, for example, the surface density of protrusions on an electrode may range from about 0.001 to about 1.1 protrusions per square micrometer. Those of skill in the art will recognize that the surface density of protrusions on an electrode may have any value within this range, e.g., about 0.015 protrusions per square micrometer.

Similarly, in some embodiments, an electrode may have a surface density of recesses ranging from about 0.0001 recesses per square micrometer (recesses/μm²) to about 10 recesses per square micrometer (recesses/μm²). In some embodiments, the surface density of recesses on an electrode may be at least 0.0001, at least 0.0005, at least 0.001, at least 0.002, at least 0.003, at least 0.004, at least 0.005, at least 0.006, at least 0.007, at least 0.008, at least 0.009, at least 0.01, at least 0.02, at least 0.03, at least 0.04, at least 0.05, at least 0.06, at least 0.07, at least 0.08, at least 0.09, at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 2, at least 3, at least 4, at least 5, at least 6, at least 7, at least 8, at least 9, or at least 10 recesses per square micrometer. In some embodiments, the surface density of recesses on an electrode may be at most 10, at most 9, at most 8, at most 7, at most 6, at most 5, at most 4, at most 3, at most 2, at most 1.5, at most 1.4, at most 1.3, at most 1.2, at most 1.1, at most 1, at most 0.9, at most 0.8, at most 0.7, at most 0.6, at most 0.5, at most 0.4, at most 0.3, at most 0.2, at most 0.1, at most 0.09, at most 0.08, at most 0.07, at most 0.06, at most 0.05, at most 0.04, at most 0.03, at most 0.02, at most 0.01, at most 0.009, at most 0.008, at most 0.007, at most 0.006, at most 0.005, at most 0.004, at most 0.003, at most 0.002, at most 0.001, at most 0.0005, or at most 0.0001 recesses per square micrometer. Any of the lower and upper values described in this paragraph may be combined to form a range included within the present disclosure, for example, the surface density of recesses on an electrode may range from about 0.005 to about 1.6 recesses per square micrometer. Those of skill in the art will recognize that the surface density of recesses on an electrode may have any value within this range, e.g., about 0.68 recesses per square micrometer.

In some embodiments, the surface of an electrode may be smooth. In some embodiments, the surface of an electrode may have a surface roughness. A surface roughness may be uniform across the surface of an electrode. A portion of the surface of an electrode may have a surface roughness, such as a top portion of the electrode, or a bottom portion of the electrode. An electrode may have alternating rows of smooth and rough portions.

In some embodiments, a surface roughness may be about 5, 10, 15, 20, 25, 30, 35, 50, 100, 200, 300, 400, 500, 600, 700, 800, 900, 1000, 1500, 2000, 2500, 3000 nanometers (nm) or more. In some embodiments, a surface roughness may be from about 5 to about 50 nm. In some embodiments, a surface roughness may be from about 5 to about 100 nm. In some embodiments, a surface roughness may be from about 5 to about 500 nm. In some embodiments, a surface roughness may be from about 10 to about 50 nm. In some embodiments, a surface roughness may be from about 10 to about 100 nm. In some embodiments, a surface roughness may be from about 10 to about 500 nm.

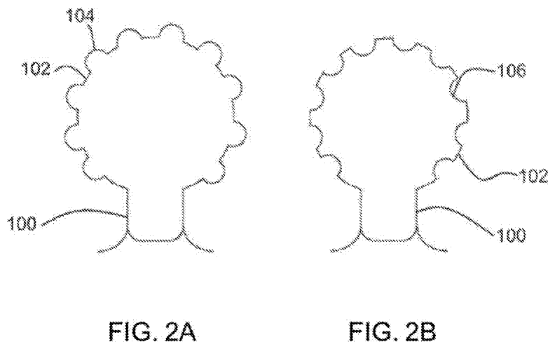

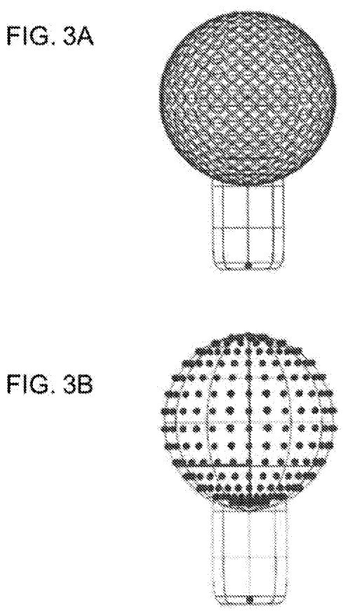

FIGS. 2A and 2B show schematic cross-sectional views of suitable electrode structures for use with embodiments of the disclosed cell-based sensor devices. In FIG. 2A, the electrode structure has a generally spherical form, standing on columnar support 100. The sphere surface 102 has an array of rounded protrusions 104. In FIG. 2B, the electrode has a corresponding format, except that protrusions 104 are replaced by depressions 106. The effect of this is to provide additional surface area and surface features for interaction with cells, which may facilitate detection of electrical signals. FIGS. 3A and 3B correspond to FIGS. 2A and 2B, showing front views of suitable electrode structures with depressions or protrusions, respectively.

In some embodiments, the electrodes of the microelectrode array (or plurality of electrodes associated with the plurality of chambers containing cells with the device) may be used to stimulate cells as well as record electrical signals generated by the cells in response to ligand binding. For example, in some embodiments, one or more electrodes in each chamber may be used to trigger action potentials in neurons in order to calibrate the electrical signals recorded by the measurement electrodes and/or normalize the electrical signal levels recorded for different chambers or for chambers comprising neurons expressing different levels and/or different types of cell surface receptors. In some embodiments, one or more electrodes in each chamber may be used to stimulate the cells to assay the health of the cells, to measure an increase in the impedance of the cell-electrode interface, or to establish a baseline reading for that particular electrode to determine what a spike train signal for stimulated cells might look like in a detection event (i.e., to establish how many cells are in close proximity or contact with the electrode, what the electrical signal waveforms from these cells look like, to prepare for bursting behavior, etc.).

In some embodiment, the cell-based sensor device may be “tuned” to improve the detection sensitivity for a specific compound or mixture of compounds, e.g., by controlling the types of receptors on the array and/or their position within the array of chambers. The type of neuron chosen for use in expressing a given receptor, e.g., an odorant receptor, may be selected on the basis of different background receptor expression levels and/or different background electrical signals (e.g., firing frequencies).

In some embodiments, the detection sensitivity of the disclosed cell-based sensor devices, or of the sensor panels and detection systems comprising said devices, may be adjusted by any of a variety of techniques known to those of skill in the art. Examples include, but are not limited to: (i) addition of one or more “odorant binding proteins” (e.g., soluble proteins that specific odorant molecules and improve their solubility and/or facilitate interaction with an odorant receptor) to the liquid medium bathing the cells in the device, (ii) addition of one or more compound stabilization additives (e.g., colloidal zinc) that stabilize the solubility of volatile organic compounds in solution to the liquid medium bathing the cells, (iii) by genetically engineering one or more of the receptors expressed by the cells within the device to enhance binding affinity and/or the electrical response of the cell, (iv) by overexpressing or underexpressing the receptors in one or more of the cell types within the device, (v) by genetically engineering one or more components of the intracellular signaling pathway to tune the sensitivity and electrical response of the cells within the device, (vi) by addition or genetic engineering of one or more synthetic signaling components to enhance the sensitivity and electrical response of the cells within the device, or (vii) by genetically deleting one or more naturally-occurring signaling components within the cells.

In some embodiments, the cell-based sensor device may comprise a processor for processing the patterns of electrical signals (or fingerprints) detected by the plurality of electrodes within the device. In some embodiments, the processor may be external to the cell-based sensor device. In some embodiments, machine learning-based processing of the patterns of electrical signals may be used to improve the sensitivity and/or specificity of the cell-based sensor device for detection of specific compounds or mixtures of compounds, e.g., using a machine learning algorithm that has been trained using training data sets comprising paired sets of the patterns of electrical signals (or “fingerprints”) measured in response to exposure of the cell-based sensor device to specific compounds or mixtures of compounds at known concentrations. In some embodiments, the machine learning-based analysis may allow correcting for systematic bias in the detection sensitivity for different compounds arising from, e.g., differences in the solubility of different compounds in the medium bathing the cells, variations in the numbers of cell surface receptors expressed in different cell types, etc. Examples of suitable machine learning-based algorithms and training data sets will be described in more detail below.

The cell-based sensor devices and sensor panels of the present disclosure may be fabricated using any of a variety of techniques and materials known to those of skill in the art. In general, the sensor devices or sensor panels, or components thereof, may be fabricated either as monolithic parts or as an assembly of two or more separate parts that are subsequently mechanically clamped, fastened, or permanently bonded together. Examples of suitable fabrication techniques include, but are not limited to, conventional machining, CNC machining, injection molding, 3D printing, alignment and lamination of one or more layers of laser or die-cut polymer films, or any of a number of microfabrication techniques such as photolithography and wet chemical etching, dry etching, deep reactive ion etching, or laser micromachining, or any combination of these techniques. Once the sensor device or sensor panel part(s) have been fabricated, they may be fastened together using any of a variety of fasteners, e.g., screws, clips, pins, brackets, and the like, or may be bonded together using any of a variety of techniques known to those of skill in the art (depending on the choice of materials used), for example, through the use of anodic bonding, thermal bonding, ultrasonic welding, or any of a variety of adhesives or adhesive films, including epoxy-based, acrylic-based, silicone-based, UV curable, polyurethane-based, or cyanoacrylate-based adhesives.

The cell-based sensor devices and sensor panels of the present disclosure may be fabricated using a variety of materials known to those of skill in the art. Examples of suitable materials include, but are not limited to, silicon, fused-silica, glass, any of a variety of polymers, e.g., polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, metals (e.g., aluminum, stainless steel, copper, nickel, chromium, and titanium), or any combination of these materials.

In some embodiments, the cell-based sensor devices of the present disclosure, or one or more individual chambers of the plurality of chambers contained therein, may further comprise one or more additional components for use in regulating the microenvironment of the cells within the sensor device and maintaining cell viability. Examples include, but are not limited to, heating elements, cooling elements, temperature sensors, pH sensors, gas sensors (e.g., O2 sensors, CO2 sensors), glucose sensors, optical sensors, electrochemical sensors, opto-electric sensors, piezoelectric sensors, magnetic stirring/mixing components (e.g., micro stir bars or magnetic beads that are driven by an external magnetic field), etc., or any combination thereof. In some embodiments, the cell-based sensors of the present disclosure may further comprise additional components or features, e.g., transparent optical windows, microlens components, or light-guiding features to facilitate microscopic observation or spectroscopic monitoring techniques, inlet and outlet ports for making connections to perfusion systems, electrical connections for connecting electrodes or sensors to external processors or power supplies, etc. In some embodiments, the cell-based sensors of the present disclosure may further comprise a grid of LEDs positioned underneath the cells, e.g., neurons, within the plurality of chambers which may be used to stimulate the neurons optogenetically to assay cell health in situations where the health or response accuracy of the cells may be suspect. In some embodiments, the disclosed sensor devices may further comprise a controller (separately or in addition to the processor discussed above) configured to control heating and/or cooling elements, and/or to send instructions to and/or read data from one or more sensors.

In some embodiments, the disclosed cell-based sensor devices, or sensor panels comprising grids of cell-based sensor devices, may detect a presence or an absence of a compound in a liquid sample at a concentration detection limit ranging from about 10 millimolar (mM) to about 1 picomolar (pM), or less. In some embodiments, the concentration detection limit may be better than 10 mM, better than 5 mM, better than 1 mM, better than 100 micromolar (uM), better than 50 uM, better than 10 uM, better than 5 uM, better than 1 uM, 100 nanomolar (nM), better than 50 nM, better than 10 nM, better than 5 nM, better than 1 nM, better than 100 pM, better than 50 pM, better than 10 pM, better than 5 pM, or better than 1 pM. In some embodiments, the concentration detection limit may be compound specific.

In some embodiments, the disclosed cell-based sensor devices, or sensor panels comprising grids of cell-based sensor devices, may detect a presence or absence of a compound in a gas or air sample with a detection limit ranging from 100 parts per million (ppm) to 0.1 parts per billion (ppb), or less. In some embodiments, the detection limit may be better than 100 ppm, better than 10 ppm, better than 1 ppm, better than 100 ppb, better than 10 ppb, better than 1 ppb, or better than 0.1 ppb. In some embodiments, the concentration detection limit may be compound specific.

Sensitivity may refer to a value calculated according to the formula TP)/(TP+FN), where TP is the number of true positive measurements (e.g., correctly detecting a presence of a compound in an environment or sample) and FN is the number of false negative measurements (e.g., incorrectly detecting an absence of a compound in an environment or sample). In some embodiments, the disclosed cell-based sensor devices, or sensor panels comprising grids of cell-based sensor devices, may detect a presence or an absence of one or more compounds at a sensitivity of greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds. In some cases, increasing the number of unique odorant receptors within the microelectrode array sensor device may increase the sensitivity of detection for one or more compounds.

Specificity may refer to a value calculated according to the formula TN/(TN+FP), where TN is the number of true negative measurements (e.g., correctly detecting an absence of a compound in an environment or sample) and FP is the number of false positive measurements (e.g., incorrectly detecting a presence of a compound in an environment or sample). In some embodiments, the disclosed cell-based sensor devices, or sensor panels comprising grids of cell-based sensor devices, may detect a presence or an absence of one or more compounds at a specificity of greater than about: 70%, 75%, 80%, 85%, 86%, 87%, 88%, 89%, 90%, 91%, 92%1, 93%, 94%, 95%, 96%, 97%, 98%, 99%, or 99.5% for the one or more compounds. In some cases, increasing the number of unique odorant receptors within the microelectrode array sensor device may increase the sensitivity of detection for one or more compounds.

Positive Predictive Value (PPV) may refer to a value calculated according to the formula TP/(TP+FP). A PPV value may be the proportion of samples with positive test results that correctly detect a presence or an absence of a compound. In some embodiments, the disclosed cell-based sensor devices, or sensor panels comprising grids of cell-based sensor devices, may detect a presence or an absence of one or more compounds at a PPV of greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds.

Negative Predictive Value (NPV) may refer to a value calculated according to the formula TN/(TN+FN). In some embodiments, the disclosed cell-based sensor devices, or sensor panels comprising grids of cell-based sensor devices, may detect a presence or an absence of one or more compounds at an NPV of greater than about: 70%, 75%, 80%, 85%, 86%, 87%, 88%, 89%, 90%, 91%%, 92%, 93%, 94%, 95%, 96%, 97%, 98%, 99%, or 99.5% for the one or more compounds.

In some embodiments, the disclosed cell-based sensor devices, or sensor panels comprising grids of cell-based sensor devices, may detect a presence or an absence of one or more compounds at an accuracy of greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds.

In some embodiments, the disclosed cell-based sensor devices, or sensor panels comprising grids of cell-based sensor devices, may detect a presence or an absence of one or more compounds at a confidence level of greater than about 70%, 75%, 80%, 85%, 86%, 87%, 88%, 89%, 90%, 91%, 92%, 93%, 94%, 95%, 96%, 97%, 98%, 99%, or 99.5% for the one or more compounds.

In some embodiments, the disclosed cell-based sensor devices, or sensor panels comprising grids of cell-based sensor devices, may detect a presence or an absence of one or more compounds at one or more of a sensitivity, a specificity, a PPV, an NPV, an accuracy, a confidence level, or any combination thereof at greater than about: 70%, 75%, 80%, 85%, 86%, 87%, 88%, 89%, 90%, 91%, 92%, 93%, 94%, 95%, 96%, 97%, 98%, 99%, or 99.5% for the one or more compounds.

Sensor panels: Also disclosed herein are sensor panels comprising two or more individual cell-based sensor devices, wherein each cell-based sensor device has been designed and/or optimized (e.g., by virtue of choosing the types of cells and/or cell surface receptors expressed in each of the plurality of chambers within each cell-based sensor device) to detect a different compound or mixture of compounds, such that the sensor panel is designed and/or optimized to detect two or more different compounds or mixtures of compounds.

In some embodiments, a sensor panel may comprise a single cell-based sensor device, e.g., when deployed as part of a detection system comprising two or more sensor panels positioned at different locations, as will be described in more detail below.

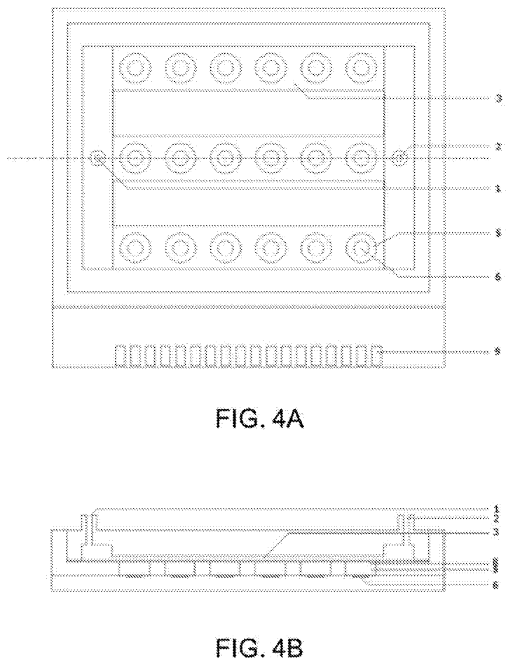

FIGS. 4A-B provide schematic illustrations (top and side views, respectively) of one non-limiting example of a cell-based sensor device of the present disclosure comprising a 3×6 grid of individual chambers or microwells within which one or more cells are compartmentalized. Cell culture medium enters the device through medium inlet 1, is delivered to cells in the microwells 5 via microfluidic channels 3, and exits the device via medium outlet 2. Each microwell 5 comprises an active electrode region 6, e.g., one or more electrodes that collectively constitute the microelectrode array component of the individual cell-based sensor device, as illustrated in FIG. 1. The device may comprise an anti-shear stress membrane 8, as well as a contact for complementary electronics 9. In some embodiments, a plurality of these cell-based sensor devices may be used to fabricate a sensor panel of the present disclosure, wherein the sensor panel comprises an array or grid of cell-based sensor devices. In some embodiments, the individual cell-based sensor devices within a sensor panel may all be in fluid communication with each other. In some embodiments, only a subset of the individual cell-based sensor devices within a sensor panel may be in fluid communication with each other. In some embodiments, none of the individual cell-based sensor devices within a sensor panel may be in fluid communication with each other.

In some embodiments, a sensor panel may comprise two individual cell-based sensor devices. In some embodiments, a sensor panel may comprise any number of individual cell-based sensor devices in the range from about 2 to about 100. In some embodiments, the number of cell-based sensor devices in the sensor panel may be at least 2, at least 5, at least 10, at least 20, at least 30, at least 40, at least 50, at least 60, at least 70, at least 80, at least 90, or at least 100. In some embodiments, the number of cell-based sensor devices in the sensor panel may be at most 100, at most 90, at most 80, at most 70, at most 60, at most 50, at most 40, at most 30, at most 20, at most 10, at most 5, or at most 2. Any of the lower and upper values described in this paragraph may be combined to form a range included within the present disclosure, for example, the number of cell-based sensor devices in the sensor panel may range from about 5 to about 20. Those of skill in the art will recognize that number of cell-based sensor devices in the sensor panel may have any value within this range, e.g., 25.

In some embodiments, the individual cell-based sensor devices may be randomly distributed across a substantially planar substrate or support component that defines the architecture of the sensor panel. In some embodiments, the individual cell-based sensor devices may be regularly arrayed across a substantially planar substrate or support component. In some embodiments, the individual cell-based sensor device may be arrayed in circular, spiral, triangular, rectangular, or square array patterns (or any other regular geometric pattern). For example, the individual cell-based sensor devices may be arrayed as a 2×2 array, a 3×3 array, a 4×4 array, a 5×5 array, a 6×6 array, a 7×7 array, an 8×8 array, a 9×9 array, or a 10×10 array, etc. In some embodiments, the individual cell-based sensor devices may be positioned on a non-planar, three-dimensional support structure, e.g., on the faces of a cubical, rectangular cuboid, or spherical structure, or on the face(s) of any other regular or free-form three-dimensional structure.

In some embodiments, each individual cell-based sensor device may comprise a processor for processing the patterns of electrical signals detected by plurality of electrodes in each device. In some embodiments, the sensor panel may comprise a processor for processing the patterns of electrical signals detected by the plurality of electrodes in all cell-based sensor devices of the panel. In many embodiments, the processor for each individual cell-based sensor device or for the sensor panel may also provide a time-stamp for the electrical signal data collected by each cell-based sensor device in the panel. As noted above for the cell-based sensor devices, in some embodiments, machine learning-based processing of the patterns of electrical signals recorded by the plurality of electrodes in each of the cell-based sensor devices of the panel may be used to improve the sensitivity and/or specificity of the sensor panel for detection of specific compounds or mixtures of compounds, while correcting for systematic detection biases due, e.g., to differences in compound solubility in the cell culture medium, and minimizing signal cross-talk between the individual cell-based sensor devices. Examples of suitable machine learning-based algorithms and training data sets will be described in more detail below.

As with the individual cell-based sensor devices described above, in some embodiments the sensor panels may further comprise one or more additional components for use in regulating the microenvironment of the cells within the sensor device and maintaining cell viability. Examples include, but are not limited to, heating elements, cooling elements, temperature sensors, pH sensors, gas sensors (e.g., O2 sensors, CO2 sensors), glucose sensors, optical sensors, electrochemical sensors, opto-electric sensors, piezoelectric sensors, magnetic stirring/mixing components (e.g., micro stir bars or magnetic beads that are driven by an external magnetic field), etc., or any combination thereof. In some embodiments, the sensor panels of the present disclosure may further comprise additional components or features, e.g., transparent optical windows, microlens components, or light-guiding features to facilitate microscopic observation or spectroscopic monitoring techniques, inlet and outlet ports for making connections to perfusion systems, electrical connections for connecting electrodes or sensors to external processors or power supplies, etc. In some embodiments, the disclosed sensor panels may further comprise a controller (separately or in addition to the processors discussed above) configured to control heating and/or cooling elements, and/or to send instructions to and/or read data from one or more sensors.

C. Air Sampling Devices:

In some embodiments, the devices, systems and methods disclosed herein may comprise air sampling devices, or the use thereof, for facilitating transport of compounds, e.g., volatile compounds, from air into one or more cell-based sensor devices, e.g., into the one or more cell-based sensor devices of a sensor panel array that constitutes a detection system of the present disclosure. In general, these air sampling devices may employ any of a variety of strategies for enhancing transport of compounds from air into the cell-based sensor device, as will be discussed in more detail below.

In addition to air, devices of this disclosure can test liquids or solids. For example, liquids or solids can be put into contact with OR-expressing cells, e.g., in a multiwell plate, and a response determined.

1. Strategy a—Increasing the Surface Area of the Liquid/Gas Interface:

One approach to facilitating the transfer of volatile compounds from a gas, e.g., air, to a liquid, e.g., the cell culture medium bathing the cell in the cell-based sensor devices of the present disclosure, is to design air sampling and/or sensor devices that provide a liquid/gas interface having a large surface area across which diffusion may take place. Examples of suitable approaches include, but are not limited to, the use of semipermeable membrane-based devices, gas perfusion chambers, atomization, or any combination thereof.

2. Devices Comprising a Semi-Permeable Gas Exchange Membrane:

In some embodiments, cell culture medium may be perfused through an air-sampling device, e.g., a structure or panel, having a high surface area-to-volume ratio that is integrated with or positioned upstream of the cell-based sensor device or sensor panel. In some embodiments, the air-sampling device may consist of a series of microchannels that collectively present a large surface area for diffusion, where the liquid/gas interface is mediated by a semi-permeable gas exchange membrane (e.g., a PTFE membrane that has been engineered to be permeable to the volatile compound of interest but impermeable to the culture medium) that constitutes one boundary wall of the series of microchannels, thereby allowing for the exchange of volatile compounds between the air and the perfused medium. In some embodiments, the semi-permeable gas exchange membrane may comprise a hydrophobic or hydrophilic PTFE membrane of thickness ranging between about 10 micrometers to about 100 micrometers. In some embodiments, the thickness of the hydrophobic or hydrophilic PTFE membrane may be at least 10, at least 20, at least 30, at least 40, at least 50, at least 60, at least 70, at least 80, at least 90, or at least 100 micrometers. In some embodiments, the thickness of the hydrophobic or hydrophilic PTFE membrane may be at most 100, at most 90, at most 80, at most 70, at most 60, at most 50, at most 40, at most 30, at most 20, or at most 10 micrometers. Any of the lower and upper values described in this paragraph may be combined to form a range included within the present disclosure, for example, the thickness of the hydrophobic or hydrophilic PTFE membrane may range from about 20 to about 80 micrometers. Those of skill in the art will recognize that the thickness of the hydrophobic or hydrophilic PTFE membrane may have any value within this range, e.g., about 95 micrometers.

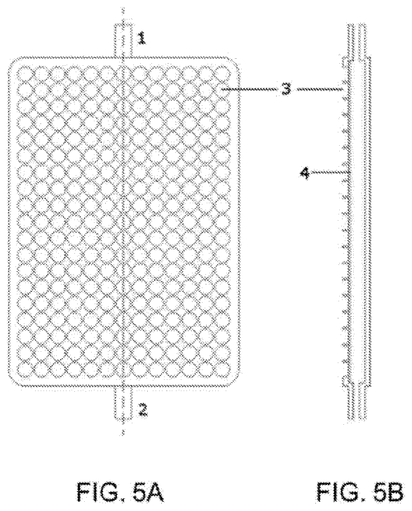

FIGS. 5A-B provide non-limiting schematic illustrations (top and side views, respectively) of an air-sampling device comprising a semi-permeable gas exchange membrane. Cell culture medium flows into the device via liquid inlet 1 and exits via liquid outlet 2. Openings 3 in a surface of the device allow gas or air samples to access the semi-permeable gas exchange membrane 4 and collectively provide for a large surface area in which volatile compounds may diffuse across the membrane and dissolve in the medium. The compound-containing culture medium is then transferred to a cell-based sensor device or sensor panel positioned downstream, e.g., by means of a microfluidics-based perfusion system.

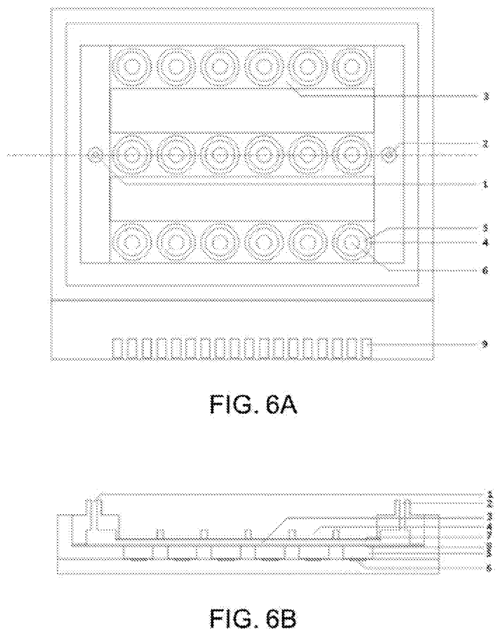

FIGS. 6A-B provide non-limiting schematic illustrations of a cell-based sensor device comprising an integrated semi-permeable gas exchange membrane. FIG. 6A provides a top view of the device. FIG. 6B provides is a side view of the device. In this example, the cell culture medium enters the device via liquid inlet 1, is delivered to the cells in microwells 5 via microfluidic channels 3, and exits via liquid outlet 2. Gas exchange occurs within openings 4 centered on the microwells 5 across semi-permeable membrane 7. The active electrode region is indicated as 6. The device also comprises an anti-shear stress membrane 8, and a contact for complementary electronics 9. In some embodiments, the layer of culture medium positioned between the cells (e.g., neurons) and the surface of the semi-permeable membrane may be no deeper than about 10 microns, about 20 microns, about 30 microns, about 40 microns, about 50 microns, about 100 microns, about 200 microns, about 300 microns, about 400 microns, or about 500 microns to minimize the path length that the volatile compound may need to traverse to reach the requisite receptors, e.g., odorant receptors, while still providing the cell layer with enough nutrients for long-term survival. New medium may be constantly perfused at a slow rate into the sensor device to introduce fresh nutrients and proteins, while old medium flows out to remove waste products, such as carbon dioxide, as well as dissolved compounds or particulates from previous exposures to a gas or air sample. In some embodiments, a plurality of such cell-based sensor devices may be arrayed to form a sensor panel. In some embodiments, by keeping all of the microelectrode array-based sensor devices on one panel in the same medium bath, the stability of the system may be increased and the ability of the medium to buffer any potentially deleterious changes in pH, dissolved oxygen concentration, and temperature may be improved.

In some embodiments, e.g., those in which a semi-permeable gas exchange membrane is incorporated into an air sampling device or integrated directly with a cell-based sensor device or sensor panel, the surface area-to-volume ratio for the semi-permeable membrane and the volume of liquid medium in contact with the semi-permeable membrane at a given instant may be greater than 1 cm⁻¹, 10 cm⁻¹, 100 cm⁻¹, or 1,000 cm⁻¹. The use of higher surface area-to-volume ratios in the device may facilitate efficient gas exchange and dissolution of volatile compounds into the cell culture medium.

Devices comprising a gas perfusion chamber: In some embodiments, the gas or air containing the volatile compounds of interest may be injected into cell culture medium contained using a micro bubbler within a small mixing chamber that is part of an air-sampling device positioned upstream of the cell-based sensor device or sensor panel. In some embodiments, a gas perfusion chamber and microbubbler may be directly integrated with a cell-based sensor device or sensor panel of the present disclosure.

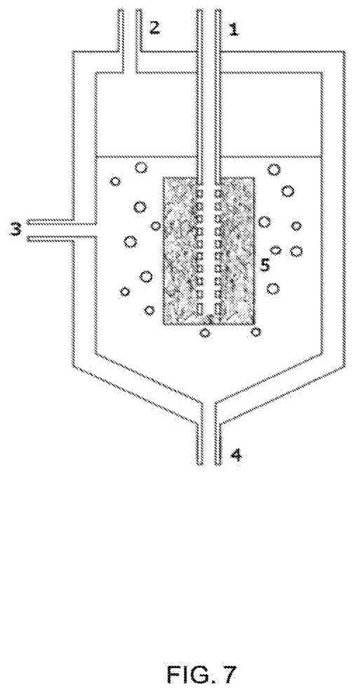

FIG. 7 provides a non-limiting schematic illustration of an air-sampling device comprising a perfusion chamber. The gas or air sample enters the device at gas inlet 1 and is forced to permeate through porous matrix 5 of the micro bubbler positioned in a small volume of cell culture medium entering the device via liquid inlet 3, thereby generating very fine bubbles that collectively comprise a large aggregate gas/liquid interfacial surface area and promote diffusive transfer of volatile compounds within the gas or air sample into the cell culture medium. The gas or air sample exits the device via gas outlet 2, and the loaded culture medium exits the device via liquid outlet 4 to be delivered, after appropriate degassing, to a cell-based sensor device or sensor panel located downstream from the perfusion chamber.

3. Devices Comprising an Atomizer:

In some embodiments, the gas or air containing the volatile compounds of interest may be injected into a small mixing chamber within the air-sampling device where it is atomized using ultrasonic frequencies in a technique commonly used in cool gas stream humidification. The resultant vapor may then be recondensed and injected into the culture medium that flows into the cell-based sensor device or sensor panel. In some embodiments, the mixing chamber and atomizer may be directly integrated with a cell-based sensor device or sensor panel of the present disclosure.

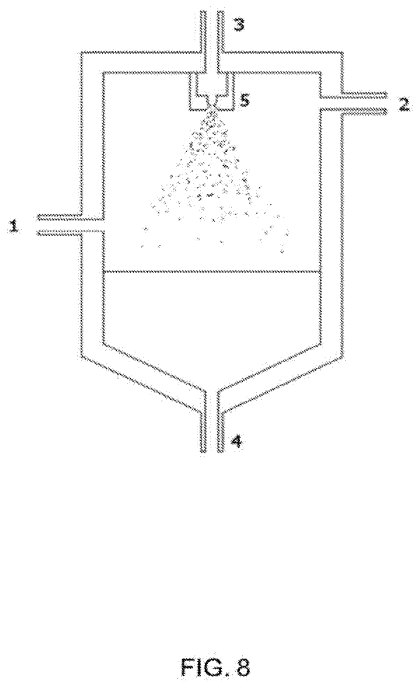

FIG. 8 provides a non-limiting schematic illustration of an air-sampling device comprising an atomizer. A gas or air sample comprising volatile compounds of interest enters the device via gas inlet 1 and exits via gas outlet 2. Culture medium enters the device via liquid inlet 3, and is forced through spray nozzle 5 that vibrates at ultrasonic frequencies to create a fine mist or vapor. The gas or air sample mixes with the vapor, which collectively comprises a large aggregate gas/liquid interfacial surface area and promotes diffusive transfer of volatile compounds within the gas or air sample into the vapor, following which the vapor is then condensed and the compound-loaded medium then exits the device via liquid outlet 4.

4. Strategy B—Increasing the Solubility of Volatile Compounds:

Another example of an approach to facilitate the transfer of volatile compounds from a gas, e.g., air, to a liquid, e.g., the cell culture medium bathing the cell in the cell-based sensor devices of the present disclosure, is to utilize methods for increasing the solubility of the compounds in the cell culture medium. Examples of suitable approaches include, but are not limited to, the use of a pressurized gas phase, heating the liquid phase, increasing the air velocity or pressure over the surface of a gas exchange membrane (e.g., by the inclusion of a fan), or any combination thereof.

5. Devices Comprising a Pressurized Gas Phase:

In some embodiments, the gas or air sample may be compressed and placed in contact with the cell culture medium within a closed mixing chamber that is part of an air-sampling device positioned upstream of a cell-based sensor device or sensor panel. Pressurization of the gas or air sample serves to increase the partial pressure of volatile compounds, thereby increasing the solubility of the volatile compounds in the cell culture solution according to Henry's law. The mixture may then be depressurized and delivered to the cell-based sensor device or sensor panel.

6. Devices Comprising a Heated Liquid Phase:

In some embodiments, the cell culture medium in which the volatile compounds are to be solubilized can be heated within an air-sampling device to increase the solubility of the compounds. The cell culture medium may then be cooled again to the specified temperature (e.g., 37 degrees C.) before reintroduction to a cell-based sensor device or sensor panel.

7. Devices Comprising a Dedicated Solvent:

In some embodiments, the volatile compounds may be dissolved in a liquid phase solvent that is different from the cell culture medium. For example, many organic volatiles may be far more soluble in polar, aprotic solvents like DMSO or acetone than in typical aqueous solutions used in cell culture. Gas or air samples comprising the volatile compounds of interest may be mixed with a solvent within an air-sampling device positioned upstream of a cell-based sensor device or sensor panel. In some embodiments, the loaded solvent may then be neutralized with another solution to create a nontoxic, biocompatible suspension prior to re-introduction into the stream of culture medium entering the cell-based sensor device or sensor panel.

In some embodiments, air-sampling devices of the present disclosure may utilize any combination of the strategies and approaches outline above to create a number of different final system configurations.

D. Detection Systems:

Also disclosed herein are detection systems which comprise one or a plurality of the cell-based sensor panels described above, where the detection systems provide a means for monitoring the air in a given space (e.g., an outdoor environment or an indoor/enclosed environment) for the presence of volatile compounds, e.g., volatile markers or taggants of explosive materials. In most embodiments, the two or more sensor panels of the detection system may be positioned at known locations within or around the environment to be monitored, and time-stamped data for the patterns of electrical signals recorded by each of the sensor devices in each sensor panel may be used, along with the known locations of the sensor devices/panels from which they arose, to both detect the presence of, and identify, a compound of mixture of compounds of interest, but also to locate the position of the source of the compound or mixture of compounds within the space.

In some embodiments, the detection systems of the present disclosure may comprise between 2 and about 200 panels, or more. In some embodiments, the detection system may comprise at least 2 sensor panels, at least 4 sensor panels, at least 6 sensor panels, at least 8 sensor panels, at least 10 sensor panels, at least 15 sensor panels, at least 20 sensor panels, at least 40 sensor panels, at least 60 sensor panels, at least 80 sensor panels, at least 100 sensor panels, or at least 200 sensor panels. In some embodiments, the detection system may comprise at most 200 sensor panels, at most 100 sensor panels, at most 80 sensor panels, at most 60 sensor panels, at most 40 sensor panels, at most 20 sensor panels, at most 15 sensor panels, at most 10 sensor panels, at most 8 sensor panels, at most 6 sensor panels, at most 4 sensor panels, or at most 2 sensor panels. Any of the lower and upper values described in this paragraph may be combined to form a range included within the present disclosure, for example, the number of sensor panels in the detection system may range from about 4 to about 80. Those of skill in the art will recognize that the number of sensor panels in the detection system may have any value within this range, e.g., 152.

In some embodiments, the two or more sensor panels may comprise the same complement of cell-based sensor devices, i.e., a set of cell-based sensor devices designed and/or optimized for detection of the same set of compounds or mixtures of compounds. In some embodiments, the two or more sensor panels may comprise different complements of cell-based sensor devices, i.e., sets of cell-based sensor devices designed and/or optimized for detection of a different set of compounds or mixtures of compounds.

In some embodiments, the detection system may further comprise two or more air sampling devices as described above, wherein each air sampling device is in fluid communication with one of the two or more sensor panels, and wherein each air sampling device is configured to facilitate the transfer compounds present in the air to the culture medium that bathes the cells in each of the chambers in each cell-based sensor device of the corresponding sensor panel.

In some embodiments, a detection system of the present disclosure may comprise a single air sampling device, two air sampling devices, three air sampling device, four air sampling devices, five air sampling devices, or more. In some embodiments, a detection system of the present disclosure may comprise at least one air sampling device for each sensor panel of the system. In some embodiments, a detection system of the present disclosure may comprise two or more air sampling devices for each sensor panel of the system. In some embodiments, detection systems comprising two or more air sampling devices may comprise two or more of the same type of air sampling device, or two or more different types of air sampling devices. Any combination of different air sampling devices may be used in the detection systems of the present disclosure.

In some embodiments, the detection system may comprise a controller comprising one or more processors configured to receive the electrical signals measured by the plurality of electrodes in each cell-based sensor device of the two or more sensor panels. In some embodiments, the controller stores and processes a pattern of electrical signals associated with a compound or mixture of compounds that is generated by at least one of the cell-based sensor devices in each of the two or more sensor panels (which are positioned at known locations) to identify the compound or mixture of compounds and provide a spatial location of a source of the compound or mixture of compounds within an outdoor or indoor (enclosed) environment, as will be discussed in more detail below. In some embodiments, the controller may further provide control signals and data acquisition capabilities for controlling heating elements, cooling elements, cell culture medium perfusion systems, air collection systems (e.g., blowers, fans, etc.), humidity control systems, etc., as well as reading data provided by one or more sensors, e.g., temperature sensors, pH sensors, gas sensors (e.g., O2 sensors, CO2 sensors), glucose sensors, optical sensors, electrochemical sensors, opto-electric sensors, piezoelectric sensors, etc.

In some embodiments, the detection system may further comprise heating systems, cooling systems, cell culture medium perfusion systems, gas perfusion systems, air collection systems (e.g., blowers, fans, etc.), humidity control systems, motion dampening systems, one or more computers and computer memory storage devices, etc.

1. Triangulation of Sensor Signals to Locate Sources of Volatile Compounds:

As noted above, one important feature of the disclosed detection systems is the ability to process time-stamped sensor data provided by two or more sensor panels positioned at known locations within or around the environment to be monitored, and both detect and identify a volatile compound or mixture of volatile compounds of interest as well as identify the location of the source of the volatile compound(s) within the space being monitored. In some embodiments, a detection system comprising two sensor panels positioned at known locations, e.g., along a linear corridor, may be used to detect volatile compound(s) and estimate the position of a stationary source of the compounds (e.g., by monitoring the time difference between detection by the first sensor panel and detection by the second panel), and/or to determine the direction of travel of a moving source (e.g. by monitoring signals over time). In some embodiments, a detection system comprising three or more sensor panels positioned at known locations, e.g., at multiple positions along a linear corridor, or at multiple positions around an enclosed environment such as an airport terminal space, to detect volatile compound(s) and make a more accurate determination of the location of the source of the compound(s) and/or the direction of travel of the source.

In some embodiments, this may require knowledge of the diffusion coefficients in air for the one or more volatile compounds to be detected. The difference between the time that a signal is detected by a first sensor panel and the time(s) it is detected by at least a second sensor panel may then be used, along with the known separation distance(s) for the sensor panels and the diffusion coefficient(s) for the compound(s) detected, to calculate the position of the source relative to the locations of the sensor panels. Furthermore, monitoring of the time-dependent signals arising from each sensor panel permits tracking of any motion of the source.

In some embodiments, the use of triangulation techniques to locate and monitor the position of a source of volatile compound(s) may also require knowledge of the detection sensitivities and response times of the cell-based sensor devices used to monitor the space. This information can then be used to correct estimates for distances between the position of the source and the locations of the sensor panels in order to make a more accurate determination of the position of the source.

In some embodiments, the accuracy of the detection systems for determining the position of the source may be further enhanced through the use of machine learning-based processing of the sensor signals. Machine learning algorithms that have been trained using sensor signal data sets generated using control samples of one or a mixture of known compound(s), samples comprising one or a mixture of known compound(s) at varying concentration levels, and wherein the control samples are positioned at known locations with the space being monitored while collecting the training sensor signal data, may then be used to map a given test sensor signal input data set to an output data set comprising a determination of compound identity, compound mixture identity, estimates of compound concentration(s), location of compound source(s) within the space, or any combination thereof. In some cases, a machine learning approach may also provide improved accuracy for determining a source location within the space where air movement is an issue (e.g., by training the machine learning algorithm under conditions where air movement is controlled but representative of the range of air movements typically observed within the space). Examples of suitable machine learning-based algorithms and training data sets will be described in more detail below.

In some embodiments, the disclosed detection systems may be used to detect and identify volatile compounds or mixtures of compounds in any of a variety of spaces or environments. Examples include, but are not limited to, residential spaces, office spaces, commercial spaces, manufacturing facilities, hospital facilities, airport facilities, and the like. In some embodiments, the disclosed detection systems may be used to detect and identify volatile compounds or mixtures of compounds in outdoor environments, e.g., enclosed courtyards and the like.

In some embodiments, the disclosed detection systems may provide a determination of the spatial location of a source of volatile compound(s) within a monitored space with an accuracy ranging from about 0.001 meters to about 10 meters in any dimension. In some embodiments, the location of the source may be determined to within at least 10 meters, at least 5 meters, at least 1.0 meters, at least 0.1 meters, at least 0.01 meters, or at least 0.001 meters in any dimension.

2. Systems with Cartridge Interface

Detection systems can comprise an interface for accepting and engaging a cell-based sensor as described herein. Once engaged, the interface forms various connections with the cell-based sensor. The interface can include a locking mechanism to hold the sensor in place. For example, the sensor can have holes through which pins in the interface engage the sensor, and locking devices, such as screws or clamps, can secure the sensor. The detection system can comprise a source of cell culture medium. The source can be in fluidic communication with chambers of the cell-based sensor that comprise cells through fluidic conduits, such as tubes. One or more pumps can move fluids into and out of the chambers through such conduits. The system also can comprise an electrical system. The system also can include a blowing device configured to move gas, e.g., air, across a surface in the sensor in gas communication with the cells, for example, through a gas-permeable membrane. The blowing device could be a pump, vacuum or motorized fan that directs the gas. Electrodes in the cell-based sensor can be put into electrical communication with the electrical system when the interface engages the sensor, for example, through physical contact between an electrode in the sensor and an electrical terminal in the system. Alternatively, in system that detects an optical signal from the sensor, the system can comprise an optical sub-system comprising an optical train that includes a source of light for illuminating cells, optics for directing the light, and a detector for detecting light from the compartments. The optical subsystem can be configured to put a source of light, such as an LED in optical communication with cells of a cartridge engaged with the device, and to put a light detector, such as a CCD array, in optical communication with cells producing a light signal.

In such a system, cells can remain alive for at least one week, at least one month or at least three months. Under such conditions, a plurality of assays can be performed using the same sensor, that is, without dis-engaging the sensor from the interface between assays. Accordingly, a plurality of assays can be performed using the same sensor, which assays are spaced apart by at least one day, at least 7 days, at least one month or at least three months.

### III. Machine Learning-Based Sensor Signal Processing

Any of a variety of machine learning algorithms known to those of skill in the art may be suitable for use in processing the sensor signals generated by the disclosed cell-based sensor devices and systems. Examples include, but are not limited to, supervised learning algorithms, unsupervised learning algorithms, semi-supervised learning algorithms, reinforcement learning algorithms, deep learning algorithms, or any combination thereof. In one preferred embodiment, a support vector machine learning algorithm may be used. In another preferred embodiment, a deep learning machine learning algorithm may be used.

A. Supervised Learning Algorithms:

In the context of the present disclosure, supervised learning algorithms are algorithms that rely on the use of a set of labeled, paired training data examples (e.g., sets of sensor signal patterns, and the corresponding known compound identities and concentrations for control samples) to infer the relationship between compound identity and sensor signal pattern.

B. Unsupervised Learning Algorithms:

In the context of the present disclosure, unsupervised learning algorithms are algorithms used to draw inferences from training data sets consisting of sensor signal patterns that are not paired with labeled compound identity data. The most commonly used unsupervised learning algorithm is cluster analysis, which is often used for exploratory data analysis to find hidden patterns or groupings in process data.

C. Semi-Supervised Learning Algorithms:

In the context of the present disclosure, semi-supervised learning algorithms are algorithms that make use of both labeled and unlabeled data for training (typically using a relatively small amount of labeled data with a large amount of unlabeled data).

D. Reinforcement Learning Algorithms:

In the context of the present disclosure, reinforcement learning algorithms are algorithms which are used, for example, to determine a set of sensor signal processing steps that should be taken so as to maximize a compound identification reward function. Reinforcement learning algorithms are commonly used for optimizing Markov decision processes (i.e., mathematical models used for studying a wide range of optimization problems where future behavior cannot be accurately predicted from past behavior alone, but rather also depends on random chance or probability). Q-learning is an example of a class of reinforcement learning algorithms. Reinforcement learning algorithms differ from supervised learning algorithms in that correct training data input/output pairs are never presented, nor are sub-optimal actions explicitly corrected. These algorithms tend to be implemented with a focus on real-time performance through finding a balance between exploration of possible outcomes (e.g. correct compound identification) based on updated input data and exploitation of past training.

E. Deep Learning Algorithms:

In the context of the present disclosure, deep learning algorithms are algorithms inspired by the structure and function of the human brain called artificial neural networks (ANNs), and specifically large neural networks comprising multiple hidden layers, that are used to map an input data set (e.g. a sensor signal pattern) to, for example, a determination of compound identity. Artificial neural networks and deep learning algorithms will be discussed in more detail below.

F. Support Vector Machine Learning Algorithms:

Support vector machines (SVMs) are supervised learning algorithms that analyze data used for classification and regression analysis. Given a set of training data examples (e.g., a sensor electrical signals), each marked as belonging to one or the other of two categories (e.g., compound detected or compound not detected), an SVM training algorithm builds a linear or non-linear classifier model that assigns new data examples to one category or the other.

G. Artificial Neural Networks & Deep Learning Algorithms:

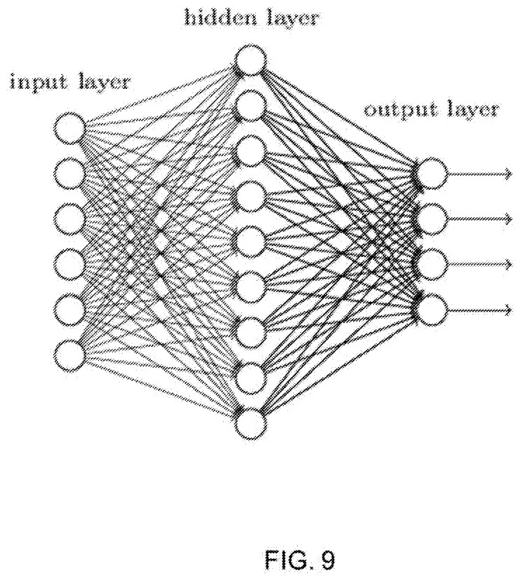

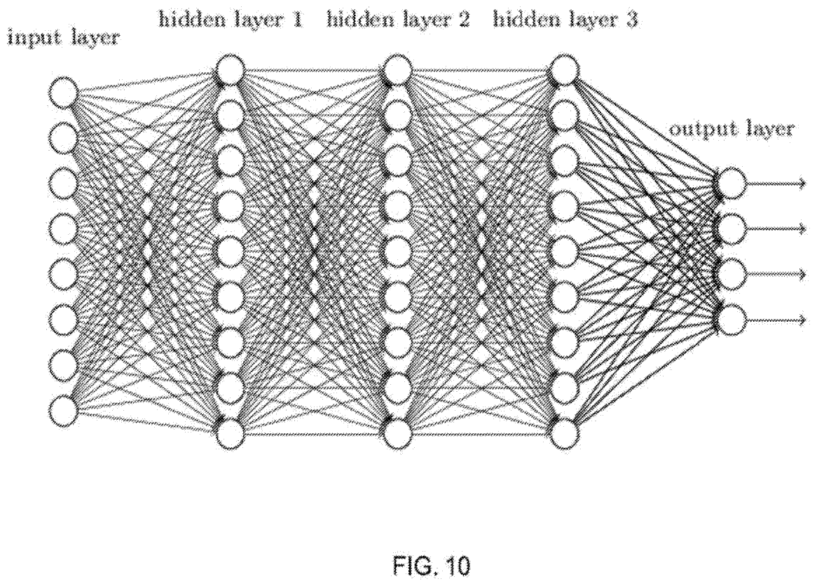

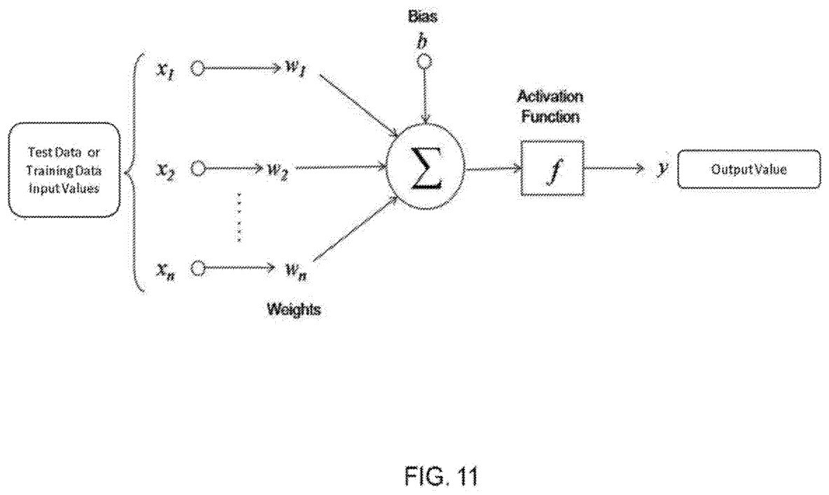

Artificial neural networks (ANN) are machine learning algorithms that may be trained to map an input data set (e.g., sensor signal patterns) to an output data set (e.g., compound identification, etc.), where the ANN comprises an interconnected group of nodes organized into multiple layers of nodes (FIG. 9). For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. As used herein, a deep learning algorithm (DNN) is an ANN comprising a plurality of hidden layers, e.g., two or more hidden layers (FIG. 10). Each layer of the neural network comprises a number of nodes (or “neurons”). A node receives input that comes either directly from the input data (e.g., sensor signals or signal patterns) or the output of nodes in previous layers, and performs a specific operation, e.g., a summation operation. In some cases, a connection from an input to a node is associated with a weight (or weighting factor). In some cases, the node may sum up the products of all pairs of inputs, xi, and their associated weights (FIG. 11). In some cases, the weighted sum is offset with a bias, b, as illustrated in FIG. 11. In some cases, the output of a node or neuron may be gated using a threshold or activation function, f, which may be a linear or non-linear function. The activation function may be, for example, a rectified linear unit (ReLU) activation function, a Leaky ReLu activation function, or other function such as a saturating hyperbolic tangent, identity, binary step, logistic, arcTan, softsign, parametric rectified linear unit, exponential linear unit, softPlus, bent identity, softExponential, Sinusoid, Sinc, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) (e.g., a determination of compound identity and/or the position coordinates of the source of the compound) that the ANN computes are consistent with the examples included in the training data set. The parameters may be obtained from a back propagation neural network training process that may or may not be performed using the same computer system hardware as that used for performing the cell-based sensor signal processing methods disclosed herein.

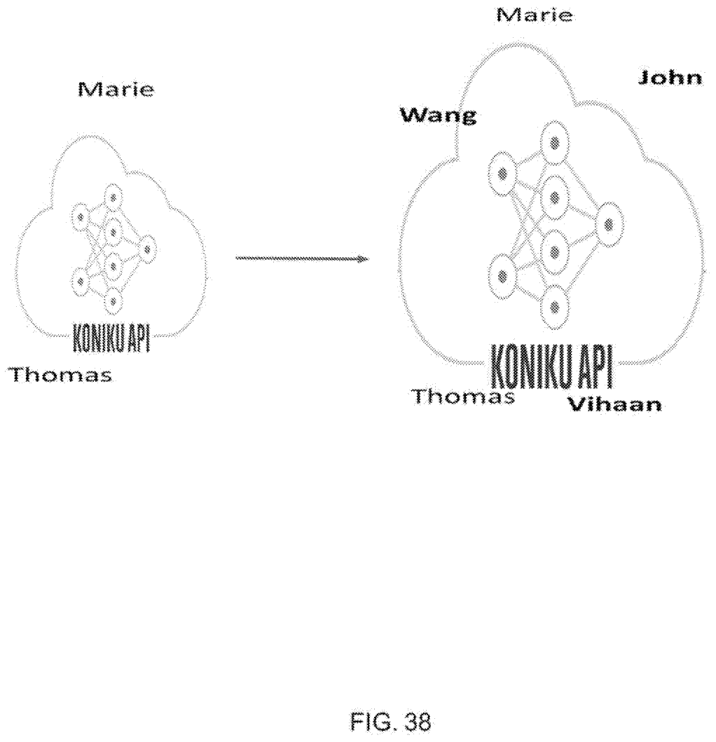

Any of a variety of neural networks known to those of skill in the art may be suitable for use in processing the sensor signals generated by the cell-based sensor devices and systems of the present disclosure. Examples include, but are not limited to, feedforward neural networks, radial basis function networks, recurrent neural networks, or convolutional neural networks, and the like. In some embodiments, the disclosed sensor signal processing methods may employ a pre-trained ANN or deep learning architecture. In some embodiments, the disclosed sensor signal processing methods may employ an ANN or deep learning architecture wherein the training data set is continuously updated with real-time detection system sensor data generated for control samples by a single local detection system, from a plurality of local detection systems, or from a plurality of geographically-distributed detection systems that are connected through the internet. FIG. 38 shows a non-limiting example of a continuous learning process of the relevant algorithm.

In general, the number of nodes used in the input layer of the ANN or DNN (which may enable input of data from multiple electrodes, cell-based sensor devices, or sensor panels) may range from about 10 to about 100,000 nodes. In some instances, the number of nodes used in the input layer may be at least 10, at least 50, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10,000, at least 20,000, at least 30,000, at least 40,000, at least 50,000, at least 60,000, at least 70,000, at least 80,000, at least 90,000, or at least 100,000. In some instances, the number of node used in the input layer may be at most 100,000, at most 90,000, at most 80,000, at most 70,000, at most 60,000, at most 50,000, at most 40,000, at most 30,000, at most 20,000, at most 10,000, at most 9000, at most 8000, at most 7000, at most 6000, at most 5000, at most 4000, at most 3000, at most 2000, at most 1000, at most 900, at most 800, at most 700, at most 600, at most 500, at most 400, at most 300, at most 200, at most 100, at most 50, or at most 10. Those of skill in the art will recognize that the number of nodes used in the input layer may have any value within this range, for example, about 512 nodes.

In some instance, the total number of layers used in the ANN or DNN (including input and output layers) may range from about 3 to about 20. In some instance the total number of layers may be at least 3, at least 4, at least 5, at least 10, at least 15, or at least 20. In some instances, the total number of layers may be at most 20, at most 15, at most 10, at most 5, at most 4, or at most 3. Those of skill in the art will recognize that the total number of layers used in the ANN may have any value within this range, for example, 8 layers.

In some instances, the total number of learnable or trainable parameters, e.g., weighting factors, biases, or threshold values, used in the ANN or DNN may range from about 1 to about 10,000. In some instances, the total number of learnable parameters may be at least 1, at least 10, at least 100, at least 500, at least 1,000, at least 2,000, at least 3,000, at least 4,000, at least 5,000, at least 6,000, at least 7,000, at least 8,000, at least 9,000, or at least 10,000. Alternatively, the total number of learnable parameters may be any number less than 100, any number between 100 and 10,000, or a number greater than 10,000. In some instances, the total number of learnable parameters may be at most 10,000, at most 9,000, at most 8,000, at most 7,000, at most 6,000, at most 5,000, at most 4,000, at most 3,000, at most 2,000, at most 1,000, at most 500, at most 100 at most 10, or at most 1. Those of skill in the art will recognize that the total number of learnable parameters used may have any value within this range, for example, about 2,200 parameters.

ANN or DNN training data sets: The input data for training of the ANN or deep learning algorithm may comprise a variety of input values depending whether the machine learning algorithm is used for processing sensor signal data for a single cell-based sensor device, a sensor panel, or a detection system of the present disclosure. For processing sensor signals generated by individual cell-based sensor devices or sensor panels, the input data of the training data set may comprise single timepoint data or multi-timepoint (i.e., kinetic) data for the electrical signals (e.g., voltages or currents) recorded by one or more electrodes in one or more cell-based sensor devices, or in one or more sensor panels, along with the compound identities and concentrations of control samples to which the sensor devices or panels have been exposed. For processing sensor signals generated by the disclosed detection systems, the input data of the training data set may comprise single timepoint or kinetic data for the electrical signals recorded by one or more electrodes in one or more cell-based sensor devices of each panel, along with the time-stamp data associated with the electrical signal data, the position coordinates for the known locations of the sensor panels, and the compound identities, diffusion coefficients, concentrations, and position coordinates for the known locations of the control samples to which the sensor panels of the detection system have been exposed. In general, the ANN or deep learning algorithm may be trained using one or more training data sets comprising the same or different sets of input and paired output (e.g., compound identity and/or source location) data.

H. Distributed Data Processing Systems and Cloud-Based Training Databases:

In some embodiments, the machine learning-based methods for cell-based sensor signal processing disclosed herein may be used for processing sensor data on one or more computer systems that reside at a single physical/geographical location. In some embodiments, they may be deployed as part of a distributed system of computers that comprises two or more computer systems residing at two or more physical/geographical locations. Different computer systems, or components or modules thereof, may be physically located in different workspaces and/or worksites (i.e., in different physical/geographical locations), and may be linked via a local area network (LAN), an intranet, an extranet, or the internet so that training data and/or sensor data from, e.g., air samples, to be processed may be shared and exchanged between the sites.

In some embodiments, training data may reside in a cloud-based database that is accessible from local and/or remote computer systems on which the machine learning-based sensor signal processing algorithms are running. As used herein, the term “cloud-based” refers to shared or sharable storage of electronic data. The cloud-based database and associated software may be used for archiving electronic data, sharing electronic data, and analyzing electronic data. In some embodiments, training data generated locally may be uploaded to a cloud-based database, from which it may be accessed and used to train other machine learning-based detection systems at the same site or a different site. In some embodiments, sensor device and system test results generated locally may be uploaded to a cloud-based database and used to update the training data set in real time for continuous improvement of sensor device and detection system test performance.

### IV. Processors and Computer Systems

The present disclosure provides computer control systems that are programmed to implement methods of the disclosure. The computer system may be programmed or otherwise configured to direct electrodes to measure one or more electrical signals, to receive one or more electrical signals from one or more electrodes, to generate a pattern of electrical signals, to store patterns of electrical signals or electrical signals in a database, to compare a pattern of electrical signals to a pattern stored in a database, or any combination thereof. The computer system may regulate various aspects of data collection, data analysis, and data storage, of the present disclosure, such as, for example, directing electrical signal measurements, comparing of patterns based of electrical signals measured, generating patterns based on electrical signal data, any combinations thereof, and others. The computer system may be an electronic device of a user or a computer system that is remotely located with respect to the electronic device. The electronic device can be a mobile electronic device.

In some embodiments, the hardware and software code of the computer system may be built around a field-programmable gate array (FPGA) architecture. Unlike microprocessors, which process a fixed set of instructions using a corresponding hard-wired block of logic gates, an FPGA doesn't have any hard-wired logic blocks. Rather, the logic blocks are programmed by the user, which constitutes the “programming” of an FPGA (the code is essentially a hardware change). FPGAs have the advantage of being much faster than microprocessors for performing specific sets of instructions.

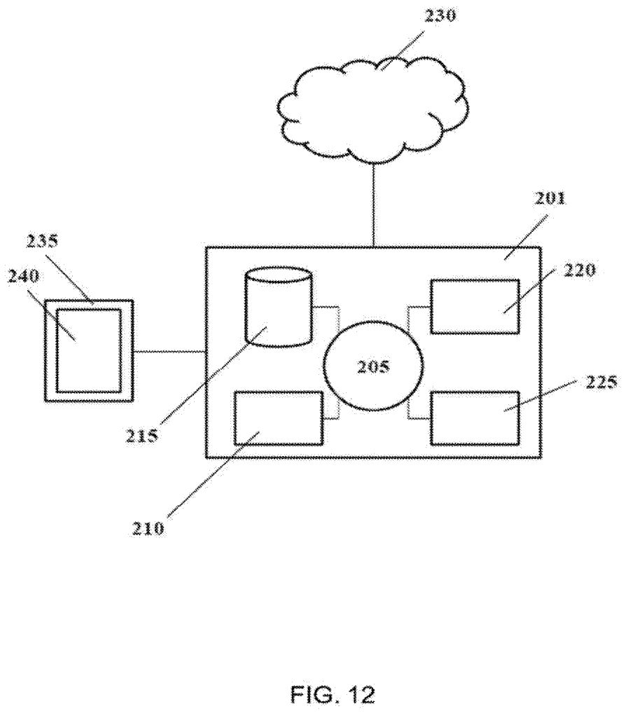

In some embodiments, the computer system may comprise a central processing unit (CPU). FIG. 12 shows a computer system that may include a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the computer system 201, can implement a peer-to-peer network, which may enable devices coupled to the computer system 201 to behave as a client or a server.

Such systems can be connected through a communications network to the Internet. The communications network can be any available network that connects to the Internet. The communication network can utilize, for example, a high-speed transmission network including, without limitation, Digital Subscriber Line (DSL), Cable Modem, Fiber, Wireless, Satellite and, Broadband over Powerlines (BPL).

The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user (e.g., portable PC, tablet PC, Smart phones). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, a confirmation of a presence or a likelihood of a presence of a compound, such as a volatile compound. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, generate a pattern based on electrical signals received from one or more electrodes, such as a matrix of electrical signals, compare a pattern generated by the control system to one or more patterns stored in a database of the system, make a confirmation of a presence or a likelihood of a presence of a compound in sample, or any combination thereof, and others.

### V. Applications

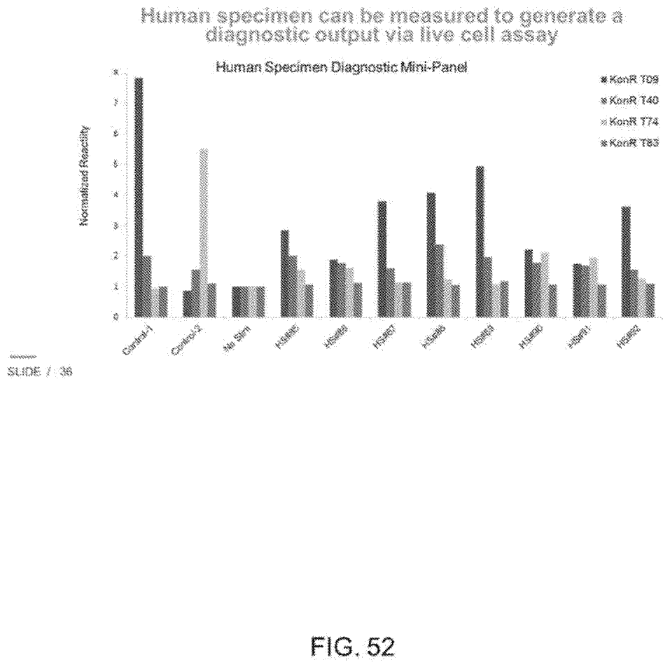

The cell-based sensor devices and detection systems disclosed herein may be applied to a variety of sensing applications, and in particular, to volatile compound sensing applications. Examples include, but are not limited to, monitoring produce to determine the degree of ripeness of fruit; to detect spoilage in vegetables or other food products; to detect and diagnose disease states in patients (e.g., diabetic patients); to detect the presence of airborne toxic compounds in residential, office, or commercial spaces; or to detect taggants or volatile markers for explosive materials, e.g., in airport facilities. In some cases, the disclosed sensor devices and detection systems may be used for detecting a specific odorant such as TNT and related compounds (e.g., precursor compounds, degradation products, etc.). FIG. 52 shows a non-limiting example that human specimen can be measured to generate a diagnostic output via live cell assay.

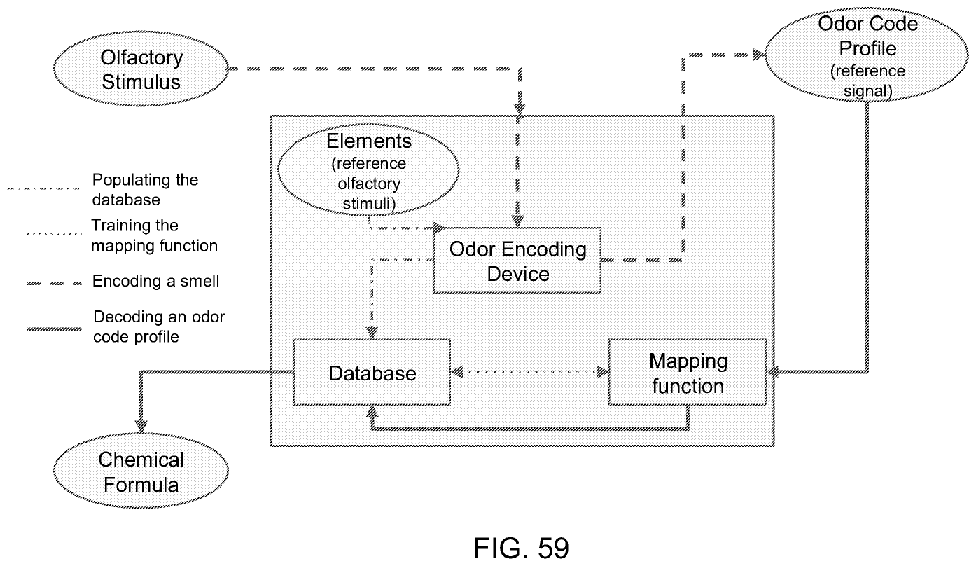

Referring to FIG. 59 methods are provided herein for encoding and decoding an olfactory stimulus. A mapping function is produced using machine learning. Various elements, e.g., compounds or olfactory stimuli, alone or in combination, and at different relative concentrations, are each provided to an odor encoding device to generate a plurality of odor code profiles. The odor code profiles indicate a quantitative measure (number, range, relative amount, etc.) of response by each olfactory receptor in the device. The collection of odor code profiles is used as training set to train a machine learning algorithm to produce a mapping function, which may be a regressor or a classifier, depending on context, that predicts, from a test odor code profile, a formula from the collection of elements that produces the odor code profile approximating the odor code profile of the test olfactory stimulus. The formula typically will include both the identity and relative amounts of the elements in the formula.

To encode a test olfactory stimulus, the stimulus is provided to the odor encoding device to generate an odor code profile of the test stimulus.

To decode the olfactory stimulus, the odor code profile is provided to the mapping function which, in turn, generates one or more formulae predicted to have odor code profiles matching or approximating the odor code profile of the test stimulus.

### VI. Universal Odor Code Systems

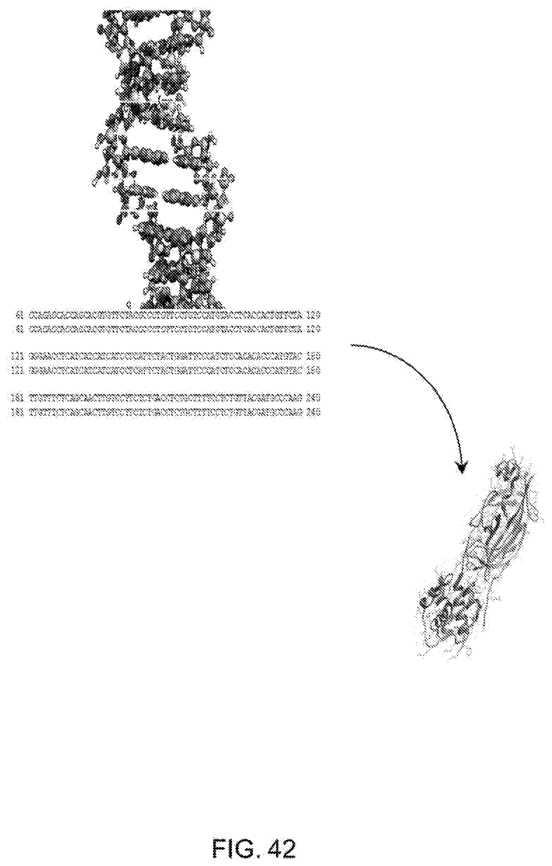

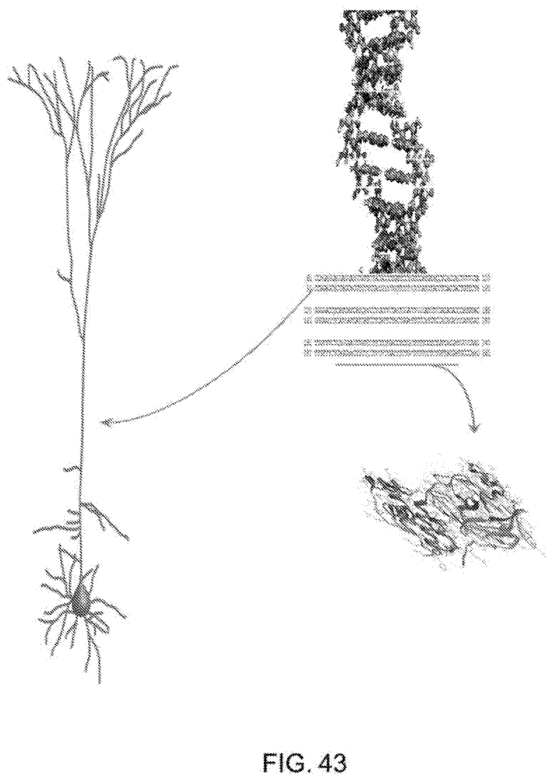

The odor encoding device may be used to create a universal odor code system. Within the universal odor code system, any odor may be characterized by its unique “olfactory receptor (“hOR”) intensity fingerprint (also referred to as an “odor code profile”). FIG. 42 shows that an olfactory receptor has a DNA code. The DNA may determine the receptors on the surface of a cell. For example, a neuron may be engineered with a DNA for a receptor detecting TNT, and the cell may be capable of detecting TNT. In another example, a neuron may be engineered with a DNA for a receptor detecting DNT, and the cell may be capable of detecting DNT. FIG. 43 shows that the DNA can make the neuron to produce receptors.

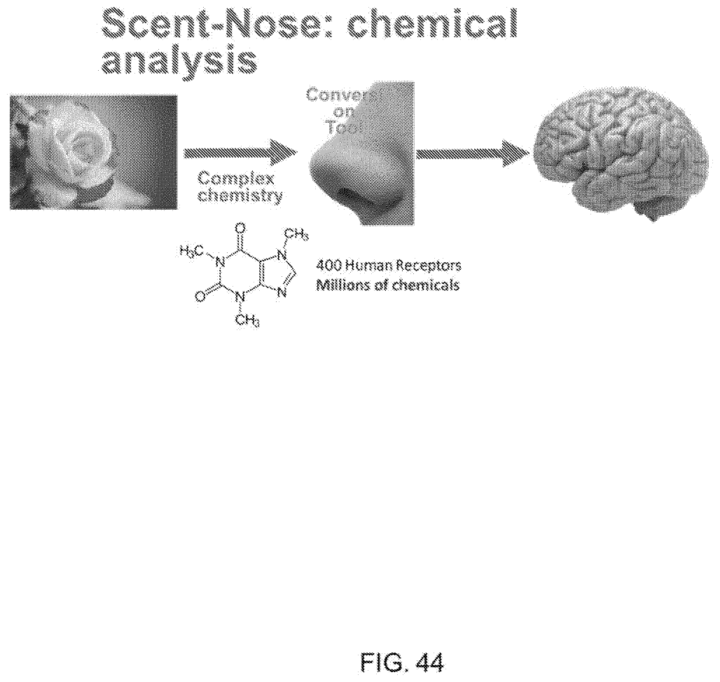

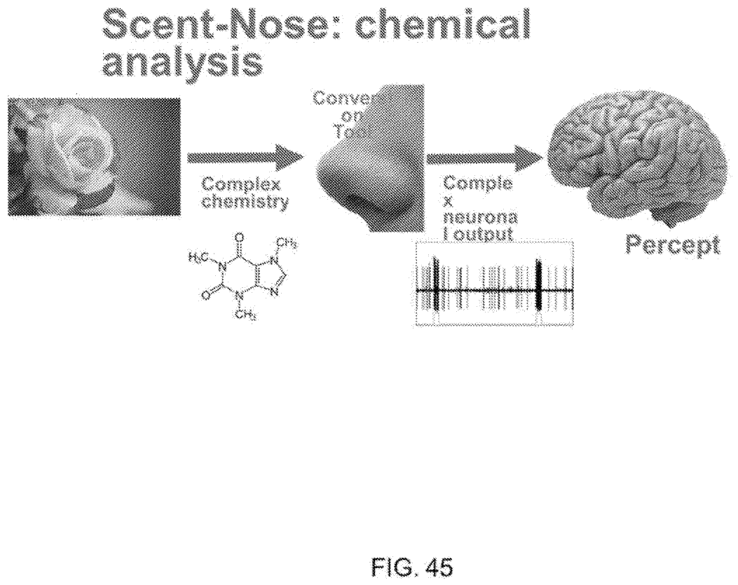

Within the universal odor code system, odors may be encoded into a hOR space. The hOR space may include any information associated with a hOR. The information related to a hOR may be a code or identity of a hOR, a neural response associated with a hOR, or a physiological state associated with an event triggering a hOR. The hOR may also include any information associated with an odor, a compound, or a mixture of compounds that triggers a hOR. FIG. 44 shows a non-limiting example of a human's neural response to an odor. FIG. 45 shows another non-limiting example of a human's neural responses to an odor.

A. Databases of Odor Code Profiles

The universal odor code system may comprise a database. The database can be stored in computer readable format. The database may comprise the information regarding a plurality of elements. The element may be a stimulus. The element may be an odorant. Each of the plurality of elements may be a compound. Each of the plurality of elements may be a mixture of compounds. Each element may bind to a cell surface receptor. Upon binding, the element may activate series of intracellular signaling proteins or pathways and may trigger an action potential by the neuron. Each element may trigger one hOR. The chemical reactions between the different elements may be negligible. Each of the plurality of elements may be smelt and/or tasted by humans. At least one of the pluralities of elements may be a conjugate element. The conjugate element may be a compound that principally triggers one hOR. The conjugate element may be a mixture of compounds that principally triggers one hOR. The number of the plurality of elements in the database may be at least about 1, 10, 50, 80, 100, 130, 150, 180, 200, 210, 230, 250, 280, 300, 310, 330, 350, 380, 400, 410, 430, 450, 480, 500, 510, 530, 550, 580, 600, 700, 800, 900, 1000 or greater.

The universal odor code system may comprise a computer readable memory. In some embodiments, information regarding the plurality of elements may be stored on an electronic storage device on computer readable memory. In some cases, information regarding the plurality of elements may not be stored on an electronic storage device on computer readable memory. The information regarding each one of the plurality of elements may be encrypted and encoded in a code. The information regarding the plurality of elements may include, but not limited to, the carbon atom number, the molecular weight, the number of carbon-carbon bond, the number of functional groups, the aromaticity index, the maximal electrotopological negative variation, the number of benzene-like rings, the number of aromatic hydroxyls, the average span R, the number of carboxylic group, the number of double bonds. The code may be stored on an electronic storage device on computer readable memory.

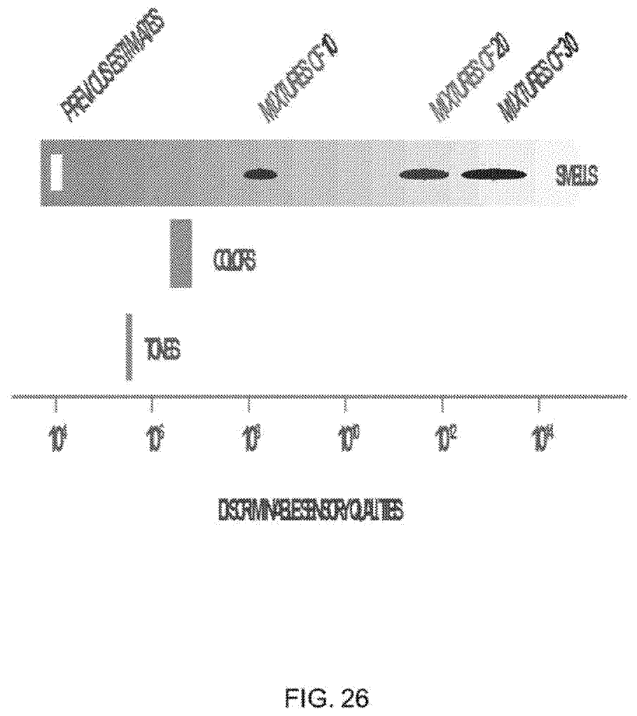

Within the universal odor system, two different compounds may have the same code if they result in the same odor for a human subject. Within the universal odor system, two different mixtures of compounds may have the same code if they result in the same odor for a human subject. Within the universal odor system, mixtures with different compounds having different codes may result in different odors for a human subject. The code may be universal. The universal odor system may encode any odor by the combination of odors for each hOR. The universal odor system may reproduce any human smell/taste. The process of reproduction may be executed by triggering all the combinations of hORs with their conjugate elements from the database. FIG. 26 shows a non-limiting example of the numeric scale of the smells.

In some cases, the universal odor code system may comprise computer readable memory storing information regarding a plurality of elements and a computer processor. In some cases, a computer processor may access information regarding a plurality of elements stored in the computer readable memory. In some cases, a computer system may be used to build the database. The process of building the database may comprise pre-selecting a plurality of compounds. The compounds may be non-harmful compounds. The compounds may be known to have different odors. The process of building the database may further comprise determining one or more hORs associated with each compound by screening method. The screening method may comprise transfecting hORs in in-vitro cells. The screening method may comprise providing an odor encoding device and using the odor encoding device to detect the compound. The screening method may comprise providing a cell-based sensor and using the cell-based sensor to detect the compound.

A process for building a database can begin with a collection or palette of elements, which can be individual compounds or compositions. The palette can have tens, hundreds or thousands of different elements or compounds. Combinations of elements can, themselves, be considered elements. Elements from the palette are tested alone and in combinations of 2, 3, 4, 5, 6, 7, 8, 9, 10 or more elements to generate an odor code profile for each element or combination measured. Elements in the palette and combinations thereof can be tested in various relative or absolute concentrations, as the response or odor code profile may be a function of concentration. The resulting database can include information about (1) composition of the element or combination (e.g., chemical formula, name of compound or compounds in a mixture); (2) absolute and/or relative concentrations or amounts each compound in a composition to be tested; and (3) odor code profile of the composition tested. So, for example, for a collection of ten elements, the database could contain odor code profiles of each element, individually, at each of one or a plurality of different concentrations; odor code profiles of each pairwise or tuple (3-, 4-5—etc.) of elements, wherein each combination is tested at a variety of different relative concentrations. The resulting database has several uses, including as a training dataset and as a reference database for odor recreation.

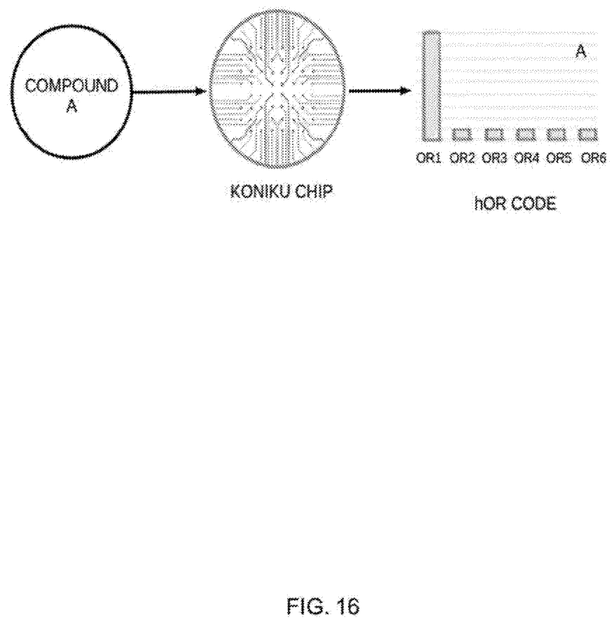

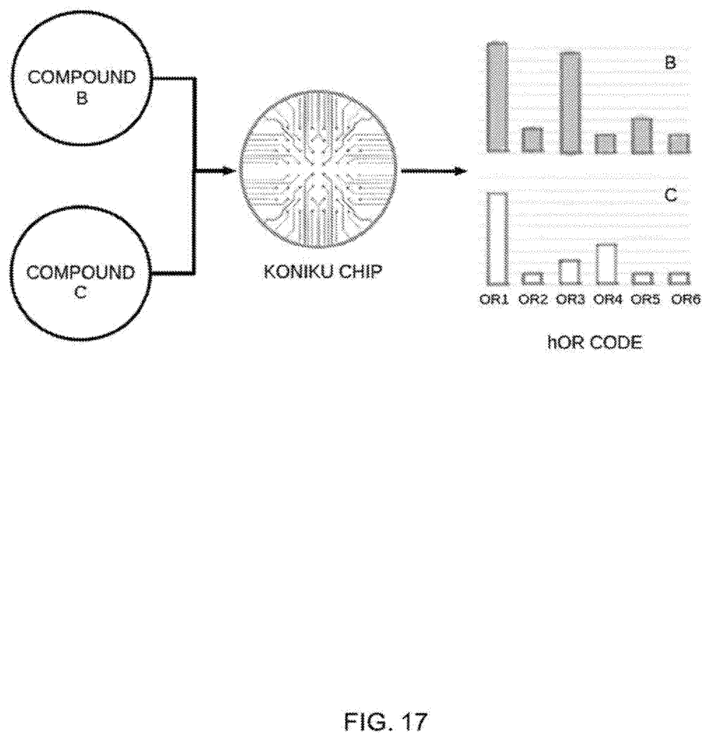

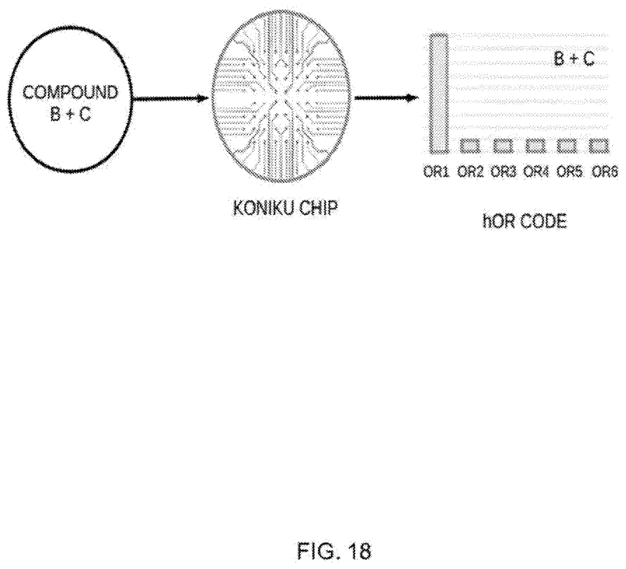

The process of building the database may further comprise selecting a subset of the plurality of compounds. Each of compounds in the subset may trigger a single OR. The process of building the database may further comprise adding the subset to the database. Each compound in the subset may be an element of the database. FIG. 16 shows an example of the process of detecting one compound. In the illustrated example, compound A is screened by the above-mentioned device. The computer system may then yield a code profile for the compound A. In the illustrated example, compound A is principally associated with OR1. The process of building the database may further comprise determining a mixture of compounds that trigger a single OR through theoretical models and/or experimental verifications. FIG. 17 shows an example of the process of detecting a mixture of compounds that trigger multiple ORs. In the illustrated example, compound B and compound C have different odor code profiles. FIG. 18 shows an example of the process of detecting a mixture of compounds that trigger a single OR. In the illustrated example, compounds B and C mixture has a single odor code profile. In the illustrated example, compounds B and C mixture is principally associated with OR1. The process of building the database may further comprise, for the subset of compounds associated with each OR, selecting one or more compounds in the subset that have negligible integration between each other. Each of the selected one or more compounds may be a conjugate element. Each of the selected one or more compounds may be an element. The information regarding each one of the elements may be encrypted and encoded in a code. The code may reveal the element's odor code profile.

The code and the odor code profile may be stored remotely or internally on the database. The data may be mined using Artificial Intelligence tools for stratification. In some cases, the universal odor code system may comprise a transmitting component for transmitting a result. The transmitting component may be wired or wireless component. Examples of wired communication transmitting component can include a Universal Serial Bus (USB) connection, a coaxial cable connection, an Ethernet cable such as a Cat5 or Cat6 cable, a fiber optic cable, or a telephone line. Examples or wireless communication transmitting component can include a Wi-Fi receiver, a means for accessing a mobile data standard such as a 3G, 4G or 5G LTE data signal, or a Bluetooth receiver. In some cases, the universal odor code system may communicate with an external database. In some embodiments, the transmitting component can transmit data to a database or server. A database or server can be a cloud server or database. In some embodiments, the transmitting component can transmit data wirelessly via a Wi-Fi, or Bluetooth connection. In some aspects, a transmitting component described herein can comprise centralized data processing, that could be cloud-based, internet-based, locally accessible network (LAN)-based, or a dedicated reading center using pre-existent or new platforms.

In some aspects, a transmitting component can comprise software. A software can rely on structured computation, for example providing registration, segmentation and other functions, with the centrally-processed output made ready for downstream analysis. In some aspects, the software would rely on unstructured computation, artificial intelligence or deep learning. In a variation of this aspect, the software would rely on unstructured computation, such that data could be iteratively. In a further variation of this aspect, the software can rely on unstructured computation, so-called “artificial intelligence” or “deep learning.” Computer readable memory can be employed for storing data obtained from an odor encoding device.

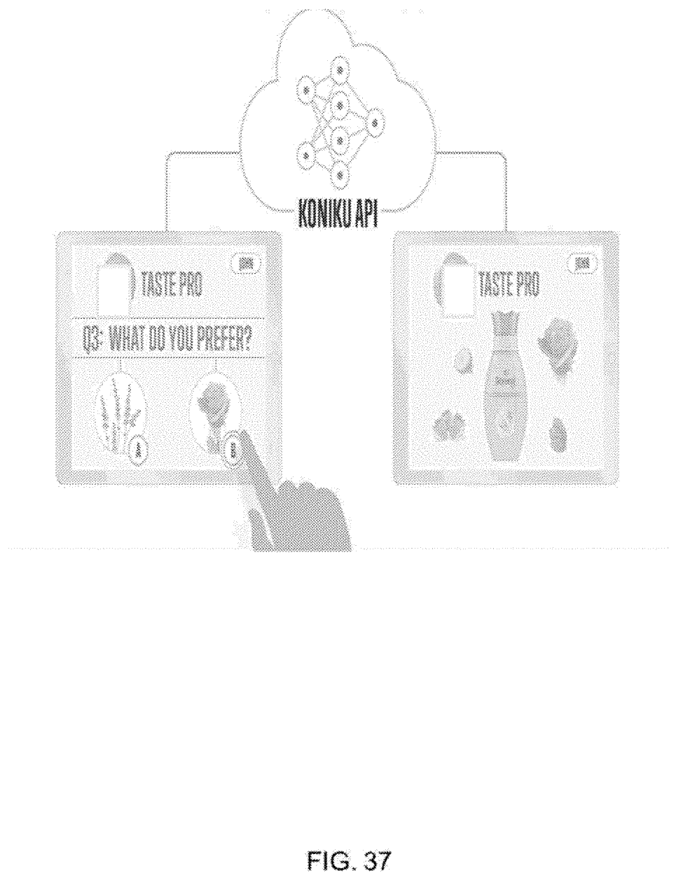

In some aspects, the universal odor code system may comprise a displaying component. The display component may be configured to display a code to a user of the universal odor code system. In some embodiments, the code may be displayed via an interface such as a webpage, application, program, or any appropriate software. The display component can be a monitor, a computer (e.g., laptop computer, desktop computer), a mobile device (e.g., smartphone, tablet, pager, personal digital assistant (PDA)), a vending machine. In some instances, the display component may comprise one or more processors natively embedded in the display component. The display component may optionally be portable. The display component may be handheld. The display screen of the display component may be a liquid crystal display (LCD), cathode ray tube (CRT), light emitting diode (LED) display, touchscreen, electronic paper (e-paper) display, or a display on a separate computing device. FIG. 37 shows non-limiting examples of user interfaces of an application related to the disclosure herein. In the illustrated example, the app may contain a personalized questionnaire or survey designed to target specifically the taste preference of one subject. The questions may be in the form of text, pictures and in the store even odor stimuli, sent to our API. The app may recommend the best product for the subject that answered the test. The data collected may be added to the database and reinforce the algorithm to continuously learn how to satisfy the customers better.

The code or the code profile of the element may be in a format of a table, a chart, a diagram, or a visual graphic code. The visual graphic code may be a bar code or a QR code. The barcode may be a UPC barcode, EAN barcode, Code 39 barcode, Code 128 barcode, ITF barcode, CodaBar barcode, GS1 DataBar barcode, MSI Plessey barcode, QR barcode, Datamatrix code, PDF417 code, and Aztec barcodes. The barcode may define elements such as the version, format, position, alignment, and timing of the barcode to enable reading and decoding of the barcode. The remainder of the barcode can encode various types of information in any type of suitable format, such as binary or alphanumeric information. The QR code can have various symbol sizes. The QR code can be of any image file format (e.g. EPS or SVG vector graphs, PNG, TIF, GIF, or JPEG raster graphics format). The QR code can be based on any of a number of standards. In some instances, a QR code can conform to known standards that can be read by standard QR readers. The information encoded by a QR code may be made up of four standardized types (“modes”) of data (numeric, alphanumeric, byte/binary, kanji) or, through supported extensions, virtually any type of data.

B. Mapping Functions to Predict Odor Code Profile

The odor code profile of a mixture of elements may not be the simple sum of the odor code profiles of each of the elements. This may be due to saturation issues—response is not linear as a function of concentration. It may also reflect the fact that two different elements may not produce an additive response. Databases as described herein can be used to train machine learning algorithms to generate mapping functions, e.g., regressors, that predict an odor code profile of any element or combination of elements in any relative or absolute concentrations. The mapping function (e.g., a regressor or classifier) can be referred to herein as the function “g”. Thus, the response of the ORs ([r1OR, r2OR2, r30R3, . . . rmORm]) is the function of a composition's elements and concentrations ([E1C1, E2C2, E3C3 . . . EnCn]). Thus, in OR space comprising OR1-ORm, the predicted responses, r1-rm, are a function of relative concentrations C1-Cn of each of elements E1-En in the mixture. The mapping function is useful for, among other things, predicting whether a recipe or formula for a composition will produce an odor code profile identical to or similar to the odor code profile of a target composition.

Function “g” returns the distribution of response on the OR space from any combination of concentration of primary odors. It represents a map between primary odors and OR response:

g: [symbol] ^{N}^{ₚ}→ [symbol] ^{N}^{_{hoR}}

where Np is the number of primary compounds or elements, and NhOR is the number of human ORs used to define an odor code profile.

By way of example, if Np=3 and NhOR=4: there are only 3 primary compounds: A, B, and C and only 4 Ors used (OR1, OR2, OR3, OR4). Then, if an operator wants to produce an odor code profile of a mix Mx defined as 1 mM of A and 0.5 mM of B, they can use as input Mx=(1, 0.5, 0) that correspond to a compound mix ([A]=1 mM, [B]=0.5 mM, [C]=0 mM). If the response of this mix is 1 for OR1, 0 for OR2, 2 for OR3, and 3 for OR4 then the output (or odor code profile is (1, 0, 2, 3). In summary the odor code profile is defined by Fpx=g(Mx)=g(1, 0.5, 0)=(1, 0, 2, 3).

Such a mapping is called a multiple regression and can be built with various different algorithms (linear regression, polynomial regression, sigmoid form, neural nets, regression tree, SVM, etc.) This mapping can be parametric (if the operator has defined the form of the response (e.g., sigmoid) or non-parametric (black box). A simple form of a parametric g could be a NhOR×Np matrix of real numbers. This corresponds to a linear regression where it is assumed that each OR response is independent from each other each compound contribution is independent from each other.

In training “g”, experimental results showing the OR responses for different combinations of compounds concentrations are provided. An example of such method could be (with the same set of primary smells A, B, and C and set of OR 1, 2, 3, 4). For each of the ORs, various compositions are tested. These include, for example: Response of A for Nc different concentrations; response of B for Nc different concentrations; Response of A, B for Nc different concentrations. In total 2\hat{ }(Np×Nc)−1 experiments per OR are conducted. From these results, a training set is produced which can be used to build mapping function g (adjusting the parameters in the case of a parametric functions).

The general mechanism of how to use “g” is described in the FIG. 59. However, for some complex forms of g (e.g., non-linear regressions), it may be challenging to compute g\hat{ }−1 (the primary smells that correspond to an odor code profile) because different sets of primary olfactory stimuli may give the same odor code profile. (That is, a different mix of compounds with different concentrations can give same odor code profile). An optimization method (such as a gradient descent) can be used, in which starting from an initial mix of compound and operating some small changes in this input vector (in silico), the mapping function converges towards a local optimum. That requires also the definition of a cost or distance on the R\hat{ }NhOR space.

C. Applications of the Universal Odor Code System

1. First Application—Generating an Odor Code Profile:

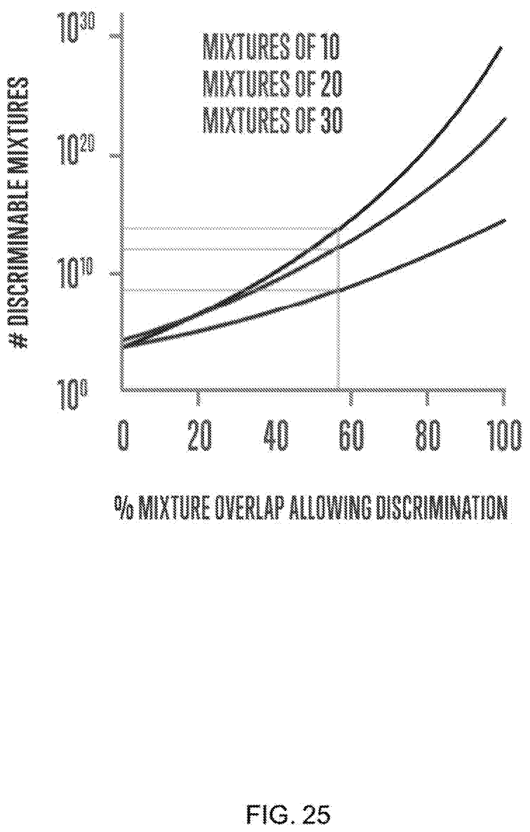

One application of the universal code system may be encoding a new compound or a mixture of new compounds, e.g., producing an odor code profile for the composition. The process of encoding the new compound may comprise providing an odor encoding device. The process of encoding the new compound may comprise mixing the new compound with a medium before the new compound is injected in the odor encoding device. The medium may be a liquid medium. The process of encoding the new compounds may further comprise providing a signal of the new compound. The signal may be optical signal. The signal may be electrical signal. The process of encoding the new compound may further comprise analyzing the signal by one or more algorithm and providing a code to the new compound based on the analysis. The one or more algorithm may comprise machine learning algorithms. The machine learning algorithms may be Support Vector Machine (SVM), Naïve Bayes (NB), Quadratic Discriminant Analysis (QDA), K-Nearest Neighbors (KNN), Linear Discriminant Analysis (LDA), or Multilayer Perceptron (MLP). FIG. 25 shows a non-limiting example of relationship between the percentage of mixture overlap allowing discrimination and the number of discriminable mixtures.

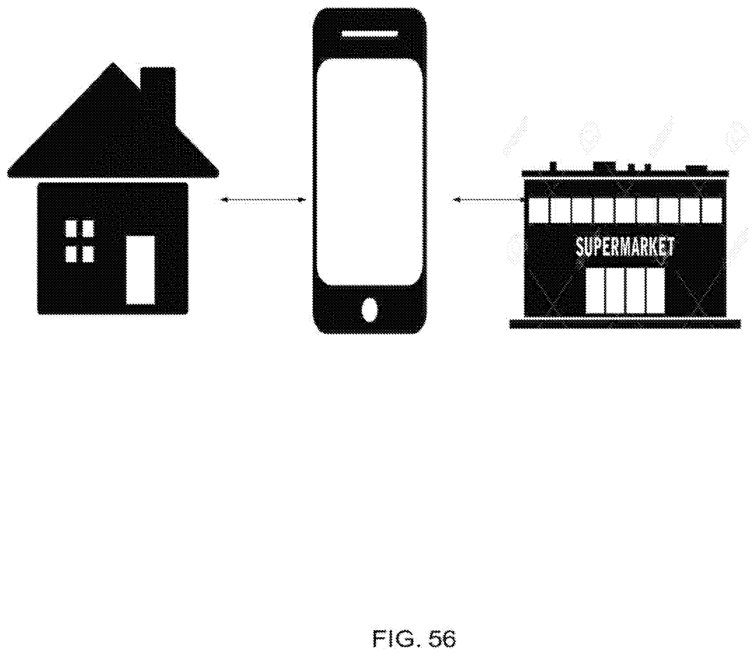

The process of encoding the new compound may further comprise returning the code of the compound to the user. The code may be provided on an electronic device. The electronic device can be a mobile electronic device. The electronic device may be a portable electronic device. The portable electronic device may be a mobile phone, tablet, smartwatch, digital camera, and personal navigation device. FIG. 56 shows that the application of the universal odor code system can be accessible through phone, computer, and any chosen store on tablets. The code or the code profile of the element may be in a format of a table, a chart, a diagram, or a visual graphic code. The code may be in the form of, but not limited to, text, voice, image, and video. The code may be text-based, HTML, image, video, audio, or avatar animation. If the code is in the form of voice or audio, the code may be read to the user through one or more smart speakers. The one or more smart speakers may comprise, but not limited to, Alexa, Google Home, Google Assistant, Clova, Microsoft Cortana, AliGenie, Ambient, Apple HomeKit, Apple Siri, and Apple Pod. The code may provide clickable features for the user to add code, images, video, audio, and animation.

2. Second Application—Mapping Physiological States:

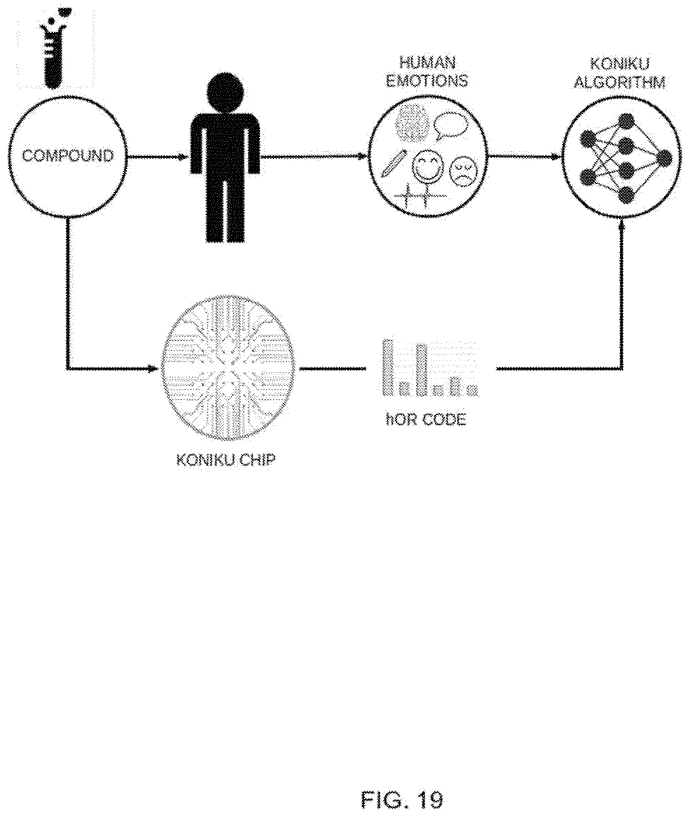

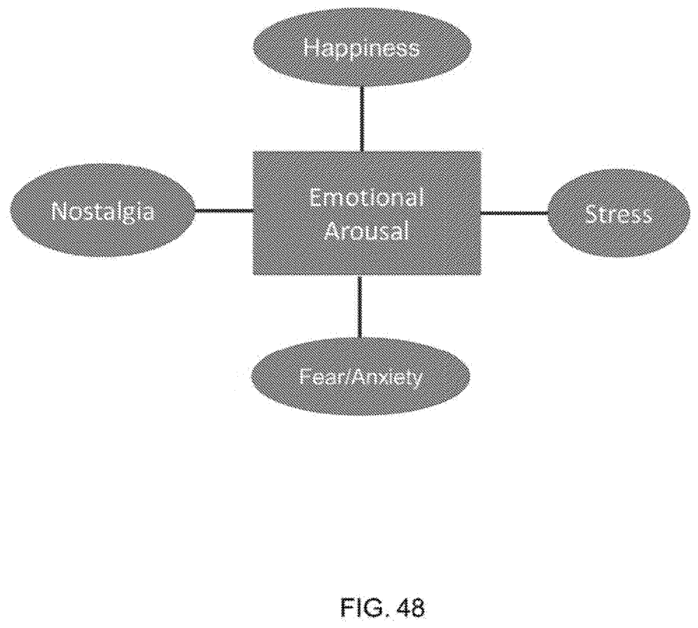

Another application of the universal code system may be mapping physiological states to each hOR or a combination of hORs. FIG. 19 shows a non-limiting example of mapping emotions to every hOR or some combinations of hORs. FIG. 48 shows a non-limiting example of a human's emotion states. The physiological states may be emotional states. The process of mapping physiological states to each hOR or a combination of hORs may comprise recruiting subjects for smelling the conjugate of each hORs. The process of mapping may comprise an objective evaluation and/or a subjective evaluation. The process of mapping may comprise assessing a physiological state of a subject in response to a stimulus. The stimulus can be an external stimulus including touch, pain, vision, smell, taste, sound, and any combinations thereof, elicited by an object. For example, the stimulus can be the smell and/or taste elicited by an object (e.g., a chemical compound). The method can access an emotional state of a subject in response to a smell and/or taste stimulus. The emotional state can comprise happiness, surprise, anger, fear, sadness, or disgust. The emotional state can be further classified into one or more levels. For example, an emotional state (e.g., happiness) can be further classified into 10 numeric levels (e.g., 1 being the lowest happiness level and 10 being the highest happiness level). The subject can be a human subject.

To evaluate the physiological state (e.g., emotional state) of a subject in response to a stimulus (e.g., the smell and/or taste of an object), the stimulus can be mapped to the physiological state using the methods and systems disclosed herein. In some cases, other stimulus, such as music, images, or text can be used in the intermediate steps to train the algorithm.

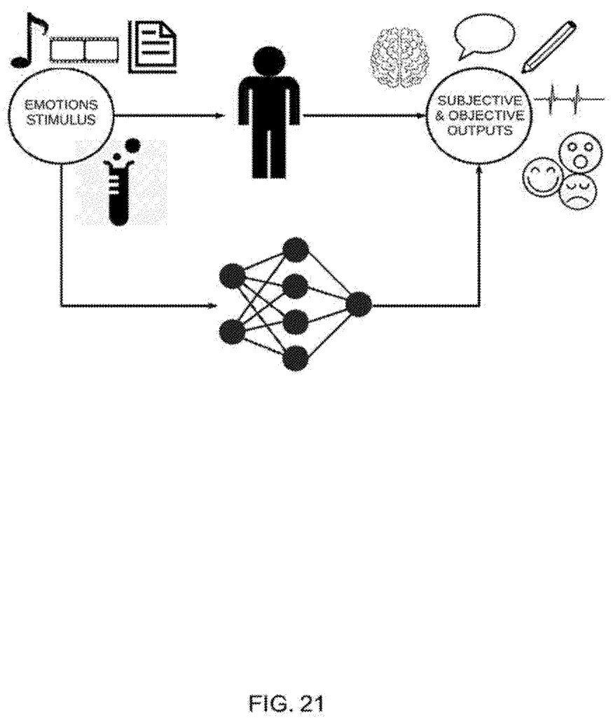

As shown in FIG. 21, the method can comprise an objective evaluation and/or a subjective evaluation. For example, the method can comprise analyzing a physiological signal from the subject in response to the stimulus. In another example, the method can comprise analyzing linguistic expressions of the subject in response to the stimulus. In yet another example, the method can comprise analyzing a physiological signal from the subject in response to the stimulus and analyzing linguistic expressions of the subject in response to the stimulus.

The method for assessing a physiological state of a subject in response to a conjugate can comprise analyzing a physiological signal from the subject. The physiological signal can be detected using a sensor. The physiological signal can be facial expressions, micro expressions, brain signals, electroencephalography (EEG) signals, functional magnetic resonance imaging (fMRI) signals, body odors, pupil dilation, skin conductance, skin potential, skin resistance, skin temperature, respiratory frequency, blood pressure, blood flow, saliva, or any combination thereof. These human emotional state markers can pick up different signal modalities from specific human organs which can yield a large amount of information about the emotional state of person, from happy to sad, with hundreds of shades between.

The method can further comprise characterizing the physiological state of the subject using the analyzed information, for instance, using a machine learning algorithm. Several machine learning algorithms can be used as emotion classifiers such as Support Vector Machine (SVM), Naïve Bayes (NB), Quadratic Discriminant Analysis (QDA), K-Nearest Neighbors (KNN), Linear Discriminant Analysis (LDA), and Multilayer Perceptron (MLP).

Facial expressions can be obtained by an image-capturing sensor, such as a camera. Facial expressions can be obtained from static images, image sequences, or video. Facial expressions can be analyzed using geometric-based approaches or appearance-based approaches. Geometric-based approaches, such as active shape model (ASM), can track the facial geometry information over time and classify expressions based on the deformation. Appearance-based approaches can describe the appearance of facial features and/or their dynamics.

In some cases, analyzing facial expressions can comprise aligning the face images (to compensate for large global motion and maintain facial feature motion detail). In some cases, analyzing facial expressions can comprise generating an avatar reference face model (e.g., Emotion Avatar Image (EAI) as a single good representation) onto which each face image is aligned to (e.g., using an iterative algorithm). In some cases, analyzing facial expressions can comprise extracting features from avatar reference face model (e.g., using Local Binary Pattern (LBP) and/or Local Phase Quantization (LPQ)). In some cases, analyzing facial expressions can comprise categorizing the avatar reference face model into a physiological state using a classifier, such as the linear kernel support vector machines (SVM).

Facial expressions, including micro expressions, can be detected using the facial action coding system (FACS). FACS can identify the muscles that produce the facial expressions and measure the muscle movements using the action unit (AU). FACS can measure the relaxation or contraction of each individual muscle and assigns a unit. One or more muscle can be grouped into an AUs. Similarly, one muscle can be divided into separate AUs. FACS can assign a score consists of duration, intensity, and/or asymmetry.

EEG, the signal from voltage fluctuations in the brain, can be used for assessing the physiological state of the subject. Emotion can be related with some structures in the center of the brain including limbic system, which includes amygdala, thalamus, hypothalamus, and hippocampus. EEG can be obtained by recording the electrical activity on the scalp using a sensor (e.g., electrode). EEG can measure voltage changes resulting from ionic current flows within the neurons of the brain. EEG can measure five major brain waves distinguished by their different frequency bands (number of waves per second), from low to high frequencies, respectively, called Delta (1-3 Hz), Theta (4-7 Hz), Alpha (8-13 Hz), Beta (14-30 Hz), and Gamma (31-50 Hz).

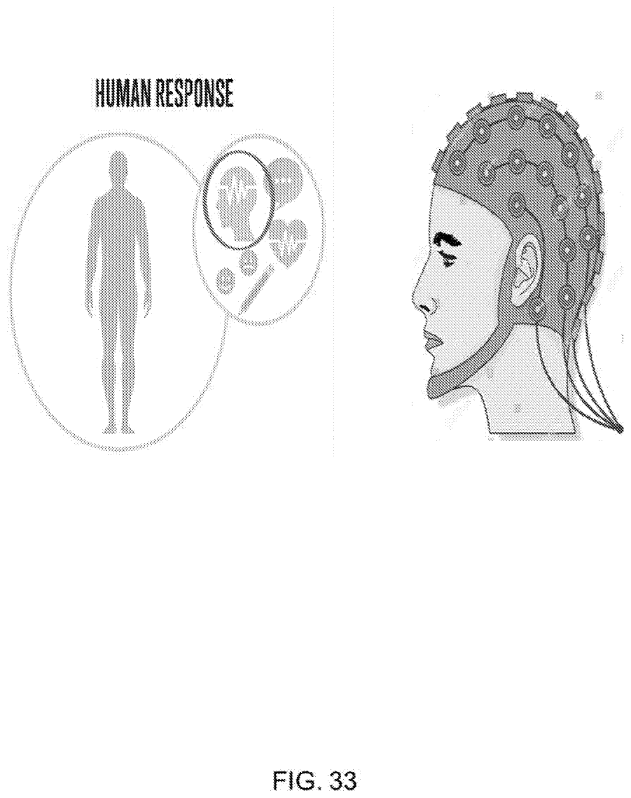

fMRI can be used for assessing the physiological state of the subject. fMRI can measure brain activity by detecting changes associated with blood flow. fMRI can use the blood-oxygen-level dependent (BOLD) contrast. Neural activity in the brain can be detected using a brain or body scan by imaging the change in blood flow (hemodynamic response) related to energy use by brain cells. fMRI can use arterial spin labeling and/or diffusion magnetic resonance imaging MRI. FIG. 33 shows a non-limiting example of detecting human physiological states through brain imaging.

Skin conditions, such as skin conductance, skin potential, skin resistance, and skin temperature can be detected and measured using electronic sensors. For example, skin conductance can be detected and measured using an EDA meter, a device that displays the change electrical conductance between two points over time. In another example, galvanic skin response can be detected and measured using a polygraph device.

Linguistic expressions of the subject can be recorded and analyzed for accessing the physiological state of the subject. The linguistic expression can be any physical form (e.g., sound, visual image or sequence thereof). The linguistic expression can be spoken, written, or signed. The linguistic expression can be classified into an emotional state such as happiness, surprise, anger, fear, sadness, or disgust. In some cases, the subjects can be asked to give their emotional states. In some cases, the subjects can be given a list of words to formulate their emotional states, thereby mapping the linguistic expressions to the emotional states in a more restricted way.

The linguistic expression may be descriptors of the odor of the conjugate. The descriptors of the odors may comprise, but not limited to, fruit, sweet, perfumery, aromatic, floral, rose, spicy, cologne, cherry, incense, orange, lavender, clove, strawberry, anise, violets, grape juice, pineapple, almond, vanilla, peach fruit, honey, pear, sickening, rancid, sour, vinegar, sulfidic, dirty linen, urine, green pepper, celery, maple syrup, caramel, woody, coconut, soupy, burnt milk, eggy, apple, light, musk, leather, wet wool, raw cucumber, chocolate, banana, coffee, yeasty, cheesy, sooty, blood, raw meat, fishy, bitter, clove, peanut butter, metallic, tea leaves, stale, mouse, seminal, dill, molasses, cinnamon, heavy, popcorn, kerosene, fecal, alcoholic, cleaning fluid, gasoline, sharp, raisins, onion, buttery, and herbal. In some cases, the emotional state of the subject can be classified using a computer algorithm. The emotional state can be further classified into one or more levels. For example, an emotional state (e.g., happiness) can be further be classified into 10 numeric levels (e.g., 1 being the lowest happiness level and 10 being the highest happiness level).

In some cases, the emotional state of the subject can be classified using a computer algorithm. The emotional state can be further classified into one or more levels. For example, an emotional state (e.g., happiness) can be further classified into 10 numeric levels (e.g., 1 being the lowest happiness level and 10 being the highest happiness level).

In some cases, the emotional state of the subject can be assigned to a grading scale. For example, the subject can be asked to choose an option (1 to 9) on the following grading scale when given a testing substance (e.g., water):
1) I would be very happy to accept this water as my everyday drinking water;
2) I would be happy to accept this water as my everyday drinking water;
3) I am sure that I could accept this water as my everyday drinking water;
4) I could accept this water as my everyday drinking water;
5) Maybe I could accept this water as my everyday drinking water;
6) I don't think I could accept this water as my everyday drinking water;
7) I could not accept this water as my every day drinking water;
8) 1 could never drink this water;
9) 1 can't stand this water in my mouth and I could never drink it.

3. Third Application—Recreating Equivalent Compounds:

Another application of the universal code system may be recreating equivalent compounds. In one embodiment, this process involves providing an odor code profile of a target composition and returning a formula or recipe identifying combinations of elements from an odor palette and their relative concentrations or amounts, that is predicted to produce a similar or identical odor code profile as that of the target composition.

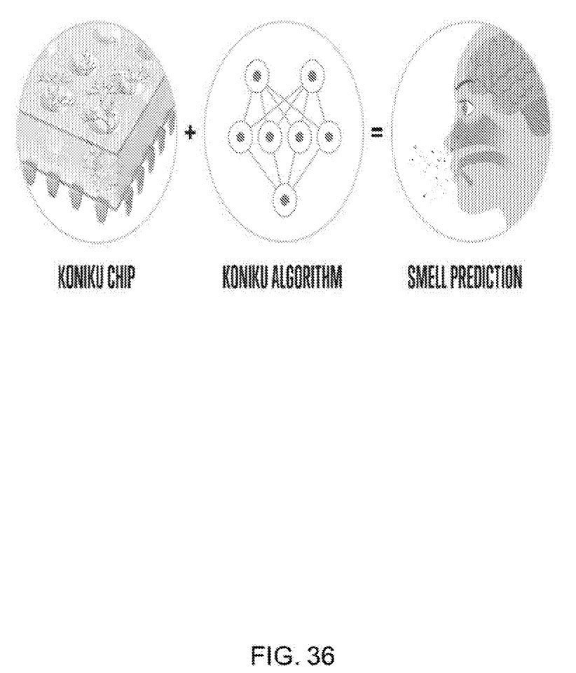

The process of recreating an equivalent compound may comprise encoding a target compound. The process of encoding the target compound may comprise mixing the target compound with a medium before injecting to the cell-sensor device. The process of encoding the new compounds may further comprise providing a signal of the new compound. The process of encoding the new compound may further comprise analyzing the signal by one or more algorithm and providing a code to the new compound based on the analysis. The process of encoding the new compound may further comprise returning the code of the compound to a user. After encoding the target compound and obtaining the code of the target compound, a copy of the target compound may be produced. FIG. 36 shows a non-limiting example of predicting, copying or reproducing any smell.

The process of recreating equivalent compounds may comprise determining compounds for detection by the odor encoding device. The compounds may be screened to identify ORs. The ORS may be modified to improve their sensitivities. The combination of cell (e.g., neuron, astrocyte or other cell) expression may be modified. ORS may be validated to accurately detect the compounds. The neurons and receptors that have been developed may be integrated in the odor encoding device platform to generate a laboratory prototype of the device. The odor encoding device may be further developed to contain smaller components assuring functional compatibility throughout. The final integration of a component may produce a market-ready, self-contained device.

For example, a mapping function (“g”) that predicts an odor code profile from concentrations or relative concentrations of elements from a palette of elements is provided. Such classifying functions are described above. An initial test formula or recipe, comprising one or a plurality of elements in the database and relative concentrations thereof, is also provided. The initial test formula can be provided by an expert in the field, or it can be generated by computer based on elements known to elicit responses from one or more olfactory receptors whose responses are known be part of the odor code profile for the target compound. Alternatively, the initial formula can be randomly generated. Then, the regressor predicts the odor code profile of a composition having the initial formula.

This predicted odor code profile is compared with the odor code profile of the target compound and a measure of difference, epsilon, is determined. There are many ways to determine epsilon. In one embodiment, epsilon is the sum of all the quantitative differences in response between the predicted odor code profile and the target odor code profile. In another embodiment, epsilon is Kullback-Leibler divergence, a Hellinger distance or a Renyi divergence. In another embodiment the measurement of distance can be 1-norm distance (Manhattan), 2-norm distance (Euclidean), p-norm distance (Minkowski), or infinity norm distance.

An acceptable level of epsilon can be set by the operator. An acceptable level may be, for example, a level at which an expert in the field, or a typical consumer, cannot distinguish a difference in smell between two different compositions. Alternatively, epsilon can be set such that between the reference product and the test product, both demonstrate substantially equivalent or equivalent market performance. (That is, produce substantially equivalent sales.) Alternatively, epsilon can be set such that between the reference product and the test product, neither shows a consumer preference (e.g., subjective consumer preference).

The computer can then engage in an iterative process of formula improvement. One such method involves making incremental changes to a test formula to produce a modified test formula, predicting an odor code profile for the modified test formula, and determining a measure of distance between the predicted odor code profile and the target odor code profile.

Alterations to a test formula can involve slightly changing concentration of one or a plurality of elements in the test formula (e.g., increasing or decreasing the concentration by no more than 50%, no more than 40%, no more than 30%, no more than 20%, by no more than 10%, no more than 50%, no more than 3%, no more than 2%, or no more than 1%. Alterations also can include adding to or subtracting from the formula no more than any of 10, 9, 8, 7, 6, 5, 4, 3, 2 or 1 elements. If the distance between the odor code profile of the target and a subsequent formula is less than the distance between the odor code profile of the target and a previous formula, this indicates that the new formula more closely approximates the target profile than the old formula. Then, the subsequent formula can be used as starting point for further modification, along the same lines. If the distance between the odor code profile of the target and a subsequent formula is more than the distance between the odor code profile of the target and a previous formula, this indicates that the subsequent formula less closely approximates the target than the previous formula. In this case, the previous formula can be used as the starting point again for modification. In this way, over many iterations, a test formula can be created, the distance of which from the target cannot be significantly improved. If, for this formula, the epsilon is within a predetermined level of tolerance, further use or testing with the final test formula can proceed. For example, a composition having the final test formula can be prepared and given to a human tester for testing and/or comparison to the target compound. Alternatively, known analytical methods can be used for the comparison, such as mass spectrometry, gas chromatography or NMR analysis.

In generating test formulas, the operator may set formula parameters. For example, it is expected that several different formulae may satisfy the level of tolerance requirements. The operator may determine to limit acceptable formula based on any of a number of criteria, including requirements include or exclude ingredients.

a) Cost

In on embodiment, the operator may set cost parameters for the formula. That is, the total cost of ingredients in the final formula may be set not to exceed a certain amount. For example, each element in the palette may have a different cost to purchase or to work with. The operator may set a parameter to select, between alternate formulae, a formula with a lower cost to produce. This may be done by swapping less expensive combinations of elements that produce the same odor code profile, for combinations of more expensive elements.

b) Health Considerations

Alternatively, certain elements in the palette may not meet standards for consumption or application to skin, for example because of toxicity or food or skin sensitivities. In this case, parameters can be set to limit amounts or to exclude from formulae, elements having undesirable characteristics.

c) Standards of production

Products may be desired that include certain ingredients. For example, it may be required that a product include fair trade or organic ingredients, or ingredients sourced from a specified geographical area (continent, country climate zone, etc.). In this case, the mapping function may be set to build formulae that reproduce a target composition and that include the required ingredients. In another example, it may be desired that certain non-meat products have the same taste as a target meat product, but without actually including meat. Accordingly, the parameters can be set to require certain meat substitutes, and a formula developed that has an aroma equivalent to or approximating (within a set epsilon) of the corresponding meat product.

A composition having a test formula of interest, for example, one chosen for testing in a product, can be produced by combining elements in the formula in amounts or relative concentrations set forth in the formula.

4. Fourth Application—Predicting Physiological States:

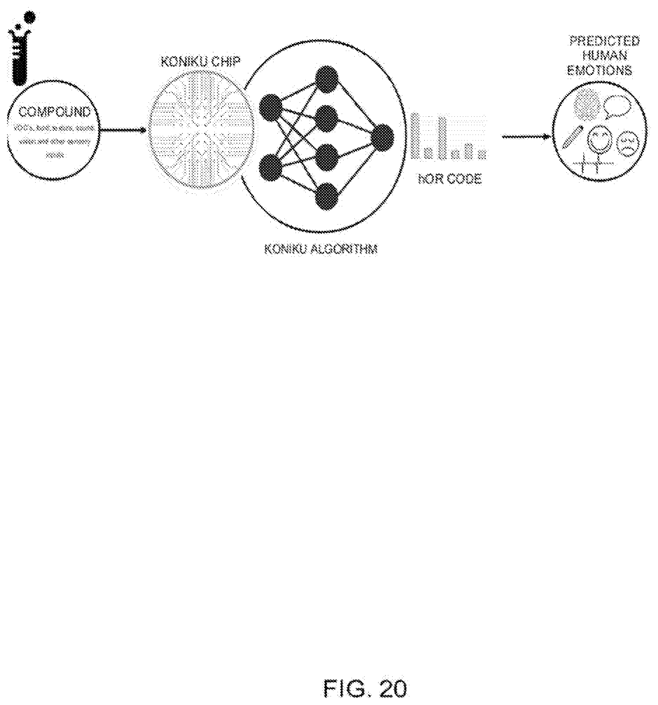

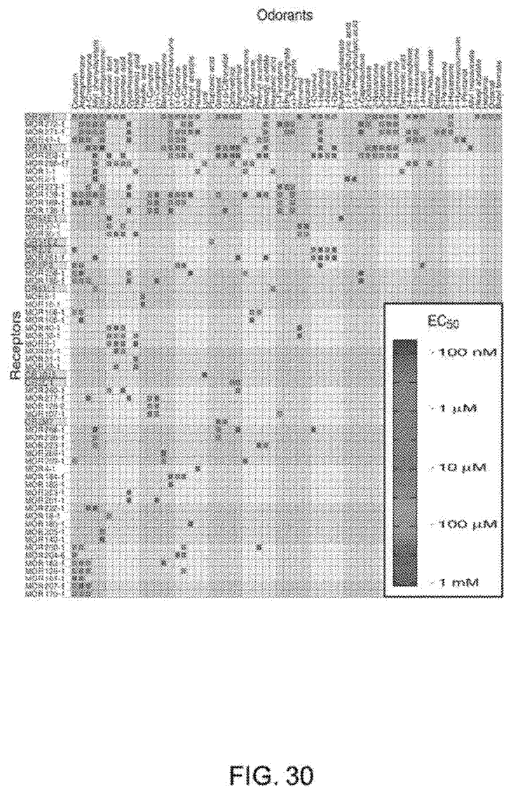

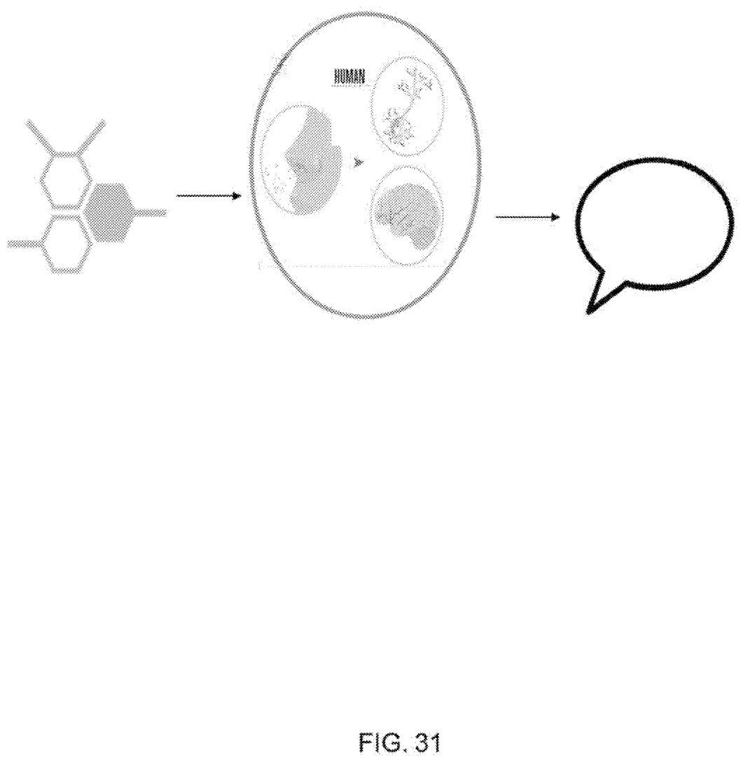

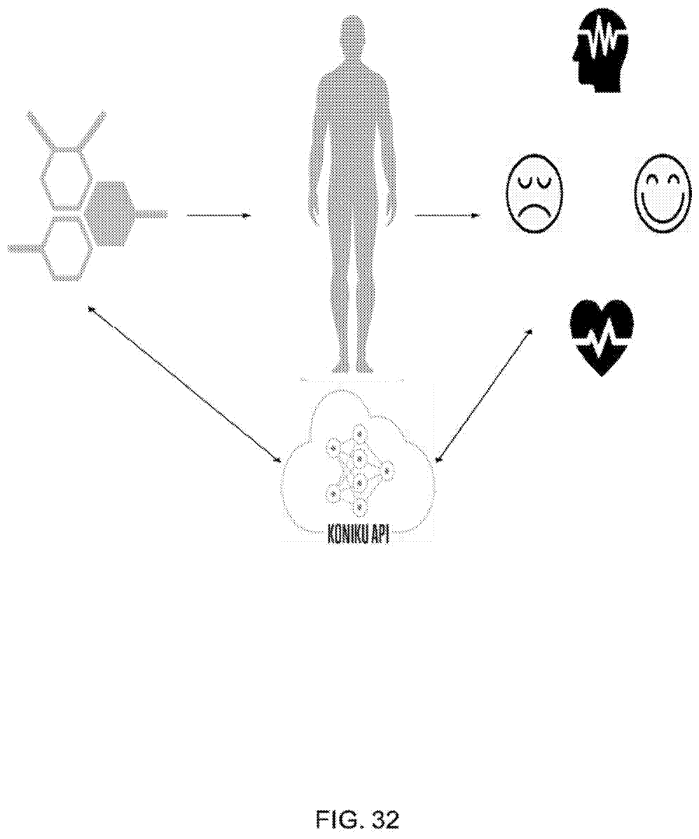

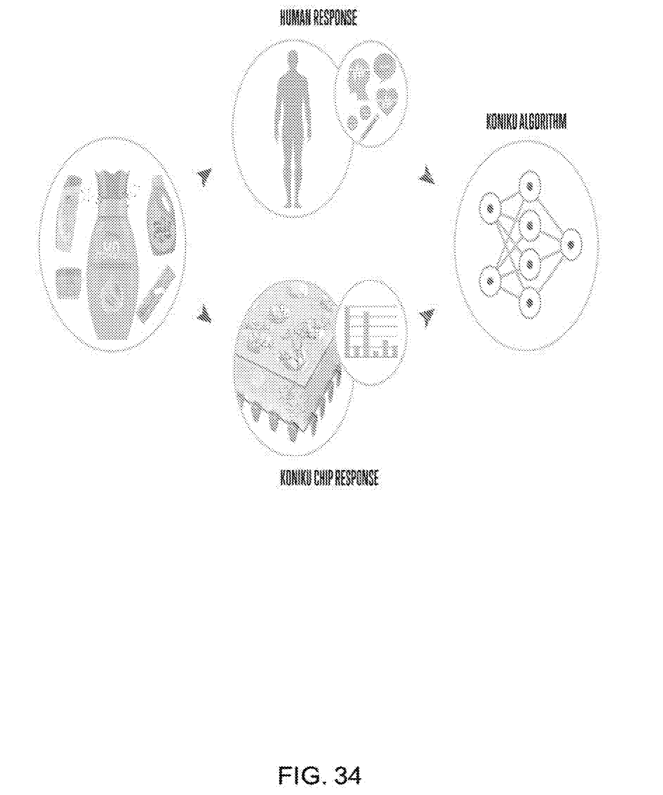

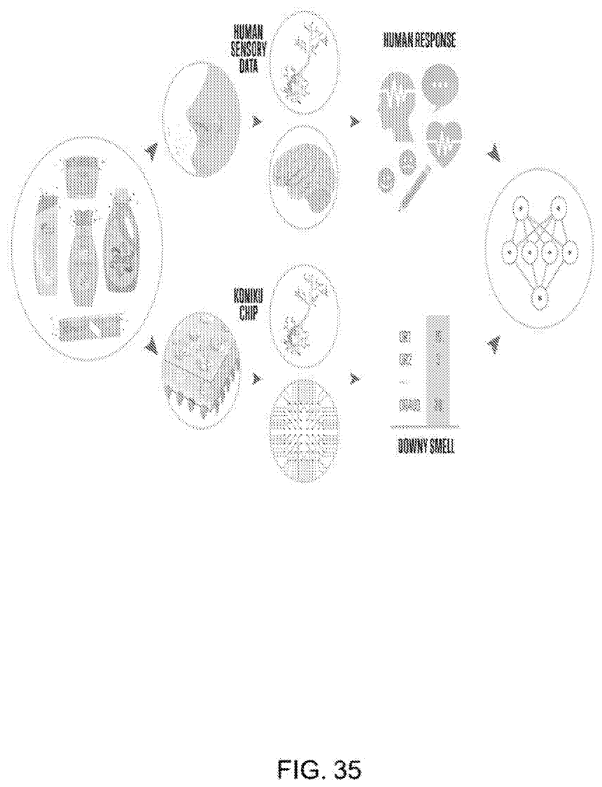

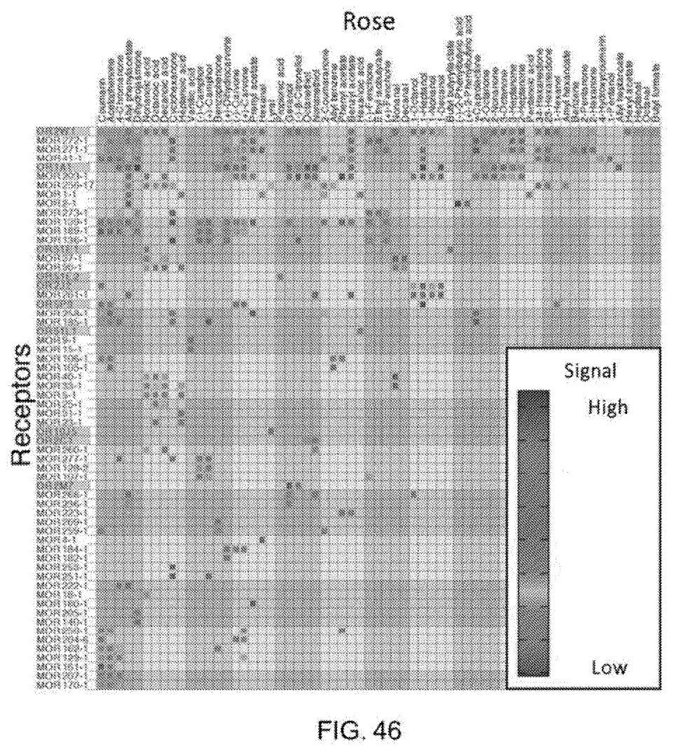

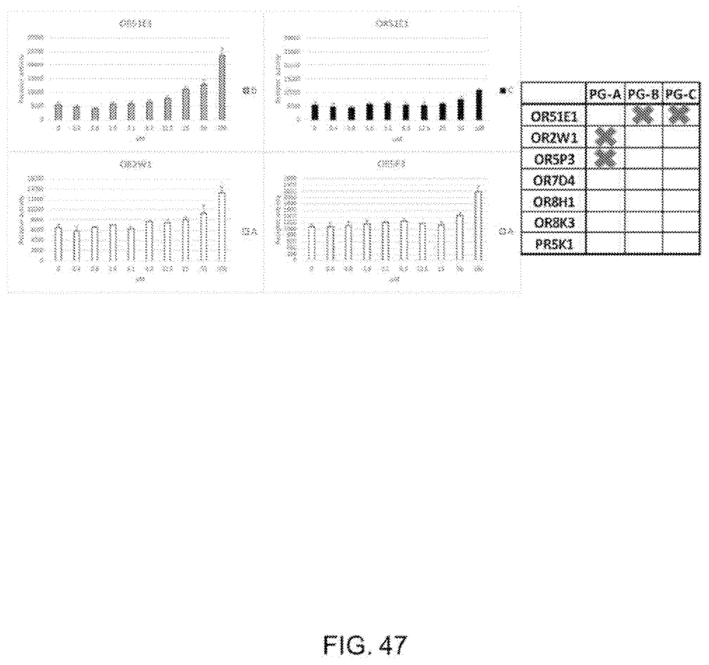

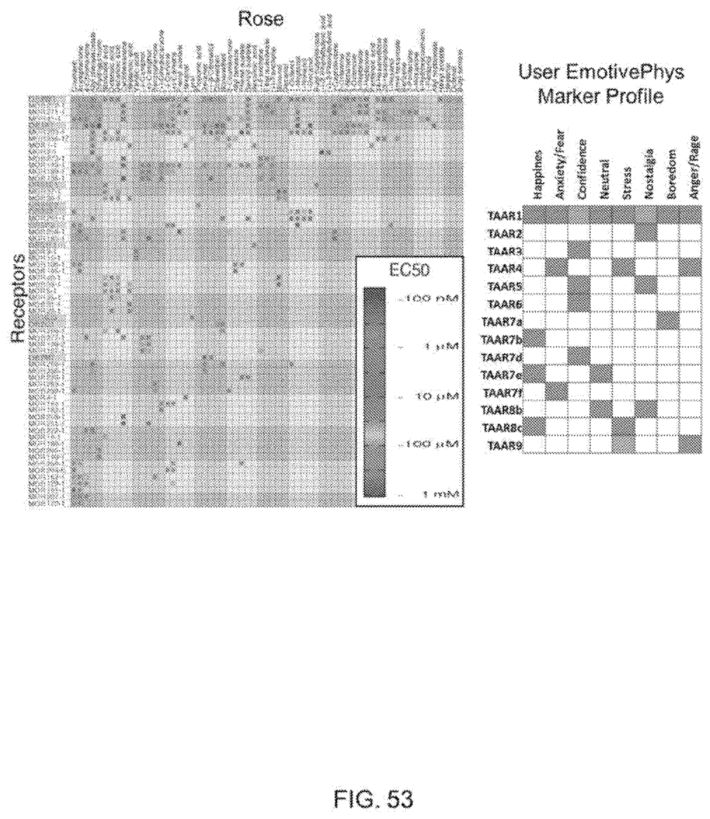

Another application of the universal code system may be predicting physiological states (e.g. emotion states) of a subject who is in contact with any compound or a mixture of compounds. The process of predicting physiological states (e.g. emotion states) of the subject may be conducted after mapping physiological states to each hOR or to a combination of hORs. FIG. 20 shows a non-limiting example of predicting emotions based on one or more compounds. FIG. 31 shows an overview of correlating physiological responses of humans to smell with the activation profile of each olfactory receptor. FIG. 32 shows a non-limiting example of correlating physiological responses of humans to smell with the activation profile of each olfactory receptor through the odor encoding device. FIG. 34 shows another non-limiting example correlating physiological responses of humans to smell with the activation profile of each olfactory receptor through the odor encoding device. FIG. 35 shows a non-limiting example of correlating physiological responses of humans to smell with the activation profile of each olfactory receptor through the odor encoding device and relevant algorithms. FIG. 30 shows a non-limiting example of mapping an odor with olfactory receptors. FIG. 46 shows a non-limiting example of mapping an odor with olfactory receptors. FIG. 47 shows a non-limiting example of mapping an odor with olfactory receptors in vertical bar format. FIG. 53 shows a non-limiting example of mapping an odor with olfactory receptors through dimensions of odor quality.

To predict physiological states, one or more algorithms may be used. The one or more algorithms may be machine learning algorithms. The one or more algorithms may be associated with statistical techniques. The one or more statistical techniques may include principal component analysis. The principal component analysis may comprise reducing the dimensionality of perceptual descriptors of the compound. The dimensionality of perceptual descriptors may be the number of perceptual descriptors. The number of physicochemical descriptors may be at least 1, 5, 10, 50, 100, 200, 300, 400, 500, 600, 700, 800, 900, 1000, 1100, 1200, 1300, 1400, 1500, 1600, 1700, 1800, or greater. The perceptual descriptors may be linguistic expressions. The perceptual descriptors may comprise, but not limited to, fruit, sweet, perfumery, aromatic, floral, rose, spicy, cologne, cherry, incense, orange, lavender, clove, strawberry, anise, violets, grape juice, pineapple, almond, vanilla, peach fruit, honey, pear, sickening, rancid, sour, vinegar, sulfidic, dirty linen, urine, green pepper, celery, maple syrup, caramel, woody, coconut, soupy, burnt milk, eggy, apple, light, musk, leather, wet wool, raw cucumber, chocolate, banana, coffee, yeasty, cheesy, sooty, blood, raw meat, fishy, bitter, clove, peanut butter, metallic, tea leaves, stale, mouse, seminal, dill, molasses, cinnamon, heavy, popcorn, kerosene, fecal, alcoholic, cleaning fluid, gasoline, sharp, raisins, onion, buttery, and herbal. The dimensionality of perceptual descriptors may be reduced to one perceptual principal component. The perceptual principal component may be pleasantness or happiness. The pleasantness or happiness may refer to the continuum from unpleasant to pleasant.

The principal component analysis may comprise reducing the dimensionality of physicochemical descriptors of the compound. The dimensionality of physicochemical descriptors may be the number of physicochemical descriptors. The number of physicochemical descriptors may be at least 1, 5, 10, 50, 100, 200, 300, 400, 500, 600, 700, 800, 900, 1000, 1100, 1200, 1300, 1400, 1500, 1600, 1700, 1800, or greater. The physicochemical descriptors may describe the molecular features of the compound. The physicochemical descriptors may include, but not limited to, the carbon atom number, the molecular weight, the number of carbon-carbon bond, the number of functional groups, the aromaticity index, the maximal electrotopological negative variation, the number of benzene-like rings, the number of aromatic hydroxyls, the average span R, the number of carboxylic group, and the number of double bonds. The dimensionality of perceptual descriptors may be reduced to one physicochemical principal component. The physicochemical principal component may be a sum of atomic van der Waals volumes.

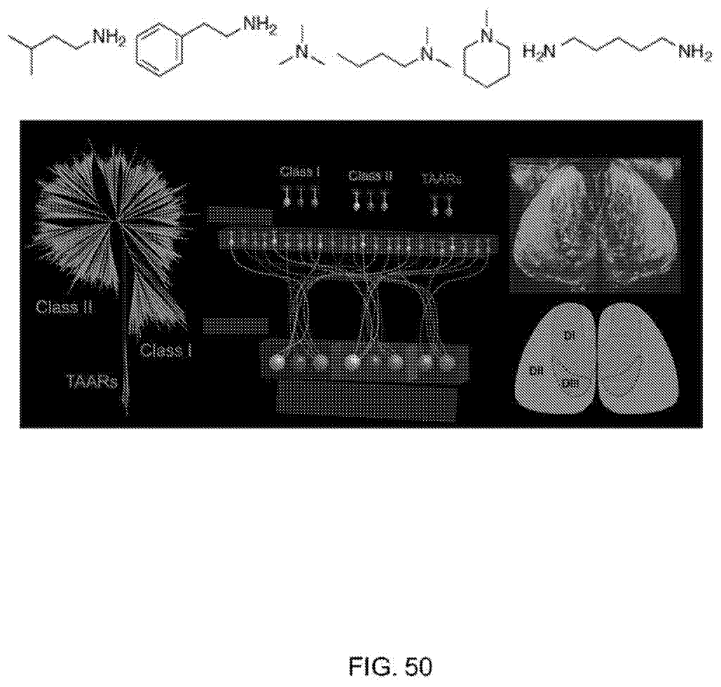

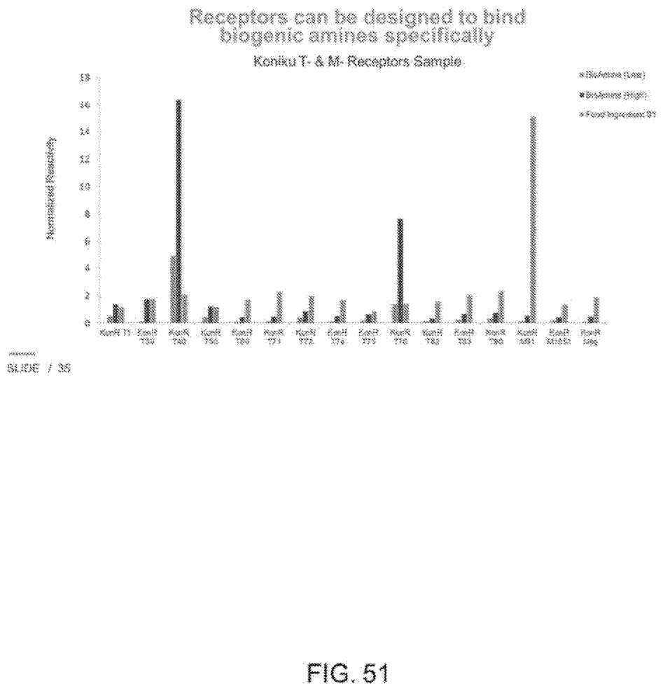

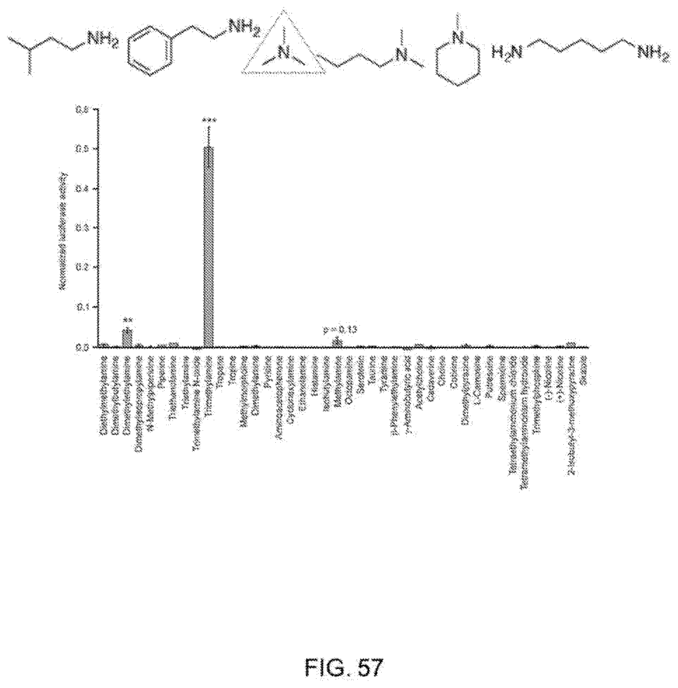

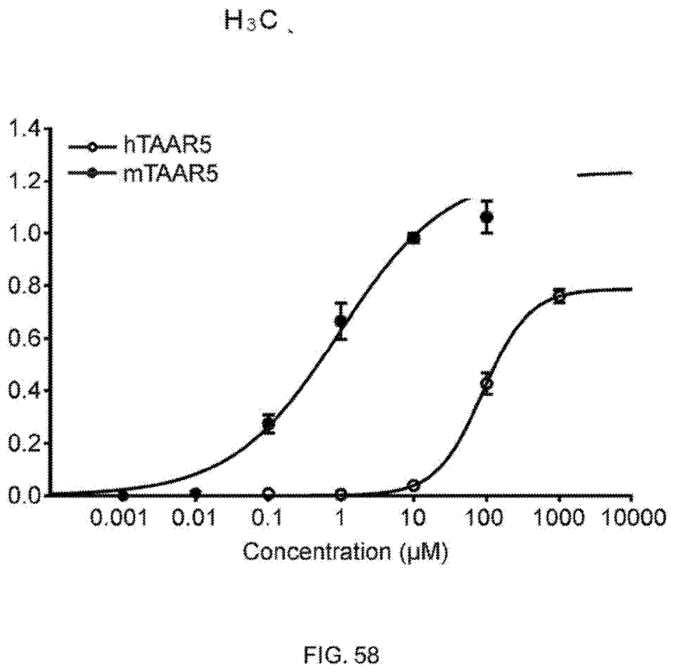

The principal component analysis may further comprise finding that perceptual principal component may have a privileged link to physicochemical principal component. The privileged link may be linear relationship between the perceptual principal component and physicochemical principal component. The privileged link may allow a single optimal axis for explaining the variance in the physicochemical data to be the best predictor of perceptual data. Predict physiological states may be used in situations such as malodorant blocker, culturally targeted product design, harmful chemicals detection, or triggering specific targeted emotions. FIG. 50 shows a non-limiting example of detecting neural responses to amine. FIG. 51 shows a non-limiting example that receptors can be designed to bind biogenic amines specifically. FIG. 57 shows a non-limiting example of detecting amines through trace amine-associated receptors. FIG. 58 shows that synthetic biology can increase the sensitivity and specificity of the trace amine-associated receptors.

The following steps may be executed to predict physiological states of the subjects.

5. Fifth Application—Quality Control:

Provided herein are methods of ensuring quality control in the production and distribution of products.

a) Ingredients in a Production Run

Product production typically includes the creation of formulas using batches of ingredients. However, ingredients can differ somewhat from batch to batch resulting in different code profiles between production runs using different batches of ingredients. Accordingly, one method of quality control involves setting an older code profile standard for a product or an ingredient to be included in a product. During production of the product, a sample of an ingredient from a batch can be tested for its odor code profile. The tested profile can be compared against the reference standard and a measure of difference can be determined. If the measured differences within an acceptable amount then, the ingredient can be included in the production run. Alternatively, if the measured difference is outside of acceptable amount then, ingredients from the batch tested are not included. A new batch may then be tested.

b) Changes in Quality Over Time

Changes in a product or samples from a product production run over time also can be determined, for example, for determining expiration dates or for removing from store shelves products that are overdue. In one embodiment, a product, such as a fruit or vegetable, e.g., a banana, can be tested to produce a reference article profile corresponding to various levels of ripeness or freshness. Then, products can be tested over time to determine a distance between their article profile and the reference odor code profile. An odor code profile from a product may indicate that the product is stale or overripe. This may be reflected in for example the fact that a distance between an odor code profile from the product and a reference article profile is greater than an acceptable level of tolerance. Alternatively, code profile indicating staleness or over ripeness can be used as a reference. When a distance between a tested odor code profile and a reference article profile comes with any stage or degree of difference, the product may be considered past its shelf life. Such products can be removed from the shelf. In a related method, a test product can be tested over time at varying levels of freshness/staleness or ripeness/over-ripeness. The degree of product this can be determined using external standards such as expert sampling of the product. The time for a product to produce and odor code profile consistent with a set degree of staleness or over-ripeness can be determined in such time can be used in the determination of a “sell by” date.

c) Comparisons of Products from Different Production Facilities

Quality control can involve uniformity between products produced a different production facilities. This may be a reflection of inclusion of different batches of ingredients at such different facilities. Accordingly, a quality control standard of an odor code profile of a product can be produced. A measure of deviation from the standard odor code profile can be set, outside of which a product is considered unfit for sale or consumption. At a plurality of different production facilities in which a product is produced, products from one or a plurality of production runs can be tested for their order code profile. These profiles can be compared with the reference odor code profile and a degree of difference determined. If a product of a production run at a facility satisfies the quality control standard, that product can be designated for distribution into the supply chain that ends with customers. If a product of a production run at a facility does not satisfy the quality control standard because its article profile is too deviant from the order code profile standard, that production run is designated for non-release or for some other use than sale and consumption.

6. Sixth Application—Odor Control

Restrooms and various other environments can be malorodous. Malorodousness can be allevitated as follows. An oror code print of a malorodrous environment is created. A mapping function as disclosure herein is used to predict a formula of elements which, released into the environment, alter the malodorous smell to one more pleasing, for example, the smell of flowers or lavender.

7. Optimization for Positive Emotion

After determining an emotional response to a olfactory stimulus, the formula for the stimulus can be modified to change the emotional response (e.g., more happy, more energize, less anxious, etc.). The mapping function can identify a modification to a formula for a product predicted to elicit the different emotional response.

8. Formulae for Similar Compostions

Certain aspects of an odor—sweetness, intensity, may be desired to be maintained while changing an underlying aspect of an odor, for example, maintaining sweetness of strawberry while changing smell to raspberry. In this case, the mapping function can be set to maintain the desired characteristics, while changing the other characteristics.

9. Sending and Receiving Odor Information

An individual, such as a customer, a person communication in a social networking context, can request an odor code remotely, e.g., via a user interface of a computer through a website. A host can receive the query and transmit a formula for a compostions that produces the requested odor. The receiver can then generate the composition that produces the odor.

Alternatively, a person can encode an odor, for example product or a body odor using a device as disclosed herein. The person can transmit the odor code profile over a communications network to a remote location, where the odor can be reproduced using a mapping function as disclosed herein.

D. Preparation:

Human subjects can be individually surveyed (to not influence each other). A number of external parameters, such as position of the subject, temperature of the room, light in the room, sound in the room (no background sound), can be maintained constant to cancel body signal variations coming from other senses than taste and/or smell. In some cases, the subject can perform a meditation, eat a meal, and/or take a shower under controlled conditions to cancel body signal variations.

1. Baseline Measurement:

Physiological signals can be detected and/or measured from the non-stimulated subject in order to have a baseline before stimulus. In some cases, the subject can take a control substance (e.g., air or water) to access the subject's physiological state without the inducement of the stimulus.

Emotions Reference Measurement:

The sensors can be used to detect and/or measure physiological signals of the subject that is reacting to different stimulus associated with targeted emotions.

Classical stimuli, such as music, images, movie scenes, and video games can be used to train the computer algorithm to make the correct connection between the physiological signals when given classical stimuli and the corresponding classical emotions (e.g., happiness, sadness). For example, images known to elicit happiness can be given to the subjects, and then the physiological signals measured from the subject can be linked to the target emotional state, e.g., happiness.

Synesketch algorithms can be used to analyze emotional content of text sentences in terms of emotional types (e.g., happiness, sadness, anger, fear, disgust, and surprise), weights (how intense the emotion is), and/or a valence (is it positive or negative). The recognition technique can be grounded on a refined keyword spotting method which can employ a set of heuristic rules, a WordNet-based word lexicon, and/or a lexicon of emoticons and common abbreviations.

Articles linked with classical emotions (e.g., happiness, sadness), but also emotions more taste and/or smell related can be used. These articles can be taken from database (for comparison with similar studies) for classical emotions and can be used to generate taste and/or smell related emotions.

2. Compounds Responses:

Evaluation can be made on base compounds. The base compound can be a smelling and/or tasting reference compound with expected results. For example, sweet reference compound can be expected to be associated with joy. Evaluation can also be made on compounds with unknown results.

3. Features Extraction and Features Engineering:

Different features can be extracted from the physiological signals. These features can be engineered (e.g. remove baseline) and used as input to a computer algorithm, such as a machine learning algorithm, to match these features with the compounds.

For the analysis of the linguistic expressions, a computer algorithm (e.g., machine learning algorithm) can extract features from the voice (e.g., tone) and/or from the content.

The machine leaning algorithm can comprise linear regression, logistic regression, decision tree, support vector machines (SVM), naive bayes, k-nearest neighbors algorithm (k-NN), k-means clustering, random forest, dimensionality reduction algorithms, gradient boosting algorithms, such as gradient boosting machine (GBM), extreme gradient boosting (XGBoost), LightGBM, and CatBoost, or any combination thereof.

The combination of data sets with the presentation of taste, smell, sound, images and/or tactile signal can be used to predict a subject's physiological state (e.g., happiness or sadness). The methods can be used to design a set of optimal stimuli to provide a desired response.

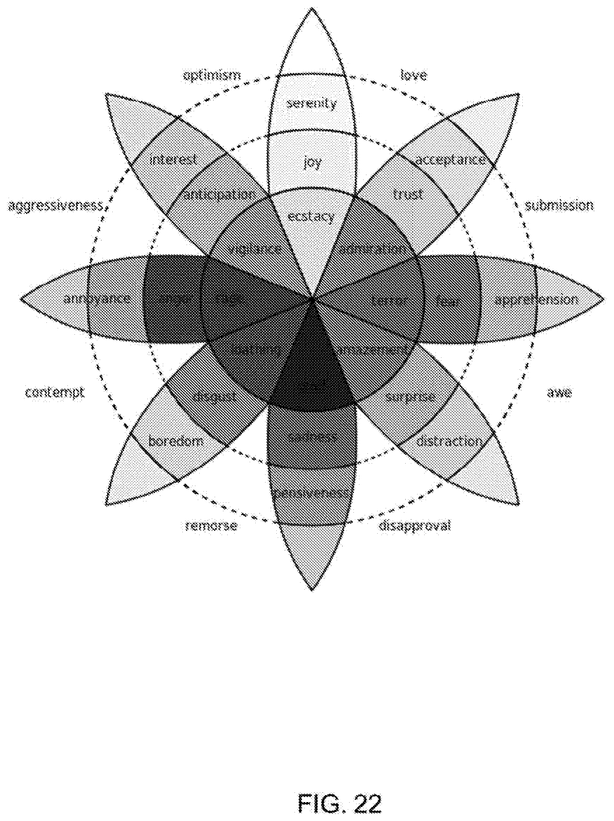

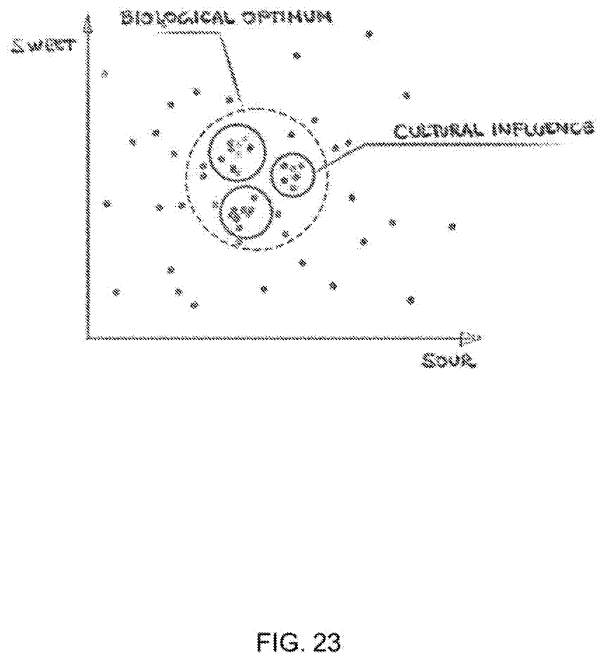

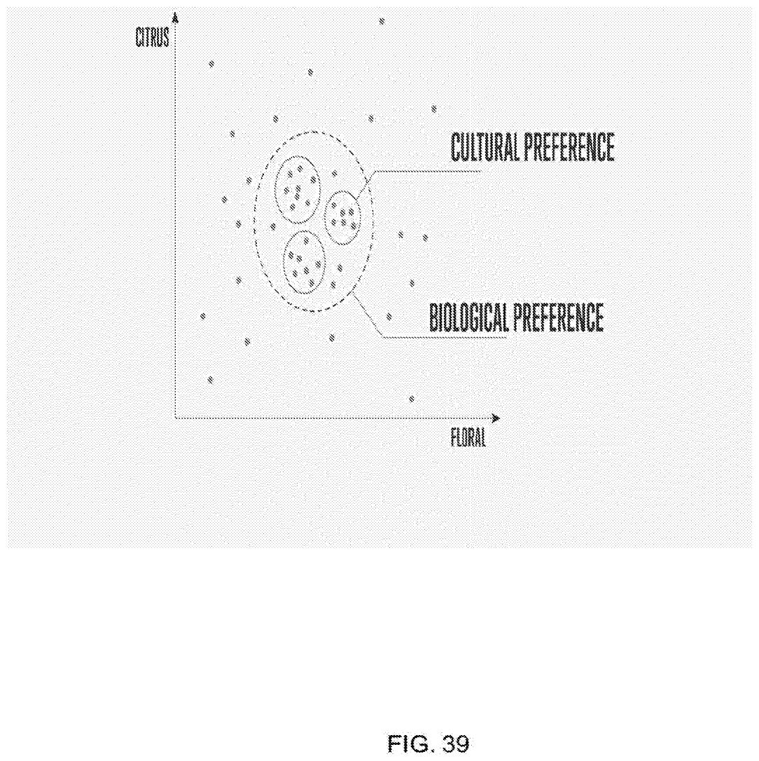

The method can be used to the creation of a precise emotions flower for general emotions (as shown in FIG. 22) and/or for smell/taste related emotions. The method can be used to map between a selected database of compounds and their corresponding emotions. The method can be applied to different group of people, such as based on ethnicities, cultures, socio-economic background, in order to get a more precise emotions map (as shown in FIGS. 23 and 39).

### Examples

These examples are provided for illustrative purposes only and not to limit the scope of the claims provided herein.

### I. Example 1—Cell-Based Sensors for Detecting a Range of Odorants, Representing a State, Such as a Ripeness State of a Single Piece of Fruit or a Batch of Fruit

In some embodiments, the disclosed cell-based sensor devices and systems may be used to detect a range of odorants associated with, for example, the ripeness state of fruit. Table 1a comprises a list of odorant compounds that are produced by fruit. Table 1b comprises a list of insect odorant receptors that may bind one or more of the compounds in Table 1a. Table 1c comprises a list olfactory compounds.

In some embodiments of the disclosed sensor devices and systems, the cells in the sensor devices or panels may be engineered to express one or more of the insect odorant receptors listed in Table 1b. In some cases, a cell may express multiple copies of a single odorant receptor. In some cases, each cell of an array of cells may express multiple copies of a single odorant receptor. In some cases, different cells may express multiple copies of a different odorant receptor. A cell-based sensor array may comprise cells where each odorant receptor may recognize one or more of the compounds in Table 1a, and thus may detect a single odorant compound or a mixture of the odorant compounds.

In some embodiments, an air-sampling device may be used in conjunction with a sensor device or sensor panel, where the air-sampling device collects an air sample from the air that is in close proximity to the fruit and facilitates transfer of any odorant compounds contained therein into the sensor device or panel using any of the air-sampling device mechanisms described above. For example, in some cases, a cell-based sensor device may comprise a semi-permeable membrane such that the odorants pass through the membrane and diffuse into the liquid medium covering the neurons on the detection device. Upon binding to the odorant receptor, one or more G-protein-coupled signaling pathways are activated inside the cell, and an action potential may be triggered. In some cases, at least one cell in each element (e.g., chamber) of an array is in contact with or in close proximity to an electrode. In some cases, at least once cell in each element of an array may at least partially engulf an electrode, e.g., a three-dimensional electrode. In some cases, multiple cells in each element of an array are in contact with, in close proximity to, or at least partially engulf an electrode. In these cases, an electrical impulse generated by one or more cells of the array may be directed to a signal detector by the one or more electrodes.

An electrode may be wired such that the binding of an odorant to a particular cell results in a unique signal (based on its location in the array) such that the processor or computer used to read data from the array of electrodes may compute which cell has bound an odorant. This permits mapping back to the odorant receptor since each cell uniquely expresses a single odorant receptor. Through the decoding of odorant receptors that have generated electrical signals, one may obtain a pattern of receptors that have been activated. In some cases, a particular odorant or set of odorants may yield a particular pattern of receptor activation.

Furthermore, because the electrodes may permit measurement of sub-threshold signals (this is true for all embodiments of the disclosed sensor devices and systems described above), quantitative information may be derived from a cell, thereby yielding information related to odorant concentration. By running standard control samples across the array, a database may be generated to determine how well different compounds may be binding across the array. Furthermore, for each of these controls, detection may be performed based on a serial dilution curve, thereby allowing a pattern of electrical signals to be mapped back to the identity and concentration of a compound from an unknown sample.

That is, the pattern of compound binding and receptor activation across the array may be more than just on/off, but may also capture information related to odorant concentration levels. Thus, one can map back from the results of a test sample and may determine the identity and/or concentration of the odorant in the test sample.

In the case of multiple types of odorants binding to multiple cells on the array, a more complex signal pattern or fingerprint may be recorded for the particular mixture, since the signal pattern or fingerprint may encode compound identity information and relative concentration information with overlapping effects.

In some embodiments, the use of machine learning algorithms may be used to process sensor signals, e.g., for distinguishing between a real binding/activation event and background noise, and/or for interpreting the electrical signal pattern or fingerprint in order to improve the accuracy of compound identification or concentration determination.

TABLE 1a
Odorant compounds produced by fruit or plants.

| Compound Name | CAS # |
|---|---|
| alpha-ionone | 127-41-3 |
| alpha-phellandrene | 99-83-2 |
| alpha-pinene | 7785-70-8 |
| benzaldehyde | 100-52-7 |
| beta-ionone | 14901-07-6 |
| beta-pinene | 18172-67-3 |
| butyric acid | 107-92-6 |
| caryophyllen | 87-44-5 |
| damascenone | 23726-93-4 |
| delta-decalactone | 705-86-2 |
| e-2-hexenal | 6728-26-3 |
| ethyl butyrate | 105-54-4 |
| gamma-decalactone | 706-14-9 |
| geranial | 5392-40-5 |
| geraniol | 106-24-1 |
| hexanoic acid | 142-62-1 |
| hexyl acetate | 142-92-7 |
| limonene | 138-86-3 |
| linalool | 78-70-6 |
| mesifuran | 4077-47-8 |
| methyl anthranilate | 134-20-3 |
| methyl butyrate | 623-42-7 |
| neral | 5392-40-5 |
| nerolidol | 7212-44-4 |
| raspberry ketone | 5471-51-2 |

TABLE 1b
Odorant receptors for fruit-specific volatile compounds.

| | | | Literature | | Literture | |
|---|---|---|---|---|---|---|
| Odorant | CAS # | Organism | code | GenBank ID | Indication | Reference |
| limonene | 138-86-3 | Apolygus | AlucOR46 | NM_001190564.1 | Tuned to six plant | Zhang Z, Zhang M, Yan S, |
| | | lucorum | | | volatiles: | Wang G, Liu Y. A Female- |
| | | (Meyer-Dür) | | | (S)-(−)- Limonene, | Biased Odorant Receptor from |
| | | | | | (R)-(+)-Limonene, | Apolygus lucorum (Meyer-Dür) |
| | | | | | (E)-2- Hexenal, | Tuned to Some Plant Odors. Int |
| | | | | | (E)-3-Hexenol, | J Mol Sci. 2016 Jul 28;17(8). |
| | | | | | 1-Heptanol, and | pii: E1165. doi: |
| | | | | | (1R)-(−)-Myrtenol | 10.3390/ijms17081165. |
| | | | | | | PubMed PMID: 27483241; |
| | | | | | | PubMed Central PMCID: |
| | | | | | | PMC5000588. |
| limonene | 138-86-3 | Megoura viciae | OBP3 from M. | KT750882.1 | (E)-β-farnesene | Northey T, Venthur H, De |
| | | and Nasonovia | viciae | | (−)-α-pinene, | Biasio F, Chauviac FX, Cole A, |
| | | ribisnigri | | | β-pinene, and | Ribeiro KA Junior, Grossi G, |
| | | | | | limonene | Falabella P, Field LM, Keep |
| | | | | | | NH, Zhou JJ. Crystal Structures |
| | | | | | | and Binding Dynamics of |
| | | | | | | Odorant-Binding Protein 3 |
| | | | | | | from two aphid species |
| | | | | | | Megoura viciae and Nasonovia |
| | | | | | | ribisnigri. Sci Rep. 2016 Apr |
| | | | | | | 22;6:24739. doi: |
| | | | | | | 10.1038/srep24739. PubMed |
| | | | | | | PMID: 27102935; PubMed |
| | | | | | | Central PMCID: PMC4840437. |
| limonene | 138-86-3 | Maruca vitrata | MvitGOBP1-2 | NP_001140185.1 | MvitGOBP1-2 had | Zhou J, Zhang N, Wang P, |
| | | Fabricius | | | different binding | Zhang S, Li D, Liu K, Wang G, |
| | | (Lepidoptera: | | | affinities with 17 | Wang X, Ai H. Identification of |
| | | Crambidae) | | | volatile odorant | Host-Plant Volatiles and |
| | | | | | molecules including | Characterization of Two Novel |
| | | | | | butanoic acid butyl | General Odorant-Binding |
| | | | | | ester, limonene, 4- | Proteins from the Legume Pod |
| | | | | | ethylpropiophenone, | Borer, Maruca vitrata Fabricius |
| | | | | | 1H indol-4-ol, | (Lepidoptera: Crambidae). |
| | | | | | butanoic acid octyl | PLoS One. 2015 Oct 30;10(10): |
| | | | | | ester, and 2 methyl- | e0141208. doi: 10.1371/journal. |
| | | | | | 3-phenylpropanal | pone.0141208. eCollection |
| | | | | | | 2015. PubMed PMID: |
| | | | | | | 26517714; PubMed Central |
| | | | | | | PMCID: PMC4627759. |
| limonene | 138-86-3 | Vinegar fly | Odorant | NP_525013.2 | Single dedicated | Dweck UK, Ebrahim SA, |
| | | Drosophila | receptor Or19a | | olfactory pathway | Kromann S, Bown D, Hillbur |
| | | melanogaster | | | determines | Y, Sachse S, Hansson |
| | | | | | oviposition fruit | BS,Stensmyr MC. Olfactoly |
| | | | | | substrate choic | preference for egg laying on |
| | | | | | | citrus substrates in Drosophila. |
| | | | | | | Curr Biol. 2013 Dec 16;23(24): |
| | | | | | | 2472-80. doi: |
| | | | | | | 10.1016/j.cub.2013. 10.047. |
| | | | | | | Epub 2013 Dec 5. PubMed |
| | | | | | | PMID: 24316206. |
| linalool | 78-70-6 | Bombyx mori | BmorOR-19 | NP_001091785.1 | Tuned to the | Groβe-Wilde E, Stieber R, |
| | | | | | detection of the | Forstner M, Krieger J, Wicher |
| | | | | | plant odor linalool | D, Hansson BS. Sex-specific |
| | | | | | | odorant receptors of the tobacco |
| | | | | | | hornworm manduca sexta. |
| | | | | | | Front Cell Neurosci. 2010 Aug |
| | | | | | | 3;4. pii: 22. doi: |
| | | | | | | 10.3389/fncel.2010.00022. |
| | | | | | | eCollection 2010. PubMed |
| | | | | | | PMID: 20725598; PubMed |
| | | | | | | Central PMCID: PMC2922936. |

TABLE 1c
List of odorant compounds.

| Compound Name | CAS # |
|---|---|
| Abhexone | 698-10-2 |
| Acetophenone | 98-86-2 |
| AcetylPyridine | 1122-62-9 |
| Adoxal | 141-13-9 |
| AldehydeC-16highcon | 77-83-8 |
| AldehydeC-16lowcon | 77-83-8 |
| AldehydeC-18 | 104-61-0 |
| AllylCaproate | 123-68-2 |
| AmylAcetateiso-amylAcetat | 123-92-2 |
| AmylButyrate | 540-18-1 |
| AmylCinnamicAldehyeDiethy | 60763-41-9 |
| AmylPhenylAcetate | 102-19-2 |
| AmylValerate | 2173-56-0 |
| Andrane | 29597-36-2 |
| Anethole | 104-46-1 |
| Anisole | 100-66-3 |
| Auralva | 89-43-0 |
| Benzaldehyde | 100-52-7 |
| BenzoDihydropyrone | 119-84-6 |
| BornylAcetateiso-BornylA | 5655-61-8 |
| ButanoicAcid | 107-92-6 |
| Butanol | 71-36-3 |
| ButylQuinolineiso | 544-40-1 |
| ButylSulfide | 67634-06-4 |
| Camphordl | 464-48-2 |
| Carvone-l | 99-49-0 |
| Caryophyllene | 87-44-5 |
| Cashmeran | 33704-61-9 |
| Celeriax | 17369-59-4 |
| Chlorothymol | 89-68-9 |
| CinnamicAldehyde | 104-55-2 |
| Citral | 141-27-5 |
| Citralva | 5585-39-7 |
| Coumarin | 91-64-5 |
| Cresol-m | 108-39-4 |
| Cresol-p | 106-44-5 |
| CresylAcetate-p | 140-39-6 |
| CresylMethylEther-p | 103-93-5 |
| CresylisoButyrate-p | 104-93-8 |
| CuminicAldehyde | 122-03-2 |
| Cyclocitral-iso | 1423-46-7 |
| Cyclodithalfarol | 55704-78-4 |
| Cyclohexanedione1,2 | 765-87-7 |
| Cyclohexanol | 108-93-0 |
| Cyclotene | 80-71-7 |
| Cyclotropal | 67634-23-5 |
| Decad ienal2,4-trans-trans | 25152-84-5 |
| DecahydroNaphthalene | 91-17-8 |
| DibutylArnine | 111-92-2 |
| DiethylSulfide | 352-93-2 |
| DimethylBenzylCarbinylBut | 10094-34-5 |
| DimethylPhenylEthylCarb | 103-05-9 |
| DimethylPyrazine2,3 | 5910-89-4 |
| DimethylPyrazine2,5 | 123-32-0 |
| DimethylPyrrole2,5 | 625-84-3 |
| DimethylTrisulfide | 3658-80-8 |
| Diola | 7/3/7474 |
| EthylPropionate | 105-37-3 |
| EthylPyrazinehighconc | 13925-00-3 |
| EthylPyrazinelowconc | 13925-00-3 |
| Eucalyptol | 470-82-6 |
| Floralozone | 67634-15-5 |
| Furfural | 98-01-1 |
| FurfurylMercaptan | 98-02-2 |
| Heptanol1 | 111-70-6 |
| Hexanal | 68-25-1 |
| Hexanol1 | 111-27-3 |
| Hexanol3 | 623-37-0 |
| Hexenal-trans1 | 6728-26-3 |
| HexylArninehighconc | 111-26-2 |
| HexylArninelowconc | 111-26-2 |
| HexylCinnamicAldehyde | 101-86-0 |
| HydratropicAldehydeDiAl | 90-87-9 |
| HydroxyCitronellal | 107-75-5 |
| Indole | 120-72-9 |
| Indolene | 67801-36-9 |
| Iodoform | 75-47-8 |
| Ionone-betahig hconc | 14901-07-6 |
| Ionone-betalowconc | 14901-07-6 |
| Ironealpha | 79-69-6 |
| Limonened | 126-91-0 |
| Linalool | 138-86-3 |
| Lyral | 31906-04-4 |
| Maritima | 67258-87-1 |
| Melonal | 106-72-9 |
| Menthol-1 | 2216-51-5 |
| MethoxyNaphthalene2 | 93-04-9 |
| Methyl-iso-Borneol2 | 134-20-3 |
| Methyl-iso-Nicotinate | 462-95-3 |
| MethylAcetaldehydeDiAce | 611-13-2 |
| MethylAnthranilate | 2271-428 |
| MethylFuroate | 491-35-0 |
| MethylQuinolinepara | 2459-09-8 |
| MethylThiobutyrate | 68917-50-50 |
| Methylsalicylate | 2432-51-1 |
| MuskGalaxolide | 1222-05-5 |
| MuskTonalid | 1508-02-1 |
| Myracaldehyde | 37677-14-8 |
| NonylAcetate | 143-13-5 |
| Nootkatone | 4674-50-4 |
| Octanol1 | 111-87-5 |
| Octenol-1-3-OL | 3391-86-4 |
| PentanoicAcid | 109-52-4 |
| PentenoicAcid4 | 591-80-0 |
| PhenylAceticAcid | 103-82-2 |
| PhenylAcetylene | 536-74-3 |
| PhenylEthanolhighconc | 60-12-8 |
| PhenylEthanollowconc | 60-12-8 |
| Phorone-iso | 78-59-1 |
| Pinenealpha | 80-56-8 |
| PropylButyrate | 105-66-8 |
| PropylQuinoline-iso | 135-79-5 |
| PropylSulfide | 111-47-7 |
| Pyridine | 110-86-1 |
| Safrole | 94-59-7 |
| Sandiff | 69460-08-8 |
| Santalol | 115-71-9 |
| Skatole | 83-34-1 |
| Terpineolmostlyalpha | 10482-56-1 |
| TetrahydroThiophene | 110-01-0 |
| Tetraquinone | 91-61-2 |
| Thienopyrimidine | 3626-71-7 |
| ThioglycolicAcid | 123-93-3 |
| Thiophene | 110-02-1 |
| Thymol | 89-83-8 |
| Tolualdehyde-ortho | 529-20-4 |
| Toluenehighconc | 108-88-3 |
| Toluenelowconc | 108-88-3 |
| TrimethylAmine | 75-50-3 |
| Undecalactonegamma | 104-67-6 |
| UndecylenicAcid | 112-38-9 |
| Valeraldehyde-iso | 590-86-3 |
| ValericAcid-iso | 503-74-2 |
| Valerolactonegamma | 108-29-2 |
| Vanillin | 121-33-5 |
| Zingerone | 122-48-5 |

TABLE 2
Examples of odorant receptors.

| Gene Name | Accession Number |
|---|---|
| odorant receptor family 7 subfamily D member 4 P79L variant [Homo sapiens] | ABV66285.1 |
| odorant receptor family 7 subfamily D member 4 S84N variant [Homo sapiens] | ABV66284.1 |
| odorant receptor family 7 subfamily D member 4 WM variant [Homo sapiens] | ABV66283.1 |
| odorant receptor family 7 subfamily D member 4 RT variant [Homo sapiens] | ABV66282.1 |
| odorant receptor HOR3′beta5 [Homo sapiens] | AAG42368.1 |
| odorant receptor HOR3′beta4 [Homo sapiens] | AAG42367.1 |
| odorant receptor HOR3′beta3 [Homo sapiens] | AAG42366.1 |
| odorant receptor HOR3′beta2 [Homo sapiens] | AAG42365.1 |
| odorant receptor HOR3′beta1 [Homo sapiens] | AAG42364.1 |
| olfactory receptor 7D4 [Homo sapiens] | NP_001005191.1 |
| HOR 5′Beta1 [Homo sapiens] | AAD29426.2 |
| HOR 5′Beta3 [Homo sapiens] | AAD29425.2 |
| F20722_2 [Homo sapiens] | AAC14389.1 |
| olfactory receptor 2J3 [Homo sapiens] | NP_001005216.2 |
| olfactory receptor 2H1 [Homo sapiens] | NP_001304951.1 |
| olfactory receptor 2H1 [Homo sapiens] | NP_001304943.1 |
| olfactory receptor 2H1 [Homo sapiens] | NP_112145.1 |
| olfactory receptor 11A1 [Homo sapiens] | NP_039225.1 |
| olfactory receptor 51B4 [Homo sapiens] | NP_149419.2 |
| olfactory receptor 51B2 [Homo sapiens] | NP_149420.4 |
| olfactory receptor 2J2 [Homo sapiens] | NP_112167.2 |
| olfactory receptor 2H2 [Homo sapiens] | NP_009091.3 |
| olfactory receptor 10G4 [Homo sapiens] | NP_001004462.1 |
| olfactory receptor 12D2 [Homo sapiens] | NP_039224.2 |
| olfactory receptor 2F1 [Homo sapiens] | NP_036501.2 |
| olfactory receptor 51M1 [Homo sapiens] | NP_001004756.2 |
| olfactory receptor 51I1 [Homo sapiens] | NP_001005288.1 |
| olfactory receptor 52D1 [Homo sapiens] | NP_001005163.1 |
| olfactory receptor 51I2 [Homo sapiens] | NP_001004754.1 |
| olfactory receptor 51B5 [Homo sapiens] | NP_001005567.2 |
| olfactory receptor 3A1 [Homo sapiens] | NP_002541.2 |
| olfactory receptor 51B6 [Homo sapiens] | NP_001004750.1 |
| olfactory receptor 5V1 [Homo sapiens] | NP_110503.3 |
| olfactory receptor 12D3 [Homo sapiens] | NP_112221.1 |
| olfactory receptor 10C1 [Homo sapiens] | NP_039229.3 |
| putative olfactory receptor 2B3 [Homo sapiens] | NP_001005226.1 |
| OR1F12, partial [Homo sapiens] | ADA83722.1 |
| OR12D3, partial [Homo sapiens] | ADA83721.1 |
| OR1F12, partial [Homo sapiens] | ADA83720.1 |
| F20722_1 [Homo sapiens] | AAC14388.1 |
| olfactory receptor [Homo sapiens] | CAD31042.1 |
| olfactory receptor [Homo sapiens] | CAD31041.1 |
| olfactory receptor [Homo sapiens] | CAD31040.1 |
| olfactory receptor [Homo sapiens] | CAD31039.1 |
| olfactory receptor [Homo sapiens] | CAD31038.1 |
| olfactory receptor [Homo sapiens] | CAD31037.1 |
| Olfactory receptor 51B4; Odorant receptor HOR5′beta1 | Q9Y5P0.3 |
| Olfactory receptor 51B2; Odorant receptor HOR5′beta3; Olfactory receptor | Q9Y5P1.4 |
| 51B1 | |
| Olfactory receptor 7D4; OR19-B; Odorant receptor family subfamily D | Q8NG98.1 |
| member 4RT; Olfactory receptor OR19-7 | |
| Olfactory receptor 1D2; Olfactory receptor 17-4; OR17-4; Olfactory receptor | P34982.2 |
| OR17-6; Olfactory receptor-like protein HGMP07E | |
| Olfactory receptor 12D3; Hs6M1-27; Olfactory receptor OR6-27 | Q9UGF7.1 |
| Olfactory receptor 5V1; Hs6M1-21; Olfactory receptor OR6-26 | Q9UGF6.1 |
| Olfactory receptor 11A1; Hs6M1-18; Olfactory receptor 11A2; Olfactory | Q9GZK7.1 |
| receptor OR6-30 | |
| Olfactory receptor 2H1; Hs6M1-16; OLFR42A-9004.14/9026.2; Olfactory | Q9GZK4.1 |
| receptor 2H6; Olfactory receptor 2H8; Olfactory receptor 6-2; OR6-2; | |
| Olfactory receptor OR6-32 | |
| Olfactory receptor 2J3; Hs6M1-3; Olfactory receptor OR6-16; OR6-6; | O76001.1 |
| Olfactory receptor 6-6 | |
| Receptor expression-enhancing protein 1 | Q9H902.1 |
| Receptor expression-enhancing protein 2 | Q9BRK0.2 |
| Olfactory receptor 5H8; Olfactory receptor 5H8 pseudogene; Olfactory | P0DN80.1 |
| receptor OR3-7 | |
| Olfactory receptor 13C7 | P0DN81.1 |
| Olfactory receptor 12D1; Olfactory receptor 12D1 pseudogene | P0DN82.1 |
| Putative olfactory receptor 8G3 pseudogene; Olfactory receptor OR11-297 | P0DMU2.1 |
| Putative olfactory receptor 13C6; Olfactory receptor, family 13, subfamily C, | Q8NH95.2 |
| member 6 pseudogene; Olfactory receptor, family 13, subfamily C, member 7 | |
| pseudogene; Putative olfactory receptor 13C7 | |
| Olfactory receptor 8G5; Olfactory receptor 8G6; Olfactory receptor OR11-298 | Q8NG78.2 |
| Olfactory receptor 51M1; Odorant receptor HOR5′beta7; Olfactory receptor | Q9H341.4 |
| OR11-40 | |
| Olfactory receptor 52E5 327 aa protein | Q8NH55.2 |
| Olfactory receptor 4A5; Olfactory receptor OR11-111 | Q8NH83.4 |
| Olfactory receptor 5K1; HTPCRX10; Olfactory receptor OR3-8 | Q8NHB7.2 |
| Olfactory receptor 2C1; OLFmf3; Olfactory receptor 2C2; Olfactory receptor | O95371.3 |
| OR16-1; Olfactory receptor OR16-2 | |
| Olfactory receptor 8B3; Olfactory receptor OR11-311 | Q8NGG8.3 |
| Olfactory receptor 4M2; Olfactory receptor OR15-3 | Q8NGB6.2 |
| Olfactory receptor 2H2; Hs6M1-12; Olfactory receptor 2H3; Olfactory | O95918.2 |
| receptor-like protein FAT11 | |
| Olfactory receptor 52L1; Olfactory receptor OR11-50 | Q8NGH7.4 |
| Olfactory receptor 2A14; OST182; Olfactory receptor 2A6; Olfactory receptor | Q96R47.4 |
| OR7-12 | |
| Olfactory receptor 10C1; Hs6M1-17; Olfactory receptor 10C2 | Q96KK4.3 |
| Olfactory receptor 8S1 | Q8NH09.2 |
| Olfactory receptor 8J1; Olfactory receptor OR11-183 | Q8NGP2.2 |
| Olfactory receptor 6Q1; Olfactory receptor OR11-226 317 aa protein | Q8NGQ2.2 |
| Olfactory receptor 4S2; Olfactory receptor OR11-137 | Q8NH73.2 |
| Olfactory receptor 52N4; Olfactory receptor OR11-64 | Q8N |
| Olfactory receptor 52K1; Olfactory receptor OR11-8 | Q8NGK4.2 |
| Olfactory receptor 52J3; Olfactory receptor OR11-32 | Q8NH60.2 |
| Olfactory receptor 52E2 | Q8NGJ4.2 |
| Olfactory receptor 52A1; HPFH1OR; Odorant receptor HOR3′beta4; Olfactory | Q9UKL2.2 |
| receptor OR11-319 | |
| Olfactory receptor 51V1; Odorant receptor HOR3′beta1; Olfactory receptor | Q9H2C8.2 |
| 51A12; Olfactory receptor OR11-36 | |
| Olfactory receptor 51B5; Odorant receptor HOR5′beta5; Olfactory receptor | Q9H339.2 |
| OR11-37 | |
| Olfactory receptor 10A4; HP2; Olfactory receptor-like protein JCG5 | Q9H209.2 |
| Olfactory receptor 10J1; Olfactory receptor OR1-26; Olfactory receptor-like | P30954.2 |
| protein HGMP07J | |
| Olfactory receptor 4D1; Olfactory receptor 4D3; Olfactory receptor TPCR16 | Q15615.3 |
| Olfactory receptor 12D2; Hs6M1-20; Olfactory receptor OR6-28 | P58182.2 |
| Olfactory receptor 10AC1; Olfactory receptor OR7-5 | Q8NH08.2 |
| Putative olfactory receptor 3A4; Olfactory receptor 17-24; OR17-24; Olfactory | P47883.4 |
| receptor 3A5 | |
| Olfactory receptor 56A4; Olfactory receptor OR11-49 | Q8NGH8.2 |
| Olfactory receptor 52E8; Olfactory receptor OR11-54 | Q6IFG1.3 |
| Olfactory receptor 2A25; Olfactory receptor 2A27 | A4D2G3.2 |
| Olfactory receptor 4K17; Olfactory receptor OR14-29 | Q8NGC6.3 |
| Olfactory receptor 1L1; Olfactory receptor 1L2; Olfactory receptor 9-C; OR9- | Q8NH94.3 |
| C; Olfactory receptor OR9-27 | |
| Olfactory receptor 4A15; Olfactory receptor OR11-118 | Q8NGL6.3 |
| Olfactory receptor 13D1; Olfactory receptor OR9-15 | Q8NGV5.3 |
| Olfactory receptor 8B2; Olfactory receptor OR11-309 | Q96RD0.3 |
| Olfactory receptor 2T1; Olfactory receptor 1-25; OR1-25; Olfactory receptor | O43869.3 |
| OR1-61 | |
| Olfactory receptor 6K3; Olfactory receptor OR1-18 | Q8NGY3.2 |
| Olfactory receptor 4K15; Olfactory receptor OR14-20 | Q8NH41.2 |
| Olfactory receptor 2T4; Olfactory receptor OR1-60 | Q8NH00.2 |
| Olfactory roocptor 1L6; Olfactory receptor 1L7; Olfactory receptor OR9-30 | Q8NGR2.2 |
| Olfactory receptor 13A1; Olfactory receptor OR10-3 | Q8NGR1.2 |
| Olfactory receptor 56B1; Olfactory receptor OR11-65 | Q8N |
| Olfactory receptor 2AK2; Olfactory receptor 2AK1; Olfactory receptor OR1- | Q8NG84.2 |
| 47 335 aa protein | |
| Olfactory receptor 3A3; Olfactory receptor 17-201; OR17-201; Olfactory | P47888.3 |
| receptor 3A6; Olfactory receptor 3A7; Olfactory receptor 3A8; Olfactory | |
| receptor OR17-22 | |
| Olfactory receptor 3A2; Olfactory receptor 17-228; OR17-228; Olfactory | P47893.3 |
| recentor OR17-14 | |
| Olfactory receptor 10R2; Olfactory receptor OR1-8 | Q8NGX6.3 |
| Olfactory receptor 52H1; Olfactory receptor OR11-45 | Q8NGJ2.3 |
| Olfactory receptor 5T2; Olfactory receptor OR11-177 | Q8NGG2.3 |
| Olfactory receptor 6S1; Olfactory receptor OR14-37 | Q8NH40.2 |
| Olfactory receptor 6K6; Olfactory receptor OR1-21 | Q8NGW6.2 |
| Olfactory receptor 5H6; Olfactory receptor OR3-11 | Q8NGV6.2 |
| Olfactory receptor 2D3; Olfactory receptor OR11-89 | Q8NGH3.2 |
| Olfactory receptor 1S2; Olfactory receptor OR11-231 | Q8NGQ3.2 |
| Olfactory receptor 52R1; Olfactory receptor OR11-22 315 aa protein | Q8NGF1.2 |
| Olfactory receptor 51F2; Olfactory receptor OR11-23 | Q8NH61.2 |
| Olfactory receptor 10S1; Olfactory receptor OR11-279 | Q8NGN2.2 |
| Olfactory receptor 52B2; Olfactory receptor OR11-70 | Q96RD2.3 |
| Olfactory receptor 52I2; Olfactory receptor OR11-12 | Q8NH67.3 |
| Olfactory receptor 52B6; Olfactory receptor OR11-47 | Q8NGF0.3 |
| Putative olfactory receptor 52L2; Olfactory receptor OR11-74 | Q8NGH6.3 |
| Olfactory receptor 2C3; Olfactory receptor 2C4; Olfactory receptor 2C5; | Q8N628.3 |
| Olfactory receptor OR1-30 | |
| Olfactory receptor 5T3; Olfactory receptor OR11-178 | Q8NGG3.3 |
| Olfactory receptor 9K2; Olfactory receptor OR12-2 | Q8NGE7.2 |
| Olfactory receptor 7G1; Olfactory receptor 19-15; OR19-15; Olfactory | Q8NGA0.2 |
| receptor OR19-8 | |
| Olfactory receptor 4N4; Olfactory receptor OR15-1; Olfactory receptor | Q8N0Y3.2 |
| OR15-5 | |
| Olfactory receptor 2K2; HTPCRH06; Olfactory receptor OR9-17 | Q8NGT1.2 |
| Olfactory receptor 1S1; Olfactory receptor OR11-232 | Q8NH92.2 |
| Olfactory receptor 1N2; Olfactory receptor OR9-23 | Q8NGR9.2 |
| Olfactory receptor 52K2; Olfactory receptor OR11-7 | Q8NGK3.2 |
| Olfactory receptor 13C3; Olfactory receptor OR9-8 | Q8NGS6.2 |
| Olfactory receptor 4A47; Olfactory receptor OR11-113 309 aa protein | Q6IF82.2 |
| Olfactory receptor 11H1; Olfactory receptor OR22-1 | Q8NG94.3 |
| Olfactory receptor 5H2; Olfactory receptor OR3-10 | Q8NGV7.3 |
| Olfactory receptor 9G4; Olfactory receptor OR11-216 | Q8NGQ1.2 |
| Olfactory receptor 8A1; OST025; Olfactory receptor OR11-318 | Q8NGG7.2 |
| Olfactory receptor 4C13; Olfactory receptor OR11-260 | Q8NGP0.2 |
| Olfactory receptor 1A1; Olfactory receptor 17-7; OR17-7; Olfactory receptor | Q9P1Q5.2 |
| OR17-11 | |
| Olfactory receptor 5AU1; Olfactory receptor OR14-38 | Q8NGC0.2 |
| Olfactory receptor 52N5; Olfactory receptor OR11-62 | Q8NH56.2 |
| Olfactory receptor 11G2; Olfactory receptor OR14-34 | Q8NGC1.2 |
| Olfactory receptor 2D2; HB2; Olfactory receptor 11-610; OR11-610; Olfactory | Q9H210.4 |
| receptor 2D1; Olfactory receptor OR11-88 | |
| Olfactory receptor 51B6; Odorant receptor HOR5′beta6 | Q9H340.2 |
| Olfactory receptor 14K1; Olfactory receptor 5AY1; Olfactory receptor OR1-39 | Q8NGZ2.2 |
| Putative olfactory receptor 9A1; HSHTPRX06 | Q8NGU1.2 |
| Olfactory receptor 14A2; Olfactory receptor 5AX1; Olfactory receptor OR1-31 | Q96R54.2 |
| Olfactory receptor 56A5 | P0C7T3.1 |
| Olfactory receptor 2T7; OST723; olfactory receptor OR1-44 | P0C7T2.1 |
| Putative olfactory receptor 2W5 320 aa protein | A6NFU9.1 |
| Olfactory receptor 52W1; Olfactory receptor OR11-71 | Q6IF63.2 |
| Olfactory receptor 11H12 | B2RN74.1 |
| Olfactory receptor 51JI; Odorant receptor HOR5′beta8; Olfactory receptor | Q9H342.2 |
| 51J2 | |
| Olfactory receptor 9G9 | P0C7N8.1 |
| Olfactory receptor 8U9 | P0C7N5.1 |
| Olfactory receptor 8U8 | P0C7N1.1 |
| Olfactory receptor 11H7; Olfactory receptor OR14-32 | Q8NGC8.2 |
| Olfactory receptor 1P1; Olfactory receptor 17-208; OR17-208; Olfactory | Q8NH06.2 |
| receptor OR17-9 | |
| Olfactory receptor 1E3; Olfactory receptor 17-210; OR17-210; Olfactory | Q8WZA6.2 |
| receptor OR17-7 | |
| Olfactory receptor 8J2 | Q8NGG1.2 |
| Olfactory receptor 5G3; Olfactory receptor 5G6; Olfactory receptor OR11-213 | P0C626.1 |
| Olfactory receptor 4Q2; olfactory receptor OR14-21 | P0C623.1 |
| Olfactory receptor 4E1; Olfactory receptor OR14-43 | P0C645.1 |
| Olfactory receptor 4A8; Olfactory receptor OR11-110 | P0C604.1 |
| Olfactory receptor 5AL1; Olfactory receptor OR11-184 | P0C617.1 |
| Olfactory receptor 5AC1; Olfactory receptor OR3-2 307 aa protein | P0C628.1 |
| Olfactory receptor 52Z1 | P0C646.1 |
| Olfactory receptor 10J4 | P0C629.1 |
| Olfactory receptor 4K3; Olfactory receptor OR14-14 | Q96R72.3 |
| Olfactory receptor 2T6; OST703; Olfactory receptor 2T9 | Q8NHC8.2 |
| Olfactory receptor 1B1; Olfactory receptor 9-B; OR9-B; Olfactory receptor | Q8NGR6.2 |
| OR9-26 | |
| Olfactory receptor 10X1; Olfactory receptor OR1-14 | Q8NGY0.2 |
| Olfactory receptor 51F1 319 aa protein | A6NGY5.1 |
| Olfactory receptor 2V1 | Q8NHB1.2 |
| Olfactory receptor 4C45 | A6NMZ5.1 |
| Olfactory receptor 52A4 | A6NMU1.1 |
| Olfactory receptor 5K4 | A6NMS3.1 |
| Olfactory receptor 2AG2 | A6NM03.1 |
| Olfactory receptor 5H14 | A6NHG9.1 |
| Olfactory receptor 2T8 | A6NH00.1 |
| Olfactory receptor 6C68 | A6NDL8.2 |
| Olfactory receptor 6C6 | A6NF89.1 |
| Olfactory receptor 5K3 | A6NET4.1 |
| Olfactory receptor 5H1; HTPCRX14 | A6NKK0.1 |
| Olfactory receptor 5B21 | A6NL26.1 |
| Olfactory receptor 6C76 | A6NM76.1 |
| Olfactory receptor 6C75 | A6NL08.1 |
| Olfactory receptor 6C74 | A6NCV1.1 |
| Olfactory receptor 6C70 | A6NIJ9.1 |
| Olfactory receptor 6C65 | A6NJZ3.1 |
| Olfactory receptor 5H15 | A6NDH6.1 |
| Olfactory receptor 14I1; Olfactory receptor 5BU1 | A6ND48.1 |
| Olfactory receptor 4C46 | A6NHA9.1 |
| Olfactory receptor 2AT4; Olfactory receptor OR11-265 | A6NND4.1 |
| Olfactory receptor 4F21 | O95013.2 |
| Olfactory receptor 2M5 | A3KFT3.1 |
| Olfactory receptor 2A7; Olfactory receptor OR7-18 | Q96R45.3 |
| Olfactory receptor 3A1; Olfactory receptor 17-40; OR17-40; Olfactory | P47881.2 |
| receptor OR17-15 | |
| Olfactory receptor 2J1; Hs6M1-4; Olfactory receptor 6-5; OR6-5 | Q9GZK6.2 |
| Olfactory receptor 5K2; Olfactory receptor OR3-9 | Q8NHB8.3 |
| Olfactory receptor 4D9; Olfactory receptor OR11-253 | Q8NGE8.3 |
| Olfactory receptor 10A2; HP4; Olfactory receptor OR11-86 | Q9H208.2 |
| Olfactory receptor 7C2; Olfactory receptor 19-18; OR19-18; Olfactory receptor | O60412.4 |
| 7C3; Olfactory receptor OR19-22 | |
| Olfactory receptor 5M3; Olfactory receptor OR11-191 | Q8NGP4.2 |
| Olfactory receptor 10V1; Olfactory receptor OR11-256 | Q8N |
| Olfactory receptor 2A5; Olfactory receptor 2A26; Olfactory receptor 2A8; | Q96R48.2 |
| Olfactory receptor 7-138/7-141; OR7-138; OR7-141 | |
| Olfactory receptor 1Q1; OST226; Olfactory receptor 1Q2; Olfactory receptor | Q15612.3 |
| 1Q3; Olfactory receptor 9-A; 0R9-A; Olfactory receptor OR9-25; Olfactory | |
| receptor TPCR106 | |
| Olfactory receptor 6C3; HSA8 | Q9NZP0.2 |
| Olfactory receptor 6C2; HSA3 | Q9NZP2.2 |
| Olfactory receptor 6C1; OST267 | Q96RD1.2 |
| Olfactory receptor 2T3 | Q8NH03.2 |
| Olfactory receptor 2M2; OST423 | Q96R28.2 |
| Olfactory receptor 5AC2; HSA1 | Q9NZP5.2 |
| Olfactory receptor 6B2; Olfactory receptor OR2-1 | Q6IFH4.2 |
| Olfactory receptor 2A2; Olfactory receptor 2A17; Olfactory receptor OR7-11 | Q6IF42.2 |
| Olfactory receptor 4C16; Olfactory receptor OR11-135 | Q8NGL9.2 |
| Olfactory receptor 2W3; Olfactory receptor 2W8; Olfactory receptor OR1-49 | Q7Z3T1.2 |
| Olfactory receptor 8G1; Olfactory receptor OR11-281; Olfactory receptor | Q15617.2 |
| TPCR25 | |
| Olfactory receptor 52A5; Odorant receptor HOR3′beta5; Olfactory receptor | Q9H2C5.1 |
| OR11-33 | |
| Olfactory receptor 5W2; Olfactory receptor 5W3; Olfactory receptor OR11- | Q8NH69.1 |
| 155 | |
| Olfactory receptor 8U1 | Q8NH10.1 |
| Olfactory receptor 2T10; Olfactory receptor OR1-64 | Q8NGZ9.1 |
| Olfactory receptor 2AJ1 | Q8NGZ0.1 |
| Olfactory receptor 52M1; Olfactory receptor OR11-11 | Q8NGK5.1 |
| Olfactory receptor 9Q2 | Q8NGE9.1 |
| Olfactory receptor 2L3 | Q8NG85.1 |
| Olfactory receptor 10K2; Olfactory receptor OR1-4 | Q6IF99.1 |
| Olfactory receptor 2T2; Olfactory receptor OR1-43 | Q6IF00.1 |
| Olfactory receptor 2T5; Olfactory receptor OR1-62 | Q6IEZ7.1 |
| Olfactory receptor 4F3/4F16/4F29; Olfactory receptor OR1-1 | Q6IEY1.1 |
| Olfactory receptor 4C11; Olfactory receptor OR11-136 | Q6IEV9.1 |
| Olfactory receptor 5M10; Olfactory receptor OR11-207 | Q6IEU7.1 |
| Olfactory receptor 2G6 | Q5TZ20.1 |
| Olfactory receptor 10J3 | Q5JRS4.1 |
| Olfactory receptor 2B11 | Q5JQS5.1 |
| Putative olfactory receptor 2W6; Olfactory receptor OR6-3; Putative olfactory | Q8NHA6.1 |
| receptor 2W7 | |
| Olfactory receptor 10G6; Olfactory receptor OR11-280 | Q8NH81.1 |
| Putative olfactory receptor 10D3; HTPCRX09; Olfactory receptor OR11-293 | Q8NH80.1 |
| Olfactory receptor 11H2; Olfactory receptor OR14-1 | Q8NH07.1 |
| Olfactory receptor 2AP1; Olfactory receptor OR12-9 | Q8NGE2.1 |
| Olfactory receptor 4C5; Olfactory receptor OR11-99 | Q8NGB2.1 |
| Olfactory receptor 7E24; Olfactory receptor OR19-14 | Q6IFN5.1 |
| Olfactory receptor 8G2; Olfactory receptor 8G4; Olfactory receptor OR11-292; | Q6IF36.1 |
| Olfactory receptor TPCR120 | |
| Olfactory receptor 2T27; Olfactory receptor OR1-67 | Q8NH04.1 |
| Olfactory receptor 5T1; Olfactory receptor OR11-179 | Q8NG75.1 |
| Olfactory receptor 4D11 | Q8N |
| Olfactory receptor 4D10; Olfactory receptor OR11-251 | Q8N |
| Olfactory receptor 2T12; Olfactory receptor OR1-57 | Q8NG77.1 |
| Olfactory receptor 51D1; Olfactory receptor OR11-14 | Q8NGF3.1 |
| Olfactory receptor 2T33; Olfactory receptor OR1-56 | Q8NG76.1 |
| Olfactory receptor 1C1; Olfactory receptor OR1-42; Olfactory receptor | Q15619.4 |
| TPCR27 | |
| Olfactory receptor 52B4; Olfactory receptor OR11-3 | Q8NGK2.2 |
| Olfactory receptor 5R1; Olfactory receptor OR11-185 | Q8NH85.1 |
| Olfactory receptor 2V2; Olfactory receptor 2V3; Olfactory receptor OR5-3 | Q96R30.3 |
| Olfactory receptor 2M4; HTPCRX18; OST710; Olfactory receptor OR1-55; | Q96R27.2 |
| Olfactory receptor TPCR100 | |
| Olfactory receptor 2T34; Olfactory receptor OR1-63 | Q8NGX1.1 |
| Olfactory receptor 6A2; Olfactory receptor 11-55; OR11-55; Olfactory | O95222.2 |
| receptor 6A1; Olfactory receptor OR11-83; hP2 olfactory receptor | |
| Olfactory receptor 10W1; Olfactory receptor OR11-236 | Q8NGF6.1 |
| Olfactory receptor 10P1; Olfactory receptor 10P2; Olfactory receptor 10P3; | Q8NGE3.1 |
| Olfactory receptor OR12-7 | |
| Olfactory receptor 14C36; Olfactory receptor 5BF1; Olfactory receptor | Q8NHC7.1 |
| OR1-59 | |
| Olfactory receptor 10AG1; Olfactory receptor OR11-160 | Q8NH19.1 |
| Olfactory receptor 2T11; Olfactory receptor OR1-65 | Q8NH01.1 |
| Olfactory receptor 5M11 | Q96RB7.2 |
| Putative olfactory receptor 1F2; OLFmf2 | Q96R84.2 |
| Olfactory receptor 4F4; HS14a-1-A; Olfactory receptor OR19-3 | Q96R69.2 |
| Olfactory receptor 4C12; Olfactory receptor OR11-259 | Q96R67.2 |
| Olfactory receptor 5B2; OST073; Olfactory receptor OR11-240 | Q96R09.3 |
| Olfactory receptor 51E1; D-GPCR; G-protein coupled receptor 164; Olfactory | Q8TCB6.1 |
| receptor 52A3; Prostate-overexpressed G protein-coupled receptor; Prostate- | |
| specific G protein-coupled receptor 2 | |
| Putative olfactory receptor 14L1; Putative olfactory receptor 5AV1 | Q8NHC6.1 |
| Olfactory receptor 14A16; Olfactory receptor 5AT1; Olfactory receptor | Q8NHC5.1 |
| OR1-45 | |
| Olfactory receptor 10J5; Olfactory receptor OR1-28 | Q8NHC4.1 |
| Olfactory receptor IF12; Hs6M1-35P | Q8NHA8.1 |
| Olfactory receptor 2AE1; Olfactory receptor 2AE2 | Q8NHA4.1 |
| Olfactory receptor 1L3; Olfactory receptor 9-D; OR9-D; Olfactory receptor | Q8NH93.1 |
| OR9-28 | |
| Olfactory receptor 5AK2 | Q8NH90.1 |
| Putative olfactory receptor 5AK3 | Q8NH89.1 |
| Olfactory receptor 9G1; Olfactory receptor 9G5; Olfactory receptor OR11-114 | Q8NH87.1 |
| Olfactory receptor 6X1; Olfactory receptor OR11-270 | Q8NH79.1 |
| Olfactory receptor 56B4; Olfactory receptor OR11-67 | Q8NH76.1 |
| Olfactory receptor 10A6; Olfactory receptor OR11-96 | Q8NH74.1 |
| Olfactory receptor 4C6; Olfactory receptor OR11-138 | Q8NH72.1 |
| Olfactory receptor 4A16; Olfactory receptor OR11-117 | Q8NH70.1 |
| Olfactory receptor 51A7; Olfactory receptor OR11-27 | Q8NH64.1 |
| Olfactory receptor 51H1; Olfactory receptor OR11-25 | Q8NH63.1 |
| Putative olfactory receptor 52P1 | Q8NH57.2 |
| Olfactory receptor 56A3; Olfactory receptor 56A6 | Q8NH54.2 |
| Olfactory receptor 52N1; Olfactory receptor OR11-61 | Q8NH53.1 |
| Olfactory receptor 8K3; Olfactory receptor OR11-181 | Q8NH51.1 |
| Olfactory receptor 8K5; Olfactory receptor OR11-174 | Q8NH50.1 |
| Olfactory receptor 4X1; Olfactory receptor OR11-104 | Q8NH49.1 |
| Olfactory receptor 5B3; Olfactory receptor 5B13; Olfactory receptor OR11- | Q8NH48.1 |
| 239 | |
| Olfactory receptor 4L1; Olfactory receptor 4L2; Olfactory receptor OR14-28 | Q8NH43.1 |
| Olfactory receptor 4K13; Olfactory receptor OR14-27 | Q8NH42.1 |
| Olfactory receptor 4C3; Olfactory receptor OR11-98 | Q8NH37.2 |
| Olfactory receptor 4F5 | Q8NH21.1 |
| Olfactory receptor 4Q3; Olfactory receptor 4Q4; Olfactory receptor OR14-3 | Q8NH05.1 |
| Olfactory receptor 2T29 315 aa protein | Q8NH02.2 |
| Olfactory receptor 6F1; Olfactory receptor OR1-38 | Q8NGZ6.1 |
| Olfactory receptor 2G2; Olfactory receptor OR1-32 | Q8NGZ5.1 |
| Olfactory receptor 2G3; Olfactory receptor OR1-33 309 aa protein | Q8NGZ4.1 |
| Olfactory receptor 13G1; Olfactory receptor OR1-37 | Q8NGZ3.1 |
| Olfactory receptor 2L8; Olfactory receptor OR1-46 | Q8NGY9.1 |
| Putative olfactory receptor 10J6 | Q8NGY7.1 |
| Olfactory receptor 6N2; Olfactory receptor OR1-23 | Q8NGY6.1 |
| Olfactory receptor 6N1 | Q8NGY5.1 |
| Olfactory receptor 6K2; Olfactory receptor OR1-17 | Q8NGY2.1 |
| Olfactory receptor 10Z1; Olfactory receptor OR.1-15 | Q8NGY1.1 |
| Olfactory receptor 6P1; Olfactory receptor OR1-12 | Q8NGX9.1 |
| Olfactory receptor 6Y1; Olfactory receptor 6Y2; Olfactory receptor OR1-11 | Q8NGX8.1 |
| Olfactory receptor 10K1; Olfactory receptor OR1-6 | Q8NGX5.1 |
| Olfactory receptor 10T2; Olfactory receptor OR1-3 | Q8NGX3.1 |
| Olfactory receptor 11L1 | Q8NGX0.1 |
| Olfactory receptor 2Y1; Olfactory receptor OR5-2 | Q8NGV0.1 |
| Putative olfactory receptor 2I1; Putative olfactory receptor 2I2; Putative | Q8NGU4.1 |
| olfactory receptor 2I3; Putative olfactory receptor 2I4 | |
| Olfactory receptor 9A4; Olfactory receptor OR7-1 | Q8NGU2.1 |
| Olfactory receptor 2A1/2A42; Olfactory receptor OR7-16; Olfactory receptor | Q8NGT9.2 |
| OR7-19 | |
| Olfactory receptor 9A2; Olfactory receptor OR7-2 | Q8NGT5.1 |
| Olfactory receptor 13J1; Olfactory receptor OR9-2 | Q8NGT2.1 |
| Olfactory receptor 13C9; Olfactory receptor OR9-13 | Q8NGT0.1 |
| Olfactory receptor 13C2; Olfactory receptor OR9-12 | Q8NGS9.1 |
| Olfactory receptor 13C5; Olfactory receptor OR9-11 | Q8NGS8.1 |
| Olfactory receptor 13C8 | Q8NGS7.1 |
| Olfactory receptor 13F1; Olfactory receptor OR9-6 | Q8NGS4.1 |
| Olfactory receptor 1J1; Olfactory receptor OR9-18 | Q8NGS3.1 |
| Olfactory receptor 1J2; HSA5; HTPCRX15; OST044; Olfactory receptor 1J3; | Q8NGS2.1 |
| Olfactory receptor 1J5; Olfactory receptor OR9-19 | |
| Olfactory receptor 1J4; HTPCRX01; Olfactory receptor OR9-21 | Q8NGS1.1 |
| Olfactory receptor 1N1; Olfactory receptor 1-26; OR1-26; Olfactory receptor | Q8NGS0.1 |
| 1N3; Olfactory receptor OR9-22 | |
| Olfactory receptor 1L8; Olfactory receptor OR9-24 | Q8NGR8.1 |
| Olfactory receptor 1L4; OST046; Olfactory receptor 1L5; Olfactory receptor 9- | Q8NGR5.1 |
| E; OR9-E; Olfactory receptor OR9-29 | |
| Olfactory receptor 5C1; Olfactory receptor 5C2; Olfactory receptor 9-F; OR9-F | Q8NGR4.1 |
| Olfactory receptor 1K1 | Q8NGR3.1 |
| Olfactory receptor 9I1; Olfactory receptor OR11-228 | Q8NGQ6.1 |
| Olfactory receptor 9Q1 | Q8NGQ5.1 |
| Olfactory receptor 10Q1; Olfactory receptor OR11-233 | Q8NGQ4.1 |
| Olfactory receptor 5M1; OST050; Olfactory receptor OR11-208 | Q8NGP8.1 |
| Olfactory receptor 5M8; Olfactory receptor OR11-194 | Q8NGP6.1 |
| Olfactory receptor 5M9; Olfactory receptor OR11-190 | Q8NGP3.1 |
| Putative olfactory receptor 4A4; Olfactory receptor OR11-107 | Q8NGN8.1 |
| Putative olfactory receptor 10D4 | Q8NGN7.1 |
| Olfactory receptor 10G7; Olfactory receptor OR11-283 | Q8NGN6.1 |
| Olfactory receptor 10G8; Olfactory receptor OR11-282 | Q8NGN5.1 |
| Olfactory receptor 10G9; Olfactory receptor 10G10 | Q8NGN4.1 |
| Olfactory receptor 10G4; Olfactory receptor OR11-278 | Q8NGN3.1 |
| Olfactory receptor 6T1; Olfactory receptor OR11-277 | Q8NGN1.1 |
| Olfactory receptor 4D5; Olfactory receptor OR11-276 | Q8NGN0.1 |
| Olfactory receptor 8D4; Olfactory receptor OR11-275 | Q8NGM9.1 |
| Olfactory receptor 6M1; Olfactory receptor OR11-271 | Q8NGM8.1 |
| Olfactory receptor 4C15; Olfactory receptor OR11-127; Olfactory receptor | Q8NGM1.1 |
| OR11-134 | |
| Olfactory receptor 4P4; Olfactory receptor 4P3 | Q8NGL7.1 |
| Olfactory receptor 5D13; Olfactory receptor OR11-142; Olfactory receptor | Q8NGL4.2 |
| OR11-148 | |
| Olfactory receptor 5D14; Olfactory receptor OR11-141; Olfactory receptor | Q8NGL3.1 |
| OR11-150 | |
| Olfactory receptor 5L1; OST262; Olfactory receptor OR11-151 | Q8NGL2.1 |
| Olfactory receptor 5D18; Olfactory receptor OR11-143; Olfactory receptor | Q8NGL1.1 |
| OR11-152 | |
| Olfactory receptor 5L2; HTPCRX16; Olfactory receptor OR11-153 | Q8NGL0.1 |
| Olfactory receptor 5D16; Olfactory receptor OR11-154 | Q8NGK9.1 |
| Olfactory receptor 5211; Olfactory receptor OR11-13 | Q8NGK6.2 |
| Olfactory receptor 51G1; Olfactory receptor 51G3; Olfactory receptor OR11- | Q8NGK1.1 |
| 29 | |
| Olfactory receptor 51G2; Olfactory receptor OR11-28 | Q8NGK0.1 |
| Olfactory receptor 51T1; Olfactory receptor OR11-26 | Q8NGJ9.1 |
| Olfactory receptor 51S1; Olfactory receptor OR11-24 | Q8NGJ8.1 |
| Olfactory receptor 51A2 | Q8NGJ7.1 |
| Olfactory receptor 51A4 | Q8NGJ6.1 |
| Olfactory receptor 51LI; Olfactory receptor OR11-31 | Q8NGJ5.1 |
| Olfactory receptor 52E1 | Q8NGJ3.1 |
| Olfactory receptor 4D6; Olfactory receptor OR11-250 | Q8NGJ1.1 |
| Olfactory receptor 5A1; OST181; Olfactory receptor OR11-249 | Q8NGJ0.1 |
| Olfactory receptor 5AN1; Olfactory receptor OR11-244 | Q8N |
| Putative olfactory receptor 56B2 | Q8N |
| Olfactory receptor 52N2; Olfactory receptor OR11-57 | Q8N |
| Olfactory receptor 52E4; Olfactory receptor OR11-55 | Q8NGH9.1 |
| Olfactory receptor 8B12; Olfactory receptor OR11-317 | Q8NGG6.1 |
| Olfactory receptor 8K1; Olfactory receptor OR11-182 | Q8NGG5.1 |
| Olfactory receptor 8H1; Olfactory receptor OR11-180 | Q8NGG4.1 |
| Olfactory receptor 8J3; Olfactory receptor OR11-173 | Q8NGG0.1 |
| Olfactory receptor 4X2; Olfactory receptor OR11-105 | Q8NGF9.1 |
| Olfactory receptor 4B1; OST208; Olfactory receptor OR11-106 | Q8NGF8.1 |
| Olfactory receptor 5B17; Olfactory receptor 5B20; Olfactory receptor OR11- | Q8NGF7.1 |
| 237 | |
| Olfactory receptor 10A7; Olfactory receptor OR12-6 | Q8NGE5.1 |
| Olfactory receptor 4K14; Olfactory receptor OR14-22 | Q8NGD5.1 |
| Olfactory receptor 4K1; Olfactory receptor OR14-19 | Q8NGD4.1 |
| Olfactory receptor 4K5; Olfactory receptor OR14-16 | Q8NGD3.1 |
| Olfactory receptor 4K2; Olfactory receptor OR14-15 | Q8NGD2.1 |
| Olfactory receptor 4N2; Olfactory receptor OR14-13; Olfactory receptor | Q8NGD1.1 |
| OR14-8 | |
| Olfactory receptor 4M1; Olfactory receptor OR14-7 | Q8NGD0.1 |
| Olfactory receptor 11H4; Olfactory receptor OR14-36 | Q8NGC9.1 |
| Olfactory receptor 11H6; Olfactory receptor OR14-35 | Q8NGC7.1 |
| Olfactory receptor 6J1; Olfactory receptor 6J2 | Q8NGC5.1 |
| Olfactory receptor 10G3; Olfactory receptor OR14-40 | Q8NGC4.1 |
| Olfactory receptor 10G2 | Q8NGC3.1 |
| Olfactory receptor 4E2; Olfactory receptor OR14-42 | Q8NGC2.1 |
| Olfactory receptor 4F6; Olfactory receptor 4F12; Olfactory receptor OR15-15 | Q8NGB9.1 |
| Olfactory receptor 4F15; Olfactory receptor OR15-14 | Q8NGB8.1 |
| Olfactory receptor 4S1; Olfactory receptor OR11-100 | Q8NGB4.1 |
| Olfactory receptor 4F17; Olfactory receptor 4F11; Olfactory receptor 4F18; | Q8NGA8.1 |
| Olfactory receptor 4F19 | |
| Olfactory receptor 10H5; Olfactory receptor OR19-25; Olfactory receptor | Q8NGA6.1 |
| OR19-26 | |
| Olfactory receptor 10H4; Olfactory receptor OR19-28 | Q8NGA5.1 |
| Putative olfactory receptor 7A2; Putative olfactory receptor 7A7 | Q8NGA2.1 |
| Olfactory receptor 1M1; Olfactory receptor 19-6; OR19-6; Olfactory receptor | Q8NGA1.1 |
| OR19-5 | |
| Olfactory receptor 7G2; OST260; Olfactory receptor 19-13; OR19-13; | Q8NG99.1 |
| Olfactory receptor OR19-6 | |
| Olfactory receptor 2Z1; Olfactory receptor 2Z2; Olfactory receptor OR19-4 | Q8NG97.1 |
| Olfactory receptor 7G3; OST085; Olfactory receptor OR19-9 | Q8NG95.1 |
| Olfactory receptor 13H1; Olfactory receptor ORX-1 | Q8NG92.1 |
| Olfactory receptor 8H2; Olfactory receptor OR11-171 | Q8N162.1 |
| Olfactory receptor 6V1; Olfactory receptor OR7-3 | Q8N148.1 |
| Olfactory receptor 8H3; Olfactory receptor OR11-172 | Q8N146.1 |
| Olfactory receptor 5AS1; Olfactory receptor OR11-168 | Q8N127.1 |
| Olfactory receptor 8I2; Olfactory receptor OR11-170 | Q8N0Y5.1 |
| Putative olfactory receptor 2B8; Hs6M1-29P | P59922.1 |
| Olfactory receptor 5J2; Olfactory receptor OR11-266 | Q8NH18.1 |
| Olfactory receptor 2A12; Olfactory receptor OR7-10 | Q8NGT7.1 |
| Olfactory receptor 2M7; Olfactory receptor OR1-58 | Q8NG81.1 |
| Olfactory receptor 2L5; Olfactory receptor 2L11; Olfactory receptor OR1-53 | Q8NG80.1 |
| Olfactory receptor 2L13; Olfactory receptor 2L14 | Q8N349.1 |
| Olfactory receptor 51Q1 | Q8NH59.2 |
| Olfactory receptor 2L2; HTPCRH07; Olfactory receptor 2L12; Olfactory | Q8NH16.1 |
| receptor 2L4 | |
| Olfactory receptor 2T35; Olfactory receptor OR1-66 | Q8NGX2.1 |
| Olfactory receptor 6B3; Olfactory receptor OR2-2 | Q8NGW1.1 |
| Olfactory receptor 6C4; Olfactory receptor OR12-10 | Q8NGE1.1 |
| Olfactory receptor 10AD1; Olfactory receptor OR12-1 | Q8NGE0.1 |
| Olfactory receptor 2M3; Olfactory receptor 2M6; Olfactory receptor OR1-54 | Q8NG83.1 |
| Olfactory receptor 1D4; Olfactory receptor 17-30; OR17-30 | P47884.3 |
| Olfactory receptor 7D2; HTPCRH03; Olfactory receptor 19-4; OR19-4; | Q96RA2.2 |
| Olfactory receptor OR19-10 | |
| Olfactory receptor 13C4; Olfactory receptor OR9-7 | Q8NGS5.1 |
| Olfactory receptor 5AR1; Olfactory receptor OR11-209 | Q8NGP9.1 |
| Olfactory receptor 5A2; Olfactory receptor OR11-248 | Q8N |
| Olfactory receptor 5AP2 | Q8NGF4.1 |
| Olfactory receptor 4N5; Olfactory receptor OR14-33 | Q8IXE1.1 |
| Olfactory receptor 52E6; Olfactory receptor OR11-58 | Q96RD3.2 |
| Olfactory receptor 8B4; Olfactory receptor OR11-315 | Q96RC9.2 |
| Olfactory receptor 5B12; Olfactory receptor 5B16; Olfactory receptor OR11- | Q96R08.2 |
| 241 | |
| Olfactory receptor 5P3; Olfactory receptor OR11-94; Olfactory receptor-like | Q8WZ94.1 |
| protein JCG1 | |
| Olfactory receptor 5P2; Olfactory receptor-like protein JCG3 | Q8WZ92.1 |
| Olfactory receptor 8D1; OST004; Olfactory receptor 8D3; Olfactory receptor | Q8WZ84.1 |
| OR11-301; Olfactory receptor-like protein JCG9 | |
| Olfactory receptor 52D1; Odorant receptor HOR5′beta14; Olfactory receptor | Q9H346.1 |
| OR11-43 | |
| Olfactory receptor 51I2; Odorant receptor HOR5′beta12; Olfactory receptor | Q9H344.1 |
| OR11-38 | |
| Olfactory receptor 51I1; Odorant receptor HOR5′beta11; Olfactory receptor | Q9H343.1 |
| OR11-39 | |
| Olfactory receptor 10H1; Olfactory receptor OR19-27 | Q9Y4A9.1 |
| Olfactory receptor 2W1; Hs6M1-15; Olfactory receptor OR6-13 | Q9Y3N9.1 |
| Olfactory receptor 14J1; Hs6M1-28; Olfactory receptor 5U1; Olfactory | Q9UGF5.1 |
| receptor OR6-25 | |
| Olfactory receptor 2S2; Olfactory receptor OR9-3 | Q9NQN1.2 |
| Olfactory receptor 10A5; HP3; Olfactory receptor 10A1; Olfactory receptor | Q9H207.1 |
| 11-403; OR11-403; Olfactory receptor-like protein JCG6 | |
| Olfactory receptor 2AG1; HT3; Olfactory receptor 2AG3; Olfactory receptor | Q9H205.2 |
| OR11-79 | |
| Olfactory receptor 8D2; Olfactory receptor OR11-303; Olfactory receptor-like | Q9GZM6.1 |
| protein JCG2 | |
| Olfactory receptor 2B2; Hs6M1-10; Olfactory receptor 2B9; Olfactory receptor | Q9GZK3.1 |
| 6-1; OR6-1 | |
| Olfactory receptor 7A5; Olfactory receptor OR19-17; Olfactory receptor | Q15622.2 |
| TPCR92 | |
| Olfactory receptor 8B8; Olfactory receptor TPCR85; Olfactory-like receptor | Q15620.2 |
| JCG8 | |
| Olfactory receptor 10A3; HTPCRX12; Olfactory receptor OR11-97 | P58181.1 |
| Olfactory receptor 4D2; B-lymphocyte membrane protein BC2009; Olfactory | P58180.1 |
| receptor OR17-24 | |
| Olfactory receptor 2B6; Hs6M1-32; Olfactory receptor 2B1; Olfactory receptor | P58173.1 |
| 2B5; Olfactory receptor 5-40; OR5-40; Olfactory receptor 6-31; OR6-31; | |
| Olfactory receptor OR6-4 | |
| Olfactory receptor 1D5; Olfactory receptor 17-31; OR17-31 | P58170.1 |
| Olfactory receptor 5F1; Olfactory receptor 11-10; OR11-10; Olfactory receptor | O95221.2 |
| OR11-167 | |
| Olfactory receptor 2A4; Olfactory receptor 2A10; Olfactory receptor OR6-37 | O95047.1 |
| Olfactory receptor 6B1; Olfactory receptor 7-3; OR7-3; Olfactory receptor | O95007.1 |
| OR7-9 | |
| Olfactory receptor 2F2; Olfactory receptor 7-1; OR7-1; Olfactory receptor | O95006.1 |
| OR7-6 | |
| Olfactory receptor 7A10; OST027; Olfactory receptor OR19-18 | O76100.1 |
| Olfactory receptor 2J2; Hs6M1-6; Olfactory receptor 6-8; OR6-8; Olfactory | O76002.1 |
| receptor OR6-19 | |
| Putative olfactory receptor 2B3; Hs6M1-1; Olfactory receptor OR6-14; OR6-4; | O76000.1 |
| Olfactory receptor 6-4 | |
| Olfactory receptor 1I1; Olfactory receptor 19-20; OR19-20 | O60431.1 |
| Olfactory receptor 10H3; Olfactory receptor OR19-24 | O60404.1 |
| Olfactory receptor 10H2; Olfactory receptor OR19-23 | O60403.1 |
| Olfactory receptor 7A17 | O14581.1 |
| Olfactory receptor 2F1; Olfactory receptor 2F3; Olfactory receptor 2F4; | Q13607.2 |
| Olfactory receptor 2F5; Olfactory receptor-like protein OLF3 | |
| Olfactory receptor 1G1; Olfactory receptor 17-209; OR17-209; Olfactory | P47890.2 |
| receptor 1G2; Olfactory receptor OR17-8 | |
| Olfactory receptor 1E2; Olfactory receptor 17-93/17-135/17-136; OR17-135; | P47887.2 |
| OR17-136; OR17-93; Olfactory receptor 1E4 | |
| Olfactory receptor 1A2; Olfactory receptor 17-6; OR17-6; Olfactory receptor | Q9Y585.1 |
| OR17-10 | |
| Olfactory receptor 7C1; Olfactory receptor 7C4; Olfactory receptor OR19-16; | O76099.1 |
| Olfactory receptor TPCR86 | |
| Olfactory receptor 1F1; Olfactory receptor 16-35; OR16-35; Olfactory receptor | O43749.1 |
| 1F10; Olfactory receptor 1F4; Olfactory receptor 1F5; Olfactory receptor 1F6; | |
| Olfactory receptor 1F7; Olfactory receptor 1F8; Olfactory receptor 1F9; | |
| Olfactory receptor OR16-4 | |
| Olfactory receptor 5I1; Olfactory receptor OR11-159; Olfactory receptor-like | Q13606.1 |
| protein OLF1 | |
| Olfactory receptor 1E1; Olfactory receptor 13-66; OR13-66; Olfactory receptor | P30953.1 |
| 17-2/17-32; OR17-2; OR17-32; Olfactory receptor 1E5; Olfactory receptor | |
| 1E6; Olfactory receptor 5-85; OR5-85; Olfactory receptor OR17-18; Olfactory | |
| receptor-like protein HGMP07I | |
| Olfactory receptor 56A1; Olfactory receptor OR11-75 | Q8NGH5.3 |
| putative odorant receptor 71a [Talaromyces mameffei PM1] | KFX53697.1 |
| hypothetical protein XK86_18365 [Hafnia alvei] | KKI42162.1 |
| hypothetical protein PAST3_12155 [Propionibacterium acnes HL201PA1] | KFC15621.1 |
| hypothetical protein Odosp 2381 [Odoribacter splanchnicus DSM 20712] | ADY33373.1 |
| hypothetical protein LLB_1684 [Le Glonella longbeachae D-4968] | EEZ96489.1 |
| hypothetical protein cypCar_00040615 [Cyprinus carpio] | KTG44310.1 |
| hypothetical protein cypCar_00022850 [Cyprinus carpio] | KTF94953.1 |
| hypothetical protein cypCar_00047049 [Cyprinus carpio] | KTF88600.1 |
| hypothetical protein cypCar_00047378 [Cyprinus carpio] | KTF77827.1 |
| hypothetical protein cypCar_00040594 [Cyprinus carpio] | KTF73152.1 |

TABLE 3
Odorant receptors.

| Gene Name | Accession Number |
|---|---|
| odorant receptor [Ostrinia nubilalis] 333 aa protein | BAJ61939.1 GI: 319918821 |
| odorant receptor, partial [Ostrinia nubilalis] 419 aa protein | BAJ61937.1 GI: 319918818 |
| odorant receptor, partial [Ostrinia nubilalis] 314 aa protein | BAJ61935.1 GI: 319918814 |
| odorant receptor [Ostrinia nubilalis] 422 aa protein | BAJ61934.1 GI: 319918812 |
| odorant receptor [Ostrinia nubilalis] 408 aa protein | BAJ61933.1 GI: 319918810 |
| odorant receptor [Ostrinia nubilalis] 424 aa protein | BAJ61932.1 GI: 319918808 |
| odorant receptor [Ostrinia nubilalis] 424 aa protein | BAJ61929.1 GI: 319918797 |
| odorant receptor [Ostrinia nubilalis] 425 aa protein | BAJ61928.1 GI: 319918796 |
| odorant receptor, partial [Ostrinia nubilalis] 89 aa protein | BAJ61938.1 GI: 319918819 |
| odorant receptor, partial [Ostrinia nubilalis] 136 aa protein | BAJ61936.1 GI: 319918816 |
| odorant receptor, partial [Ostrinia nubilalis × Ostrinia scapulalis] | BAJ61931.1 GI: 319918803 |
| 200 aa protein | |
| odorant receptor, partial [Ostrinia nubilalis × Ostrinia scapulalis] | BAJ61930.1 GI: 319918801 |
| 200 aa protein | |
| odorant receptor, partial [Ostrinia palustralis] 383 aa protein | BAI66637.3 GI: 310688057 |
| odorant receptor, partial [Ostrinia nubilalis] 380 aa protein | BAI66625.3 GI: 310688051 |
| odorant receptor, partial [Ostrinia zaguliaevi] 412 aa protein | BAJ22892.1 GI: 308522556 |
| odorant receptor, partial [Ostrinia fumacalis] 406 aa protein | BAJ22891.1 GI: 308522554 |
| odorant receptor, partial [Ostrinia scapulalis] 396 aa protein | BAJ22890.1 GI: 308522552 |
| odorant receptor, partial [Ostrinia scapulalis] 406 aa protein | BAJ22889.1 GI: 308522550 |
| odorant receptor, partial [Ostrinia zealis] 408 aa protein | BAI66649.1 GI: 284010028 |
| odorant receptor, partial [Ostrinia zealis] 397 aa protein | BAI66648.1 GI: 284010026 |
| odorant receptor, partial [Ostrinia zealis] 409 aa protein | BAI66647.1 GI: 284010024 |
| odorant receptor, partial [Ostrinia zealis] 407 aa protein | BAI66646.1 GI: 284010022 |
| odorant receptor, partial [Ostrinia zealis] 409 aa protein | BAI66645.1 GI: 284010020 |
| odorant receptor, partial [Ostrinia zealis] 409 aa protein | BAI66644.1 GI: 284010018 |
| odorant receptor, partial [Ostrinia zaguliaevi] 397 aa protein | BAI66642.1 GI: 284010014 |
| odorant receptor, partial [Ostrinia zaguliaevi] 409 aa protein | BAI66640.1 GI: 284010010 |
| odorant receptor, partial [Ostrinia zaguliaevi] 406 aa protein | BAI66639.1 GI: 284010008 |
| odorant receptor, partial [Ostrinia zaguliaevi] 409 aa protein | BAI66638.1 GI: 284010006 |
| odorant receptor, partial [Ostrinia palustralis] 397 aa protein | BAI66636.1 GI: 284010002 |
| odorant receptor, partial [Ostrinia palustralis] 407 aa protein | BAI66635.1 GI: 284010000 |
| odorant receptor, partial [Ostrinia palustralis] 409 aa protein | BAI66634.1 GI: 284009998 |
| odorant receptor, partial [Ostrinia ovalipennis] 408 aa protein | BAI66633.1 GI: 284009996 |
| odorant receptor, partial [Ostrinia ovalipennis] 323 aa protein | BAI66632.1 GI: 284009994 |
| odorant receptor, partial [Ostrinia ovalipennis] 366 aa protein | BAI66631.1 GI: 284009992 |
| odorant receptor, partial [Ostrinia ovalipennis] 406 aa protein | BAI66630.1 GI: 284009990 |
| odorant receptor, partial [Ostrinia nubilalis] 397 aa protein | BAI66627.1 GI: 284009984 |
| odorant receptor, partial [Ostrinia nubilalis] 416 aa protein | BAI66626.1 GI: 284009982 |
| odorant receptor, partial [Ostrinia nubilalis] 407 aa protein | BAI66624.1 GI: 284009978 |
| odorant receptor, partial [Ostrinia nubilalis] 409 aa protein | BAI66623.1 GI: 284009976 |
| odorant receptor, partial [Ostrinia latipennis] 323 aa protein | BAI66621.1 GI: 284009972 |
| odorant receptor, partial [Ostrinia latipennis] 350 aa protein | BAI66620.1 GI: 284009970 |
| odorant receptor, partial [Ostrinia latipennis] 366 aa protein | BAI66619.1 GI: 284009968 |
| odorant receptor, partial [Ostrinia latipennis] 407 aa protein | BAI66618.1 GI: 284009966 |
| odorant receptor, partial [Ostrinia fumacalis] 408 aa protein | BAI66616.1 GI: 284009962 |
| odorant receptor, partial [Ostrinia fumacalis] 396 aa protein | BAI66615.1 GI: 284009960 |
| odorant receptor, partial [Ostrinia fumacalis] 408 aa protein | BAI66614.1 GI: 284009958 |
| odorant receptor, partial [Ostrinia fumacalis] 408 aa protein | BAI66613.1 GI: 284009956 |
| odorant receptor, partial [Ostrinia fumacalis] 407 aa protein | BAI66612.1 GI: 284009954 |
| odorant receptor, partial [Ostrinia fumacalis] 409 aa protein | BAI66611.1 GI: 284009952 |
| odorant receptor [Ostrinia scapulalis] 422 aa protein | BAI66610.1 GI: 284009950 |
| odorant receptor [Ostrinia scapulalis] 408 aa protein | BAI66609.1 GI: 284009948 |
| odorant receptor [Ostrinia scapulalis] 424 aa protein | BAI66608.1 GI: 284009946 |
| odorant receptor [Ostrinia scapulalis] 433 aa protein | BAI66607.1 GI: 284009944 |
| odorant receptor [Ostrinia scapulalis] 422 aa protein | BAI66605.1 GI: 284009940 |
| odorant receptor [Ostrinia scapulalis] 425 aa protein | BAI66604.1 GI: 284009938 |
| odorant receptor, partial [Ostrinia ovalipennis] 304 aa protein | BAI66629.3 GI: 310688055 |
| odorant receptor, partial [Ostrinia nubilalis] 275 aa protein | BAI66628.2 GI: 310688053 |
| odorant receptor, partial [Ostrinia zaguliaevi] 291 aa protein | BAI66643.1 GI: 284010016 |
| odorant receptor, partial [Ostrinia latipennis] 291 aa protein | BAI66622.1 GI: 284009974 |
| odorant receptor, partial [Ostrinia latipennis] 318 aa protein | BAI66617.1 GI: 284009964 |
| Odorant receptor coreceptor; AgOr7; Gustatory and odorant | Q7QCC7.3 GI: 158563992 |
| receptor 7 478 aa protein | |
| Odorant receptor coreceptor; Gustatory and odorant receptor 7 478 | Q178U6.1 GI: 122117922 |
| aa protein | |
| Putative odorant receptor 19b 387 aa protein | Q8IRZ5.1 GI: 55584079 |
| Putative odorant receptor 69a, isoform A 393 aa protein | Q9VU27.2 GI: 41393542 |
| Putative odorant receptor 69a, isoform B 393 aa protein | P82985.1 GI: 14285634 |
| Putative odorant receptor 65c 410 aa protein | P82984.2 GI: 108935862 |
| Putative odorant receptor 65b 406 aa protein | P82983.2 GI: 108935861 |
| Putative odorant receptor 98b 384 aa protein | Q9VAW0.3 GI: 92090622 |
| Putative odorant receptor 85e 467 aa protein | P81924.3 GI: 54041947 |
| Putative odorant receptor 71a 378 aa protein | Q9VUK5.4 GI: 50403809 |
| Putative odorant receptor 92a 408 aa protein | Q9VDM1.3 GI: 33860192 |
| Putative odorant receptor 83c 397 aa protein | Q9VNK9.2 GI: 14285641 |
| Putative odorant receptor 59c 411 aa protein | Q9W1P7.1 GI: 11387002 |
| Putative odorant receptor 85d 412 aa protein | Q9VHQ2.1 GI: 11386992 |
| Putative odorant receptor 13a [Cerapachys biroi] 194 aa protein | EZA49383.1 GI: 607354771 |
| odorant receptor 300 [Nasonia vitripennis] 395 aa protein | NP_001177714.1 GI: 299782530 |
| odorant receptor 299 [Nasonia vitripennis] 417 aa protein | NP_001177713.1 GI: 299782528 |
| odorant receptor 298 [Nasonia vitripennis] 403 aa protein | NP_001177712.1 GI: 299782526 |
| odorant receptor 289 [Nasonia vitripennis] 398 aa protein | NP_001177710.1 GI: 299782524 |
| odorant receptor 258 [Nasonia vitripennis] 383 aa protein | NP_001177709.1 GI: 299782520 |
| odorant receptor 241 [Nasonia vitripennis] 384 aa protein | NP_001177708.1 GI: 299782517 |
| odorant receptor 216 [Nasonia vitripennis] 406 aa protein | NP_001177707.1 GI: 299782515 |
| odorant receptor 198 [Nasonia vitripennis] 393 aa protein | NP_001177706.1 GI: 299782513 |
| odorant receptor 190 [Nasonia vitripennis] 396 aa protein | NP_001177705.1 GI: 299782511 |
| odorant receptor 160 [Nasonia vitripennis] 402 aa protein | NP_001177703.1 GI: 299782507 |
| odorant receptor 156 [Nasonia vitripennis] 400 aa protein | NP_001177702.1 GI: 299782503 |
| odorant receptor 149 [Nasonia vitripennis] 389 aa protein | NP_001177700.1 GI: 299782500 |
| odorant receptor 96 [Nasonia vitripennis] 385 aa protein | NP_001177699.1 GI: 299782498 |
| odorant receptor 47 [Nasonia vitripennis] 380 aa protein | NP_001177494.1 GI: 299523279 |
| odorant receptor 46 [Nasonia vitripennis] 377 aa protein | NP_001177493.1 GI: 299523277 |
| odorant receptor 45 [Nasonia vitripennis] 372 aa protein | NP_001177492.1 GI: 299523275 |
| odorant receptor 44 [Nasonia vitripennis] 373 aa protein | NP_001177491.1 GI: 299523273 |
| odorant receptor 43 [Nasonia vitripennis] 382 aa protein | NP_001177490.1 GI: 299523271 |
| odorant receptor 37 [Nasonia vitripennis] 410 aa protein | NP_001177488.1 GI: 299523269 |
| odorant receptor 28 [Nasonia vitripennis] 402 aa protein | NP_001177483.1 GI: 299523261 |
| odorant receptor 27 [Nasonia vitripennis] 419 aa protein | NP_001177482.1 GI: 299523255 |
| odorant receptor 26 [Nasonia vitripennis] 408 aa protein | NP_001177481.1 GI: 299523251 |
| odorant receptor 25 [Nasonia vitripennis] 405 aa protein | NP_001177480.1 GI: 299523248 |
| odorant receptor 24 [Nasonia vitripennis] 415 aa protein | NP_001177479.1 GI: 299523246 |
| odorant receptor 22 [Nasonia vitripennis] 397 aa protein | NP_001177477.1 GI: 299523244 |
| odorant receptor 23 [Nasonia vitripennis] 420 aa protein | NP_001177478.1 GI: 299523242 |
| odorant receptor 20 [Nasonia vitripennis] 407 aa protein | NP_001177475.1 GI: 299523240 |
| odorant receptor 21 [Nasonia vitripennis] 409 aa protein | NP_001177476.1 GI: 299523238 |
| odorant receptor 19 [Nasonia vitripennis] 405 aa protein | NP_001177474.1 GI: 299523236 |
| odorant receptor 17 [Nasonia vitripennis] 409 aa protein | NP_001177473.1 GI: 299523231 |
| odorant receptor 16 [Nasonia vitripennis] 408 aa protein | NP_001177472.1 GI: 299523229 |
| odorant receptor 15 [Nasonia vitripennis] 401 aa protein | NP_001177471.1 GI: 299523226 |
| odorant receptor 13 [Nasonia vitripennis] 431 aa protein | NP_001177469.1 GI: 299523217 |
| odorant receptor 12 [Nasonia vitripennis] 410 aa protein | NP_001177468.1 GI: 299523215 |
| odorant receptor 10 [Nasonia vitripennis] 406 aa protein | NP_001177467.1 GI: 299523212 |
| odorant receptor 9 [Nasonia vitripennis] 405 aa protein | NP_001177435.1 GI: 299523119 |
| odorant receptor 8 [Nasonia vitripennis] 399 aa protein | NP_001177434.1 GI: 299523116 |
| odorant receptor 7 [Nasonia vitripennis] 408 aa protein | NP_001177433.1 GI: 299523113 |
| odorant receptor 6 [Nasonia vitripennis] 399 aa protein | NP_001177432.1 GI: 299523110 |
| odorant receptor 5 [Nasonia vitripennis] 428 aa protein | NP_001177431.1 GI: 299523107 |
| odorant receptor 3 [Nasonia vitripennis] 435 aa protein | NP_001177430.1 GI: 299523104 |
| odorant receptor 2 [Nasonia vitripennis] 420 aa protein | NP_001177429.1 GI: 299523100 |
| odorant receptor 159 [Nasonia vitripennis] 399 aa protein | NP_001177423.1 GI: 299523072 |
| odorant receptor 292 [Nasonia vitripennis] 403 aa protein | NP_001177621.1 GI: 299522969 |
| odorant receptor 291 [Nasonia vitripennis] 402 aa protein | NP_001177620.1 GI: 299522967 |
| odorant receptor 286 [Nasonia vitripennis] 413 aa protein | NP_001177619.1 GI: 299522965 |
| odorant receptor 285 [Nasonia vitripennis] 411 aa protein | NP_001177618.1 GI: 299522963 |
| odorant receptor 281 [Nasonia vitripennis] 401 aa protein | NP_001177617.1 GI: 299522961 |
| odorant receptor 279 [Nasonia vitripennis] 403 aa protein | NP_001177616.1 GI: 299522959 |
| odorant receptor 278 [Nasonia vitripennis] 403 aa protein | NP_001177615.1 GI: 299522957 |
| odorant receptor 277 [Nasonia vitripennis] 404 aa protein | NP_001177614.1 GI: 299522955 |
| odorant receptor 273 [Nasonia vitripennis] 407 aa protein | NP_001177612.1 GI: 299522950 |
| odorant receptor 272 [Nasonia vitripennis] 400 aa protein | NP_001177611.1 GI: 299522948 |
| odorant receptor 271 [Nasonia vitripennis] 400 aa protein | NP_001177610.1 GI: 299522946 |
| odorant receptor 269 [Nasonia vitripennis] 408 aa protein | NP_001177609.1 GI: 299522944 |
| odorant receptor 268 [Nasonia vitripennis] 407 aa protein | NP_001177608.1 GI: 299522942 |
| odorant receptor 267 [Nasonia vitripennis] 407 aa protein | NP_001177607.1 GI: 299522940 |
| odorant receptor 264 [Nasonia vitripennis] 409 aa protein | NP_001177605.1 GI: 299522936 |
| odorant receptor 260 [Nasonia vitripennis] 384 aa protein | NP_001177603.1 GI: 299522932 |
| odorant receptor 257 [Nasonia vitripennis] 383 aa protein | NP_001177602.1 GI: 299522930 |
| odorant receptor 256 [Nasonia vitripennis] 386 aa protein | NP_001177601.1 GI: 299522928 |
| odorant receptor 255 [Nasonia vitripennis] 385 aa protein | NP_001177600.1 GI: 299522926 |
| odorant receptor 251 [Nasonia vitripennis] 386 aa protein | NP_001177598.1 GI: 299522922 |
| odorant receptor 250 [Nasonia vitripennis] 381 aa protein | NP_001177597.1 GI: 299522920 |
| odorant receptor 248 [Nasonia vitripennis] 384 aa protein | NP_001177596.1 GI: 299522918 |
| odorant receptor 247 [Nasonia vitripennis] 381 aa protein | NP_001177595.1 GI: 299522916 |
| odorant receptor 245 [Nasonia vitripennis] 384 aa protein | NP_001177594.1 GI: 299522914 |
| odorant receptor 236 [Nasonia vitripennis] 419 aa protein | NP_001177592.1 GI: 299522910 |
| odorant receptor 233 [Nasonia vitripennis] 397 aa protein | NP_001177591.1 GI: 299522908 |
| odorant receptor 232 [Nasonia vitripennis] 399 aa protein | NP_001177590.1 GI: 299522906 |
| odorant receptor 230 [Nasonia vitripennis] 394 aa protein | NP_001177589.1 GI: 299522904 |
| odorant receptor 229 [Nasonia vitripennis] 398 aa protein | NP_001177588.1 GI: 299522902 |
| odorant receptor 226 [Nasonia vitripennis] 399 aa protein | NP_001177586.1 GI: 299522900 |
| odorant receptor 227 [Nasonia vitripennis] 400 aa protein | NP_001177587.1 GI: 299522898 |
| odorant receptor 224 [Nasonia vitripennis] 398 aa protein | NP_001177584.1 GI: 299522896 |
| odorant receptor 225 [Nasonia vitripennis] 396 aa protein | NP_001177585.1 GI: 299522894 |
| odorant receptor 222 [Nasonia vitripennis] 396 aa protein | NP_001177583.1 GI: 299522892 |
| odorant receptor 219 [Nasonia vitripennis] 396 aa protein | NP_001177581.1 GI: 299522890 |
| odorant receptor 221 [Nasonia vitripennis] 403 aa protein | NP_001177582.1 GI: 299522888 |
| odorant receptor 218 [Nasonia vitripennis] 399 aa protein | NP_001177580.1 GI: 299522886 |
| odorant receptor 217 [Nasonia vitripennis] 412 aa protein | NP_001177579.1 GI: 299522884 |
| odorant receptor 207 [Nasonia vitripennis] 397 aa protein | NP_001177577.1 GI: 299522882 |
| odorant receptor 204 [Nasonia vitripennis] 390 aa protein | NP_001177576.1 GI: 299522880 |
| odorant receptor 203 [Nasonia vitripennis] 388 aa protein | NP_001177575.1 GI: 299522878 |
| odorant receptor 202 [Nasonia vitripennis] 390 aa protein | NP_001177574.1 GI: 299522876 |
| odorant receptor 201 [Nasonia vitripennis] 390 aa protein | NP_001177573.1 GI: 299522874 |
| odorant receptor 196 [Nasonia vitripennis] 398 aa protein | NP_001177572.1 GI: 299522872 |
| odorant receptor 194 [Nasonia vitripennis] 406 aa protein | NP_001177570.1 GI: 299522868 |
| odorant receptor 193 [Nasonia vitripennis] 398 aa protein | NP_001177569.1 GI: 299522866 |
| odorant receptor 192 [Nasonia vitripennis] 398 aa protein | NP_001177568.1 GI: 299522864 |
| odorant receptor 191 [Nasonia vitripennis] 392 aa protein | NP_001177567.1 GI: 299522860 |
| odorant receptor 187 [Nasonia vitripennis] 417 aa protein | NP_001177564.1 GI: 299522856 |
| odorant receptor 179 [Nasonia vitripennis] 402 aa protein | NP_001177560.1 GI: 299522848 |
| odorant receptor 173 [Nasonia vitripennis] 393 aa protein | NP_001177557.1 GI: 299522840 |
| odorant receptor 170 [Nasonia vitripennis] 414 aa protein | NP_001177556.1 GI: 299522836 |
| odorant receptor 167 [Nasonia vitripennis] 404 aa protein | NP_001177555.1 GI: 299522833 |
| odorant receptor 166 [Nasonia vitripennis] 406 aa protein | NP_001177554.1 GI: 299522831 |
| odorant receptor 162 [Nasonia vitripennis] 409 aa protein | NP_001177553.1 GI: 299522829 |
| odorant receptor 161 [Nasonia vitripennis] 393 aa protein | NP_001177552.1 GI: 299522827 |
| odorant receptor 158 [Nasonia vitripennis] 400 aa protein | NP_001177551.1 GI: 299522825 |
| odorant receptor 157 [Nasonia vitripennis] 395 aa protein | NP_001177550.1 GI: 299522823 |
| odorant receptor 151 [Nasonia vitripennis] 391 aa protein | NP_001177549.1 GI: 299522821 |
| odorant receptor 147 [Nasonia vitripennis] 395 aa protein | NP_001177548.1 GI: 299522819 |
| odorant receptor 146 [Nasonia vitripennis] 402 aa protein | NP_001177547.1 GI: 299522817 |
| odorant receptor 143 [Nasonia vitripennis] 391 aa protein | NP_001177545.1 GI: 299522815 |
| odorant receptor 142 [Nasonia vitripennis] 395 aa protein | NP_001177544.1 GI: 299522813 |
| odorant receptor 139 [Nasonia vitripennis] 388 aa protein | NP_001177542.1 GI: 299522809 |
| odorant receptor 137 [Nasonia vitripennis] 388 aa protein | NP_001177541.1 GI: 299522807 |
| odorant receptor 135 [Nasonia vitripennis] 386 aa protein | NP_001177540.1 GI: 299522805 |
| odorant receptor 133 [Nasonia vitripennis] 397 aa protein | NP_001177539.1 GI: 299522803 |
| odorant receptor 132 [Nasonia vitripennis] 395 aa protein | NP_001177538.1 GI: 299522801 |
| odorant receptor 128 [Nasonia vitripennis] 367 aa protein | NP_001177536.1 GI: 299522797 |
| odorant receptor 125 [Nasonia vitripennis] 370 aa protein | NP_001177534.1 GI: 299522793 |
| odorant receptor 124 [Nasonia vitripennis] 370 aa protein | NP_001177533.1 GI: 299522788 |
| odorant receptor 118 [Nasonia vitripennis] 395 aa protein | NP_001177531.1 GI: 299522781 |
| odorant receptor 117 [Nasonia vitripennis] 385 aa protein | NP_001177530.1 GI: 299522779 |
| odorant receptor 115 [Nasonia vitripennis] 414 aa protein | NP_001177529.1 GI: 299522777 |
| odorant receptor 111 [Nasonia vitripennis] 397 aa protein | NP_001177526.1 GI: 299522773 |
| odorant receptor 110 [Nasonia vitripennis] 397 aa protein | NP_001177525.1 GI: 299522769 |
| odorant receptor 107 [Nasonia vitripennis] 393 aa protein | NP_001177524.1 GI: 299522767 |
| odorant receptor 103 [Nasonia vitripennis] 394 aa protein | NP_001177522.1 GI: 299522763 |
| odorant receptor 102 [Nasonia vitripennis] 397 aa protein | NP_001177521.1 GI: 299522761 |
| odorant receptor 100 [Nasonia vitripennis] 396 aa protein | NP_001177519.1 GI: 299522759 |
| odorant receptor 101 [Nasonia vitripennis] 404 aa protein | NP_001177520.1 GI: 299522757 |
| odorant receptor 99 [Nasonia vitripennis] 397 aa protein | NP_001177518.1 GI: 299522754 |
| odorant receptor 94 [Nasonia vitripennis] 387 aa protein | NP_001177517.1 GI: 299522752 |
| odorant receptor 93 [Nasonia vitripennis] 390 aa protein | NP_001177516.1 GI: 299522748 |
| odorant receptor 89 [Nasonia vitripennis] 396 aa protein | NP_001177515.1 GI: 299522746 |
| odorant receptor 88 [Nasonia vitripennis] 391 aa protein | NP_001177514.1 GI: 299522744 |
| odorant receptor 86 [Nasonia vitripennis] 385 aa protein | NP_001177512.1 GI: 299522742 |
| odorant receptor 87 [Nasonia vitripennis] 387 aa protein | NP_001177513.1 GI: 299522740 |
| odorant receptor 79 [Nasonia vitripennis] 413 aa protein | NP_001177511.1 GI: 299522738 |
| odorant receptor 78 [Nasonia vitripennis] 402 aa protein | NP_001177510.1 GI: 299522736 |
| odorant receptor 69 [Nasonia vitripennis] 382 aa protein | NP_001177509.1 GI: 299522734 |
| odorant receptor 68 [Nasonia vitripennis] 372 aa protein | NP_001177508.1 GI: 299522732 |
| odorant receptor 66 [Nasonia vitripennis] 376 aa protein | NP_001177506.1 GI: 299522730 |
| odorant receptor 67 [Nasonia vitripennis] 379 aa protein | NP_001177507.1 GI: 299522728 |
| odorant receptor 65 [Nasonia vitripennis] 379 aa protein | NP_001177505.1 GI: 299522726 |
| odorant receptor 64 [Nasonia vitripennis] 381 aa protein | NP_001177504.1 GI: 299522724 |
| odorant receptor 62 [Nasonia vitripennis] 381 aa protein | NP_001177503.1 GI: 299522722 |
| odorant receptor 61 [Nasonia vitripennis] 402 aa protein | NP_001177502.1 GI: 299522720 |
| odorant receptor 60 [Nasonia vitripennis] 412 aa protein | NP_001177501.1 GI: 299522718 |
| odorant receptor 59 [Nasonia vitripennis] 389 aa protein | NP_001177500.1 GI: 299522716 |
| odorant receptor 51 [Nasonia vitripennis] 377 aa protein | NP_001177497.1 GI: 299522710 |
| odorant receptor 31 [Nasonia vitripennis] 400 aa protein | NP_001177485.1 GI: 299507620 |
| odorant receptor 283 [Nasonia vitripennis] 408 aa protein | NP_001164423.2 GI: 289666787 |
| odorant receptor 77 [Nasonia vitripennis] 413 aa protein | NP_001164671.1 GI: 283945552 |
| odorant receptor 76 [Nasonia vitripennis] 417 aa protein | NP_001164670.1 GI: 283945550 |
| odorant receptor 263 [Nasonia vitripennis] 384 aa protein | NP_001164420.2 GI: 283945546 |
| odorant receptor 301 [Nasonia vitripennis] 422 aa protein | NP_001164659.1 GI: 283945514 |
| odorant receptor 1 [Nasonia vitripennis] 475 aa protein | NP_001164465.1 GI: 283436213 |
| odorant receptor 253 [Nasonia vitripennis] 383 aa protein | NP_001164463.1 GI: 283436209 |
| odorant receptor 293 [Nasonia vitripennis] 371 aa protein | NP_001164462.1 GI: 283436207 |
| odorant receptor 98 [Nasonia vitripennis] 395 aa protein | NP_001164458.1 GI: 283436197 |
| odorant receptor 265 [Nasonia vitripennis] 408 aa protein | NP_001164457.1 GI: 283436195 |
| odorant receptor 280 [Nasonia vitripennis] 407 aa protein | NP_001164422.1 GI: 283436107 |
| odorant receptor 243 [Nasonia vitripennis] 382 aa protein | NP_001164417.1 GI: 283436101 |
| odorant receptor 249 [Nasonia vitripennis] 388 aa protein | NP_001164419.1 GI: 283436099 |
| odorant receptor 246 [Nasonia vitripennis] 382 aa protein | NP_001164418.1 GI: 283436097 |
| odorant receptor 168 [Nasonia vitripennis] 401 aa protein | NP_001164416.1 GI: 283436095 |
| odorant receptor 163 [Nasonia vitripennis] 409 aa protein | NP_001164411.1 GI: 283135180 |
| odorant receptor 154 [Nasonia vitripennis] 395 aa protein | NP_001164405.1 GI: 283135167 |
| odorant receptor 141 [Nasonia vitripennis] 392 aa protein | NP_001164404.1 GI: 283135164 |
| odorant receptor 105 [Nasonia vitripennis] 396 aa protein | NP_001164401.1 GI: 283135159 |
| odorant receptor 92 [Nasonia vitripennis] 391 aa protein | NP_001164399.1 GI: 283135153 |
| odorant receptor 85 [Nasonia vitripennis] 392 aa protein | NP_001164398.1 GI: 283135151 |
| odorant receptor 80 [Nasonia vitripennis] 386 aa protein | NP_001164396.1 GI: 283135146 |
| odorant receptor 82 [Nasonia vitripennis] 392 aa protein | NP_001164395.1 GI: 283135140 |
| odorant receptor 81 [Nasonia vitripennis] 387 aa protein | NP_001164394.1 GI: 283135138 |
| odorant receptor 2 [Apis mellifera] 477 aa protein | NP_001128415.1 GI: 201023349 |
| odorant receptor 180 [Nasonia vitripennis] 395 aa protein | NP_001177704.1 GI: 299782509 |
| odorant receptor 276 [Nasonia vitripennis] 403 aa protein | NP_001177613.1 GI: 299522952 |
| odorant receptor 182 [Nasonia vitripennis] 411 aa protein | NP_001177562.1 GI: 299522852 |
| odorant receptor 46a, isoform A-like [Diachasma alloeum] 654 aa | XP_015127274.1 GI: 970885192 |
| protein | |
| odorant receptor 130 [Nasonia vitripennis] 395 aa protein | NP_001177640.2 GI: 299782532 |
| odorant receptor 33b-like [Bactrocera oleae] 712 aa protein | XP_014097126.1 GI: 929374155 |
| odorant receptor coreceptor [Ceratitis capitata] 473 aa protein | NP_001266301.1 GI: 525342887 |
| odorant receptor 296 [Nasonia vitripennis] 387 aa protein | NP_001177711.1 GI: 299782522 |
| odorant receptor 153 [Nasonia vitripennis] 389 aa protein | NP_001177701.1 GI: 299782505 |
| odorant receptor 71 [Nasonia vitripennis] 384 aa protein | NP_001177698.1 GI: 299782496 |
| odorant receptor 288 [Nasonia vitripennis] 399 aa protein | NP_001177643.1 GI: 299528645 |
| odorant receptor 58 [Nasonia vitripennis] 393 aa protein | NP_001177639.1 GI: 299528533 |
| odorant receptor 36 [Nasonia vitripennis] 404 aa protein | NP_001177487.1 GI: 299523266 |
| odorant receptor 35 [Nasonia vitripennis] 422 aa protein | NP_001177486.1 GI: 299523264 |
| odorant receptor 14 [Nasonia vitripennis] 405 aa protein | NP_001177470.1 GI: 299523221 |
| odorant receptor 145 [Nasonia vitripennis] 401 aa protein | NP_001177546.1 GI: 299523210 |
| odorant receptor 294 [Nasonia vitripennis] 354 aa protein | NP_001177622.1 GI: 299522971 |
| odorant receptor 266 [Nasonia vitripennis] 407 aa protein | NP_001177606.1 GI: 299522938 |
| odorant receptor 252 [Nasonia vitripennis] 386 aa protein | NP_001177599.1 GI: 299522924 |
| odorant receptor 242 [Nasonia vitripennis] 385 aa protein | NP_001177593.1 GI: 299522912 |
| odorant receptor 195 [Nasonia vitripennis] 400 aa protein | NP_001177571.1 GI: 299522870 |
| odorant receptor 181 [Nasonia vitripennis] 405 aa protein | NP_001177561.1 GI: 299522850 |
| odorant receptor 140 [Nasonia vitripennis] 393 aa protein | NP_001177543.1 GI: 299522811 |
| odorant receptor 129 [Nasonia vitripennis] 395 aa protein | NP_001177537.1 GI: 299522799 |
| odorant receptor 126 [Nasonia vitripennis] 369 aa protein | NP_001177535.1 GI: 299522795 |
| odorant receptor 114 [Nasonia vitripennis] 409 aa protein | NP_001177528.1 GI: 299522775 |
| odorant receptor 113 [Nasonia vitripennis] 393 aa protein | NP_001177527.1 GI: 299522771 |
| odorant receptor 106 [Nasonia vitripennis] 397 aa protein | NP_001177523.1 GI: 299522765 |
| odorant receptor 53 [Nasonia vitripennis] 384 aa protein | NP_001177498.1 GI: 299522712 |
| odorant receptor 262 [Nasonia vitripennis] 385 aa protein | NP_001164460.2 GI: 283945544 |
| odorant receptor 122 [Nasonia vitripennis] 369 aa protein | NP_001164459.1 GI: 283436199 |
| odorant receptor 275 [Nasonia vitripennis] 405 aa protein | NP_001164421.1 GI: 283436105 |
| odorant receptor 41 [Nasonia vitripennis] 397 aa protein | NP_001164391.1 GI: 283135132 |
| odorant receptor 295 [Nasonia vitripennis] 370 aa protein | NP_001177623.1 GI: 299522975 |
| odorant receptor 188 [Nasonia vitripennis] 395 aa protein | NP_001177565.1 GI: 299522858 |
| odorant receptor 175 [Nasonia vitripennis] 393 aa protein | NP_001177558.1 GI: 299522844 |
| odorant receptor 56 [Nasonia vitripennis] 371 aa protein | NP_001177499.1 GI: 299522714 |
| odorant receptor 48 [Nasonia vitripennis] 395 aa protein | NP_001177495.1 GI: 299522708 |
| odorant receptor 38 [Nasonia vitripennis] 414 aa protein | NP_001177489.1 GI: 299528647 |
| odorant receptor 29 [Nasonia vitripennis] 403 aa protein | NP_001177484.1 GI: 299523259 |
| odorant receptor 261 [Nasonia vitripennis] 381 aa protein | NP_001177604.1 GI: 299522934 |
| odorant receptor 189 [Nasonia vitripennis] 395 aa protein | NP_001177566.1 GI: 299522862 |
| odorant receptor 183 [Nasonia vitripennis] 405 aa protein | NP_001177563.1 GI: 299522854 |
| odorant receptor 177 [Nasonia vitripennis] 392 aa protein | NP_001177559.1 GI: 299522846 |
| odorant receptor 119 [Nasonia vitripennis] 391 aa protein | NP_001177532.1 GI: 299522786 |
| odorant receptor 134 [Nasonia vitripennis] 395 aa protein | NP_001177696.1 GI: 299782491 |
| odorant receptor 72 [Nasonia vitripennis] 383 aa protein | NP_001177641.1 GI: 299528641 |
| odorant receptor coreceptor-like [Diuraphis noxia] 167 aa protein | XP_015371514.1 GI: 985412051 |
| odorant receptor 46a, isoform B-like [Diuraphis noxia] 251 aa | XP_015367780.1 GI: 985400241 |
| protein | |
| odorant receptor 46a, isoform B-like [Diuraphis noxia] 129 aa | XP_015367779.1 GI: 985400239 |
| protein | |
| odorant receptor 22c-like [Diuraphis noxia] 176 aa protein | XP_015367764.1 GI: 985400213 |
| odorant receptor 85b-like [Diuraphis noxia] 219 aa protein | XP_015379800.1 GI: 985390295 |
| odorant receptor coreceptor [Diachasma alloeum] 478 aa protein | XP_015126208.1 GI: 970919070 |
| odorant receptor 46a, isoform B-like [Diachasma alloeum] 391 aa | XP_015126084.1 GI: 970918843 |
| protein | |
| putative odorant receptor 65b [Diachasma alloeum] 342 aa protein | XP_015125987.1 GI: 970918672 |
| odorant receptor 13a-like [Diachasma alloeum] 417 aa protein | XP_015125770.1 GI: 970918327 |
| odorant receptor 30a-like [Diachasma alloeum] 160 aa protein | XP_015125618.1 GI: 970918086 |
| odorant receptor 30a-like [Diachasma alloeum] 125 aa protein | XP_015125102.1 GI: 970917269 |
| odorant receptor 30a-like [Diachasma alloeum] 125 aa protein | XP_015125054.1 GI: 970917185 |
| odorant receptor 13a-like [Diachasma alloeum] 395 aa protein | XP_015125011.1 GI: 970917102 |
| odorant receptor 13a-like [Diachasma alloeum] 411 aa protein | XP_015125010.1 GI: 970917100 |
| odorant receptor 13a-like [Diachasma alloeum] 412 aa protein | XP_015125009.1 GI: 970917098 |
| odorant receptor 22c-like [Diachasma alloeum] 417 aa protein | XP_015124997.1 GI: 970917076 |
| odorant receptor 4-like [Diachasma alloeum] 396 aa protein | XP_015124996.1 GI: 970917074 |
| odorant receptor 4-like [Diachasma alloeum] 191 aa protein | XP_015124994.1 GI: 970917070 |
| odorant receptor 22c-like [Diachasma alloeum] 398 aa protein | XP_015124993.1 GI: 970917068 |
| odorant receptor 67c-like isoform X3 [Diachasma alloeum] 350 aa | XP_015123023.1 GI: 970913425 |
| protein | |
| odorant receptor 13a-like isoform X1 [Diachasma alloeum] 386 aa | XP_015123021.1 GI: 970913421 |
| protein | |
| odorant receptor 46a, isoform A-like [Diachasma alloeum] 386 aa | XP_015122891.1 GI: 970913183 |
| protein | |
| odorant receptor Or1-like [Diachasma alloeum] 401 aa protein | XP_015122890.1 GI: 970913181 |
| odorant receptor 13a-like [Diachasma alloeum] 385 aa protein | XP_015122297.1 GI: 970912091 |
| odorant receptor coreceptor-like [Diachasma alloeum] 160 aa | XP_015122295.1 GI: 970912087 |
| protein | |
| odorant receptor 82a-like [Diachasma alloeum] 281 aa protein | XP_015122294.1 GI: 970912085 |
| odorant receptor 13a-like [Diachasma alloeum] 398 aa protein | XP_015122293.1 GI: 970912083 |
| odorant receptor 67c-like [Diachasma alloeum] 225 aa protein | XP_015122289.1 GI: 970912076 |
| odorant receptor 4-like isoform X2 [Diachasma alloeum] 384 aa | XP_015122288.1 GI: 970912074 |
| protein | |
| odorant receptor 4-like isoform X1 [Diachasma alloeum] 390 aa | XP_015122287.1 GI: 970912072 |
| protein | |
| odorant receptor 4-like isoform X1 [Diachasma alloeum] 390 aa | XP_015122286.1 GI: 970912070 |
| protein | |
| odorant receptor 67c-like [Diachasma alloeum] 389 aa protein | XP_015122278.1 GI: 970912056 |
| odorant receptor 4 [Diachasma alloeum] 102 aa protein | XP_015122277.1 GI: 970912054 |
| odorant receptor 82a-like, partial [Diachasma alloeum] 274 aa | XP_015122272.1 GI: 970912046 |
| protein | |
| odorant receptor 4-like [Diachasma alloeum] 378 aa protein | XP_015121583.1 GI: 970910790 |
| odorant receptor 13a-like [Diachasma alloeum] 431 aa protein | XP_015121344.1 GI: 970910338 |
| odorant receptor 13a-like [Diachasma alloeum] 458 aa protein | XP_015121343.1 GI: 970910336 |
| odorant receptor Or2-like [Diachasma alloeum] 413 aa protein | XP_015120978.1 GI: 970909674 |
| odorant receptor 82a-like [Diachasma alloeum] 116 aa protein | XP_015120698.1 GI: 970909157 |
| odorant receptor 13a-like [Diachasma alloeum] 122 aa protein | XP_015120696.1 GI: 970909155 |
| odorant receptor 46a, isoform A-like [Diachasma alloeum] 391 aa | XP_015120217.1 GI: 970908285 |
| protein | |
| odorant receptor Or1-like, partial [Diachasma alloeum] 360 aa | XP_015119834.1 GI: 970907577 |
| protein | |
| odorant receptor Or2-like [Diachasma alloeum] 193 aa protein | XP_015119359.1 GI: 970906702 |
| odorant receptor 67b-like [Diachasma alloeum] 169 aa protein | XP_015118342.1 GI: 970904876 |
| odorant receptor 22c-like [Diachasma alloeum] 431 aa protein | XP_015118336.1 GI: 970904864 |
| putative odorant receptor 71a [Diachasma alloeum] 302 aa protein | XP_015118334.1 GI: 970904862 |
| odorant receptor 2a-like [Diachasma alloeum] 419 aa protein | XP_015117540.1 GI: 970903392 |
| odorant receptor Or2-like [Diachasma alloeum] 202 aa protein | XP_015117539.1 GI: 970903390 |
| odorant receptor 85f-like [Diachasma alloeum] 392 aa protein | XP_015117537.1 GI: 970903386 |
| putative odorant receptor 85d [Diachasma alloeum] 402 aa protein | XP_015117136.1 GI: 970902641 |
| odorant receptor 22c-like [Diachasma alloeum] 402 aa protein | XP_015117110.1 GI: 970902593 |
| odorant receptor 22c-like [Diachasma alloeum] 266 aa protein | XP_015116846.1 GI: 970902114 |
| odorant receptor 49b-like [Diachasma alloeum] 279 aa protein | XP_015116845.1 GI: 970902112 |
| odorant receptor 13a-like isoform X2 [Diachasma alloeum] 321 aa | XP_015115896.1 GI: 970900372 |
| protein | |
| odorant receptor 13a-like isoform X1 [Diachasma alloeum] 325 aa | XP_015115895.1 GI: 970900370 |
| protein | |
| odorant receptor 13a-like [Diachasma alloeum] 386 aa protein | XP_015115894.1 GI: 970900368 |
| odorant receptor 46a, isoform B-like [Diachasma alloeum] 399 aa | XP_015115875.1 GI: 970900334 |
| protein | |
| odorant receptor 33a-like [Diachasma alloeum] 343 aa protein | XP_015115812.1 GI: 970900224 |
| odorant receptor 13a-like [Diachasma alloeum] 380 aa protein | XP_015115810.1 GI: 970900220 |
| odorant receptor Or1-like [Diachasma alloeum] 390 aa protein | XP_015115473.1 GI: 970899618 |
| odorant receptor 49b-like [Diachasma alloeum] 386 aa protein | XP_015114827.1 GI: 970898436 |
| putative odorant receptor 98b [Diachasma alloeum] 367 aa protein | XP_015114784.1 GI: 970898359 |
| odorant receptor 85b [Diachasma alloeum] 393 aa protein | XP_015114365.1 GI: 970897598 |
| odorant receptor 63a-like [Diachasma alloeum] 383 aa protein | XP_015114103.1 GI: 970897122 |
| odorant receptor 67c-like [Diachasma alloeum] 116 aa protein | XP_015114099.1 GI: 970897116 |
| odorant receptor 82a-like [Diachasma alloeum] 388 aa protein | XP_015114079.1 GI: 970897076 |
| odorant receptor Or2-like [Diachasma alloeum] 379 aa protein | XP_015114021.1 GI: 970896976 |
| odorant receptor Or2-like [Diachasma alloeum] 379 aa protein | XP_015114020.1 GI: 970896974 |
| odorant receptor 82a-like [Diachasma alloeum] 325 aa protein | XP_015112771.1 GI: 970894647 |
| putative odorant receptor 92a [Diachasma alloeum] 152 aa protein | XP_015112770.1 GI: 970894645 |
| odorant receptor 13a-like [Diachasma alloeum] 403 aa protein | XP_015112769.1 GI: 970894643 |
| odorant receptor 82a-like [Diachasma alloeum] 403 aa protein | XP_015112590.1 GI: 970894312 |
| odorant receptor 82a-like [Diachasma alloeum] 403 aa protein | XP_015112589.1 GI: 970894310 |
| odorant receptor 4-like [Diachasma alloeum] 409 aa protein | XP_015112587.1 GI: 970894308 |
| odorant receptor 85c-like [Diachasma alloeum] 411 aa protein | XP_015112586.1 GI: 970894306 |
| odorant receptor 82a-like [Diachasma alloeum] 412 aa protein | XP_015112585.1 GI: 970894304 |
| odorant receptor 67c-like [Diachasma alloeum] 411 aa protein | XP_015112584.1 GI: 970894302 |
| odorant receptor Or2-like [Diachasma alloeum] 415 aa protein | XP_015112582.1 GI: 970894300 |
| odorant receptor 13a-like [Diachasma alloeum] 365 aa protein | XP_015111780.1 GI: 970892852 |
| odorant receptor 13a-like [Diachasma alloeum] 407 aa protein | XP_015111777.1 GI: 970892846 |
| odorant receptor 13a-like [Diachasma alloeum] 163 aa protein | XP_015111079.1 GI: 970891543 |
| odorant receptor 42b-like [Diachasma alloeum] 134 aa protein | XP_015111029.1 GI: 970891451 |
| odorant receptor 59b-like [Diachasma alloeum] 420 aa protein | XP_015110864.1 GI: 970891147 |
| odorant receptor 33c-like [Diachasma alloeum] 428 aa protein | XP_015110846.1 GI: 970891111 |
| odorant receptor 47b-like [Diachasma alloeum] 424 aa protein | XP_015110804.1 GI: 970891036 |
| odorant receptor 33b-like [Diachasma alloeum] 415 aa protein | XP_015110803.1 GI: 970891034 |
| odorant receptor coreceptor-like [Diachasma alloeum] 430 aa | XP_015110802.1 GI: 970891032 |
| protein | |
| odorant receptor 33b-like [Diachasma alloeum] 435 aa protein | XP_015110787.1 GI: 970891004 |
| odorant receptor 46a, isoform A-like isoform X2 [Diachasma | XP_015110556.1 GI: 970890577 |
| alloeum] 358 aa protein | |
| putative odorant receptor 85d isoform X1 [Diachasma alloeum] | XP_015110555.1 GI: 970890575 |
| 395 aa protein | |
| putative odorant receptor 92a [Diachasma alloeum] 249 aa protein | XP_015110532.1 GI: 970890535 |
| odorant receptor 13a-like [Diachasma alloeum] 389 aa protein | XP_015110383.1 GI: 970890261 |
| odorant receptor 4-like [Diachasma alloeum] 231 aa protein | XP_015110343.1 GI: 970890187 |
| odorant receptor 10a-like [Diachasma alloeum] 268 aa protein | XP_015109340.1 GI: 970888368 |
| odorant receptor 4-like [Diachasma alloeum] 278 aa protein | XP_015108894.1 GI: 970887560 |
| odorant receptor 10a-like [Diachasma alloeum] 388 aa protein | XP_015108891.1 GI: 970887554 |
| odorant receptor 4-like [Diachasma alloeum] 126 aa protein | XP_015108890.1 GI: 970887552 |
| odorant receptor 67c-like [Diachasma alloeum] 381 aa protein | XP_015108889.1 GI: 970887550 |
| putative odorant receptor 85e [Diachasma alloeum] 422 aa protein | XP_015108502.1 GI: 970886846 |
| odorant receptor Or1-like isoform X3 [Diachasma alloeum] 425 aa | XP_015127537.1 GI: 970885664 |
| protein | |
| odorant receptor Or1-like isoform X2 [Diachasma alloeum] 450 aa | XP_015127536.1 GI: 970885662 |
| protein | |
| odorant receptor Or1-like isoform X1 [Diachasma alloeum] 460 aa | XP_015127535.1 GI: 970885660 |
| protein | |
| odorant receptor Or1-like [Diachasma alloeum] 350 aa protein | XP_015127275.1 GI: 970885194 |
| odorant receptor 46a, isoform A-like [Diachasma alloeum] 391 aa | XP_015127273.1 GI: 970885190 |
| protein | |
| odorant receptor Or2-like [Diachasma alloeum] 416 aa protein | XP_015127033.1 GI: 970884759 |
| odorant receptor 22c-like [Diachasma alloeum] 182 aa protein | XP_015127018.1 GI: 970884735 |
| odorant receptor 49b-like [Diachasma alloeum] 157 aa protein | XP_015127017.1 GI: 970884733 |
| odorant receptor 4-like [Diachasma alloeum] 259 aa protein | XP_015126452.1 GI: 970883704 |
| putative odorant receptor 71a [Diachasma alloeum] 334 aa protein | XP_015126451.1 GI: 970883702 |
| odorant receptor 67a-like [Diachasma alloeum] 316 aa protein | XP_015124669.1 GI: 970883032 |
| odorant receptor 43a-like [Diachasma alloeum] 185 aa protein | XP_015124656.1 GI: 970883030 |
| odorant receptor 4-like [Diachasma alloeum] 388 aa protein | XP_015117493.1 GI: 970881492 |
| odorant receptor 67c-like [Diachasma alloeum] 343 aa protein | XP_015117460.1 GI: 970881486 |
| odorant receptor 13a-like isoform X2 [Diachasma alloeum] 400 aa | XP_015117447.1 GI: 970881484 |
| protein | |
| odorant receptor 13a-like [Diachasma alloeum] 401 aa protein | XP_015117410.1 GI: 970881478 |
| odorant receptor coreceptor-like [Diachasma alloeum] 263 aa | XP_015116354.1 GI: 970881285 |
| protein | |
| odorant receptor 47a-like [Diachasma alloeum] 204 aa protein | XP_015116327.1 GI: 970881281 |
| putative odorant receptor 98b [Diachasma alloeum] 390 aa protein | XP_015116314.1 GI: 970881279 |
| odorant receptor 4-like [Diachasma alloeum] 338 aa protein | XP_015115419.1 GI: 970881109 |
| odorant receptor Or2-like [Diachasma alloeum] 454 aa protein | XP_015112971.1 GI: 970880651 |
| odorant receptor Or2-like [Diachasma alloeum] 385 aa protein | XP_015112944.1 GI: 970880647 |
| odorant receptor 49b-like [Diachasma alloeum] 427 aa protein | XP_015112917.1 GI: 970880643 |
| odorant receptor 49b-like [Diachasma alloeum] 381 aa protein | XP_015112905.1 GI: 970880641 |
| odorant receptor 13a-like [Plutella xylostella] 424 aa protein | NP_001292415.1 GI: 770075498 |
| odorant receptor 24a-like, partial [Halyomorpha halys] 99 aa | XP_014293293.1 GI: 939698236 |
| protein | |
| odorant receptor 59b-like [Halyomorpha halys] 417 aa protein | XP_014293234.1 GI: 939698126 |
| odorant receptor 85b-like [Halyomorpha halys] 399 aa protein | XP_014292083.1 GI: 939695930 |
| odorant receptor 22c-like [Halyomorpha halys] 411 aa protein | XP_014292012.1 GI: 939695795 |
| putative odorant receptor 71a, partial [Halyomorpha halys] 240 aa | XP_014291481.1 GI: 939694804 |
| protein | |
| odorant receptor 24a-like [Halyomorpha halys] 127 aa protein | XP_014290811.1 GI: 939693500 |
| odorant receptor 85b-like [Halyomorpha halys] 83 aa protein | XP_014290807.1 GI: 939693494 |
| odorant receptor 85b-like [Halyomorpha halys] 204 aa protein | XP_014290806.1 GI: 939693492 |
| odorant receptor 22c-like [Halyomorpha halys] 420 aa protein | XP_014290805.1 GI: 939693490 |
| odorant receptor 4-like [Halyomorpha halys] 420 aa protein | XP_014290804.1 GI: 939693488 |
| odorant receptor 4-like [Halyomorpha halys] 208 aa protein | XP_014290613.1 GI: 939693125 |
| odorant receptor 43a-like [Halyomorpha halys] 152 aa protein | XP_014290611.1 GI: 939693123 |
| odorant receptor 4-like [Halyomorpha halys] 366 aa protein | XP_014290317.1 GI: 939692579 |
| odorant receptor 49a-like [Halyomorpha halys] 387 aa protein | XP_014289672.1 GI: 939691321 |
| odorant receptor 24a-like [Halyomorpha halys] 419 aa protein | XP_014289234.1 GI: 939690515 |
| odorant receptor 82a-like [Halyomorpha halys] 391 aa protein | XP_014289202.1 GI: 939690459 |
| odorant receptor 82a-like [Halyomorpha halys] 391 aa protein | XP_014289201.1 GI: 939690457 |
| odorant receptor 82a-like [Halyomorpha halys] 391 aa protein | XP_014289200.1 GI: 939690455 |
| odorant receptor 43a-like [Halyomorpha halys] 319 aa protein | XP_014289020.1 GI: 939690118 |
| odorant receptor 67c-like isoform X3 [Halyomorpha halys] 156 aa | XP_014288707.1 GI: 939689526 |
| protein | |
| odorant receptor 67c-like isoform X2 [Halyomorpha halys] 319 aa | XP_014288705.1 GI: 939689524 |
| protein | |
| odorant receptor 67c-like isoform X1 [Halyomorpha halys] 349 aa | XP_014288704.1 GI: 939689522 |
| protein | |
| odorant receptor 24a [Halyomorpha halys] 414 aa protein | XP_014288393.1 GI: 939688944 |
| odorant receptor 24a [Halyomorpha halys] 414 aa protein | XP_014288391.1 GI: 939688942 |
| odorant receptor 4-like [Halyomorpha halys] 378 aa protein | XP_014287492.1 GI: 939686767 |
| putative odorant receptor 71a [Halyomorpha halys] 140 aa protein | XP_014287487.1 GI: 939686757 |
| odorant receptor 85b-like [Halyomorpha halys] 389 aa protein | XP_014287367.1 GI: 939686386 |
| odorant receptor 43b-like [Halyomorpha halys] 389 aa protein | XP_014287042.1 GI: 939685352 |
| odorant receptor 4-like [Halyomorpha halys] 389 aa protein | XP_014287040.1 GI: 939685346 |
| putative odorant receptor 92a [Halyomorpha halys] 373 aa protein | XP_014286385.1 GI: 939683385 |
| odorant receptor coreceptor-like [Halyomorpha halys] 133 aa | XP_014285169.1 GI: 939679600 |
| protein | |
| odorant receptor 4-like [Halyomorpha halys] 349 aa protein | XP_014282544.1 GI: 939671925 |
| putative odorant receptor 85d isoform X1 [Halyomorpha halys] | XP_014282484.1 GI: 939671750 |
| 270 aa protein | |
| odorant receptor 49a-like isoform X1 [Halyomorpha halys] 175 aa | XP_014282386.1 GI: 939671478 |
| protein | |
| odorant receptor 33a-like [Halyomorpha halys] 406 aa protein | XP_014281821.1 GI: 939670017 |
| odorant receptor coreceptor isoform X2 [Halyomorpha halys] 474 | XP_014279420.1 GI: 939663702 |
| aa protein | |
| odorant receptor coreceptor isoform X1 [Halyomorpha halys] 474 | XP_014279419.1 GI: 939663700 |
| aa protein | |
| odorant receptor 33a-like [Halyomorpha halys] 247 aa protein | XP_014278712.1 GI: 939661914 |
| odorant receptor 67c-like [Halyomorpha halys] 417 aa protein | XP_014278702.1 GI: 939661887 |
| odorant receptor 22c-like [Halyomorpha halys] 426 aa protein | XP_014276985.1 GI: 939657423 |
| odorant receptor 4-like [Halyomorpha halys] 284 aa protein | XP_014276746.1 GI: 939656832 |
| odorant receptor 94a-like [Halyomorpha halys] 339 aa protein | XP_014276741.1 GI: 939656820 |
| odorant receptor 22c-like [Halyomorpha halys] 400 aa protein | XP_014275988.1 GI: 939654773 |
| odorant receptor 24a-like [Halyomorpha halys] 412 aa protein | XP_014275211.1 GI: 939652560 |
| odorant receptor 30a-like [Halyomorpha halys] 389 aa protein | XP_014274900.1 GI: 939651712 |
| odorant receptor 47a-like [Halyomorpha halys] 427 aa protein | XP_014274444.1 GI: 939650476 |
| odorant receptor 67c-like [Halyomorpha halys] 154 aa protein | XP_014272634.1 GI: 939645252 |
| odorant receptor 83a-like [Halyomorpha halys] 416 aa protein | XP_014271039.1 GI: 939640611 |
| odorant receptor 4-like isoform X3 [Halyomorpha halys] 354 aa | XP_014270190.1 GI: 939638174 |
| protein | |
| odorant receptor 4-like isoform X2 [Halyomorpha halys] 376 aa | XP_014270189.1 GI: 939638171 |
| protein | |
| odorant receptor 4-like isoform X1 [Halyomorpha halys] 408 aa | XP_014270188.1 GI: 939638168 |
| protein | |
| odorant receptor 24a-like [Halyomorpha halys] 414 aa protein | XP_014270184.1 GI: 939638154 |
| odorant receptor 24a-like [Halyomorpha halys] 81 aa protein | XP_014294800.1 GI: 939638065 |
| odorant receptor 24a-like [Halyomorpha halys] 134 aa protein | XP_014294799.1 GI: 939638061 |
| odorant receptor 85b-like [Halyomorpha halys] 99 aa protein | XP_014294798.1 GI: 939638059 |
| odorant receptor 24a-like [Halyomorpha halys] 415 aa protein | XP_014294793.1 GI: 939638051 |
| odorant receptor 30a-like [Halyomorpha halys] 415 aa protein | XP_014294791.1 GI: 939638047 |
| odorant receptor 7a-like [Halyomorpha halys] 124 aa protein | XP_014294788.1 GI: 939638038 |
| odorant receptor 24a-like [Halyomorpha halys] 415 aa protein | XP_014294787.1 GI: 939638035 |
| odorant receptor 9a-like [Halyomorpha halys] 382 aa protein | XP_014294442.1 GI: 939637038 |
| odorant receptor Or1-like isoform X1 [Halyomorpha halys] 384 aa | XP_014294439.1 GI: 939637034 |
| protein | |
| odorant receptor 85b-like isoform X1 [Halyomorpha halys] 389 aa | XP_014293859.1 GI: 939635389 |
| protein | |
| odorant receptor 46a, isoform B-like [Halyomorpha halys] 390 aa | XP_014291074.1 GI: 939634209 |
| protein | |
| odorant receptor 49b-like [Halyomorpha halys] 288 aa protein | XP_014281510.1 GI: 939631411 |
| odorant receptor 4-like [Halyomorpha halys] 397 aa protein | XP_014281005.1 GI: 939631291 |
| odorant receptor 85c-like [Halyomorpha halys] 99 aa protein | XP_014280802.1 GI: 939631237 |
| odorant receptor 85b-like [Halyomorpha halys] 99 aa protein | XP_014280790.1 GI: 939631235 |
| odorant receptor 24a-like isoform X1 [Halyomorpha halys] 397 aa | XP_014280768.1 GI: 939631231 |
| protein | |
| odorant receptor 7a-like [Halyomorpha halys] 310 aa protein | XP_014280756.1 GI: 939631229 |
| odorant receptor 4-like [Halyomorpha halys] 413 aa protein | XP_014280744.1 GI: 939631227 |
| odorant receptor 82a-like [Halyomorpha halys] 411 aa protein | XP_014280723.1 GI: 939631221 |
| odorant receptor 82a [Halyomorpha halys] 419 aa protein | XP_014280696.1 GI: 939631215 |
| odorant receptor 24a-like [Halyomorpha halys] 417 aa protein | XP_014280623.1 GI: 939631200 |
| odorant receptor 24a-like [Halyomorpha halys] 431 aa protein | XP_014280612.1 GI: 939631197 |
| odorant receptor 49b-like [Halyomorpha halys] 393 aa protein | XP_014275003.1 GI: 939629564 |
| odorant receptor 7a-like [Halyomorpha halys] 123 aa protein | XP_014273506.1 GI: 939629170 |
| odorant receptor 85b-like [Halyomorpha halys] 388 aa protein | XP_014273330.1 GI: 939629125 |
| odorant receptor 85b-like [Halyomorpha halys] 386 aa protein | XP_014272535.1 GI: 939628833 |
| odorant receptor 24a-like [Halyomorpha halys] 409 aa protein | XP_014272447.1 GI: 939628818 |
| odorant receptor 4-like [Halyomorpha halys] 410 aa protein | XP_014294765.1 GI: 939628125 |
| odorant receptor 43b-like [Bactrocera oleae] 255 aa protein | XP_014101582.1 GI: 929382187 |
| odorant receptor 43b-like, partial [Bactrocera oleae] 256 aa protein | XP_014101520.1 GI: 929382078 |
| odorant receptor 88a-like, partial [Bactrocera oleae] 400 aa protein | XP_014101401.1 GI: 929381869 |
| odorant receptor 85c-like, partial [Bactrocera oleae] 277 aa protein | XP_014101291.1 GI: 929381677 |
| odorant receptor 67c-like [Bactrocera oleae] 405 aa protein | XP_014101001.1 GI: 929381148 |
| odorant receptor 43b-like, partial [Bactrocera oleae] 210 aa protein | XP_014100962.1 GI: 929381076 |
| odorant receptor 7a-like [Bactrocera oleae] 471 aa protein | XP_014100884.1 GI: 929380937 |
| odorant receptor 7a-like, partial [Bactrocera oleae] 398 aa protein | XP_014100883.1 GI: 929380935 |
| odorant receptor 59a-like [Bactrocera oleae] 249 aa protein | XP_014100068.1 GI: 929379483 |
| odorant receptor 30a-like [Bactrocera oleae] 403 aa protein | XP_014100035.1 GI: 929379425 |
| odorant receptor 88a-like [Bactrocera oleae] 410 aa protein | XP_014099351.1 GI: 929378168 |
| odorant receptor 88a-like [Bactrocera oleae] 404 aa protein | XP_014099350.1 GI: 929378166 |
| odorant receptor 42b-like [Bactrocera oleae] 256 aa protein | XP_014098809.1 GI: 929377189 |
| odorant receptor 63a-like [Bactrocera oleae] 415 aa protein | XP_014098250.1 GI: 929376191 |
| odorant receptor 45a-like [Bactrocera oleae] 365 aa protein | XP_014098074.1 GI: 929375879 |
| odorant receptor 45a-like, partial [Bactrocera oleae] 196 aa protein | XP_014098072.1 GI: 929375875 |
| odorant receptor 45a-like [Bactrocera oleae] 334 aa protein | XP_014098071.1 GI: 929375873 |
| odorant receptor la-like [Bactrocera oleae] 388 aa protein | XP_014098069.1 GI: 929375871 |
| odorant receptor 67d-like [Bactrocera oleae] 168 aa protein | XP_014097995.1 GI: 929375735 |
| odorant receptor Or2-like [Bactrocera oleae] 375 aa protein | XP_014097486.1 GI: 929374815 |
| odorant receptor Or2-like [Bactrocera oleae] 331 aa protein | XP_014097484.1 GI: 929374811 |
| odorant receptor 33b-like [Bactrocera oleae] 384 aa protein | XP_014097127.1 GI: 929374157 |
| odorant receptor 63a-like [Bactrocera oleae] 417 aa protein | XP_014096877.1 GI: 929373704 |
| odorant receptor 7a-like [Bactrocera oleae] 392 aa protein | XP_014095883.1 GI: 929371891 |
| odorant receptor 46a, isoform A [Bactrocera oleae] 388 aa protein | XP_014095654.1 GI: 929371469 |
| odorant receptor 49a-like [Bactrocera oleae] 394 aa protein | XP_014095510.1 GI: 929371206 |
| odorant receptor 94a-like [Bactrocera oleae] 337 aa protein | XP_014095326.1 GI: 929370867 |
| odorant receptor 67c [Bactrocera oleae] 404 aa protein | XP_014094968.1 GI: 929370189 |
| odorant receptor 94a-like [Bactrocera oleae] 403 aa protein | XP_014094554.1 GI: 929369423 |
| odorant receptor 94a-like [Bactrocera oleae] 392 aa protein | XP_014094548.1 GI: 929369411 |
| putative odorant receptor 85e [Bactrocera oleae] 450 aa protein | XP_014094455.1 GI: 929369240 |
| odorant receptor 13a [Bactrocera oleae] 456 aa protein | XP_014094420.1 GI: 929369174 |
| odorant receptor 63a-like [Bactrocera oleae] 275 aa protein | XP_014094225.1 GI: 929368815 |
| odorant receptor 63a-like [Bactrocera oleae] 414 aa protein | XP_014094224.1 GI: 929368813 |
| odorant receptor 63a-like [Bactrocera oleae] 357 aa protein | XP_014094223.1 GI: 929368811 |
| odorant receptor 85c-like [Bactrocera oleae] 415 aa protein | XP_014093775.1 GI: 929367999 |
| odorant receptor 85c-like [Bactrocera oleae] 211 aa protein | XP_014093774.1 GI: 929367997 |
| putative odorant receptor 85d [Bactrocera oleae] 420 aa protein | XP_014093772.1 GI: 929367993 |
| odorant receptor 7a [Bactrocera oleae] 396 aa protein | XP_014092478.1 GI: 929365645 |
| odorant receptor 94a-like [Bactrocera oleae] 192 aa protein | XP_014092468.1 GI: 929365629 |
| odorant receptor coreceptor [Bactrocera oleae] 473 aa protein | XP_014092453.1 GI: 929365601 |
| odorant receptor 83a [Bactrocera oleae] 473 aa protein | XP_014092452.1 GI: 929365599 |
| odorant receptor 7a-like [Bactrocera oleae] 394 aa protein | XP_014092042.1 GI: 929364851 |
| odorant receptor 10a [Bactrocera oleae] 402 aa protein | XP_014091911.1 GI: 929364611 |
| odorant receptor 82a [Bactrocera oleae] 398 aa protein | XP_014091900.1 GI: 929364589 |
| odorant receptor 67d-like [Bactrocera oleae] 388 aa protein | XP_014091805.1 GI: 929364416 |
| odorant receptor 67d-like [Bactrocera oleae] 388 aa protein | XP_014091792.1 GI: 929364391 |
| odorant receptor 74a [Bactrocera oleae] 402 aa protein | XP_014091648.1 GI: 929364123 |
| odorant receptor 2a-like [Bactrocera oleae] 393 aa protein | XP_014088938.1 GI: 929359208 |
| odorant receptor 94b-like [Bactrocera oleae] 402 aa protein | XP_014088795.1 GI: 929358946 |
| odorant receptor 43a [Bactrocera oleae] 378 aa protein | XP_014088559.1 GI: 929358514 |
| putative odorant receptor 69a, isoform B [Bactrocera oleae] 414 aa | XP_014088528.1 GI: 929358458 |
| protein | |
| putative odorant receptor 69a, isoform A [Bactrocera oleae] 289 aa | XP_014088513.1 GI: 929358429 |
| protein | |
| odorant receptor 43b-like [Bactrocera oleae] 337 aa protein | XP_014086206.1 GI: 929354211 |
| odorant receptor 74a-like [Bactrocera oleae] 414 aa protein | XP_014085775.1 GI: 929353423 |
| odorant receptor 35a-like [Bactrocera oleae] 417 aa protein | XP_014103705.1 GI: 929352035 |
| putative odorant receptor 92a [Bactrocera oleae] 250 aa protein | XP_014103608.1 GI: 929351857 |
| odorant receptor 7a-like [Bactrocera oleae] 384 aa protein | XP_014103552.1 GI: 929351755 |
| odorant receptor 2a-like [Bactrocera oleae] 384 aa protein | XP_014103551.1 GI: 929351753 |
| odorant receptor 85a-like [Bactrocera oleae] 254 aa protein | XP_014103550.1 GI: 929351751 |
| odorant receptor 22c [Bactrocera oleae] 400 aa protein | XP_014103181.1 GI: 929351088 |
| odorant receptor 24a [Bactrocera oleae] 397 aa protein | XP_014103094.1 GI: 929350935 |
| odorant receptor 49b-like [Bactrocera oleae] 371 aa protein | XP_014102906.1 GI: 929345515 |
| odorant receptor 47b [Bactrocera oleae] 423 aa protein | XP_014096040.1 GI: 929345200 |
| odorant receptor 59a-like [Bactrocera oleae] 378 aa protein | XP_014097365.1 GI: 929344966 |
| odorant receptor 59a-like [Bactrocera oleae] 380 aa protein | XP_014096236.1 GI: 929344964 |
| odorant receptor 53 [Microplitis mediator] 387 aa protein | AKO90017.1 GI: 861722551 |
| odorant receptor 52 [Microplitis mediator] 406 aa protein | AKO90016.1 GI: 861722548 |
| odorant receptor 51 [Microplitis mediator] 410 aa protein | AKO90015.1 GI: 861722545 |
| odorant receptor 50 [Microplitis mediator] 395 aa protein | AKO90014.1 GI: 861722542 |
| odorant receptor 49 [Microplitis mediator] 393 aa protein | AKO90013.1 GI: 861722539 |
| odorant receptor 48 [Microplitis mediator] 401 aa protein | AKO90012.1 GI: 861722536 |
| odorant receptor 47 [Microplitis mediator] 404 aa protein | AKO90011.1 GI: 861722533 |
| odorant receptor 46 [Microplitis mediator] 423 aa protein | AKO90010.1 GI: 861722530 |
| odorant receptor 45 [Microplitis mediator] 393 aa protein | AKO90009.1 GI: 861722527 |
| odorant receptor 44 [Microplitis mediator] 395 aa protein | AKO90008.1 GI: 861722524 |
| odorant receptor 43 [Microplitis mediator] 377 aa protein | AKO90007.1 GI: 861722521 |
| odorant receptor 42 [Microplitis mediator] 381 aa protein | AKO90006.1 GI: 861722518 |
| odorant receptor 41 [Microplitis mediator] 384 aa protein | AKO90005.1 GI: 861722515 |
| odorant receptor 40 [Microplitis mediator] 406 aa protein | AKO90004.1 GI: 861722506 |
| odorant receptor 39 [Microplitis mediator] 349 aa protein | AKO90003.1 GI: 861722503 |
| odorant receptor 38 [Microplitis mediator] 388 aa protein | AKO90002.1 GI: 861722500 |
| odorant receptor 37 [Microplitis mediator] 384 aa protein | AKO90001.1 GI: 861722497 |
| odorant receptor 36 [Microplitis mediator] 382 aa protein | AKO90000.1 GI: 861722494 |
| odorant receptor 35 [Microplitis mediator] 377 aa protein | AKO89999.1 GI: 861722490 |
| odorant receptor 34 [Microplitis mediator] 395 aa protein | AKO89998.1 GI: 861722487 |
| odorant receptor 33 [Microplitis mediator] 384 aa protein | AKO89997.1 GI: 861722484 |
| odorant receptor 32 [Microplitis mediator] 420 aa protein | AKO89996.1 GI: 861722481 |
| odorant receptor 31 [Microplitis mediator] 375 aa protein | AKO89995.1 GI: 861722478 |
| odorant receptor 30 [Microplitis mediator] 383 aa protein | AKO89994.1 GI: 861722475 |
| odorant receptor 29 [Microplitis mediator] 413 aa protein | AKO89993.1 GI: 861722471 |
| odorant receptor 28 [Microplitis mediator] 402 aa protein | AKO89992.1 GI: 861722468 |
| odorant receptor 27 [Microplitis mediator] 403 aa protein | AKO89991.1 GI: 861722465 |
| odorant receptor 26 [Microplitis mediator] 397 aa protein | AKO89990.1 GI: 861722462 |
| odorant receptor 25 [Microplitis mediator] 376 aa protein | AKO89989.1 GI: 861722459 |
| odorant receptor 24 [Microplitis mediator] 260 aa protein | AKO89988.1 GI: 861722456 |
| odorant receptor 23 [Microplitis mediator] 373 aa protein | AKO89987.1 GI: 861722453 |
| odorant receptor 22 [Microplitis mediator] 369 aa protein | AKO89986.1 GI: 861722450 |
| odorant receptor 21 [Microplitis mediator] 392 aa protein | AKO89985.1 GI: 861722447 |
| odorant receptor 20 [Microplitis mediator] 372 aa protein | AKO89984.1 GI: 861722444 |
| odorant receptor 19 [Microplitis mediator] 396 aa protein | AKO89983.1 GI: 861722441 |
| odorant receptor 18 [Microplitis mediator] 390 aa protein | AKO89982.1 GI: 861722438 |
| odorant receptor 17 [Microplitis mediator] 402 aa protein | AKO89981.1 GI: 861722435 |
| odorant receptor 16 [Microplitis mediator] 378 aa protein | AKO89980.1 GI: 861722432 |
| odorant receptor 15 [Microplitis mediator] 385 aa protein | AKO89979.1 GI: 861722429 |
| odorant receptor 14 [Microplitis mediator] 411 aa protein | AKO89978.1 GI: 861722426 |
| odorant receptor 13a-like [Plutella xylostella] 404 aa protein | NP_001296037.1 GI: 822092756 |
| odorant receptor 83b [Spodoptera litura] 473 aa protein | AFN22085.1 GI: 393757441 |
| odorant receptor 50 [Nasonia vitripennis] 373 aa protein | NP_001177496.1 GI: 299522706 |
| Gustatory and odorant receptor 21a 454 aa protein | Q9VPT1.3 GI: 158523347 |
| Gustatory and odorant receptor 63a 512 aa protein | Q9VZL7.1 GI: 20454944 |
| odorant receptor OR83b, partial [Chilo suppressalis] 338 aa | ACJ07125.1 GI: 210108262 |
| protein | |
| odorant receptor OR83b, partial [Sesamia inferens] 275 aa protein | ACJ07124.1 GI: 210108191 |
| odorant receptor coreceptor-like [Diuraphis noxia] 127 aa protein | XP_015378374.1 GI: 985425779 |
| gustatory and odorant receptor 63a-like [Halyomorpha halys] 403 | XP_014293240.1 GI: 939698138 |
| aa protein | |
| gustatory and odorant receptor 24-like isoform X2 [Halyomorpha | XP_014282243.1 GI: 939671105 |
| halys] 395 aa protein | |
| gustatory and odorant receptor 63a-like [Halyomorpha halys] 374 | XP_014281820.1 GI: 939670013 |
| aa protein | |
| gustatory and odorant receptor 63a-like [Halyomorpha halys] 395 | XP_014281153.1 GI: 939668267 |
| aa protein | |
| gustatory and odorant receptor 22-like [Halyomorpha halys] 134 | XP_014272022.1 GI: 939643445 |
| aa protein | |
| gustatory and odorant receptor 22-like [Halyomorpha halys] 199 | XP_014271847.1 GI: 939642935 |
| aa protein | |
| gustatory and odorant receptor 22-like isoform X2 [Halyomorpha | XP_014271842.1 GI: 939642921 |
| halys] 312 aa protein | |
| gustatory and odorant receptor 24-like isoform X1 [Halyomorpha | XP_014271841.1 GI: 939642918 |
| halys] 395 aa protein | |
| gustatory and odorant receptor 24-like isoform X4 [Halyomorpha | XP_014271840.1 GI: 939642916 |
| halys] 316 aa protein | |
| gustatory and odorant receptor 24-like isoform X3 [Halyomorpha | XP_014271839.1 GI: 939642913 |
| halys] 366 aa protein | |
| gustatory and odorant receptor 24-like isoform X2 [Halyomorpha | XP_014271838.1 GI: 939642910 |
| halys] 395 aa protein | |
| gustatory and odorant receptor 24-like isoform X1 [Halyomorpha | XP_014271836.1 GI: 939642908 |
| halys] 399 aa protein | |
| gustatory and odorant receptor 21a-like [Bactrocera oleae] 445 aa | XP_014101222.1 GI: 929381551 |
| protein | |
| gustatory and odorant receptor 21a-like, partial [Bactrocera oleae] | XP_014101212.1 GI: 929381535 |
| 367 aa protein | |
| gustatory and odorant receptor 21a-like, partial [Bactrocera oleae] | XP_014101173.1 GI: 929381464 |
| 278 aa protein | |
| gustatory and odorant receptor 21a-like, partial [Bactrocera oleae] | XP_014100623.1 GI: 929380462 |
| 200 aa protein | |
| gustatory and odorant receptor 21a [Bactrocera oleae] 456 aa | XP_014097799.1 GI: 929375381 |
| protein | |
| gustatory and odorant receptor 22-like [Bactrocera oleae] 129 aa | XP_014097326.1 GI: 929374521 |
| protein | |
| odorant receptor 67c-like, partial [Bactrocera oleae] 235 aa protein | XP_014096202.1 GI: 929372471 |
| gustatory and odorant receptor 63a [Bactrocera oleae] 485 aa | XP_014095104.1 GI: 929347538 |
| protein | |
| odorant receptor Or83b [Helicoverpa zea] 472 aa protein | AAX14773.1 GI: 60207120 |
| odorant receptor Or83b [Ceratitis capitata] 473 aa protein | AAX14775.1 GI: 60207191 |
| odorant receptor Or83b [Anopheles gambiae] 478 aa protein | AAX14774.1 GI: 60207155 |
| putative odorant receptor [Bombyx mori] 472 aa protein | BAD69585.1 GI: 55583295 |
| Gustatory and odorant receptor 24 457 aa protein | Q7PYF4.4 GI: 384872698 |
| Gustatory and odorant receptor 22 467 aa protein | Q7PMG3.1 GI: 74799392 |
| odorant receptor 7 [Culex pipiens pallens] 480 aa protein | AMQ13062.1 GI: 1005652053 |
| putative odorant receptor [Sesamia inferens] 442 aa protein | AGY14587.2 GI: 670657525 |
| putative odorant receptor [Sesamia inferens] 426 aa protein | AGY14585.2 GI: 670657520 |
| putative odorant receptor [Sesamia inferens] 432 aa protein | AGY14579.2 GI: 670657516 |
| putative odorant receptor [Sesamia inferens] 473 aa protein | AGY14565.1 GI: 550848914 |
| putative odorant receptor SinfOR18, partial [Sesamia inferens] 153 | AIF79425.1 GI: 665823788 |
| aa protein | |
| putative odorant receptor SinfOR17, partial [Sesamia inferens] 82 | AIF79424.1 GI: 665823786 |
| aa protein | |
| putative odorant receptor, partial [Sesamia inferens] 162 aa protein | AGY14595.1 GI: 550848974 |
| putative odorant receptor [Sesamia inferens] 402 aa protein | AGY14593.1 GI: 550848970 |
| putative odorant receptor, partial [Sesamia inferens] 225 aa protein | AGY14592.1 GI: 550848968 |
| putative odorant receptor, partial [Sesamia inferens] 230 aa protein | AGY14591.1 GI: 550848966 |
| putative odorant receptor, partial [Sesamia inferens] 380 aa protein | AGY14590.1 GI: 550848964 |
| putative odorant receptor, partial [Sesamia inferens] 250 aa protein | AGY14589.1 GI: 550848962 |
| putative odorant receptor, partial [Sesamia inferens] 275 aa protein | AGY14588.1 GI: 550848960 |
| putative odorant receptor, partial [Sesamia inferens] 268 aa protein | AGY14586.1 GI: 550848956 |
| putative odorant receptor, partial [Sesamia inferens] 203 aa protein | AGY14584.1 GI: 550848952 |
| putative odorant receptor, partial [Sesamia inferens] 223 aa protein | AGY14583.1 GI: 550848950 |
| putative odorant receptor, partial [Sesamia inferens] 247 aa protein | AGY14582.1 GI: 550848948 |
| putative odorant receptor, partial [Sesamia inferens] 254 aa protein | AGY14581.1 GI: 550848946 |
| putative odorant receptor, partial [Sesamia inferens] 224 aa protein | AGY14578.1 GI: 550848940 |
| putative odorant receptor, partial [Sesamia inferens] 368 aa protein | AGY14577.1 GI: 550848938 |
| putative odorant receptor, partial [Sesamia inferens] 67 aa protein | AGY14576.1 GI: 550848936 |
| putative odorant receptor, partial [Sesamia inferens] 88 aa protein | AGY14575.1 GI: 550848934 |
| putative odorant receptor, partial [Sesamia inferens] 84 aa protein | AGY14574.1 GI: 550848932 |
| putative odorant receptor, partial [Sesamia inferens] 69 aa protein | AGY14573.1 GI: 550848930 |
| putative odorant receptor, partial [Sesamia inferens] 118 aa protein | AGY14572.1 GI: 550848928 |
| putative odorant receptor, partial [Sesamia inferens] 95 aa protein | AGY14571.1 GI: 550848926 |
| putative odorant receptor, partial [Sesamia inferens] 161 aa protein | AGY14570.1 GI: 550848924 |
| putative odorant receptor, partial [Sesamia inferens] 82 aa protein | AGY14568.1 GI: 550848920 |
| putative odorant receptor, partial [Sesamia inferens] 115 aa protein | AGY14567.1 GI: 550848918 |
| putative odorant receptor, partial [Sesamia inferens] 98 aa protein | AGY14566.1 GI: 550848916 |
| odorant receptor 7 [Plutella xylostella] 424 aa protein | AGK43829.1 GI: 484354001 |
| odorant receptor 6 [Plutella xylostella] 424 aa protein | AGK43828.1 GI: 484353999 |
| odorant receptor 5 [Plutella xylostella] 404 aa protein | AGK43827.1 GI: 484353997 |
| odorant receptor 4 [Plutella xylostella] 402 aa protein | AGK43826.1 GI: 484353995 |
| odorant receptor 3 [Plutella xylostella] 403 aa protein | AGK43825.1 GI: 484353993 |
| odorant receptor 1 [Plutella xylostella] 422 aa protein | AGK43824.1 GI: 484353991 |
| odorant receptor 2 [Cnaphalocrocis medinalis] 473 aa protein | AFG73001.1 GI: 383215098 |
| putative odorant receptor [Bombyx mori] 430 aa protein | BAD69586.1 GI: 55583297 |
| putative odorant receptor, partial [Reticulitermes speratus] 382 aa | BAU20249.1 GI: 966774588 |
| protein | |
| putative odorant receptor [Reticulitermes speratus] 199 aa protein | BAU20248.1 GI: 966774586 |
| putative odorant receptor [Reticulitermes speratus] 293 aa protein | BAU20247.1 GI: 966774584 |
| putative odorant receptor, partial [Reticulitermes speratus] 125 aa | BAU20246.1 GI: 966774582 |
| protein | |
| putative odorant receptor [Reticulitermes speratus] 481 aa protein | BAU20245.1 GI: 966774580 |
| putative odorant receptor [Reticulitermes speratus] 218 aa protein | BAU20244.1 GI: 966774578 |
| putative odorant receptor, partial [Reticulitermes speratus] 119 aa | BAU20243.1 GI: 966774576 |
| protein | |
| putative odorant receptor, partial [Reticulitermes speratus] 240 aa | BAU20242.1 GI: 966774574 |
| protein | |
| putative odorant receptor, partial [Reticulitermes speratus] 359 aa | BAU20241.1 GI: 966774572 |
| protein | |
| putative odorant receptor co-receptor [Reticulitermes speratus] 472 | BAU20240.1 GI: 966774570 |
| aa protein | |
| putative odorant receptor, partial [Reticulitermes speratus] 224 aa | BAU20239.1 GI: 966774568 |
| protein | |
| putative odorant receptor, partial [Reticulitermes speratus] 235 aa | BAU20238.1 GI: 966774566 |
| protein | |
| putative odorant receptor [Reticulitermes speratus] 491 aa protein | BAU20237.1 GI: 966774564 |
| putative odorant receptor [Reticulitermes speratus] 461 aa protein | BAU20236.1 GI: 966774562 |
| putative odorant receptor, partial [Reticulitermes speratus] 211 aa | BAU20235.1 GI: 966774560 |
| protein | |
| putative odorant receptor [Reticulitermes speratus] 406 aa protein | BAU20234.1 GI: 966774558 |
| putative odorant receptor, partial [Reticulitermes speratus] 411 aa | BAU20233.1 GI: 966774556 |
| protein | |
| putative odorant receptor, partial [Reticulitermes speratus] 469 aa | BAU20232.1 GI: 966774554 |
| protein | |
| putative odorant receptor, partial [Reticulitermes speratus] 334 aa | BAU20231.1 GI: 966774552 |
| protein | |
| putative odorant receptor 3, partial [Reticulitermes speratus] 429 | BAU20230.1 GI: 966774550 |
| aa protein | |
| putative odorant receptor 2 [Reticulitermes speratus] 153 aa | BAU20229.1 GI: 966774548 |
| protein | |
| putative odorant receptor 1, partial [Reticulitermes speratus] 101 | BAU20228.1 GI: 966774546 |
| aa protein | |
| putative odorant receptor, partial [Sesamia inferens] 156 aa protein | AGY14594.1 GI: 550848972 |
| putative odorant receptor, partial [Sesamia inferens] 107 aa protein | AGY14580.1 GI: 550848944 |
| putative odorant receptor, partial [Sesamia inferens] 70 aa protein | AGY14569.1 GI: 550848922 |
| putative odorant receptor, partial [Sesamia inferens] 188 aa protein | AGY14564.1 GI: 550848912 |
| Odorant receptor coreceptor; Odorant receptor 83b 486 aa protein | Q9VNB5.2 GI: 14285640 |
| Odorant receptor 22a 397 aa protein | P81909.1 GI: 12643687 |
| Odorant receptor 59b 398 aa protein | Q9W1P8.1 GI: 11387003 |
| Odorant receptor 67a 407 aa protein | Q9VT08.2 GI: 14285630 |
| Odorant receptor 43a 376 aa protein | P81917.2 GI: 12643691 |
| Odorant receptor 22b 397 aa protein | P81910.3 GI: 221222515 |
| Odorant receptor 35a 409 aa protein | Q9V3Q2.3 GI: 48429266 |
| Odorant receptor 67d 391 aa protein | Q9VT92.3 GI: 47117341 |
| Odorant receptor 46a, isoform A 381 aa protein | P81919.4 GI: 39932724 |
| Odorant receptor 85b 390 aa protein | Q9VHQ7.2 GI: 14285638 |
| Odorant receptor 67b 421 aa protein | Q9VT20.2 GI: 14285631 |
| Odorant receptor 46a, isoform B 384 aa protein | Q9V3N2.2 GI: 14285623 |
| Odorant receptor 47a 385 aa protein | P81921.1 GI: 12643694 |
| Odorant receptor 10a 406 aa protein | Q9VYZ1.1 GI: 11387000 |
| Odorant receptor 24a 398 aa protein | P81913.4 GI: 251757500 |
| Odorant receptor 47b 412 aa protein | P81922.2 GI: 47606742 |
| Odorant receptor 30a 377 aa protein | Q9VLE5.4 GI: 41019523 |
| Odorant receptor 45a 378 aa protein | Q9V568.3 GI: 37999962 |
| Odorant receptor 42a 406 aa protein | Q9V9I2.3 GI: 22096371 |
| Odorant receptor 49a 396 aa protein | Q9V6A9.3 GI: 22096370 |
| Odorant receptor 1a 392 aa protein | Q9W5G6.2 GI: 14285651 |
| Odorant receptor 13a 418 aa protein | Q9VXL0.2 GI: 14285650 |
| Odorant receptor 88a 401 aa protein | Q9VFN2.2 GI: 14285649 |
| Odorant receptor 98a 397 aa protein | Q9VAZ3.2 GI: 14285647 |
| Odorant receptor 19a 387 aa protein | Q9I816.2 GI: 14285645 |
| Odorant receptor 83a 453 aa protein | Q9VNB3.2 GI: 14285639 |
| Odorant receptor 85c 389 aa protein | Q9VHQ6.2 GI: 14285637 |
| Odorant receptor 85f 392 aa protein | Q9VHE6.1 GI: 14285636 |
| Odorant receptor 82a 385 aa protein | P82986.1 GI: 14285635 |
| Odorant receptor 63a 420 aa protein | Q9VZW8.2 GI: 14285633 |
| Odorant receptor 67c 404 aa protein | Q9VT90.2 GI: 14285632 |
| Odorant receptor 42b 399 aa protein | Q9V9I4.2 GI: 14285628 |
| Odorant receptor 56a 419 aa protein | Q9V8Y7.2 GI: 14285626 |
| Odorant receptor 65a 417 aa protein | P82982.1 GI: 14285620 |
| Odorant receptor 43b 403 aa protein | P81918.3 GI: 14285618 |
| Odorant receptor 22c 402 aa protein | P81911.2 GI: 14285616 |
| Odorant receptor 49b 375 aa protein | Q9V6H2.1 GI: 12643916 |
| Odorant receptor 59a 397 aa protein | P81923.2 GI: 12643696 |
| Odorant receptor 2a 397 aa protein | O46077.2 GI: 12643564 |
| Odorant receptor 7a 413 aa protein | Q9W3I5.1 GI: 11387005 |
| Odorant receptor 9a 392 aa protein | Q9W2U9.1 GI: 11387004 |
| Odorant receptor 74a 404 aa protein | Q9VVF3.1 GI: 11386999 |
| Odorant receptor 85a 397 aa protein | Q9VHS4.1 GI: 11386993 |
| Odorant receptor 94a 387 aa protein | Q9VCS9.1 GI: 11386991 |
| Odorant receptor 94b 383 aa protein | Q9VCS8.1 GI: 11386990 |
| Odorant receptor 45b 396 aa protein | Q9V589.1 GI: 11386985 |
| Odorant receptor 33c 384 aa protein | P81916.1 GI: 11386980 |
| Odorant receptor 33b 379 aa protein | P81915.1 GI: 11386979 |
| Odorant receptor 33a 378 aa protein | P81914.1 GI: 11386978 |
| Odorant receptor 23a 379 aa protein | P81912.1 GI: 11386977 |
| hypothetical protein X777_04609 [Cerapachys biroi] 397 aa | EZA62900.1 GI: 607368794 |
| protein | |
| hypothetical protein X777_04608 [Cerapachys biroi] 399 aa | EZA62899.1 GI: 607368793 |
| protein | |
| hypothetical protein X777_07630, partial [Cerapachys biroi] 109 | EZA62813.1 GI: 607368701 |
| aa protein | |
| hypothetical protein X777_07612, partial [Cerapachys biroi] 305 | EZA62796.1 GI: 607368684 |
| aa protein | |
| hypothetical protein X777_07611 [Cerapachys biroi] 376 aa | EZA62795.1 GI: 607368683 |
| protein | |
| hypothetical protein X777_10236 [Cerapachys biroi] 402 aa | EZA62605.1 GI: 607368492 |
| protein | |
| hypothetical protein X777_03408 [Cerapachys biroi] 389 aa | EZA62373.1 GI: 607368259 |
| protein | |
| hypothetical protein X777_03407 [Cerapachys biroi] 390 aa | EZA62372.1 GI: 607368258 |
| protein | |
| hypothetical protein X777_03406 [Cerapachys biroi] 391 aa | EZA62371.1 GI: 607368257 |
| protein | |
| hypothetical protein X777_03405 [Cerapachys biroi] 391 aa | EZA62370.1 GI: 607368256 |
| protein | |
| hypothetical protein X777_03404 [Cerapachys biroi] 382 aa | EZA62369.1 GI: 607368255 |
| protein | |
| hypothetical protein X777_03402 [Cerapachys biroi] 390 aa | EZA62368.1 GI: 607368254 |
| protein | |
| hypothetical protein X777_03401 [Cerapachys biroi] 392 aa | EZA62367.1 GI: 607368253 |
| protein | |
| hypothetical protein X777_09391 [Cerapachys biroi] 395 aa | EZA61770.1 GI: 607367631 |
| protein | |
| hypothetical protein X777_09390 [Cerapachys biroi] 396 aa | EZA61769.1 GI: 607367630 |
| protein | |
| hypothetical protein X777_09389 [Cerapachys biroi] 399 aa | EZA61768.1 GI: 607367629 |
| protein | |
| hypothetical protein X777_09388 [Cerapachys biroi] 394 aa | EZA61767.1 GI: 607367628 |
| protein | |
| hypothetical protein X777_09387 [Cerapachys biroi] 397 aa | EZA61766.1 GI: 607367627 |
| protein | |
| hypothetical protein X777_09305 [Cerapachys biroi] 394 aa | EZA61684.1 GI: 607367545 |
| protein | |
| hypothetical protein X777_07969 [Cerapachys biroi] 350 aa | EZA61634.1 GI: 607367487 |
| protein | |
| hypothetical protein X777_07966 [Cerapachys biroi] 396 aa | EZA61633.1 GI: 607367486 |
| protein | |
| hypothetical protein X777_07964 [Cerapachys biroi] 395 aa | EZA61631.1 GI: 607367484 |
| protein | |
| hypothetical protein X777_12474 [Cerapachys biroi] 375 aa | EZA61376.1 GI: 607367226 |
| protein | |
| hypothetical protein X777_08255 [Cerapachys biroi] 397 aa | EZA61043.1 GI: 607366887 |
| protein | |
| hypothetical protein X777_14254, partial [Cerapachys biroi] 255 | EZA60648.1 GI: 607366483 |
| aa protein | |
| hypothetical protein X777_13392 [Cerapachys biroi] 417 aa | EZA60303.1 GI: 607366132 |
| protein | |
| hypothetical protein X777_16159 [Cerapachys biroi] 396 aa | EZA59956.1 GI: 607365776 |
| protein | |
| hypothetical protein X777_16072 [Cerapachys biroi] 382 aa | EZA59870.1 GI: 607365690 |
| protein | |
| hypothetical protein X777_00307 [Cerapachys biroi] 382 aa | EZA59464.1 GI: 607365266 |
| protein | |
| hypothetical protein X777_00306 [Cerapachys biroi] 382 aa | EZA59463.1 GI: 607365265 |
| protein | |
| hypothetical protein X777_00305 [Cerapachys biroi] 379 aa | EZA59462.1 GI: 607365264 |
| protein | |
| hypothetical protein X777_00304 [Cerapachys biroi] 379 aa | EZA59461.1 GI: 607365263 |
| protein | |
| hypothetical protein X777_00303 [Cerapachys biroi] 380 aa | EZA59460.1 GI: 607365262 |
| protein | |
| hypothetical protein X777_00693, partial [Cerapachys biroi] 388 | EZA58872.1 GI: 607364664 |
| aa protein | |
| hypothetical protein X777_00565, partial [Cerapachys biroi] 110 | EZA58870.1 GI: 607364661 |
| aa protein | |
| hypothetical protein X777_00710, partial [Cerapachys biroi] 374 | EZA58868.1 GI: 607364658 |
| aa protein | |
| hypothetical protein X777_00709, partial [Cerapachys biroi] 393 | EZA58867.1 GI: 607364657 |
| aa protein | |
| hypothetical protein X777_00705, partial [Cerapachys biroi] 391 | EZA58866.1 GI: 607364656 |
| aa protein | |
| hypothetical protein X777_00703, partial [Cerapachys biroi] 390 | EZA58865.1 GI: 607364655 |
| aa protein | |
| hypothetical protein X777_00702, partial [Cerapachys biroi] 373 | EZA58864.1 GI: 607364654 |
| aa protein | |
| hypothetical protein X777_00701, partial [Cerapachys biroi] 390 | EZA58863.1 GI: 607364653 |
| aa protein | |
| hypothetical protein X777_00700, partial [Cerapachys biroi] 390 | EZA58862.1 GI: 607364652 |
| aa protein | |
| hypothetical protein X777_14780, partial [Cerapachys biroi] 298 | EZA58612.1 GI: 607364400 |
| aa protein | |
| hypothetical protein X777_14779 [Cerapachys biroi] 368 aa | EZA58611.1 GI: 607364399 |
| protein | |
| hypothetical protein X777_14777 [Cerapachys biroi] 368 aa | EZA58610.1 GI: 607364398 |
| protein | |
| hypothetical protein X777_14776 [Cerapachys biroi] 373 aa | EZA58609.1 GI: 607364397 |
| protein | |
| hypothetical protein X777_14775 [Cerapachys biroi] 374 aa | EZA58608.1 GI: 607364396 |
| protein | |
| hypothetical protein X777_14774 [Cerapachys biroi] 372 aa | EZA58607.1 GI: 607364395 |
| protein | |
| hypothetical protein X777_14773 [Cerapachys biroi] 367 aa | EZA58606.1 GI: 607364394 |
| protein | |
| hypothetical protein X777_14772 [Cerapachys biroi] 372 aa | EZA58605.1 GI: 607364393 |
| protein | |
| hypothetical protein X777_14770 [Cerapachys biroi] 370 aa | EZA58604.1 GI: 607364392 |
| protein | |
| hypothetical protein X777_14769 [Cerapachys biroi] 371 aa | EZA58603.1 GI: 607364391 |
| protein | |
| hypothetical protein X777_14768 [Cerapachys biroi] 369 aa | EZA58602.1 GI: 607364390 |
| protein | |
| hypothetical protein X777_14767 [Cerapachys biroi] 365 aa | EZA58601.1 GI: 607364389 |
| protein | |
| hypothetical protein X777_14766, partial [Cerapachys biroi] 362 | EZA58600.1 GI: 607364388 |
| aa protein | |
| hypothetical protein X777_01291 [Cerapachys biroi] 417 aa | EZA58334.1 GI: 607364113 |
| protein | |
| hypothetical protein X777_01236 [Cerapachys biroi] 391 aa | EZA58279.1 GI: 607364058 |
| protein | |
| hypothetical protein X777_01205 [Cerapachys biroi] 403 aa | EZA58248.1 GI: 607364027 |
| protein | |
| hypothetical protein X777_01932, partial [Cerapachys biroi] 212 | EZA58117.1 GI: 607363890 |
| aa protein | |
| hypothetical protein X777_01931, partial [Cerapachys biroi] 386 | EZA58116.1 GI: 607363889 |
| aa protein | |
| hypothetical protein X777_01929 [Cerapachys biroi] 387 aa | EZA58115.1 GI: 607363888 |
| protein | |
| hypothetical protein X777_01925, partial [Cerapachys biroi] 387 | EZA58113.1 GI: 607363886 |
| aa protein | |
| hypothetical protein X777_01917, partial [Cerapachys biroi] 388 | EZA58112.1 GI: 607363885 |
| aa protein | |
| hypothetical protein X777_01492, partial [Cerapachys biroi] 346 | EZA58111.1 GI: 607363883 |
| aa protein | |
| hypothetical protein X777_01491 [Cerapachys biroi] 394 aa | EZA58110.1 GI: 607363882 |
| protein | |
| hypothetical protein X777_01490 [Cerapachys biroi] 392 aa | EZA58109.1 GI: 607363881 |
| protein | |
| hypothetical protein X777_02093, partial [Cerapachys biroi] 368 | EZA57554.1 GI: 607363317 |
| aa protein | |
| hypothetical protein X777_02092 [Cerapachys biroi] 377 aa | EZA57553.1 GI: 607363316 |
| protein | |
| hypothetical protein X777_02090 [Cerapachys biroi] 395 aa | EZA57552.1 GI: 607363315 |
| protein | |
| hypothetical protein X777_02089 [Cerapachys biroi] 387 aa | EZA57551.1 GI: 607363314 |
| protein | |
| hypothetical protein X777_02088 [Cerapachys biroi] 393 aa | EZA57550.1 GI: 607363313 |
| protein | |
| hypothetical protein X777_02087 [Cerapachys biroi] 381 aa | EZA57549.1 GI: 607363312 |
| protein | |
| hypothetical protein X777_02086 [Cerapachys biroi] 378 aa | EZA57548.1 GI: 607363311 |
| protein | |
| hypothetical protein X777_02085 [Cerapachys biroi] 387 aa | EZA57547.1 GI: 607363310 |
| protein | |
| hypothetical protein X777_02082 [Cerapachys biroi] 396 aa | EZA57546.1 GI: 607363309 |
| protein | |
| hypothetical protein X777_02081 [Cerapachys biroi] 377 aa | EZA57545.1 GI: 607363308 |
| protein | |
| hypothetical protein X777_02080 [Cerapachys biroi] 385 aa | EZA57544.1 GI: 607363307 |
| protein | |
| hypothetical protein X777_02079 [Cerapachys biroi] 382 aa | EZA57543.1 GI: 607363306 |
| protein | |
| hypothetical protein X777_02078 [Cerapachys biroi] 384 aa | EZA57542.1 GI: 607363305 |
| protein | |
| hypothetical protein X777_02075 [Cerapachys biroi] 385 aa | EZA57540.1 GI: 607363303 |
| protein | |
| hypothetical protein X777_02074 [Cerapachys biroi] 387 aa | EZA57539.1 GI: 607363302 |
| protein | |
| hypothetical protein X777_02073 [Cerapachys biroi] 383 aa | EZA57538.1 GI: 607363301 |
| protein | |
| hypothetical protein X777_02072 [Cerapachys biroi] 385 aa | EZA57537.1 GI: 607363300 |
| protein | |
| hypothetical protein X777_02071 [Cerapachys biroi] 382 aa | EZA57536.1 GI: 607363299 |
| protein | |
| hypothetical protein X777_02070 [Cerapachys biroi] 384 aa | EZA57535.1 GI: 607363298 |
| protein | |
| hypothetical protein X777_02069, partial [Cerapachys biroi] 367 | EZA57534.1 GI: 607363297 |
| aa protein | |
| hypothetical protein X777_02068 [Cerapachys biroi] 384 aa | EZA57533.1 GI: 607363296 |
| protein | |
| hypothetical protein X777_02067 [Cerapachys biroi] 384 aa | EZA57532.1 GI: 607363295 |
| protein | |
| hypothetical protein X777_02065 [Cerapachys biroi] 379 aa | EZA57531.1 GI: 607363294 |
| protein | |
| hypothetical protein X777_01603, partial [Cerapachys biroi] 388 | EZA56997.1 GI: 607362752 |
| aa protein | |
| hypothetical protein X777_01602, partial [Cerapachys biroi] 390 | EZA56996.1 GI: 607362751 |
| aa protein | |
| hypothetical protein X777_01601, partial [Cerapachys biroi] 389 | EZA56995.1 GI: 607362750 |
| aa protein | |
| hypothetical protein X777_01600, partial [Cerapachys biroi] 379 | EZA56994.1 GI: 607362749 |
| aa protein | |
| hypothetical protein X777_02369 [Cerapachys biroi] 375 aa | EZA56762.1 GI: 607362510 |
| protein | |
| hypothetical protein X777_02368 [Cerapachys biroi] 368 aa | EZA56761.1 GI: 607362509 |
| protein | |
| hypothetical protein X777_02340 [Cerapachys biroi] 398 aa | EZA56735.1 GI: 607362483 |
| protein | |
| hypothetical protein X777_03347 [Cerapachys biroi] 391 aa | EZA56560.1 GI: 607362304 |
| protein | |
| hypothetical protein X777_03346, partial [Cerapachys biroi] 377 | EZA56559.1 GI: 607362303 |
| aa protein | |
| hypothetical protein X777_03345 [Cerapachys biroi] 392 aa | EZA56558.1 GI: 607362302 |
| protein | |
| hypothetical protein X777_03344 [Cerapachys biroi] 372 aa | EZA56557.1 GI: 607362301 |
| protein | |
| hypothetical protein X777_03736, partial [Cerapachys biroi] 383 | EZA56086.1 GI: 607361821 |
| aa protein | |
| hypothetical protein X777_04121 [Cerapachys biroi] 383 aa | EZA55902.1 GI: 607361626 |
| protein | |
| hypothetical protein X777_04120 [Cerapachys biroi] 383 aa | EZA55901.1 GI: 607361625 |
| protein | |
| hypothetical protein X777_03929 [Cerapachys biroi] 388 aa | EZA55755.1 GI: 607361477 |
| protein | |
| hypothetical protein X777_03928, partial [Cerapachys biroi] 335 | EZA55754.1 GI: 607361476 |
| aa protein | |
| hypothetical protein X777_04285 [Cerapachys biroi] 388 aa | EZA55491.1 GI: 607361197 |
| protein | |
| hypothetical protein X777_04264 [Cerapachys biroi] 396 aa | EZA55471.1 GI: 607361177 |
| protein | |
| hypothetical protein X777_04263 [Cerapachys biroi] 393 aa | EZA55470.1 GI: 607361176 |
| protein | |
| hypothetical protein X777_04768 [Cerapachys biroi] 299 aa | EZA55372.1 GI: 607361071 |
| protein | |
| hypothetical protein X777_04767 [Cerapachys biroi] 395 aa | EZA55371.1 GI: 607361070 |
| protein | |
| hypothetical protein X777_04765 [Cerapachys biroi] 412 aa | EZA55369.1 GI: 607361068 |
| protein | |
| hypothetical protein X777_05311 [Cerapachys biroi] 388 aa | EZA55133.1 GI: 607360814 |
| protein | |
| hypothetical protein X777_05310 [Cerapachys biroi] 391 aa | EZA55132.1 GI: 607360813 |
| protein | |
| hypothetical protein X777_05285, partial [Cerapachys biroi] 347 | EZA55110.1 GI: 607360791 |
| aa protein | |
| hypothetical protein X777_05369 [Cerapachys biroi] 395 aa | EZA55090.1 GI: 607360767 |
| protein | |
| hypothetical protein X777_05368 [Cerapachys biroi] 398 aa | EZA55089.1 GI: 607360766 |
| protein | |
| hypothetical protein X777_05462 [Cerapachys biroi] 391 aa | EZA54938.1 GI: 607360613 |
| protein | |
| hypothetical protein X777_05757 [Cerapachys biroi] 404 aa | EZA54481.1 GI: 607360141 |
| protein | |
| hypothetical protein X777_05686, partial [Cerapachys biroi] 347 | EZA54448.1 GI: 607360105 |
| aa protein | |
| hypothetical protein X777_05685 [Cerapachys biroi] 394 aa | EZA54447.1 GI: 607360104 |
| protein | |
| hypothetical protein X777_05682, partial [Cerapachys biroi] 344 | EZA54446.1 GI: 607360103 |
| aa protein | |
| hypothetical protein X777_05679, partial [Cerapachys biroi] 385 | EZA54444.1 GI: 607360101 |
| aa protein | |
| hypothetical protein X777_05674, partial [Cerapachys biroi] 346 | EZA54439.1 GI: 607360096 |
| aa protein | |
| hypothetical protein X777_05671, partial [Cerapachys biroi] 307 | EZA54437.1 GI: 607360094 |
| aa protein | |
| hypothetical protein X777_05670, partial [Cerapachys biroi] 86 aa | EZA54436.1 GI: 607360093 |
| protein | |
| hypothetical protein X777_05669 [Cerapachys biroi] 401 aa | EZA54435.1 GI: 607360092 |
| protein | |
| hypothetical protein X777_05668 [Cerapachys biroi] 393 aa | EZA54434.1 GI: 607360091 |
| protein | |
| hypothetical protein X777_05665 [Cerapachys biroi] 353 aa | EZA54432.1 GI: 607360089 |
| protein | |
| hypothetical protein X777_05664 [Cerapachys biroi] 393 aa | EZA54431.1 GI: 607360088 |
| protein | |
| hypothetical protein X777_05662 [Cerapachys biroi] 399 aa | EZA54429.1 GI: 607360086 |
| protein | |
| hypothetical protein X777_05661 [Cerapachys biroi] 396 aa | EZA54428.1 GI: 607360085 |
| protein | |
| hypothetical protein X777_05660 [Cerapachys biroi] 394 aa | EZA54427.1 GI: 607360084 |
| protein | |
| hypothetical protein X777_05658 [Cerapachys biroi] 394 aa | EZA54426.1 GI: 607360083 |
| protein | |
| hypothetical protein X777_05656, partial [Cerapachys biroi] 347 | EZA54424.1 GI: 607360081 |
| aa protein | |
| hypothetical protein X777_05655 [Cerapachys biroi] 396 aa | EZA54423.1 GI: 607360080 |
| protein | |
| hypothetical protein X777_05651 [Cerapachys biroi] 394 aa | EZA54421.1 GI: 607360078 |
| protein | |
| hypothetical protein X777_05650 [Cerapachys biroi] 394 aa | EZA54420.1 GI: 607360077 |
| protein | |
| hypothetical protein X777_05649 [Cerapachys biroi] 395 aa | EZA54419.1 GI: 607360076 |
| protein | |
| hypothetical protein X777_05648 [Cerapachys biroi] 394 aa | EZA54418.1 GI: 607360075 |
| protein | |
| hypothetical protein X777_07020, partial [Cerapachys biroi] 305 | EZA53552.1 GI: 607359176 |
| aa protein | |
| hypothetical protein X777_07019 [Cerapachys biroi] 352 aa | EZA53551.1 GI: 607359175 |
| protein | |
| hypothetical protein X777_08120 [Cerapachys biroi] 391 aa | EZA52637.1 GI: 607358221 |
| protein | |
| hypothetical protein X777_08119 [Cerapachys biroi] 389 aa | EZA52636.1 GI: 607358220 |
| protein | |
| hypothetical protein X777_08118 [Cerapachys biroi] 359 aa | EZA52635.1 GI: 607358219 |
| protein | |
| hypothetical protein X777_08117 [Cerapachys biroi] 354 aa | EZA52634.1 GI: 607358218 |
| protein | |
| hypothetical protein X777_08040 [Cerapachys biroi] 387 aa | EZA52558.1 GI: 607358142 |
| protein | |
| hypothetical protein X777_08039 [Cerapachys biroi] 398 aa | EZA52557.1 GI: 607358141 |
| protein | |
| hypothetical protein X777_08037 [Cerapachys biroi] 402 aa | EZA52556.1 GI: 607358140 |
| protein | |
| hypothetical protein X777_08034 [Cerapachys biroi] 387 aa | EZA52555.1 GI: 607358139 |
| protein | |
| hypothetical protein X777_08033 [Cerapachys biroi] 395 aa | EZA52554.1 GI: 607358138 |
| protein | |
| hypothetical protein X777_08032 [Cerapachys biroi] 404 aa | EZA52553.1 GI: 607358137 |
| protein | |
| hypothetical protein X777_08610 [Cerapachys biroi] 393 aa | EZA52503.1 GI: 607358081 |
| protein | |
| hypothetical protein X777_08609 [Cerapachys biroi] 393 aa | EZA52502.1 GI: 607358080 |
| protein | |
| hypothetical protein X777_08608, partial [Cerapachys biroi] 348 | EZA52501.1 GI: 607358079 |
| aa protein | |
| hypothetical protein X777_08641 [Cerapachys biroi] 403 aa | EZA52498.1 GI: 607358074 |
| protein | |
| hypothetical protein X777_08639 [Cerapachys biroi] 405 aa | EZA52497.1 GI: 607358073 |
| protein | |
| hypothetical protein X777_08638 [Cerapachys biroi] 415 aa | EZA52496.1 GI: 607358072 |
| protein | |
| hypothetical protein X777_08637 [Cerapachys biroi] 410 aa | EZA52495.1 GI: 607358071 |
| protein | |
| hypothetical protein X777_08636 [Cerapachys biroi] 387 aa | EZA52494.1 GI: 607358070 |
| protein | |
| hypothetical protein X777_08635 [Cerapachys biroi] 412 aa | EZA52493.1 GI: 607358069 |
| protein | |
| hypothetical protein X777_08634, partial [Cerapachys biroi] 296 | EZA52492.1 GI: 607358068 |
| aa protein | |
| hypothetical protein X777_08633, partial [Cerapachys biroi] 296 | EZA52491.1 GI: 607358067 |
| aa protein | |
| hypothetical protein X777_08535 [Cerapachys biroi] 369 aa | EZA52460.1 GI: 607358030 |
| protein | |
| hypothetical protein X777_08647 [Cerapachys biroi] 409 aa | EZA52136.1 GI: 607357695 |
| protein | |
| hypothetical protein X777_09139, partial [Cerapachys biroi] 67 aa | EZA52127.1 GI: 607357681 |
| protein | |
| hypothetical protein X777_09138 [Cerapachys biroi] 403 aa | EZA52126.1 GI: 607357680 |
| protein | |
| hypothetical protein X777_09136 [Cerapachys biroi] 404 aa | EZA52125.1 GI: 607357679 |
| protein | |
| hypothetical protein X777_09135 [Cerapachys biroi] 404 aa | EZA52124.1 GI: 607357678 |
| protein | |
| hypothetical protein X777_09133 [Cerapachys biroi] 398 aa | EZA52123.1 GI: 607357677 |
| protein | |
| hypothetical protein X777_09132 [Cerapachys biroi] 408 aa | EZA52122.1 GI: 607357676 |
| protein | |
| hypothetical protein X777_09131 [Cerapachys biroi] 410 aa | EZA52121.1 GI: 607357675 |
| protein | |
| hypothetical protein X777_09130 [Cerapachys biroi] 410 aa | EZA52120.1 GI: 607357674 |
| protein | |
| hypothetical protein X777_09129 [Cerapachys biroi] 410 aa | EZA52119.1 GI: 607357673 |
| protein | |
| hypothetical protein X777_09127 [Cerapachys biroi] 413 aa | EZA52118.1 GI: 607357672 |
| protein | |
| hypothetical protein X777_09126 [Cerapachys biroi] 405 aa | EZA52117.1 GI: 607357671 |
| protein | |
| hypothetical protein X777_09124 [Cerapachys biroi] 402 aa | EZA52116.1 GI: 607357670 |
| protein | |
| hypothetical protein X777_09123 [Cerapachys biroi] 406 aa | EZA52115.1 GI: 607357669 |
| protein | |
| hypothetical protein X777_09298, partial [Cerapachys biroi] 316 | EZA52015.1 GI: 607357565 |
| aa protein | |
| hypothetical protein X777_08804 [Cerapachys biroi] 397 aa | EZA51620.1 GI: 607357154 |
| protein | |
| hypothetical protein X777_08803 [Cerapachys biroi] 395 aa | EZA51619.1 GI: 607357153 |
| protein | |
| hypothetical protein X777_08801 [Cerapachys biroi] 394 aa | EZA51618.1 GI: 607357152 |
| protein | |
| hypothetical protein X777_08800 [Cerapachys biroi] 395 aa | EZA51617.1 GI: 607357151 |
| protein | |
| hypothetical protein X777_08799 [Cerapachys biroi] 394 aa | EZA51616.1 GI: 607357150 |
| protein | |
| hypothetical protein X777_08798 [Cerapachys biroi] 394 aa | EZA51615.1 GI: 607357149 |
| protein | |
| hypothetical protein X777_08797 [Cerapachys biroi] 394 aa | EZA51614.1 GI: 607357148 |
| protein | |
| hypothetical protein X777_08795 [Cerapachys biroi] 397 aa | EZA51612.1 GI: 607357146 |
| protein | |
| hypothetical protein X777_08794 [Cerapachys biroi] 395 aa | EZA51611.1 GI: 607357145 |
| protein | |
| hypothetical protein X777_08793 [Cerapachys biroi] 353 aa | EZA51610.1 GI: 607357144 |
| protein | |
| hypothetical protein X777_08790 [Cerapachys biroi] 394 aa | EZA51607.1 GI: 607357141 |
| protein | |
| hypothetical protein X777_08789 [Cerapachys biroi] 394 aa | EZA51606.1 GI: 607357140 |
| protein | |
| hypothetical protein X777_08788 [Cerapachys biroi] 394 aa | EZA51605.1 GI: 607357139 |
| protein | |
| hypothetical protein X777_08787 [Cerapachys biroi] 390 aa | EZA51604.1 GI: 607357138 |
| protein | |
| hypothetical protein X777_08786 [Cerapachys biroi] 394 aa | EZA51603.1 GI: 607357137 |
| protein | |
| hypothetical protein X777_08785 [Cerapachys biroi] 394 aa | EZA51602.1 GI: 607357136 |
| protein | |
| hypothetical protein X777_08783, partial [Cerapachys biroi] 109 | EZA51600.1 GI: 607357134 |
| aa protein | |
| hypothetical protein X777_09662 [Cerapachys biroi] 379 aa | EZA51393.1 GI: 607356910 |
| protein | |
| hypothetical protein X777_10267, partial [Cerapachys biroi] 277 | EZA51181.1 GI: 607356681 |
| aa protein | |
| hypothetical protein X777_10879 [Cerapachys biroi] 350 aa | EZA50908.1 GI: 607356383 |
| protein | |
| hypothetical protein X777_10878 [Cerapachys biroi] 388 aa | EZA50907.1 GI: 607356382 |
| protein | |
| hypothetical protein X777_10877 [Cerapachys biroi] 393 aa | EZA50906.1 GI: 607356381 |
| protein | |
| hypothetical protein X777_10876 [Cerapachys biroi] 394 aa | EZA50905.1 GI: 607356380 |
| protein | |
| hypothetical protein X777_10874 [Cerapachys biroi] 393 aa | EZA50904.1 GI: 607356379 |
| protein | |
| hypothetical protein X777_10873 [Cerapachys biroi] 393 aa | EZA50903.1 GI: 607356378 |
| protein | |
| hypothetical protein X777_10872 [Cerapachys biroi] 394 aa | EZA50902.1 GI: 607356377 |
| protein | |
| hypothetical protein X777_10850 [Cerapachys biroi] 343 aa | EZA50881.1 GI: 607356356 |
| protein | |
| hypothetical protein X777_10849 [Cerapachys biroi] 331 aa | EZA50880.1 GI: 607356355 |
| protein | |
| hypothetical protein X777_10848 [Cerapachys biroi] 361 aa | EZA50879.1 GI: 607356354 |
| protein | |
| hypothetical protein X777_10661, partial [Cerapachys biroi] 286 | EZA50468.1 GI: 607355920 |
| aa protein | |
| hypothetical protein X777_10602 [Cerapachys biroi] 409 aa | EZA50409.1 GI: 607355861 |
| protein | |
| hypothetical protein X777_11257 [Cerapachys biroi] 393 aa | EZA50334.1 GI: 607355781 |
| protein | |
| hypothetical protein X777_11279 [Cerapachys biroi] 393 aa | EZA50278.1 GI: 607355719 |
| protein | |
| hypothetical protein X777_11081 [Cerapachys biroi] 365 aa | EZA50243.1 GI: 607355683 |
| protein | |
| hypothetical protein X777_11573 [Cerapachys biroi] 368 aa | EZA50143.1 GI: 607355570 |
| protein | |
| hypothetical protein X777_11736 [Cerapachys biroi] 398 aa | EZA50071.1 GI: 607355491 |
| protein | |
| hypothetical protein X777_11513 [Cerapachys biroi] 402 aa | EZA50024.1 GI: 607355443 |
| protein | |
| hypothetical protein X777_11512 [Cerapachys biroi] 373 aa | EZA50023.1 GI: 607355442 |
| protein | |
| hypothetical protein X777_11511 [Cerapachys biroi] 399 aa | EZA50022.1 GI: 607355441 |
| protein | |
| hypothetical protein X777_11510 [Cerapachys biroi] 400 aa | EZA50021.1 GI: 607355440 |
| protein | |
| hypothetical protein X777_11509 [Cerapachys biroi] 422 aa | EZA50020.1 GI: 607355439 |
| protein | |
| hypothetical protein X777_11508 [Cerapachys biroi] 410 aa | EZA50019.1 GI: 607355438 |
| protein | |
| hypothetical protein X777_11507 [Cerapachys biroi] 410 aa | EZA50018.1 GI: 607355437 |
| protein | |
| hypothetical protein X777_11506 [Cerapachys biroi] 405 aa | EZA50017.1 GI: 607355436 |
| protein | |
| hypothetical protein X777_l 1880 [Cerapachys biroi] 394 aa | EZA49382.1 GI: 607354770 |
| protein | |
| hypothetical protein X777_11877 [Cerapachys biroi] 400 aa | EZA49381.1 GI: 607354769 |
| protein | |
| hypothetical protein X777_12371 [Cerapachys biroi] 478 aa | EZA49341.1 GI: 607354726 |
| protein | |
| hypothetical protein X777_12369, partial [Cerapachys biroi] 376 | EZA49339.1 GI: 607354724 |
| aa protein | |
| hypothetical protein X777_12360 [Cerapachys biroi] 391 aa | EZA49330.1 GI: 607354715 |
| protein | |
| hypothetical protein X777_12504 [Cerapachys biroi] 391 aa | EZA49105.1 GI: 607354473 |
| protein | |
| hypothetical protein X777_12787 [Cerapachys biroi] 379 aa | EZA49075.1 GI: 607354438 |
| protein | |
| hypothetical protein X777_12783 [Cerapachys biroi] 352 aa | EZA49074.1 GI: 607354437 |
| protein | |
| hypothetical protein X777_12649, partial [Cerapachys biroi] 108 | EZA49046.1 GI: 607354404 |
| aa protein | |
| hypothetical protein X777_12820 [Cerapachys biroi] 366 aa | EZA49011.1 GI: 607354363 |
| protein | |
| hypothetical protein X777_12819 [Cerapachys biroi] 392 aa | EZA49010.1 GI: 607354362 |
| protein | |
| hypothetical protein X777_12817 [Cerapachys biroi] 404 aa | EZA49009.1 GI: 607354361 |
| protein | |
| hypothetical protein X777_12816 [Cerapachys biroi] 395 aa | EZA49008.1 GI: 607354360 |
| protein | |
| hypothetical protein X777_12814 [Cerapachys biroi] 402 aa | EZA49006.1 GI: 607354358 |
| protein | |
| hypothetical protein X777_12813 [Cerapachys biroi] 403 aa | EZA49005.1 GI: 607354357 |
| protein | |
| hypothetical protein X777_12906 [Cerapachys biroi] 393 aa | EZA48943.1 GI: 607354289 |
| protein | |
| hypothetical protein X777_12905 [Cerapachys biroi] 395 aa | EZA48942.1 GI: 607354288 |
| protein | |
| hypothetical protein X777_12904 [Cerapachys biroi] 393 aa | EZA48941.1 GI: 607354287 |
| protein | |
| hypothetical protein X777_12903 [Cerapachys biroi] 393 aa | EZA48940.1 GI: 607354286 |
| protein | |
| hypothetical protein X777_12902 [Cerapachys biroi] 393 aa | EZA48939.1 GI: 607354285 |
| protein | |
| hypothetical protein X777_12901 [Cerapachys biroi] 395 aa | EZA48938.1 GI: 607354284 |
| protein | |
| hypothetical protein X777_12900 [Cerapachys biroi] 394 aa | EZA48937.1 GI: 607354283 |
| protein | |
| hypothetical protein X777_12898 [Cerapachys biroi] 394 aa | EZA48935.1 GI: 607354281 |
| protein | |
| hypothetical protein X777_12897 [Cerapachys biroi] 394 aa | EZA48934.1 GI: 607354280 |
| protein | |
| hypothetical protein X777_12960 [Cerapachys biroi] 391 aa | EZA48918.1 GI: 607354259 |
| protein | |
| hypothetical protein X777_14095 [Cerapachys biroi] 393 aa | EZA48295.1 GI: 607353550 |
| protein | |
| hypothetical protein X777_14075 [Cerapachys biroi] 403 aa | EZA48275.1 GI: 607353530 |
| protein | |
| hypothetical protein X777_14074 [Cerapachys biroi] 395 aa | EZA48274.1 GI: 607353529 |
| protein | |
| hypothetical protein X777_14073 [Cerapachys biroi] 400 aa | EZA48273.1 GI: 607353528 |
| protein | |
| hypothetical protein X777_14071 [Cerapachys biroi] 398 aa | EZA48271.1 GI: 607353526 |
| protein | |
| hypothetical protein X777_14069 [Cerapachys biroi] 395 aa | EZA48269.1 GI: 607353524 |
| protein | |
| hypothetical protein X777_14157 [Cerapachys biroi] 393 aa | EZA48257.1 GI: 607353504 |
| protein | |
| hypothetical protein X777_14156 [Cerapachys biroi] 393 aa | EZA48256.1 GI: 607353503 |
| protein | |
| hypothetical protein X777_14154 [Cerapachys biroi] 294 aa | EZA48254.1 GI: 607353501 |
| protein | |
| hypothetical protein X777_14153 [Cerapachys biroi] 393 aa | EZA48253.1 GI: 607353500 |
| protein | |
| hypothetical protein X777_14152 [Cerapachys biroi] 390 aa | EZA48252.1 GI: 607353499 |
| protein | |
| hypothetical protein X777_14151 [Cerapachys biroi] 393 aa | EZA48251.1 GI: 607353498 |
| protein | |
| hypothetical protein X777_14150 [Cerapachys biroi] 393 aa | EZA48250.1 GI: 607353497 |
| protein | |
| hypothetical protein X777_14149 [Cerapachys biroi] 390 aa | EZA48249.1 GI: 607353496 |
| protein | |
| hypothetical protein X777_14148 [Cerapachys biroi] 422 aa | EZA48248.1 GI: 607353495 |
| protein | |
| hypothetical protein X777_14146 [Cerapachys biroi] 397 aa | EZA48247.1 GI: 607353494 |
| protein | |
| hypothetical protein X777_14145 [Cerapachys biroi] 397 aa | EZA48246.1 GI: 607353493 |
| protein | |
| hypothetical protein X777_14144 [Cerapachys biroi] 400 aa | EZA48245.1 GI: 607353492 |
| protein | |
| hypothetical protein X777_14142 [Cerapachys biroi] 392 aa | EZA48243.1 GI: 607353490 |
| protein | |
| hypothetical protein X777_14137 [Cerapachys biroi] 395 aa | EZA48239.1 GI: 607353486 |
| protein | |
| hypothetical protein X777_14135 [Cerapachys biroi] 370 aa | EZA48238.1 GI: 607353485 |
| protein | |
| hypothetical protein X777_14134 [Cerapachys biroi] 393 aa | EZA48237.1 GI: 607353484 |
| protein | |
| hypothetical protein X777_14133 [Cerapachys biroi] 393 aa | EZA48236.1 GI: 607353483 |
| protein | |
| hypothetical protein X777_14132 [Cerapachys biroi] 393 aa | EZA48235.1 GI: 607353482 |
| protein | |
| hypothetical protein X777_14128 [Cerapachys biroi] 393 aa | EZA48232.1 GI: 607353479 |
| protein | |
| hypothetical protein X777_14127 [Cerapachys biroi] 392 aa | EZA48231.1 GI: 607353478 |
| protein | |
| hypothetical protein X777_14238, partial [Cerapachys biroi] 110 | EZA48207.1 GI: 607353449 |
| aa protein | |
| hypothetical protein X777_14325 [Cerapachys biroi] 391 aa | EZA48143.1 GI: 607353377 |
| protein | |
| hypothetical protein X777_14324 [Cerapachys biroi] 391 aa | EZA48142.1 GI: 607353376 |
| protein | |
| hypothetical protein X777_14322 [Cerapachys biroi] 391 aa | EZA48140.1 GI: 607353374 |
| protein | |
| hypothetical protein X777_14321 [Cerapachys biroi] 332 aa | EZA48139.1 GI: 607353373 |
| protein | |
| hypothetical protein X777_14320 [Cerapachys biroi] 319 aa | EZA48138.1 GI: 607353372 |
| protein | |
| hypothetical protein X777_14319 [Cerapachys biroi] 394 aa | EZA48137.1 GI: 607353371 |
| protein | |
| hypothetical protein X777_14318, partial [Cerapachys biroi] 245 | EZA48136.1 GI: 607353370 |
| aa protein | |
| hypothetical protein X777_14166 [Cerapachys biroi] 410 aa | EZA48057.1 GI: 607353283 |
| protein | |
| hypothetical protein X777_14448 [Cerapachys biroi] 397 aa | EZA48027.1 GI: 607353226 |
| protein | |
| hypothetical protein X777_14454, partial [Cerapachys biroi] 110 | EZA48023.1 GI: 607353220 |
| aa protein | |
| hypothetical protein X777_15046, partial [Cerapachys biroi] 347 | EZA47950.1 GI: 607353115 |
| aa protein | |
| hypothetical protein X777_15045 [Cerapachys biroi] 277 aa | EZA47949.1 GI: 607353114 |
| protein | |
| hypothetical protein X777_15044 [Cerapachys biroi] 395 aa | EZA47948.1 GI: 607353113 |
| protein | |
| hypothetical protein X777_14494 [Cerapachys biroi] 393 aa | EZA47923.1 GI: 607353084 |
| protein | |
| hypothetical protein X777_14493, partial [Cerapachys biroi] 338 | EZA47922.1 GI: 607353083 |
| aa protein | |
| hypothetical protein X777_14490 [Cerapachys biroi] 392 aa | EZA47921.1 GI: 607353082 |
| protein | |
| hypothetical protein X777_14489 [Cerapachys biroi] 391 aa | EZA47920.1 GI: 607353081 |
| protein | |
| hypothetical protein X777_14488 [Cerapachys biroi] 357 aa | EZA47919.1 GI: 607353080 |
| protein | |
| hypothetical protein X777_14486 [Cerapachys biroi] 405 aa | EZA47917.1 GI: 607353078 |
| protein | |
| hypothetical protein X777_14484, partial [Cerapachys biroi] 367 | EZA47916.1 GI: 607353077 |
| aa protein | |
| hypothetical protein X777_15215 [Cerapachys biroi] 400 aa | EZA47891.1 GI: 607353036 |
| protein | |
| hypothetical protein X777_15213 [Cerapachys biroi] 396 aa | EZA47890.1 GI: 607353035 |
| protein | |
| hypothetical protein X777_15211 [Cerapachys biroi] 400 aa | EZA47889.1 GI: 607353034 |
| protein | |
| hypothetical protein X777_15210 [Cerapachys biroi] 402 aa | EZA47888.1 GI: 607353033 |
| protein | |
| hypothetical protein X777_15208 [Cerapachys biroi] 395 aa | EZA47887.1 GI: 607353032 |
| protein | |
| hypothetical protein X777_15207 [Cerapachys biroi] 391 aa | EZA47886.1 GI: 607353031 |
| protein | |
| hypothetical protein X777_15257 [Cerapachys biroi] 367 aa | EZA47872.1 GI: 607353013 |
| protein | |
| hypothetical protein X777_15254, partial [Cerapachys biroi] 203 | EZA47870.1 GI: 607353011 |
| aa protein | |
| hypothetical protein X777_15516 [Cerapachys biroi] 392 aa | EZA47757.1 GI: 607352872 |
| protein | |
| hypothetical protein X777_16325, partial [Cerapachys biroi] 347 | EZA47405.1 GI: 607352456 |
| aa protein | |
| hypothetical protein X777_16550 [Cerapachys biroi] 399 aa | EZA47250.1 GI: 607352245 |
| protein | |
| hypothetical protein X777_16549, partial [Cerapachys biroi] 387 | EZA47249.1 GI: 607352244 |
| aa protein | |
| hypothetical protein X777_16545, partial [Cerapachys biroi] 387 | EZA47248.1 GI: 607352243 |
| aa protein | |
| hypothetical protein X777_16583, partial [Cerapachys biroi] 115 | EZA47246.1 GI: 607352234 |
| aa protein | |
| hypothetical protein X777_16640, partial [Cerapachys biroi] 381 | EZA47175.1 GI: 607352154 |
| aa protein | |
| hypothetical protein X777_16639 [Cerapachys biroi] 388 aa | EZA47174.1 GI: 607352153 |
| protein | |
| hypothetical protein X777_16686 [Cerapachys biroi] 390 aa | EZA47122.1 GI: 607352100 |
| protein | |
| hypothetical protein X777_16765, partial [Cerapachys biroi] 257 | EZA47082.1 GI: 607352037 |
| aa protein | |
| hypothetical protein X777_17009, partial [Cerapachys biroi] 290 | EZA47033.1 GI: 607351973 |
| aa protein | |
| hypothetical protein X777_17008 [Cerapachys biroi] 395 aa | EZA47032.1 GI: 607351972 |
| protein | |
| hypothetical protein X777_17007 [Cerapachys biroi] 395 aa | EZA47031.1 GI: 607351971 |
| protein | |
| hypothetical protein X777_17006 [Cerapachys biroi] 357 aa | EZA47030.1 GI: 607351970 |
| protein | |
| hypothetical protein X777_17005 [Cerapachys biroi] 394 aa | EZA47029.1 GI: 607351969 |
| protein | |
| hypothetical protein X777_17004 [Cerapachys biroi] 394 aa | EZA47028.1 GI: 607351968 |
| protein | |
| hypothetical protein X777_17037 [Cerapachys biroi] 392 aa | EZA47010.1 GI: 607351937 |
| protein | |
| hypothetical protein X777_16829 [Cerapachys biroi] 394 aa | EZA46975.1 GI: 607351833 |
| protein | |
| hypothetical protein X777_16828 [Cerapachys biroi] 377 aa | EZA46974.1 GI: 607351832 |
| protein | |
| hypothetical protein X777_16827 [Cerapachys biroi] 395 aa | EZA46973.1 GI: 607351831 |
| protein | |
| hypothetical protein X777_16826 [Cerapachys biroi] 396 aa | EZA46972.1 GI: 607351830 |
| protein | |
| hypothetical protein X777_16825 [Cerapachys biroi] 390 aa | EZA46971.1 GI: 607351829 |
| protein | |
| hypothetical protein X777_16823 [Cerapachys biroi] 390 aa | EZA46970.1 GI: 607351828 |
| protein | |
| hypothetical protein X777_16822 [Cerapachys biroi] 390 aa | EZA46969.1 GI: 607351827 |
| protein | |
| hypothetical protein X777_00574 [Cerapachys biroi] 394 aa | EZA46944.1 GI: 607351788 |
| protein | |
| hypothetical protein X777_00572, partial [Cerapachys biroi] 284 | EZA46933.1 GI: 607351766 |
| aa protein | |
| hypothetical protein X777_00683, partial [Cerapachys biroi] 390 | EZA46927.1 GI: 607351750 |
| aa protein | |
| hypothetical protein X777_01187 [Cerapachys biroi] 395 aa | EZA46822.1 GI: 607351544 |
| protein | |
| hypothetical protein X777_01186 [Cerapachys biroi] 237 aa | EZA46821.1 GI: 607351543 |
| protein | |
| hypothetical protein X777_01182 [Cerapachys biroi] 393 aa | EZA46818.1 GI: 607351530 |
| protein | |
| hypothetical protein X777_01496, partial [Cerapachys biroi] 310 | EZA46816.1 GI: 607351520 |
| aa protein | |
| hypothetical protein X777_01992, partial [Cerapachys biroi] 104 | EZA46773.1 GI: 607351376 |
| aa protein | |
| hypothetical protein X777_02013 [Cerapachys biroi] 391 aa | EZA46772.1 GI: 607351374 |
| protein | |
| hypothetical protein X777_02041 [Cerapachys biroi] 391 aa | EZA46770.1 GI: 607351360 |
| protein | |
| hypothetical protein X777_02040 [Cerapachys biroi] 399 aa | EZA46769.1 GI: 607351359 |
| protein | |
| hypothetical protein X777_02038 [Cerapachys biroi] 394 aa | EZA46768.1 GI: 607351358 |
| protein | |
| hypothetical protein X777_02037 [Cerapachys biroi] 395 aa | EZA46767.1 GI: 607351357 |
| protein | |
| hypothetical protein X777_02036 [Cerapachys biroi] 351 aa | EZA46766.1 GI: 607351356 |
| protein | |
| hypothetical protein X777_02035 [Cerapachys biroi] 391 aa | EZA46765.1 GI: 607351355 |
| protein | |
| hypothetical protein X777_02034 [Cerapachys biroi] 391 aa | EZA46764.1 GI: 607351354 |
| protein | |
| hypothetical protein X777_02033 [Cerapachys biroi] 395 aa | EZA46763.1 GI: 607351353 |
| protein | |
| hypothetical protein X777_02154, partial [Cerapachys biroi] 388 | EZA46750.1 GI: 607351325 |
| aa protein | |
| hypothetical protein X777_02182, partial [Cerapachys biroi] 115 | EZA46748.1 GI: 607351315 |
| aa protein | |
| hypothetical protein X777_02391 [Cerapachys biroi] 395 aa | EZA46731.1 GI: 607351243 |
| protein | |
| hypothetical protein X777_02373 [Cerapachys biroi] 392 aa | EZA46714.1 GI: 607351226 |
| protein | |
| hypothetical protein X777_02372 [Cerapachys biroi] 313 aa | EZA46713.1 GI: 607351225 |
| protein | |
| hypothetical protein X777_03732 [Cerapachys biroi] 243 aa | EZA46661.1 GI: 607350938 |
| protein | |
| hypothetical protein X777_04259, partial [Cerapachys biroi] 110 | EZA46601.1 GI: 607350634 |
| aa protein | |
| hypothetical protein X777_04309, partial [Cerapachys biroi] 70 aa | EZA46600.1 GI: 607350630 |
| protein | |
| hypothetical protein X777_00022, partial [Cerapachys biroi] 114 | EZA46571.1 GI: 607349175 |
| aa protein | |
| hypothetical protein X777_00149, partial [Cerapachys biroi] 64 aa | EZA46447.1 GI: 607348111 |
| protein | |
| hypothetical protein X777_00236, partial [Cerapachys biroi] 230 | EZA46364.1 GI: 607347729 |
| aa protein | |
| Odorant receptor Or1; AgOr1 417 aa protein | Q8WTE7.1 GI: 44888255 |
| Odorant receptor Or2; AgOr2 378 aa protein | Q8WTE6.1 GI: 44888254 |
| uncharacterized protein LOC107040665 [Diachasma alloeum] | XP_015116341.1 GI: 970881283 |
| 1186 aa protein | |
| uncharacterized protein LOC107038380 [Diachasma alloeum] | XP_015112930.1 GI: 970880645 |
| 1173 aa protein | |
| uncharacterized protein LOC107040682 [Diachasma alloeum] 417 | XP_015116364.1 GI: 970881287 |
| aa protein | |
| uncharacterized protein LOC107038389 [Diachasma alloeum] 770 | XP_015112957.1 GI: 970880649 |
| aa protein | |
| Sensory neuron membrane protein 1; Short = SNMP1Dmel 551 aa | Q9VDD3.2 GI: 74868468 |
| protein | |
| General odorant-binding protein lush; Flags: Precursor 153 aa | O02372.1 GI: 61214421 |
| protein | |
| uncharacterized protein LOC107171897, partial [Diuraphis noxia] | XP_015377642.1 GI: 985424240 |
| 135 aa protein | |
| uncharacterized protein LOC107171052, partial [Diuraphis noxia] | XP_015376771.1 GI: 985422638 |
| 131 aa protein | |
| uncharacterized protein LOC107166471 [Diuraphis noxia] 403 aa | XP_015370631.1 GI: 985410377 |
| protein | |
| uncharacterized protein LOC107167838 [Diuraphis noxia] 113 aa | XP_015372525.1 GI: 985386955 |
| protein | |
| uncharacterized protein LOC107047710 [Diachasma alloeum] 134 | XP_015125998.1 GI: 970918690 |
| aa protein | |
| uncharacterized protein LOC107046523 [Diachasma alloeum] 435 | XP_015124630.1 GI: 970916388 |
| aa protein | |
| uncharacterized protein LOC107045828 [Diachasma alloeum] 131 | XP_015123693.1 GI: 970914661 |
| aa protein | |
| uncharacterized protein LOC107045792 [Diachasma alloeum] 274 | XP_015123634.1 GI: 970914552 |
| aa protein | |
| uncharacterized protein LOC107045791 [Diachasma alloeum] 377 | XP_015123633.1 GI: 970914550 |
| aa protein | |
| uncharacterized protein LOC107045316 isoform X2 [Diachasma | XP_015123022.1 GI: 970913423 |
| alloeum] 352 aa protein | |
| uncharacterized protein LOC107044760 [Diachasma alloeum] 389 | XP_015122273.1 GI: 970912048 |
| aa protein | |
| uncharacterized protein LOC107043060 [Diachasma alloeum] 234 | XP_015119847.1 GI: 970907601 |
| aa protein | |
| uncharacterized protein LOC107041505 [Diachasma alloeum] 154 | XP_015117568.1 GI: 970903454 |
| aa protein | |
| uncharacterized protein LOC107041471 [Diachasma alloeum] 392 | XP_015117541.1 GI: 970903394 |
| aa protein | |
| uncharacterized protein LOC107041468 [Diachasma alloeum] 284 | XP_015117538.1 GI: 970903388 |
| aa protein | |
| uncharacterized protein LOC107041058 [Diachasma alloeum] 171 | XP_015116902.1 GI: 970902214 |
| aa protein | |
| uncharacterized protein LOC107041057 [Diachasma alloeum] 173 | XP_015116901.1 GI: 970902212 |
| aa protein | |
| uncharacterized protein LOC107040351 [Diachasma alloeum] 394 | XP_015115892.1 GI: 970900366 |
| aa protein | |
| uncharacterized protein LOC107040339 [Diachasma alloeum] 313 | XP_015115876.1 GI: 970900336 |
| aa-protein | |
| uncharacterized protein LOC107039160 isoform X3 [Diachasma | XP_015114121.1 GI: 970897155 |
| alloeum] 335 aa protein | |
| uncharacterized protein LOC107039160 isoform X2 [Diachasma | XP_015114120.1 GI: 970897153 |
| alloeum] 383 aa protein | |
| uncharacterized protein LOC107039160 isoform X1 [Diachasma | XP_015114119.1 GI: 970897151 |
| alloeum] 391 aa protein | |
| uncharacterized protein LOC107039144 isoform X2 [Diachasma | XP_015114102.1 GI: 970897120 |
| alloeum] 327 aa protein | |
| uncharacterized protein LOC107039144 isoform X1 [Diachasma | XP_015114101.1 GI: 970897118 |
| alloeum] 383 aa protein | |
| uncharacterized protein LOC107038269 [Diachasma alloeum] 398 | XP_015112765.1 GI: 970894635 |
| aa protein | |
| uncharacterized protein LOC107038076 [Diachasma alloeum] 416 | XP_015112442.1 GI: 970894044 |
| aa protein | |
| uncharacterized protein LOC107038024 [Diachasma alloeum] 115 | XP_015112374.1 GI: 970893921 |
| aa protein | |
| uncharacterized protein LOC107037155 [Diachasma alloeum] 144 | XP_015111030.1 GI: 970891453 |
| aa protein | |
| uncharacterized protein LOC107037131 [Diachasma alloeum] 393 | XP_015111001.1 GI: 970891401 |
| aa protein | |
| uncharacterized protein LOC107037008 [Diachasma alloeum] 428 | XP_015110801.1 GI: 970891030 |
| aa protein | |
| uncharacterized protein LOC107036995 [Diachasma alloeum] 399 | XP_015110788.1 GI: 970891006 |
| aa protein | |
| uncharacterized protein LOC107036721 [Diachasma alloeum] 203 | XP_015110342.1 GI: 970890185 |
| aa protein | |
| uncharacterized protein LOC107036608 [Diachasma alloeum] 126 | XP_015110155.1 GI: 970889843 |
| aa protein | |
| uncharacterized protein LOC107036569 [Diachasma alloeum] 322 | XP_015110092.1 GI: 970889731 |
| aa protein | |
| uncharacterized protein LOC107036562 [Diachasma alloeum] 245 | XP_015110086.1 GI: 970889719 |
| aa protein | |
| uncharacterized protein LOC107035960 [Diachasma alloeum] 410 | XP_015109114.1 GI: 970887963 |
| aa protein | |
| uncharacterized protein LOC107048689 [Diachasma alloeum] 172 | XP_015127489.1 GI: 970885577 |
| aa protein | |
| uncharacterized protein LOC107048383 [Diachasma alloeum] 221 | XP_015127016.1 GI: 970884731 |
| aa protein | |
| uncharacterized protein LOC107048083 [Diachasma alloeum] 255 | XP_015126543.1 GI: 970883864 |
| aa protein | |
| uncharacterized protein LOC107041464 [Diachasma alloeum] 339 | XP_015117536.1 GI: 970881499 |
| aa protein | |
| uncharacterized protein LOC107041401 isoform X1 [Diachasma | XP_015117436.1 GI: 970881482 |
| alloeum] 405 aa protein | |
| uncharacterized protein LOC107040856 [Diachasma alloeum] 386 | XP_015116611.1 GI: 970881336 |
| aa protein | |
| uncharacterized protein LOC107040848 [Diachasma alloeum] 391 | XP_015116602.1 GI: 970881334 |
| aa protein | |
| uncharacterized protein LOC106692217, partial [Halyomorpha | XP_014293607.1 GI: 939698834 |
| halys] 120 aa protein | |
| uncharacterized protein LOC106692125, partial [Halyomorpha | XP_014293519.1 GI: 939698671 |
| halys] 120 aa protein | |
| uncharacterized protein LOC106691728, partial [Halyomorpha | XP_014293070.1 GI: 939697820 |
| halys] 309 aa protein | |
| uncharacterized protein LOC106690974 [Halyomorpha halys] 381 | XP_014292085.1 GI: 939695932 |
| aa protein | |
| uncharacterized protein LOC106690972 [Halyomorpha halys] 334 | XP_014292082.1 GI: 939695928 |
| aa protein | |
| uncharacterized protein LOC106690969 [Halyomorpha halys] 280 | XP_014292080.1 GI: 939695923 |
| aa protein | |
| uncharacterized protein LOC106690968 isoform X2 | XP_014292079.1 GI: 939695921 |
| [Halyomorpha halys] 356 aa protein | |
| uncharacterized protein LOC106690968 isoform X1 | XP_014292078.1 GI: 939695919 |
| [Halyomorpha halys] 381 aa protein | |
| uncharacterized protein LOC106690056 [Halyomorpha halys] 419 | XP_014290808.1 GI: 939693496 |
| aa protein | |
| uncharacterized protein LOC106689927 isoform X2 | XP_014290638.1 GI: 939693169 |
| [Halyomorpha halys] 402 aa protein | |
| uncharacterized protein LOC106689927 isoform X1 | XP_014290637.1 GI: 939693167 |
| [Halyomorpha halys] 430 aa protein | |
| uncharacterized protein LOC106689925 [Halyomorpha halys] 430 | XP_014290634.1 GI: 939693163 |
| aa protein | |
| uncharacterized protein LOC106689759 [Halyomorpha halys] 126 | XP_014290404.1 GI: 939692739 |
| aa protein | |
| uncharacterized protein LOC106689626, partial [Halyomorpha | XP_014290195.1 GI: 939692350 |
| halys] 361 aa protein | |
| uncharacterized protein LOC106689110 [Halyomorpha halys] 395 | XP_014289383.1 GI: 939690780 |
| aa protein | |
| uncharacterized protein LOC106689027, partial [Halyomorpha | XP_014289256.1 GI: 939690551 |
| halys] 349 aa protein | |
| uncharacterized protein LOC106688861 [Halyomorpha halys] 354 | XP_014289023.1 GI: 939690122 |
| aa protein | |
| uncharacterized protein LOC106688860 [Halyomorpha halys] 402 | XP_014289021.1 GI: 939690120 |
| aa protein | |
| uncharacterized protein LOC106688858 [Halyomorpha halys] 401 | XP_014289019.1 GI: 939690116 |
| aa protein | |
| uncharacterized protein LOC106688856 isoform X3 | XP_014289018.1 GI: 939690114 |
| [Halyomorpha halys] 291 aa protein | |
| uncharacterized protein LOC106688856 isoform X3 | XP_014289017.1 GI: 939690112 |
| [Halyomorpha halys] 291 aa protein | |
| uncharacterized protein LOC106688856 isoform X2 | XP_014289016.1 GI: 939690110 |
| [Halyomorpha halys] 349 aa protein | |
| uncharacterized protein LOC106688856 isoform X1 | XP_014289015.1 GI: 939690108 |
| [Halyomorpha halys] 391 aa protein | |
| uncharacterized protein LOC106688855 [Halyomorpha halys] 122 | XP_014289014.1 GI: 939690106 |
| aa protein | |
| uncharacterized protein LOC106688854 [Halyomorpha halys] 355 | XP_014289013.1 GI: 939690104 |
| aa protein | |
| uncharacterized protein LOC106688852 [Halyomorpha halys] 402 | XP_014289011.1 GI: 939690100 |
| aa protein | |
| uncharacterized protein LOC106688565 [Halyomorpha halys] 399 | XP_014288559.1 GI: 939689255 |
| aa protein | |
| uncharacterized protein LOC106688504 [Halyomorpha halys] 415 | XP_014288483.1 GI: 939689114 |
| aa protein | |
| uncharacterized protein LOC106688133 isoform X2 | XP_014287937.1 GI: 939688097 |
| [Halyomorpha halys] 264 aa protein | |
| uncharacterized protein LOC106688133 isoform X1 | XP_014287936.1 GI: 939688095 |
| [Halyomorpha halys] 299 aa protein | |
| uncharacterized protein LOC106687951 [Halyomorpha halys] 205 | XP_014287635.1 GI: 939687191 |
| aa protein | |
| uncharacterized protein LOC106687745 [Halyomorpha halys] 117 | XP_014287267.1 GI: 939686093 |
| aa protein | |
| uncharacterized protein LOC106687729 [Halyomorpha halys] 450 | XP_014287248.1 GI: 939686031 |
| aa protein | |
| uncharacterized protein LOC106687584 [Halyomorpha halys] 355 | XP_014287044.1 GI: 939685359 |
| aa protein | |
| uncharacterized protein LOC106687583 [Halyomorpha halys] 254 | XP_014287043.1 GI: 939685355 |
| aa protein | |
| uncharacterized protein LOC106687100 [Halyomorpha halys] 438 | XP_014286277.1 GI: 939683074 |
| aa protein | |
| uncharacterized protein LOC106686230 [Halyomorpha halys] 410 | XP_014284895.1 GI: 939678679 |
| aa protein | |
| uncharacterized protein LOC106686225 isoform X2 | XP_014284891.1 GI: 939678662 |
| [Halyomorpha halys] 382 aa protein | |
| uncharacterized protein LOC106686225 isoform X1 | XP_014284890.1 GI: 939678658 |
| [Halyomorpha halys] 419 aa protein | |
| uncharacterized protein LOC106684746 isoform X2 | XP_014282485.1 GI: 939671752 |
| [Halyomorpha halys] 235 aa protein | |
| uncharacterized protein LOC106684678 isoform X2 | XP_014282387.1 GI: 939671481 |
| [Halyomorpha halys] 140 aa protein | |
| uncharacterized protein LOC106684574 [Halyomorpha halys] 142 | XP_014282214.1 GI: 939671038 |
| aa protein | |
| uncharacterized protein LOC106684269 [Halyomorpha halys] 211 | XP_014281731.1 GI: 939669764 |
| aa protein | |
| uncharacterized protein LOC106682571 [Halyomorpha halys] 373 | XP_014278976.1 GI: 939662570 |
| aa protein | |
| uncharacterized protein LOC106682449 isoform X2 | XP_014278794.1 GI: 939662115 |
| [Halyomorpha halys] 322 aa protein | |
| uncharacterized protein LOC106682449 isoform X1 | XP_014278793.1 GI: 939662112 |
| [Halyomorpha halys] 323 aa protein | |
| uncharacterized protein LOC106682407, partial [Halyomorpha | XP_014278714.1 GI: 939661920 |
| halys] 265 aa protein | |
| uncharacterized protein LOC106681266 [Halyomorpha halys] 432 | XP_014276987.1 GI: 939657430 |
| aa protein | |
| uncharacterized protein LOC106681101, partial [Halyomorpha | XP_014276742.1 GI: 939656824 |
| halys] 182 aa protein | |
| uncharacterized protein LOC106681099 [Halyomorpha halys] 323 | XP_014276740.1 GI: 939656818 |
| aa protein | |
| uncharacterized protein LOC106680703 [Halyomorpha halys] 228 | XP_014276067.1 GI: 939654990 |
| aa protein | |
| uncharacterized protein LOC106680013 [Halyomorpha halys] 197 | XP_014274955.1 GI: 939651857 |
| aa protein | |
| uncharacterized protein LOC106679982 [Halyomorpha halys] 398 | XP_014274899.1 GI: 939651709 |
| aa protein | |
| uncharacterized protein LOC106679017 isoform X3 | XP_014273409.1 GI: 939647459 |
| [Halyomorpha halys] 392 aa protein | |
| uncharacterized protein LOC106679017 isoform X2 | XP_014273408.1 GI: 939647457 |
| [Halyomorpha halys] 402 aa protein | |
| uncharacterized protein LOC106679017 isoform X1 | XP_014273407.1 GI: 939647455 |
| [Halyomorpha halys] 427 aa protein | |
| uncharacterized protein LOC106678921 isoform X2 | XP_014273270.1 GI: 939647059 |
| [Halyomorpha halys] 385 aa protein | |
| uncharacterized protein LOC106678921 isoform X1 | XP_014273269.1 GI: 939647056 |
| [Halyomorpha halys] 437 aa protein | |
| uncharacterized protein LOC106678586 [Halyomorpha halys] 317 | XP_014272658.1 GI: 939645318 |
| aa protein | |
| uncharacterized protein LOC106678578 [Halyomorpha halys] 169 | XP_014272643.1 GI: 939645272 |
| aa protein | |
| uncharacterized protein LOC106678240 [Halyomorpha halys] 388 | XP_014272140.1 GI: 939643797 |
| aa protein | |
| uncharacterized protein LOC106677363 isoform X2 | XP_014270733.1 GI: 939639742 |
| [Halyomorpha halys] 417 aa protein | |
| uncharacterized protein LOC106677363 isoform X1 | XP_014270732.1 GI: 939639738 |
| [Halyomorpha halys] 419 aa protein | |
| uncharacterized protein LOC106677357 isoform X2 | XP_014270726.1 GI: 939639710 |
| [Halyomorpha halys] 370 aa protein | |
| uncharacterized protein LOC106677357 isoform X1 | XP_014270725.1 GI: 939639708 |
| [Halyomorpha halys] 404 aa protein | |
| uncharacterized protein LOC106677356 [Halyomorpha halys] 355 | XP_014270724.1 GI: 939639706 |
| aa protein | |
| uncharacterized protein LOC106693012 [Halyomorpha halys] 415 | XP_014294797.1 GI: 939638057 |
| aa protein | |
| uncharacterized protein LOC106693004 [Halyomorpha halys] 297 | XP_014294789.1 GI: 939638041 |
| aa protein | |
| uncharacterized protein LOC106692786 isoform X2 | XP_014294440.1 GI: 939637036 |
| [Halyomorpha halys] 348 aa protein | |
| uncharacterized protein LOC106692425 isoform X2 | XP_014293860.1 GI: 939635393 |
| [Halyomorpha halys] 378 aa protein | |
| uncharacterized protein LOC106687938 [Halyomorpha halys] 358 | XP_014287615.1 GI: 939633287 |
| aa protein | |
| uncharacterized protein LOC106684029 isoform X1 | XP_014281345.1 GI: 939631378 |
| [Halyomorpha halys] 400 aa protein | |
| uncharacterized protein LOC106683667 isoform X2 | XP_014280777.1 GI: 939631233 |
| [Halyomorpha halys] 345 aa protein | |
| uncharacterized protein LOC106683640 [Halyomorpha halys] 353 | XP_014280732.1 GI: 939631224 |
| aa protein | |
| uncharacterized protein LOC106683586, partial [Halyomorpha | XP_014280634.1 GI: 939631202 |
| halys] 400 aa protein | |
| uncharacterized protein LOC106681860 [Halyomorpha halys] 402 | XP_014277890.1 GI: 939630417 |
| aa protein | |
| uncharacterized protein LOC106681850 [Halyomorpha halys] 358 | XP_014277878.1 GI: 939630413 |
| aa protein | |
| uncharacterized protein LOC106681777 [Halyomorpha halys] 428 | XP_014277766.1 GI: 939630367 |
| aa protein | |
| uncharacterized protein LOC106678083 [Halyomorpha halys] 426 | XP_014271872.1 GI: 939628624 |
| aa protein | |
| uncharacterized protein LOC106692148 [Halyomorpha halys] 372 | XP_014293574.1 GI: 939627857 |
| aa protein | |
| olfactory receptor 3 [Bombyx mori] 439 aa protein | NP_001036925.1 GI: 112982950 |
| olfactory receptor 9 [Plutella xylostella] 449 aa protein | ALV82554.1 GI: 971834990 |
| olfactory receptor 2 [Bombyx mori] 472 aa protein | NP_001037060.1 GI: 112983084 |
| olfactory receptor 1 [Bombyx mori] 430 aa protein | NP_001036875.1 GI: 1 12983558 |
| Chain A, Structure Of Pheromone-binding Protein 1 In Complex | 4INX_A GI: 459358923 |
| With (z,z)-11,13-Hexadecadienol 140 aa protein | |
| Chain A, Structure Of Pheromone-binding Protein 1 In Complex | 4INW_A GI: 459358922 |
| With (11z,13z)-Hexadecadienal 140 aa protein | |
| olfactory receptor [Ostrinia fumacalis] 424 aa protein | BAH57982.1 GI: 229365469 |
| olfactory receptor [Ostrinia latipennis] 424 aa protein | BAH57981.1 GI: 229365467 |
| olfactory receptor [Ostrinia nubilalis] 424 aa protein | BAH57980.1 GI: 229365465 |
| olfactory receptor [Ostrinia ovalipennis] 424 aa protein | BAH57979.1 GI: 229365463 |
| olfactory receptor [Ostrinia palustralis] 424 aa protein | BAH57978.1 GI: 229365461 |
| olfactory receptor [Ostrinia zealis] 424 aa protein | BAH57977.1 GI: 229365459 |
| olfactory receptor [Ostrinia zaguliaevi] 424 aa protein | BAH57976.1 GI: 229365457 |
| olfactory receptor [Ostrinia scapulalis] 424 aa protein | BAH57975.1 GI: 229365455 |
| Sequence 10 from patent U.S. Pat. No. 7,601,829 486 aa protein | ADA08702.1 GI: 281014387 |
| Sequence 6 from patent U.S. Pat. No. 7,601,829 478 aa protein | ADA08700.1 GI: 281014385 |
| Sequence 4 from patent U.S. Pat. No. 7,601,829 472 aa protein | ADA08699.1 GI: 281014384 |
| Sequence 2 from patent U.S. Pat. No. 7,601,829 473 aa protein | ADA08698.1 GI: 281014383 |
| Sequence 10 from patent U.S. Pat. No. 7,550,574 486 aa protein | ACW03545.1 GI: 259184438 |
| Sequence 6 from patent U.S. Pat. No. 7,550,574 478 aa protein | ACW03543.1 GI: 259184436 |
| Sequence 4 from patent U.S. Pat. No. 7,550,574 472 aa protein | ACW03542.1 GI: 259184435 |
| Sequence 2 from patent U.S. Pat. No. 7,550,574 473 aa protein | ACW03541.1 GI: 259184434 |
| putative chemosensory receptor 2 [Antheraea pernyi] 472 aa | CAD88205.1 GI: 32399809 |
| protein | |
| hypothetical protein TcasGA2_TC032780 [Tribolium castaneum] | KYB27892.1 GI: 1004400598 |
| 1096 aa protein | |
| olfactory receptor 65 [Bombyx mori] 239 aa protein | NP_001166622.1 GI: 290560867 |
| olfactory receptor 13 [Bombyx mori] 385 aa protein | NP_001166603.1 GI: 290559921 |
| olfactory receptor-like [Bombyx mori] 410 aa protein | NP_001159623.1 GI: 261245107 |
| olfactory receptor 4 [Bombyx mori] 424 aa protein | NP_001036926.1 GI: 112982926 |
| AKH receptor variant AKHR3 isoform AKHR-B [Pseudoregma | AKH80290.1 GI: 822549471 |
| bambucicola] 591 aa protein | |
| olfactory receptor 10 [Bombyx mori] 388 aa protein | NP_001104819.1 GI: 162462631 |
| olfactory receptor 6 [Bombyx mori] 407 aa protein | NP_001036928.1 GI: 112982988 |
| olfactory receptor 5 [Bombyx mori] 417 aa protein | NP_001036927.1 GI: 112982948 |
| olfactory receptor 2 [Chilo suppressalis] 474 aa protein | AFQ94048.1 GI: 402746958 |
| unknown [Dendroctonus ponderosae] 382 aa protein | AEE63423.1 GI: 332376567 |
| unknown [Dendroctonus ponderosae] 396 aa protein | AEE63326.1 GI: 332376372 |
| unknown [Dendroctonus ponderosae] 404 aa protein | AEE63155.1 GI: 332376029 |
| unknown [Dendroctonus ponderosae] 394 aa protein | AEE62970.1 GI: 332375658 |
| unknown [Dendroctonus ponderosae] 395 aa protein | AEE62637.1 GI: 332374992 |
| unknown [Dendroctonus ponderosae] 400 aa protein | AEE62488.1 GI: 332374694 |
| unknown [Dendroctonus ponderosae] 480 aa protein | AEE62122.1 GI: 332373962 |
| unknown [Dendroctonus ponderosae] 396 aa protein | AEE61493.1 GI: 332372702 |
| unknown [Dendroctonus ponderosae] 403 aa protein | AEE61404.1 GI: 332372524 |
| olfactory receptor [Dendroctonus ponderosae] 480 aa protein | AFI45064.1 GI: 385200032 |
| putative olfactory receptor 18 [Spodoptera littoralis] 398 aa protein | ACL81189.1 GI: 220715234 |
| putative olfactory receptor 18 [Mamestra brassicae] 400 aa protein | ACL81188.1 GI: 220715232 |
| putative olfactory receptor 18 [Helicoverpa armigera] 398 aa | ACL81187.1 GI: 220715230 |
| protein | |
| putative olfactory receptor 18 [Helicoverpa zea] 398 aa protein | ACL81186.1 GI: 220715228 |
| putative olfactory receptor 18 [Agrotis segetum] 400 aa protein | ACL81185.1 GI: 220715226 |
| putative olfactory receptor 18 [Sesamia nonagrioides] 400 aa | ACL81184.1 GI: 220715224 |
| protein | |
| Sequence 18 from patent U.S. Pat. No. 7,601,829 33 aa protein | ADA08710.1 GI: 281014395 |
| Sequence 17 from patent U.S. Pat. No. 7,601,829 43 aa protein | ADA08709.1 GI: 281014394 |
| Sequence 16 from patent U.S. Pat. No. 7,601,829 43 aa protein | ADA08708.1 GI: 281014393 |
| Sequence 15 from patent U.S. Pat. No. 7,601,829 43 aa protein | ADA08707.1 GI: 281014392 |
| Sequence 14 from patent U.S. Pat. No. 7,601,829 43 aa protein | ADA08706.1 GI: 281014391 |
| Sequence 13 from patent U.S. Pat. No. 7,601,829 11 aa protein | ADA08705.1 GI: 281014390 |
| Sequence 12 from patent U.S. Pat. No. 7,601,829 16 aa protein | ADA08704.1 GI: 281014389 |
| Sequence 11 from patent U.S. Pat. No. 7,601,829 498 aa protein | ADA08703.1 GI: 281014388 |
| Sequence 8 from patent U.S. Pat. No. 7,601,829 486 aa protein | ADA08701.1 GI: 281014386 |
| Sequence 18 from patent U.S. Pat. No. 7,550,574 33 aa protein | ACW03553.1 GI: 259184446 |
| Sequence 17 from patent U.S. Pat. No. 7,550,574 43 aa protein | ACW03552.1 GI: 259184445 |
| Sequence 16 from patent U.S. Pat. No. 7,550,574 43 aa protein | ACW03551.1 GI: 259184444 |
| Sequence 15 from patent U.S. Pat. No. 7,550,574 43 aa protein | ACW03550.1 GI: 259184443 |
| Sequence 14 from patent U.S. Pat. No. 7,550,574 43 aa protein | ACW03549.1 GI: 259184442 |
| Sequence 13 from patent U.S. Pat. No. 7,550,574 11 aa protein | ACW03548.1 GI: 259184441 |
| Sequence 12 from patent U.S. Pat. No. 7,550,574 16 aa protein | ACW03547.1 GI: 259184440 |
| Sequence 11 from patent U.S. Pat. No. 7,550,574 498 aa protein | ACW03546.1 GI: 259184439 |
| Sequence 8 from patent U.S. Pat. No. 7,550,574 486 aa protein | ACW03544.1 GI: 259184437 |
| Sequence 6 from patent U.S. Pat. No. 7,541,155 486 aa protein | ACS10701.1 GI: 239686039 |
| Sequence 4 from patent U.S. Pat. No. 7,541,155 376 aa protein | ACS10700.1 GI: 239686038 |
| unnamed protein product [Drosophila melanogaster] 379 aa | CAY86014.1 GI: 237677885 |
| protein | |
| unnamed protein product [Drosophila melanogaster] 376 aa | CAY86011.1 GI: 237677879 |
| protein | |
| unnamed protein product [Drosophila melanogaster] 467 aa | CAY86010.1 GI: 237677877 |
| protein | |
| unnamed protein product, partial [Drosophila melanogaster] 153 aa | CAY86009.1 GI: 237677875 |
| protein | |
| Sequence 104 from patent U.S. Pat. No. 7,241,881 486 aa protein | ABU34893.1 GI: 155712034 |
| Sequence 100 from patent U.S. Pat. No. 7,241,881 392 aa protein | ABU34891.1 GI: 155712032 |
| Sequence 98 from patent U.S. Pat. No. 7,241,881 406 aa protein | ABU34890.1 GI: 155712031 |
| Sequence 78 from patent U.S. Pat. No. 7,241,881 378 aa protein | ABU34880.1 GI: 155712021 |
| Sequence 70 from patent U.S. Pat. No. 7,241,881 392 aa protein | ABU34876.1 GI: 155712017 |
| Sequence 68 from patent U.S. Pat. No. 7,241,881 397 aa protein | ABU34875.1 GI: 155712016 |
| Sequence 66 from patent U.S. Pat. No. 7,241,881 413 aa protein | ABU34874.1 GI: 155712015 |
| Sequence 50 from patent U.S. Pat. No. 7,241,881 398 aa protein | ABU34866.1 GI: 155712007 |
| Sequence 40 from patent U.S. Pat. No. 7,241,881 412 aa protein | ABU34861.1 GI: 155712002 |
| Sequence 34 from patent U.S. Pat. No. 7,241,881 383 aa protein | ABU34858.1 GI: 155711999 |
| Sequence 30 from patent U.S. Pat. No. 7,241,881 396 aa protein | ABU34856.1 GI: 155711997 |
| Sequence 28 from patent U.S. Pat. No. 7,241,881 375 aa protein | ABU34855.1 GI: 155711996 |
| Sequence 24 from patent U.S. Pat. No. 7,241,881 385 aa protein | ABU34853.1 GI: 155711994 |
| Sequence 20 from patent U.S. Pat. No. 7,241,881 379 aa protein | ABU34851.1 GI: 155711992 |
| Sequence 18 from patent U.S. Pat. No. 7,241,881 378 aa protein | ABU34850.1 GI: 155711991 |
| Sequence 16 from patent U.S. Pat. No. 7,241,881 379 aa protein | ABU34849.1 GI: 155711990 |
| Sequence 12 from patent U.S. Pat. No. 7,241,881 379 aa protein | ABU34847.1 GI: 155711988 |
| Sequence 10 from patent U.S. Pat. No. 7,241,881 397 aa protein | ABU34846.1 GI: 155711987 |
| Sequence 8 from patent U.S. Pat. No. 7,241,881 397 aa protein | ABU34845.1 GI: 155711986 |
| Sequence 6 from patent U.S. Pat. No. 7,241,881 376 aa protein | ABU34844.1 GI: 155711985 |
| Sequence 4 from patent U.S. Pat. No. 7,241,881 467 aa protein | ABU34843.1 GI: 155711984 |
| Sequence 2 from patent U.S. Pat. No. 7,241,881 397 aa protein | ABU34842.1 GI: 155711983 |
| olfactory receptor-like receptor [Bombyx mori] 407 aa protein | BAD89570.1 GI: 59796989 |
| olfactory receptor-like receptor [Bombyx mori] 417 aa protein | BAD89569.1 GI: 59796987 |
| olfactory receptor-like receptor [Bombyx mori] 424 aa protein | BAD89568.1 GI: 59796985 |
| olfactory receptor-like receptor [Bombyx mori] 439 aa protein | BAD89567.1 GI: 59796983 |
| putative chemosensory receptor 2, partial [Tenebrio molitor] 206 | CAD88247.1 GI: 32400236 |
| aa protein | |
| putative chemosensory receptor 2, partial [Calliphora vicina] 208 | CAD88246.1 GI: 32400234 |
| aa protein | |
| putative chemosensory receptor 2, partial [Apis mellifera] 210 aa | CAD88245.1 GI: 32399813 |
| protein | |
| putative chemosensory receptor 2 [Bombyx mori] 472 aa protein | CAD88206.1 GI: 32399811 |

TABLE 4

OR1A1 (Homo sapiens)
MOR106-1 (Mus musculus)
OR51E1 (Homo sapiens)
OR10J5 (Homo sapiens)
OR51E2 (Homo sapiens)
MOR9-1 (Mus musculus)
MOR18-1 (Mus musculus)
MOR272-1 (Mus musculus)
MOR31-1 (Mus musculus)
MOR136-1 (Mus musculus)

### I. Example 2—Detection System for Identification of Volatile Compounds and Determination of Source Location

In some embodiments, a detection system comprising multiple cell-based sensor panels positioned at known locations in a space (e.g., a room, passageway, parking garage, or other place) may be used to monitor air samples for the presence of volatile compounds, e.g., volatile markers of or taggants used in the manufacture of explosive materials. Each sensor panel may be assigned a set of known 3-dimensional coordinates (x, y, z) which may be used by a sensor signal processing algorithm to not only detect and identify one or more volatile compounds of interest, but also to determine the location of the source of the volatile compound(s) within the space. As described above, the signal processing algorithm can be used to differentially detect a gradient of a compound and correlate the local compound concentration with the (x, y, z) coordinates of the sensor panel at each location, thus, permitting generation of a 3-dimensional map. By collecting multiple readings over time, a 4-dimensional map (x, y, z, t) (where t=time) may be created such that a detection system can map increasing and decreasing chemical concentrations across space and time. By tracking an increase in compound concentration over time, one can detect a path for the chemical gradient, thereby permitting the detection of the location of a fixed position chemical source, or the mapping of the path of a moving chemical source.

Such detection systems may be applied to a variety of different scenarios, such as detection of explosives in an airport environment. Examples of specific airport detection scenarios in which the disclosed detection systems may be applied include: (a) parking garage locations with outside airflow; (b) passenger entry-way vestibules; (c) passenger boarding pass and baggage check-in counters; (d) passenger screening in open spaces or passages by the Transportation Security Administration (TSA); (e) gate open spaces; (f) boarding or off-loading passenger gate pathways onto an airplane; (g) train station platforms within or entering the airport, including spaces that comprise multi-level (elevator, escalator or stairway) transport.

In some cases, the airport environment may be akin to that in other large buildings with public access, e.g., shopping malls, train stations, or office building lobbies. These locations are similar in that they typically comprise large enclosed spaces, often with significant human traffic flow, which cannot be easily monitored due to excessive movement and/or the size of the open space.

In some embodiments, a 3-dimensional grid of sensor panels may be located around the entire airport space. In some embodiments a 3-dimensional grid of sensor panels may be confined to localized areas of the airport. For rough position coordinate estimates, the GPS grid may be used, but the resolution of the disclosed detection systems for location of an odorant source (which is determined in part by the accuracy of determining the position coordinates of the sensor panels) may be more fine-grained than that achievable by Global Positioning System (GPS) readings (approximately 3-4 meters horizontally). Therefore, a higher resolution mapping of the grid of sensor panels within the space may be required. For example, in some cases, one may be able to identify the locations of the detectors and the odorant source to within about 2 meters in any dimensions. In some cases, one may be able to identify the locations of the detectors and the odorant source to within about 1 meter in any dimension. In some cases, one may be able to identify the locations of the detectors and the odorant source to within about 0.5 meters in any dimension. In some cases, one may be able to identify the locations of the detectors and odorant source to within about 0.1 meters in any dimensions. In some cases, one may be able to identify the locations of the detectors and the odorant source to within about 0.05 meters in any dimension. In some cases, one may be able to identify the locations of the detectors and odorant source to within about 0.01 meters in any dimension, or better.

Consider a vestibule through which a stream of passengers may enter an airport. The vestibule may comprise a long hallway, or a short entryway with revolving doors, or a short passageway with two sets of sliding glass doors (one at each end). As a specific example, consider a 3-dimensional grid of sensor panels assigned to a passageway. This passageway may be assigned ID=#23 in the detection system's system control software. Coordinates of the sensor panel detectors may be entered into the system control software in units of meters. Three evenly spaced detectors may be placed along the passageway. Both the 3D coordinates and the gross location of each of the detectors may be entered in the system software. For example, detector #1 may be located in southwest entryway #23 at location (x=75, y=190, z=1); detector #2 may be located in southwest entryway #23 at location (x=75, y=192, z=1); and detector #3 may be located in southwest entryway #23 at location (x=75, y=194, z=1).

These coordinates indicate that detectors are spaced about 2 meters apart (based on 2 m increments in y) and about 1 meter above the floor of the passage way #23.

Each sensor panel or detector may comprise an array of cell-based sensors, each of which comprises an array of neurons, with different odorant receptors assigned to different locations on the array. The detector may comprise a certain amount of redundancy such that a given receptor may reside in more than one neuron or more than one position on the array. In some cases, a single receptor may be over-expressed in each neuron. This may permit successful mapping of the neuron activation back to a single odorant receptor, and thus to a single predetermined set of odorants that may be detected by that receptor. Each detector array may be trained for different odorants such that a specific signal pattern across receptors on the array may be associated with each odorant. Some receptors may be more specific for binding of a specific compound, and thus may specifically detect some odorants. Other receptors may be more general or promiscuous in their binding of odorants, and thus may exhibit activation responses to a wider range of odorants. The pattern of electrical signals induced upon binding of specific odorants can be determined for the detector array beforehand.

In some cases, a specific odorant may bind to a set number of receptors at different levels based on concentration. For example, when tested during training, DNT (dinitrotoluene, a chemical precursor of the explosive trinitrotoluene (TNT)) may bind to receptors 7, 9, and 47 on the array, thereby providing a DNT fingerprint on a specific detector array. Because these detectors may be able to detect sub-threshold (sub-action potential threshold) binding, one can map different signals to different concentrations of the volatile compound detected.

In some cases, a single detector array may be able to detect binding events for the odorant(s) of interest. For example, in some cases, an odorant may bind to detector array neurons 7, 9, and 47, thereby allowing one to refer to a lookup table and determine that the odorant may be likely DNT. Locally, with that single detector, one can predict a likelihood that DNT was detected.

In determining the likelihood of having detected a specific compound, one can give a higher score or weighting factor to responses measured for narrowly-focused odorant receptors that are more likely to respond to the specific odorant, while factoring in partial scores or additional weighting factors for responses measured for more promiscuous receptors.

In some cases, e.g., where the detection system comprises multiple detectors connected to a single computing source (such as a server), one may detect the odorant at different locations and at different concentrations over time, thereby tracking the source of the odorant.

For example, if a passenger carrying TNT-based explosives were to enter the passageway at time t=0 seconds, then:

At t=5 seconds, when the passenger may be passing detector #1, a detection event for DNT may occur by observing increased signal for neurons 7, 9 and 47. The server can detect the event.

At t=10 seconds, when the passenger may be passing detector #2, a detection event may occur for detector #2.

At t=15 seconds, when the passenger may be passing detector #3, a detection event may occur for detector #3.

A computer server tracking signal activity at detectors #1, #2 and #3 may be alerted as the detectors respond to the presence of the odorant compound, and the algorithm may trigger an alert that an initial detection event has occurred in vestibule #237 at coordinates (75, 190, 1), after which it may perform a search for detection events for nearby detectors over a period of seconds such that a vector of increased detection events nearby (due to increasing local concentration of the DNT) can be tracked. As soon as a second detection event is identified by a nearby detector, the highest level of alert is triggered since there is little likelihood that a false positive event has occurred.

From the time-stamped data for the detection events, the computer can detect a direction of travel for the passenger carrying the explosive, and security measures may be taken by airport personnel (e.g., more detailed, directed video surveillance, locking of doors, and alerts to personnel directing them to intercept potential passengers).

A. “Smart Tunnel” Configuration:

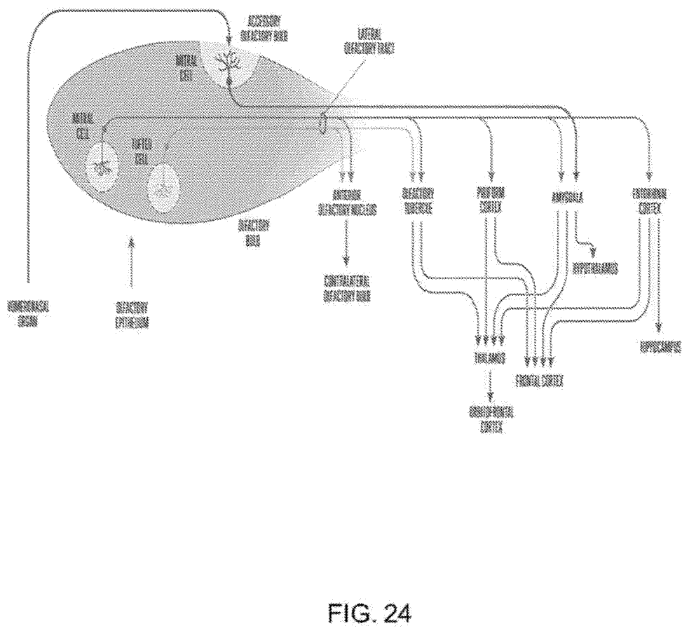

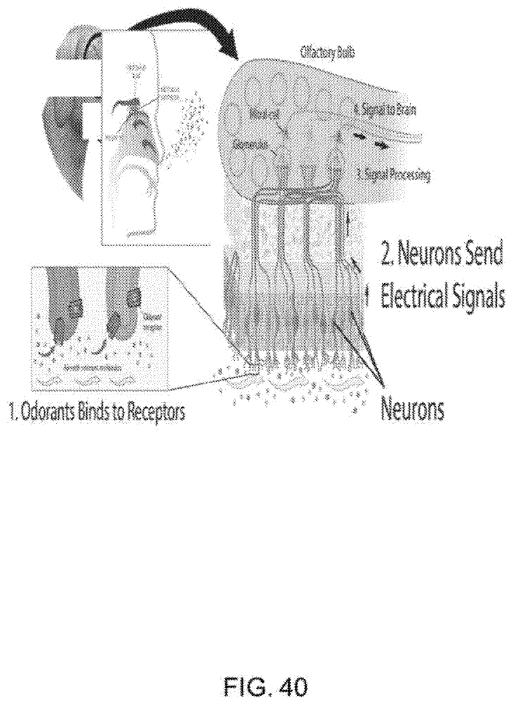

In some embodiments, the detection systems described herein may comprise a “smart tunnel” for high-throughput, high-precision detection of explosives and other volatiles carried by passengers at airport security checkpoints. In some embodiments, one wall of the smart tunnel may be populated with several grids of cell-based sensor devices (i.e., bio-electronic chips) that may be able to detect explosive compounds with extremely high precision. The passengers may proceed down the tunnel past a detection system optimized for delivering volatile compounds emanating from a passenger to the functional detection component of the chip, which may be a genetically engineered neural cell. FIG. 24 shows a non-limiting example of neural system of a human subject for sensing an odor. FIG. 40 shows a non-limiting example of a human's neural system responding to an odor. Airport security personnel may be immediately alerted if a passenger appears to be carrying explosives detected by the cells with the sensor devices. The bioelectronic chips may comprise an array of neurons in contact with or in close proximity to an array of microelectrodes that are capable of capturing the electrical signals generated by the neurons, e.g., action potentials, which constitute a response to a volatile chemical present in the environment. Each neural cell may be engineered to express a single type of odorant receptor that may be specifically responsive to a single kind of ligand. The cell surface receptor, via a series of signaling proteins may internally trigger an action potential by the neuron. This electrical signal from the cell may be measured by the electrode (e.g., as a current or voltage pulse) and then processed by a machine learning back end that determines if the electrical signal pattern generated by the cells constitutes a detection event. In aggregate, the cells may differentially detect an array of compounds or mixtures of compounds, which collectively yield a signal “fingerprint” of detection. In some embodiments, for example, the tunnel may comprise four sensor panels, each with an adaptive sensitivity parameter to ensure robust detection of a range of volatile compounds of interest with a low rate of false positive events. Because this detection system takes advantage of the specificity of receptor-ligand binding interactions and the signal amplification that is inherent in intracellular signaling pathways, it may be able to detect compounds of interest at concentrations down to the parts per billion (ppb) range, with extremely high selectivity, such as concentrations of less than about 500 ppb, less than about 200 ppb, less than about 100 ppb, less than about 50 ppb, less than about 10 ppb, less than about 1 ppb, or less than about 0.1 ppb.

B. Tunnel Design and Four Stage Voting System:

The passenger may proceed down a tunnel that may be, for example, about 1 meter wide past four separate sensor panels, each with one ‘vote’ as to whether or not the passenger may be carrying an explosive. In order for a detection event to be triggered, the detection system may require that all four panels form a positive consensus. Each panel may comprise an m×n grid of cell-based microelectrode array sensors. Each cell-based sensor device within the sensor panel may be engineered to be responsive to one compound of interest, and may comprise at least 128 separate neurons genetically engineered to express a cell surface odorant receptor that can bind to the explosive in question. That is, all or a portion of those neurons may be dedicated to responding to one species of volatile compound. If a significant proportion of these neurons begin firing in response to the volatile compounds or particulates emanating from a specific tunnel occupant, then the system may have detected a compound of interest. The next cell-based microelectrode array sensor in the grid may be comprised of neurons expressing a different set of receptors, which respond to a different compound. In this manner, each of the cell-based sensors in the array of sensor comprising the sensor panel may be designed and/or optimized for detection of a particular compound of interest, and each sensor panel may be able to respond to all compounds of interest.

The sensor panels may be intelligent, and may adapt in response to information from the preceding sensor panel. If the first sensor panel indicates that the passenger is likely to be carrying an explosive (i.e., one of the cell-based microelectrode array sensors has reached a positive consensus about one of the m×n detectable compounds), then the sensitivity of the second sensor panel can be immediately increased to verify this result. Following this second confirmation, the sensitivity can then be increased in the third and fourth subsequent sensor panels. As noted above, the sensitivity of individual cell-based sensor devices, and thus of the sensor panel comprising said devices, may be adjusted in a variety of ways, e.g., by addition of odorant binding proteins or compound stabilization additives in the culture medium bathing the cells. In some embodiments, sensitivity may also be adjusted by changing the threshold for signaling an alert, by altering airflow across the sensor devices of the panel, or by adjusting other environment control systems (e.g., temperature, humidity, electrical stimulation, etc.).

If the first sensor panel doesn't detect a compound of interest, the sensitivity of the second panel may remain unchanged. However, if the second sensor panel makes a positive detection, then the sensitivity of the third sensor panel may be updated to verify the result of the second sensor panel. This procedure may eliminate false positives and may ensure robust and reliable detection of every compound of interest that the tunnel has been designed to respond to.

C. Cell-Based Sensor Devices:

Each single sensor panel within the smart tunnel may comprise a grid of cell-based sensor devices (i.e., cell-based microelectrode array sensors), as previously discussed. Each cell-based microelectrode array sensor comprises a grid of neural cells which have been transfected with exogenous odorant receptors that are known to be responsive to a particular volatile or explosive. Non-limiting examples of volatile markers for and taggants used with explosive materials are listed in Table 5. The odorant receptors are proteins that the cell is constantly generating and trafficking to the cell surface. When the correct compound of interest binds to form a complex with the receptor protein, a bio-amplification cascade is triggered within the cell in which the signal is amplified by several thousand-fold, eventually resulting in the depolarization of the cellular membrane by calcium and potassium ion exchange. This depolarization appears as an electrical signal called an action potential that can be detected by the one or more microelectrodes positioned within each chamber of the cell-based sensor device and translated into a digital signal by an analog to digital converter. From there, machine learning-based back-end signal processing comprising the use of, for example, a support vector machine, will determine if the level of firing is sufficient to constitute a detection event. The neurons are expected to have a low level of background action potential firing even in the absence of any appropriate stimuli. This will be taken as a baseline, and an appropriate level of deviation above this baseline will constitute the detection of the compound of interest. In some cases, the type of neuron or excitable cell used to express the odorant receptors may be selected or modified, e.g., genetically modified, to minimize background action potential firing. By using separate dedicated cell-based microelectrode array sensors for each individual compound of interest, security personnel will immediately be alerted to the fact that a passenger is carrying or has come into contact with an explosive, but will also be informed as to precisely which explosive has been detected. In the event that the passenger is carrying or has been in contact with multiple explosives, it is therefore trivial for the smart tunnel to identify all of them simultaneously.

TABLE 5
non-limiting examples of volatile markers and taggants for explosive materials.

| Compound | Description |
|---|---|
| 2,3-dimethy1-2,3-dinitrobutane | Taggant used in the U.S. for marking plastic |
| (DMDNB/DMNB)) | explosives (detectable by dogs at 0.5 ppb in air) |
| ethylene glycol dinitrate (EGDN) | Taggant used to mark Semtex |
| ortho-mononitrotoluene (o-MNT) | Taggant used for marking plastic explosives |
| para-mononitrotoluene (p-MNT) | Taggant used for marking plastic explosives |
| Dinitrotoluene (DNT) | Chemical precurson of the explosive |
| | trinitrotoluene (TNT) |
| Trinitrotoluene (TNT) | Explosive material |
| Triacetone triperoxide (TATP) | Trimer of acetone peroxide (AP)-explosive material |

D. Air Sampling:

As discussed previously, in many embodiments an air-sampling device may be integrated with the cell-based sensor devices or sensor panels, or may be used in conjunction with said devices and panels to facilitate efficient transfer of volatile compounds from air within the tunnel into the liquid medium bathing the cells within the sensors. In a first option, neural sensor devices such as those shown in FIGS. 6A-B may be mounted on a wall or ceiling of the tunnel, and may comprise a semi-permeable gas exchange membrane that allows diffusive transport of volatile compounds through the membrane to the cell medium.

In a second option, as shown in FIG. 7, the air surrounding the current tunnel occupant may be drawn into a gas perfusion device and bubbled through an exact volume of cellular media, thus trapping the compound of interest. Turbines or fans may collect air samples from the vicinity of the current tunnel occupant as he or she enters the tunnel, and delivers it to the gas perfusion device, where it is bubbled through the liquid medium at a rate of about 2 liters per second. Through the use of a microfluidics-based perfusion system, the medium currently residing in the cell-based sensor device, which corresponds to the air sample drawn for the last tunnel occupant, may be flushed out and replaced with the medium now containing volatile or particulate matter from the air sample drawn for the current tunnel occupant. This air sampling, gas perfusion, and medium exchange process may occur in cycles lasting less than about 20, 15, 10, 9, 8, 7, 6, 5, 4, 3, 2, 1, 0.5, or 0.1 seconds, and the process may be repeated for each sensor panel that the passenger may walk past. This may ensure that volatile compounds of interest are efficiently introduced into the medium and reach the cell surface, as the diffusion path length from source to cell surface through the liquid medium has been identified a potentially confounding factor in previous research. The presently disclosed systems and method may eliminate this problem.

A third option for air sampling may be to perfuse the air surrounding the current tunnel occupant through a solvent rather than cell culture medium (e.g., using a device similar to that illustrated in FIG. 7), where the solvent may be chosen for its ability to dissolve even extremely volatile compounds. For example, this may be a polar aprotic solvent such as dimethyl sulfoxide or acetone, which may be less cytotoxic than other organic solvents, and are known to solvate compounds like TNT well. This compound-containing solution may then be aerosolized via an ultrasonic humidification device which uses high frequency vibration to form a vapor or mist. This vapor can then be blown over the surface of the semipermeable membrane of the cell-based sensor device. In some cases, the medium may not need to be constantly perfused through the sensor device with this approach, but may instead be changed at regular intervals that are determined empirically based on the needs of the cell population. The time course of air sampling, gas perfusion, and medium exchange events may be similar to that described above.

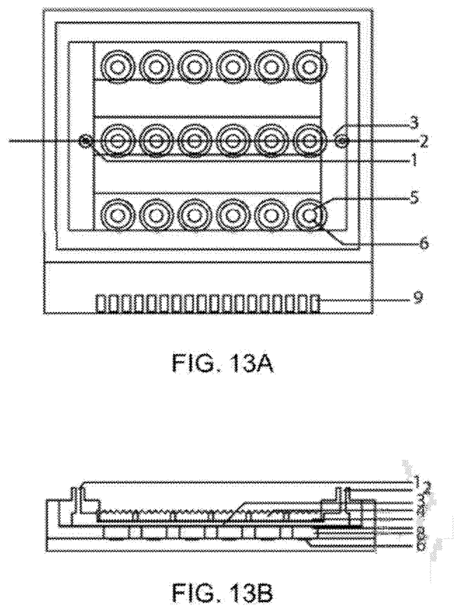

In a fourth option, as shown in the sensor devices illustrated in FIGS. 13A-B, the solvent perfusion/aerosol approach described for option 3 may be modified to pass the compound-loaded vapor over a highly texturized gas exchange membrane which, like the nasal cavity of any smelling organism, can form air currents and eddies which may facilitate entrapment of dissolved compounds or particulates, thereby increasing dwell time and the overall proportion of volatiles that diffuse through the membrane into the medium bathing the cells. This bio-inspired architecture for the semi-permeable gas exchange membrane may increase the proportion of compounds or particulates that end up in the cell medium and may therefore facilitate detection by the cells. In FIGS. 13A-B, medium enters the sensor device via medium inlet 1 and is delivered to the cells within each microwell 5 via microfluidic channels 3, before exiting the device via medium outlet 2. Air samples, or compound-loaded vapor, accesses the semi-permeable gas exchange membrane 7 via openings 4. Each microwell 5 comprises an active electrode region 6 (e.g., comprising one or more electrodes). The sensor device may further comprise an anti-shear stress membrane 8, and a contact for complementary electronics 9.

In another approach to air sampling, a passenger may walk past a large air-sampling device such as that illustrated in FIGS. 5A-B that comprises a series of microchannels containing only cell culture medium and covered by a very thin semi-permeable gas exchange membrane, from which the medium outlet may feed into a cell-based microelectrode array sensor device or sensor panel positioned downstream for detection. The purpose of this air-sampling device is to maximize the surface area over which volatile compounds or particulates emanating from a passenger passing through the tunnel can diffuse into the medium. In some cases, for example, the total amount of cell culture medium needed to fill the air-sampling device may be about 1 ml. The panel may be approximately 20 cm×20 cm×25 microns deep, for example, so that even very low concentrations of the volatile compound have a high probability of diffusion across the gas exchange membrane into the cell culture medium. This medium may then be pumped, e.g., via a microfluidic perfusion system, into the cell-based microelectrode array sensor device or panels positioned downstream. In this approach, the cell-based sensor devices or sensor panels may not require an integrated gas exchange membrane as the compounds or particulates of interest may already be dissolved by the medium.

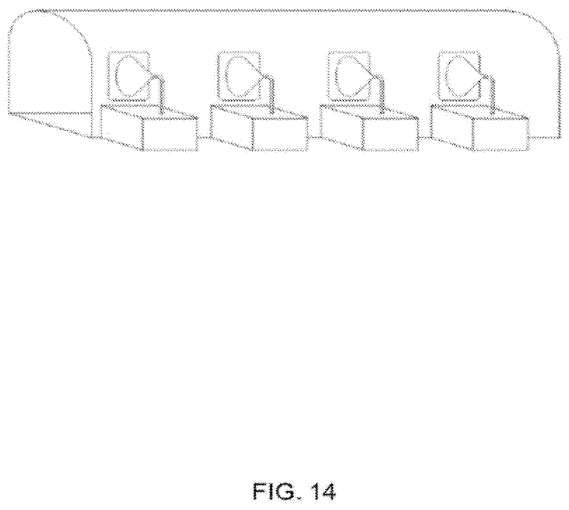

FIG. 14 provides a non-limiting illustration of one embodiment the entire smart tunnel, including a four-stage detection system that incorporates the neural cell-based sensor devices.

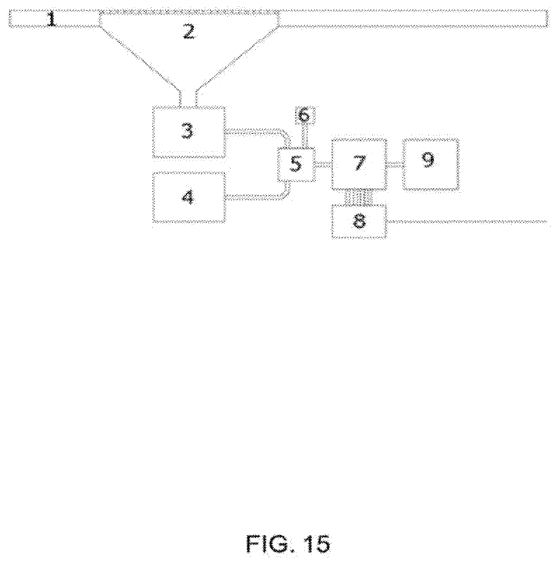

FIG. 15 shows a top view of one stage of the four-stage detection system illustrated in FIG. 14. Air intake 2 is mounted on one wall is 1 of the tunnel. An air pump 3 delivers air samples drawn from the tunnel to a liquid/gas exchange apparatus 5. A cell culture medium reservoir 4 is also connected to the liquid/gas exchange apparatus 5. Air passing through the liquid/gas exchange apparatus is vented through air exhaust is 6, while the compound-containing medium is delivered to the bioelectric sensor panel 7. A computer and/or connections to other system units are interfaced with the detection system through connector 8. The waste medium is collected in reservoir 9. In this system configuration, ambient air containing volatile compounds of interest can be injected into a small mixing chamber where, for example, a device atomizes the gas with the medium. The resultant vapor may then be recondensed and injected into the medium reservoir of an impermeable (sealed) cell-based sensor panel. Top and side views of one such neural sensor panel, comprising an m×n grid (e.g., a 3×6 grid) of cell-based microelectrode array sensors, each of which further comprise genetically-engineered neurons in contact with a microelectrode array that are responsive to a range of explosive or volatile compounds, are shown in FIGS. 4A-B.

### III. Example 3—Encoding a Reference Odor

The method can be used for encoding olfactory stimuli (e.g., reference odors) to create a library of reference odors with reference signals. A reference odor can be exposed to the device described herein. The device can have an artificial array comprising one or more chambers (e.g., 6, 12, 24, 48, 96, 384, or 1536 sample wells). In some cases, the artificial array can have 50,000 chambers. Each chamber can comprise a human neuron expressing an odorant receptor. Some of the human neurons can also express multiple odorant receptors. When an olfactory stimulus binds an odorant receptor, the neuron can produce an electrical or optical signal in response to the binding event.

The electrical or optical signal can be detected by a detector. For example, the optical signal can be detected by a microscope. The detector can detect and record the signal intensity in each chamber, which represents the signal intensity at each of the odorant receptors. The intensity can be proportional to the amount of the olfactory stimulus.

The olfactory stimulus and its reference signal can be encoded to create a reference signal and entered into the library of reference odors. The reference signal can comprise the readout at each odorant receptor. For example, on an artificial array can comprise three odorant receptors: MOR106-1 (OR1), MOR9-1 (OR2), and MOR18-1 (OR3). The artificial array can comprise at least one odorant receptor such as at least 1, 2, 3, 4, 5, 6, 7, 8, 9, 10, 20, 30, 40, 50, 60, 70, 80, 90, 100, 200, 300, 400, 500, 600, 700, 800, 900, 1,000 (1k), 5k, 10k, 20k, 30k, 40k, or 50k odorant receptors.

In one preferred case, the reference signal is from a pure olfactory stimulus, reference odor A, which only produces an electrical or optical signal in one of the odorant receptors, such as OR1=10, OR2=0, and OR3=0. In another preferred case, another pure olfactory stimulus, reference odor B, produces an electrical or optical signal of OR1=0, OR2=20, and OR3=0. In yet another preferred case, another pure olfactory stimulus, reference odor C, produces an electrical or optical signal of OR1=0, OR2=0, and OR3=25. In some cases, olfactory stimulus, reference odor D, which produces an electrical or optical signal in more than one of the odorant receptors, such as, OR1=10, OR2=20, and OR3=0, can also be used as reference signals. This step can be repeated with a library of reference odors to build a database of reference odors with their respective reference signals. These reference signals can be further used to replicate or decode odor from unknown compounds.

### IV. Example 4—Replicating an Odor

The database of reference odors with their respective reference signals can be used to replicate an odor from any compound. For example, the odor from an unknown compound, test compound X, is tested on the artificial array described in the previous example and produces an electrical or optical signal of OR1=10, OR2=20, and OR3=25.

In one case, the odor from test compound X can be replicated by mixing the reference odor A (OR1=10, OR2=0, and OR3=0), B (OR1=0, OR2=20, and OR3=0), and C (OR1=0, OR2=0, and OR3=25).

In another case, the odor from test compound X can be replicated by mixing the reference odor C (OR1=0, OR2=0, and OR3=25) and D (OR1=10, OR2=20, and OR3=0).

Because the signal intensity is proportional to the amount of the odor, the amount of the reference odors can be modulated to any odor from a known or unknown source (e.g., compound) by using a comprehensive database of reference odors.

### V. Example 5—Decoding an Odor

This method can also be used to make a prediction of the components of an unknown source (e.g., compound) by using a comprehensive database of reference odors. For example, by using the same method in the previous example, the method can decode the odor of the test compound X and predict that test compound X is a mixture of A, B, and C. The method can also predict an alternative composition of mixture of C and D. Because the signal intensity is proportional to the amount of the odor, the method can also predict amount of each component.

### VI. Example 6—Stratifying an Odor into a Reference Emotional State

In addition to encoding the reference odor, a smelling assay can be performed on a subject to create a database of reference odors and its corresponding emotional state. For example, a subject is given the reference odor C (OR1=0, OR2=0, and OR3=25) and is asked to rate his/her happiness in response to the smell of the reference odor C on a scale of 1-10. Multiple subjects can be tested with the reference odor C and the average happiness is 5. The same can be done for reference odor D (OR1=10, OR2=20, and OR3=0) and the average happiness is 9.

A database of reference odors and its corresponding emotional state (e.g., happiness in this case) can be built using this method. Additional attributes can be included in the database. For example, a sub-group of subjects in U.S. may rate the reference odor D to have an average happiness of 9.5, while another sub-group of subjects in Europe may rate the reference odor D to have an average happiness of 8.5. Therefore, based on the additional attribute (e.g., geolocation, nationality, gender, age, and so on), the reference odors and its corresponding emotional state for specific groups of subjects can be obtained and stored in the database.

### VII. Example 7—Assessing an Emotional State of a Subject in Response to an Odor

In this example, if the odor of an unknown source, test compound Y, produces an electrical or optical signal of OR1=10, OR2=20, and OR3=0, which is the same as the reference odor D, the method can predict that the test compound Y will produce an average happiness of 9 in the general population. As discussed above, the method can also predict that the test compound Y will produce an average happiness of 9.5 in U.S. subjects and 8.5 in European subjects.

Euler's trapezoidal method is used to calculate and compare the response(s) of each cell to odorant (see, e.g., Wolever et al. (1991)Am. J Clin. Nutr. 54:846). Specifically, area Aₜₒₜₐₗ under the total response curve between to and t=, defined for a total of 30 seconds prior and post to the response maxima, is calculated by summing the net response rₙ and rₙ₊₁ multiplied by the duration of the response. The responses are added in single second increments, so that the trapezoid method sums the response underneath the curve between any two consecutive seconds (i.e. between t₁ and t₂, the area A₁ would equal (r₁+r₂)*t, where t=1. To normalize Aₜₒₜₐₗ against the background of each cell, a baseline curve is drawn using the average baseline response r_{base} of the first four of the 20 rₙ values, which are the net response values before cell stimulation. r_{base} is next multiplied by 20 seconds, which is the total time interval monitored for each cell to obtain A_{base}. This baseline area A_{base} is subtracted from the net response of each cell calculated using the trapezoid method, providing an Aₙₑₜ value that defines only the area underneath the response curve. Data analysis is conducted using, e.g., Image J, Igor Pro, and Microsoft Excel.

Calcium imaging with HEK293T cells is performed as described (see, e.g., Ishimaru et al. (2006) Proc. Natl. Acad. Sci. USA 103:12569.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The phrase “at least one” includes “one or more” and “one or a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

We claim:

1. A method for encoding an olfactory stimulus, comprising:
a) contacting the olfactory stimulus with an artificial array comprising one or more chambers, wherein the one or more chambers comprise one or more cells expressing one or more cell-surface receptors, wherein the one or more cell-surface receptors generate one or more signals in response to a binding event between the olfactory stimulus and the one or more cell-surface receptors;
b) recording an intensity of one or more signals of one of the cells; and c) encoding the olfactory stimulus by creating a reference signal, wherein the reference signal comprises the intensity of the one or more signals.

2. The method of claim 1, wherein the one or more cells are neurons.

3. The method of claim 2, wherein the neurons are human neurons.

4. The method of claim 1, wherein the one or more cells are modified to express the one or more cell-surface receptors.

5. The method of claim 4, wherein the one or more cells are genetically modified to express the one or more cell-surface receptors.

6. The method of claim 1, wherein at least one of the one or more cell-surface receptors is an odorant receptor.

7. The method of claim 6, wherein at least one of the one or more cells expresses one odorant receptor.

8. The method of claim 6, wherein at least one of the one or more cells expresses a plurality of odorant receptors.

9. The method of claim 6, wherein the odorant receptors comprise OR1A1, MOR106-1, OR51E1, OR10J5, OR51E2, MOR9-1, MOR18-1, MOR272-1, MOR31-1, MOR136-1; any fragment thereof, or any combination thereof.

10. The method of claim 1, wherein the one or more signals are electrical signals, optical signals, or a combination thereof.

11. The method of claim 10, wherein the one or more signals are electrical signals comprising an action potential.

12. The method of claim 10, wherein the one or more signals are electrical signals comprising an excited signal that is below a threshold for an action potential.

13. The method of claim 10, wherein the one or more signals are electrical signals comprising a cell membrane depolarization.

14. The method of claim 1, wherein the intensity of one or more signals is detected by a detector.

15. The method of claim 1, wherein the intensity of one or more signals is proportional of the amount of the olfactory stimulus.

16. The method of claim 1, further comprising applying one or more attributes of the olfactory stimulus and the reference signal to a machine learning algorithm.

17. The method of claim 16, wherein the machine learning algorithm comprises Support Vector Machine (SVM), Naive Bayes (NB), Quadratic Discriminant Analysis (QDA), K-Nearest Neighbors (KNN), Linear Discriminant Analysis (LDA), Multilayer Perceptron (MLP), or any combination thereof.

18. A method for replicating an olfactory stimulus, comprising:
a) contacting the olfactory stimulus with an artificial array comprising one or more chambers, wherein the one or more chambers comprise one or more cells expressing one or more cell-surface receptors, wherein the one or more cell-surface receptors generate one or more signals in response to a binding event between the olfactory stimulus and the one or more cell-surface receptors;
b) recording an intensity of one or more signals of one of the cells;
c) encoding the olfactory stimulus by creating a target signal, wherein the target signal comprises the intensity of the one or more signals; and
d) replicating the target signal of the olfactory stimulus by mixing two or more reference olfactory stimuli, each of which has a reference signal, wherein the reference signals of the two or more reference olfactory stimuli have a combined signal that is similar to the target signal.

19. The method of claim 18, wherein the one or more cells are neurons.

20. The method of claim 19, wherein the neurons are human neurons.

21-143. (canceled)
